(19) United States
(12) Patent Application Publication
(10) Pub. No.: US 2006/0218010 A1
(43) Pub. Date: Sep. 28, 2006

(54) Systems and methods for obtaining, storing, processing and utilizing immunologic information of individuals and populations

(75) Inventors: Francis Michon, Bethesda, MD (US); Samuel L. Moore, Sykesville, MD (US); Peter C. Fusco, Silver Spring, MD (US); Samuel J. Wohlstadter, Madison, VA (US); Charles Quentin Davis, Frederick, MD (US); David Coleman, Gaithersburg, MD (US); Suraj Shetty, Arlington, VA (US); Kamini Joshi, Ashburn, VA (US)

Correspondence Address: KRAMER LEVIN NAFTALIS & FRANKEL LLP;INTELLECTUAL PROPERTY DEPARTMENT, 1177 AVENUE OF THE AMERICAS, NEW YORK, NY, 10036, US

(73) Assignee: BioVeris Corporation, Gaithersburg, MD (US)

(21) Appl. No.: 11/255,161

(22) Filed: Oct. 18, 2005

Related U.S. Application Data
(60) Provisional application No. 60/620,038, filed on Oct. 18, 2004.
(60) Provisional application No. 60/620,037, filed on Oct. 19, 2004.
(60) Provisional application No. 60/623,187, filed on Oct. 29, 2004.

(51) Int. Cl. G06F 19/00; G01N 33/48

(52) U.S. Cl. 705/3; 702/19

(57) ABSTRACT

A system and method for obtaining, storing, processing and utilizing immunologic information of individuals and populations is presented. In exemplary embodiments of the present invention, a biological sample can be taken from one or more individuals and the sample submitted to one or more panels of assays. The results of the assays can be stored and analyzed, and such analysis can include (i) calculating derived quantities which take the results of the assays as inputs, and (ii) submitting the results and the derived quantities to a set of rules, each of which has a defined output state. In exemplary embodiments of the present invention, based upon the output state of the rules, an appropriate recommendation as to one or more immunization or other interventions can be generated and incorporated with provider and patient reminders. In exemplary embodiments of the present invention the results of the assays and the recommendation, as well as additional information specific to the individual can be stored for further analysis. In exemplary embodiments of the present invention the assay panel or panels can be chosen as a function of a defined demographic group or enterprise affinity into which the individual corresponds. In exemplary embodiments a database can be maintained for storing and further processing of all immunologic informatics collected according to the methods of the present invention, and can be further processed or used to optimize the delivery of products and/or services in various commercial, research and governmental contexts.

### CROSS-REFERENCE TO RELATED APPLICATIONS

This application claims the benefit of the following three United States Provisional patent applications (the “Provisional Applications”), each of which is hereby incorporated herein by this reference: U.S. Ser. No. 60/620,038, filed on Oct. 18, 2004, U.S. Ser. No. 60/620,037, filed on Oct. 19, 2004, and U.S. Ser. No. 60/623,187, filed on Oct. 29, 2004.

### TECHNICAL FIELD

The present invention relates to immunological informatics, and more particularly to systems and methods for acquiring, storing and utilizing immunologic information of individuals and populations in various commercial, research and governmental contexts.

### BACKGROUND OF THE INVENTION

Personalized medicine is the wave of the future. A personalized medicine approach seeks to identify whether a given individual needs a given treatment or intervention prior to administering it, rather than relying on “standards” representing an average person in a group or population. This approach is based on the well known fact that some individuals in a demographic population have naturally low or naturally high values which are not best measured against a statistical mean for the demographic population, but against that individual's own measured history.

For example, vaccines are a immunologic prophylactic whose frequency and dose is determined at the population level. Vaccines are approved for routine human use by regulatory agencies from different countries where the vaccines are to be applied. For example, in the U.S. the Food and Drug Administration (FDA) is responsible. After approval, population-wide recommendations for use are made by various medical agencies, such as the Advisory Committee on Immunization Practices (ACIP), whose members represent experts in the vaccine field. The ACIP is a U.S. committee, selected by the Secretary of Health and Human Services, to assist and advise the Secretary, as well as the Centers for Disease Control and Prevention (CDC), on how to best implement vaccination strategies to prevent disease. Written recommendations are developed with immunization schedules that are published and updated as needed for both pediatric and adult populations. From these recommendations, certain vaccines are mandated for school entry and government-sponsored programs.

Although such mandated schedules are the norm, the need for them varies across populations. Immunity to disease wanes over time, but may be maintained at low levels or recalled, through immunologic memory, upon subsequent exposure to the corresponding infectious agent or cross-reactive antigens in the environment. For inactivated or subunit-based, T cell-dependent vaccines, however, protective immunity may not last beyond 10 years.

For example, the protective responses to diphtheria, tetanus, and pertussis vaccines (DTaP, Td) have been shown to be absent after about 10 years, which is why Td (tetanus and diphtheria) boosters are recommended every 10 years. For T cell-independent vaccines, such as pneumococcal and meningococcal polysaccharides, there is no immunologic memory, and immunity may be gone in only 3 to 5 years. The result of these facts is that vaccinating only according to a standard protocol can often result in either under-vaccinating or over-vaccinating.

Result of Over-Vaccinating: Type III Hypersensitivity Reactions

“Certain vaccines produce increased rates of local or systemic reactions in certain recipients when administered too frequently . . . Such reactions are thought to result from the formation of antigen-antibody complexes.”
P.4 In Centers for Disease Control and Prevention. General recommendations on immunization: recommendations of the Advisory Committee on Immunization Practices and the American Academy of Family Physicians. MMWR 2002; 51 (No. RR-2):1-36

Thus, while generalizations about the timing for boosters, whether at 3 or 5 or 10 years, represent one approach to the problem of maintaining long-term immunity, other problems may arise if the duration of immunity does not follow the expected pattern. For example, it is well known that Td boosters for adults (e.g., given in emergency rooms to prevent tetanus after someone steps on a rusty nail), often lead to local adverse reactions at the injection site, particularly if the last booster was not too many years earlier. It may be difficult to determine when the last immunization was received for tetanus, so when in doubt, one is inclined to err on the side of caution by boosting.

While this general booster approach may readily prevent tetanus, the possibility of high levels of circulating antibodies may lead to an Arthus reaction, which is a local type III hypersensitivity reaction due to the development of immune complexes composed of IgG antibodies and the vaccine antigen. The immune complexes activate complement which binds to complement receptors on the mast cells to cause the release of granules and increased vascular permeability. This can ultimately lead to tissue damage. In an extreme case, a more generalized or systemic reaction might occur, where immune complexes are deposited in the kidneys and joints, leading to arthritis and glomerulonephritis. Subsequent cellular immune responses and tissue damage with respect to the glomerulus can lead to permanent loss of kidney function.

A solution for the prevention of such a type III hypersensitivity problem resulting from over vaccination, would be to assess a person's immune status with respect to the offending antigen, and make an existential determination of when to optimally administer the vaccine booster. For example, concerning vaccinations for internationally adopted children of unknown immune status, the CDC states: “If avoiding unnecessary injections is desired, judicious use of serologic testing might be helpful in determining which immunizations are needed.” Regarding DTaP vaccinations specifically, the CDC also states: “If a revaccination approach is adopted and a severe local reaction occurs, serologic testing for specific IgG antibody to tetanus and diphtheria toxins can be measured before administering additional doses.” (see pages 20 and 21 of the CDC 2002 reference cited at the beginning of this section.) In this way, serologic testing could be used to determine whether an antibody level is low enough to warrant further boosting of the immune system for a specific antigen, minimizing the risk of adverse reactions from over vaccinations.

Result of Under-Vaccinating: Increased Susceptibility to Infection

Certain individuals may be genetically predisposed to infections as a result of a compromised immune system. For example, there are people that have been identified to be at greater risk of meningococcal disease due to late-stage complement deficiency, since complement usually mediates antibody-dependent killing of meningococci. Others have been shown to be susceptible to a variety of diseases (e.g., leprosy, salmonellosis, Pseudomonas aeruginosa infections, Yersinia infections, Listeria monocytogenes infections, streptococcal diseases, tuberculosis, Lyme disease, Chlamydia trachomatis infections, Helicobacter pylori infections, HIV disease, and various other viral infections) that appear to be correlated to a different HLA haplotypes. Still others have been shown to have increased susceptibility to certain diseases (e.g., Haemophilus influenzae type b meningitis in Eskimos, Apaches, and Navajos) because their immune systems respond with a less effective antibody repertoire based on variable-region gene haplotypes. A solution to this susceptibility problem would be to screen people for the appropriate biologic or genetic markers and vaccinate accordingly. Vaccinations would help to enhance the compromised immune systems with higher levels of specific antibodies that could enable other immune mechanisms (e.g., opsonophagocytosis instead of complement-mediated lysis), overcome low antibody avidity with greater antibody numbers, or alter the relative balance of antibody repertoires. In addition, continuous serologic testing (e.g., annually) of the immune status would allow for optimum timing of vaccinations to counter the relentless waning of immunity over time, and still avoid the potential problems of over immunizing.

Determination of the immune status of individuals to vaccine-preventable diseases requires an assay system that can detect antibodies that may be present at very low levels, especially when natural or vaccine exposure may have been many years previously. In addition, such an assay system could be used to assess an individual's immune competence at different stages of that individual's life, and also measure the vaccine status of individuals with varying special needs and requirements (e.g. military personnel or travelers).

What is thus needed in the art is a system and method for measuring and processing immunologic information of individuals and populations at various times in their lives so as to better track each individual's immune status and make appropriate diagnostic, prophylactic and therapeutic recommendations.

What is further needed in the art is a supporting structure to conveniently store the results of such screenings for easy access and processing, for data mining purposes as well as for use in a variety of commercial, research and governmental applications where a knowledge of the immunological indicia of customers, subjects and citizens can create efficiencies and optimizations, as well as allow for the exploitation of commercial opportunities and improve the quality of life.

### SUMMARY OF THE INVENTION

A system and method for obtaining, storing, processing and utilizing immunologic information of individuals and populations is presented. In exemplary embodiments of the present invention, a biological sample can be taken from one or more individuals and the sample submitted to one or more panels of assays. The results of the assays can be stored and analyzed, and such analysis can include (i) calculating derived quantities which take the results of the assays as inputs, and (ii) submitting the results and the derived quantities to a set of rules, each of which has a defined output state. In exemplary embodiments of the present invention, based upon the output state of the rules, an appropriate recommendation as to one or more immunization or other interventions can be generated and incorporated with provider and patient reminders. In exemplary embodiments of the present invention the results of the assays and the recommendation, as well as additional information specific to the individual can be stored for further analysis. In exemplary embodiments of the present invention the assay panel or panels can be chosen as a function of a defined demographic group or enterprise affinity into which the individual corresponds. In exemplary embodiments a database can be maintained for storing and further processing of all immunologic informatics collected according to the methods of the present invention, and can be further processed or used to optimize the delivery of products and/or services in various commercial, research and governmental contexts.

### BRIEF DESCRIPTION OF THE DRAWINGS

FIG. 1 depicts generalized exemplary process flow according to exemplary embodiments of the present invention;

FIG. 2 depicts a system overview according to exemplary embodiments of the present invention;

FIG. 2A depicts an alternative system overview according to exemplary embodiments of the present invention;

FIGS. 3A and 3B depict various exemplary configurations for assaying a patient sample according to an exemplary embodiment of the present invention.

FIG. 4 is a graph depicting the incidence of Hepatitis B in the United States from 1978 through 2002;

FIG. 4A is a graph of age-related immunity to tetanus;

FIG. 4B depicts the rate of pneumococcal disease by age group;

FIG. 4C depicts U.S. Army hospital admissions during various wars;

FIG. 4D depicts anthrax cases in the United States 1951-2002;

FIG. 4E depicts B-cell memory to smallpox vaccination over time;

FIG. 4F depicts risk for Lyme disease as a function of geographic area in the Unites States;

FIG. 4G depicts recommended adult immunizations;

FIG. 4H depicts recommended adult immunizations by medical condition;

FIG. 4I illustrates certain interactions between polarized Th1 and Th2 responses;

FIG. 4J depicts a model used to illustrate the complex balance between Th1 and Th2 cells as dictated by the Th1-Th2 paradigm;

FIG. 4K depicts the role of cytokines in the induction and function of regulatory T cells;

FIG. 4L depict exemplary interactions involving cytokine secretion and regulatory functions and differentiation among different cell types;

FIG. 5 depicts a detailed system diagram according to an exemplary embodiment of the present invention;

FIG. 6 depicts example assay results in an exemplary database according to the present invention;

FIG. 7 depicts exemplary diagnostic module recommendation types according to an exemplary embodiment of the present invention;

FIG. 8 illustrates an example perceptron network which implements a rule for a normal individual using as inputs the results of an exemplary menigicoccal diagnostic panel;

FIG. 8A illustrates the example perceptron network of FIG. 8 implementing a similar rule for an abnormal individual;

FIG. 9 depicts an XML representation of the example perceptron networks of FIGS. 8 and 8A;

FIG. 10 depicts an exemplary symbology for diagnostic goals which can be used to articulate diagnostic goals in an exemplary embodiment of the present invention;

FIG. 11 illustrates exemplary diagnostic goals using the symbology of FIG. 10;

FIG. 12 illustrates an example database schema, patient information according to an exemplary embodiment of the present invention;

FIG. 13 illustrates an example database schema, visit information according to an exemplary embodiment of the present invention;

FIG. 14 illustrates an example database schema, test results according to an exemplary embodiment of the present invention;

FIG. 15 depicts the example patient age intervals used in an exemplary database described in Section II according to an exemplary embodiment of the present invention;

FIG. 16 is a plot illustrating female antibody comparison over a number of years according to an exemplary embodiment of the present invention.

FIG. 17 is a plot of a comparison of two individual females, one vaccinated and one not vaccinated according to an exemplary embodiment of the present invention;

FIG. 18 is a plot of antibody levels in a compliment-deficient individual according to an exemplary embodiment of the present invention;

FIG. 19 is a plot of antibody levels in a healthy individual according to an exemplary embodiment of the present invention;

FIG. 20 is an example SQL query according to an exemplary embodiment of the present invention; and

FIG. 21 is a table illustrating the correlation among antibody levels in an exemplary female population according to an exemplary embodiment of the present invention;

FIG. 22 is a process flow diagram for use in a healthcare management embodiment according to the present invention;

FIG. 23 is a subset of the process flow depicted in FIG. 22;

FIG. 24 is an alternative process flow chart for healthcare management according to the exemplary embodiment of the present invention;

FIG. 24A is a more detailed process flow chart similar to that of FIG. 22;

FIG. 25 is an alternative process flow chart for managing healthcare according the exemplary embodiment of the present invention;

FIG. 25A is the process flow chart of FIG. 25 with an additional optional element;

FIG. 26 is an alternative process flow chart for managing healthcare according to the exemplary embodiment of the present invention;

FIG. 26A is an alternative version of the process flow of FIG. 26 with greater detail;

FIG. 27 is a process flow chart for cervical cancer prevention according to the exemplary embodiment of the present invention;

FIG. 28 is a process flow chart for managing the care of women of childbearing age according to the exemplary embodiment of the present invention;

FIG. 29 is a process flow chart for an exemplary “vaccino mat” application according to an exemplary embodiment of the present invention;

FIG. 29A is a system diagram of entities involved in the vaccine distribution application according to an exemplary embodiment of the present invention;

FIG. 29B illustrates the necessary connectivity for the vaccine distribution application illustrated in FIG. 29A;

FIG. 29C is the connectivity displayed in that FIG. 29B recast by use of an interapplication connectivity provider according to an exemplary embodiment of the present invention;

FIG. 30 is an exemplary flow chart for use in a life insurance optimization application according to an exemplary embodiment of the present invention;

FIG. 31 is an exemplary process flow chart for use in Immunosenescence managment application according to an exemplary embodiment of the present invention;

FIG. 32 is an exemplary process flow chart for a disaster management application according to an exemplary embodiment of the present invention;

FIG. 33 is an alternative process flow chart for the psychological aspects of disaster response for a disaster response application according to an exemplary embodiment of the present invention; and

FIG. 34 depicts exemplary process flow in an immunogenicity discovery application according to an exemplary embodiment of the present invention.

### TABLE OF CONTENTS

General Overview

SECTION I EXEMPLARY ASSAY PANELS
   A. COLLEGE STUDENT DIAGNOSTIC PANELS
      1. Meningococcal Diagnostic Panel
      2. Sexually Transmitted Diseases Assay Panel
      3. Persistent Immunity Induced by Childhood Vaccines
   B. ADULT DIAGNOSTIC PANELS
      1. Measurement of Immunity Induced By Vaccines for Military Personnel
      2. ImmunoScore Measurement of Immunity Induced By Vaccines for Travelers
      3. ImmunoScore Measurement of Immune Status in Adults
      4. ImmunoScore Measurement of Immunity in Health Care Workers
      5. ImmunoScore Analyses and Bioterrorism
      6. ImmunoScore Analyses for Infection and Chronic Disease
      7. Th1-Th2 Paradigm
         7.1 Th1-Th2 Based Diagnostic Panel
         7.2 Infectious and Neoplastic Diseases
         7.3 Th1-Th2 and Immunologic Diseases (Allergy/Atopy and Autoimmunity/Inflammatory Disease)
         7.4 Autoimmunity/Inflammatory Disease
      8. ImmunoScore Analyses for Immigrants and Internationally Adopted Children
   C. IMMUNOSCORE EXEMPLARY SUPERPANELS
      1. Middle School Student ImmunoPrint Super Diagnostic Panel
         1.1 Persistent Immunity Induced by Childhood Vaccines
         1.2 Sexually Transmitted Disease (STD) Diagnostic Panel
      2. Women of Child-Bearing Years ImmunoScore Super Diagnostic Panel
         2.1 Recommended tests for ImmunoScore Measurement of Immunity
         2.2 Persistent Immunity Induced by Childhood Vaccines Diagnostic Panel
         2.3 Sexually Transmitted Disease (STD) Diagnostic Panel
SECTION II IMMUNOSCORE SYSTEM DATABASE
   A. General Overview
   B. Example Illustrative Database
      1. Overall Description
      2. Impact of Data Mining
      3. Diagnostic Module
         3.1 Overview
         3.2 Perceptron algorithms
         3.3 Alternate algorithmic approaches
            3.3.1 Additional input data
            3.3.2 Decision rule algorithms
      4. Data Mining Module
         4.1 Overview
         4.2 Sample Data
         4.3. Exemplary Use of the Data Mining Module
SECTION III USES OF IMMUNOSCORE INFORMATION IN VARIOUS COMMERCIAL, RESEARCH AND GOVERNMENTAL CONTEXTS
   A. Health Insurance Underwriting/Health Care Provision Optimization
   B. Veterans Health Care Management (Variant of Health Care)
   C. Socialized Medicine Management
   D. Supplemental Insurance (AFLAC Model)
   E. ImmunoScore and the Wellness Industry
   F. Women of Childbearing Age/Screening of Pregnant Women
   G. Vaccine-o-Mat/Vaccine Distribution Network
   H. Consumer Accessibility to Immunologic Information
   I. Immunoscore Connectivity Via Interapplication Translator/Data Integrator
   J. Immunologic Informatics Based Life Insurance Underwriting
   K. Diagnosing and Managing Immunosenescence in the Elderly
   L. Frozen Storage of Naive Immune Cells (IRP Considerations)
   M. Vaccine Use Outcome/Design
   N. Research Services
   O. Immigration Consulting
   P. Disaster Survivors: Immunizations, Recovery, Prognosis and Treatment
   Q. Monitor Adoptive Immunotherapy/Transplants
   R. Elective Surgery
   S. Services to Charitable Foundations Promoting Immunological Well Being
   T. Discovery of Unwanted Immunogenicity of Therapeutics

### DETAILED DESCRIPTION OF THE INVENTION

General Overview

In what follows, systems and methods of the present invention will be often referred to as the “ImmunoScore” system, method and/or database, as the case may be. “ImmunoScore” is a trademark and/or service mark currently envisioned by the assignee hereof to be utilized in connection with exemplary embodiments of the present invention.

The present invention is directed to the collection, processing, and use of immunologic information. Immunologic information is to be understood in a broad sense, including any information which may be useful as an indicator of any immunological function of a mammalian body. More specifically, the present invention includes acquiring information that is indicative of the immune status of an individual, processing that information, storing the raw information as well as the outputs from the processing stage, and of that information at various times and in various ways to recommend various actions such as prophylactic or further diagnostic interventions, or abstention from action, for individual or population. The present invention exploits a number of advances in technology as well as advances in how people think about medical treatment. In exemplary embodiments of the present invention, a number of immunological or immunological related (in a broad sense) assays can be administered to an individual. Using modern technology such as, for example, the M1M Analyzer marketed by BioVeris™ Corporation of Gaithersburg, Md., one can run a large number of assays, such as, for example 20, 40 or 60, and obtain results therefrom in a relatively short period of time. Moreover, these assay results can be stored in a memory, either locally or at one or more central servers or in associated databases, and can be operated upon by various algorithms or rules which can generate information as to that individual's immune status as well as recommendations for further augmenting that immune status or taking further action in response to the information acquired, from the assays and their processing. This information can be used in a variety of commercial, research, and healthcare contexts. Thus a variety of business methods or opportunities can be created or facilitated using the information obtained according to the methods of the present invention.

The present invention will be described in three distinct sections. The first section describes the scientific background and motivation for creating various assay panels to be administered, singularly or in combination with other assay panels, to different individuals in different populations at different times in each individual's life cycle. This discussion culminates in suggested or exemplary assay super panels which can be administered in various contexts to various individuals.

The next section describes how information obtained from results of the administered assays can be stored, processed, and utilized. This discussion comprises, inter alia, a description of an exemplary database in which (i) results from numerous assays can be stored along with (ii) individual-specific information and (iii) the outputs of various algorithms which operate upon the assay results of that individual.

In a final section, a variety of business and commercial methods are described in which information from the assay panels as stored in the database and further processed can be used to increase business efficiencies, create new markets and opportunities, and/or provide useful tools for research and development.

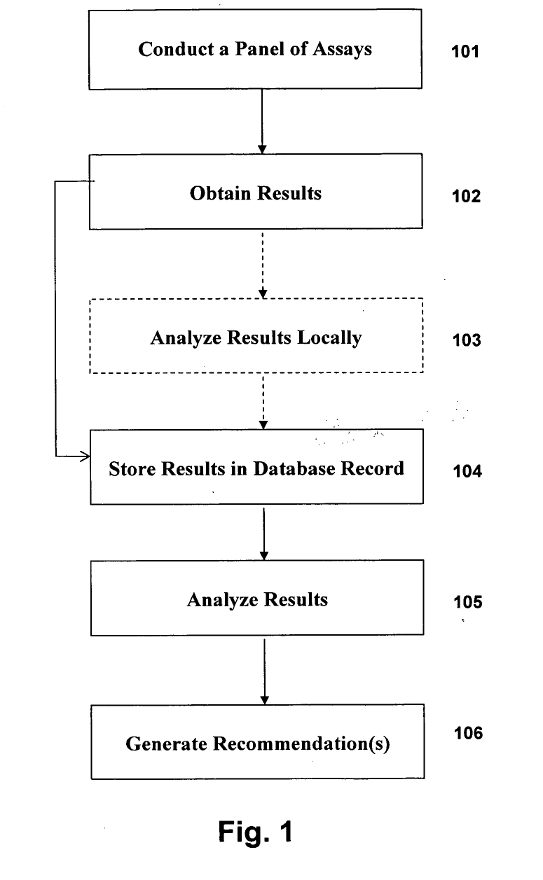

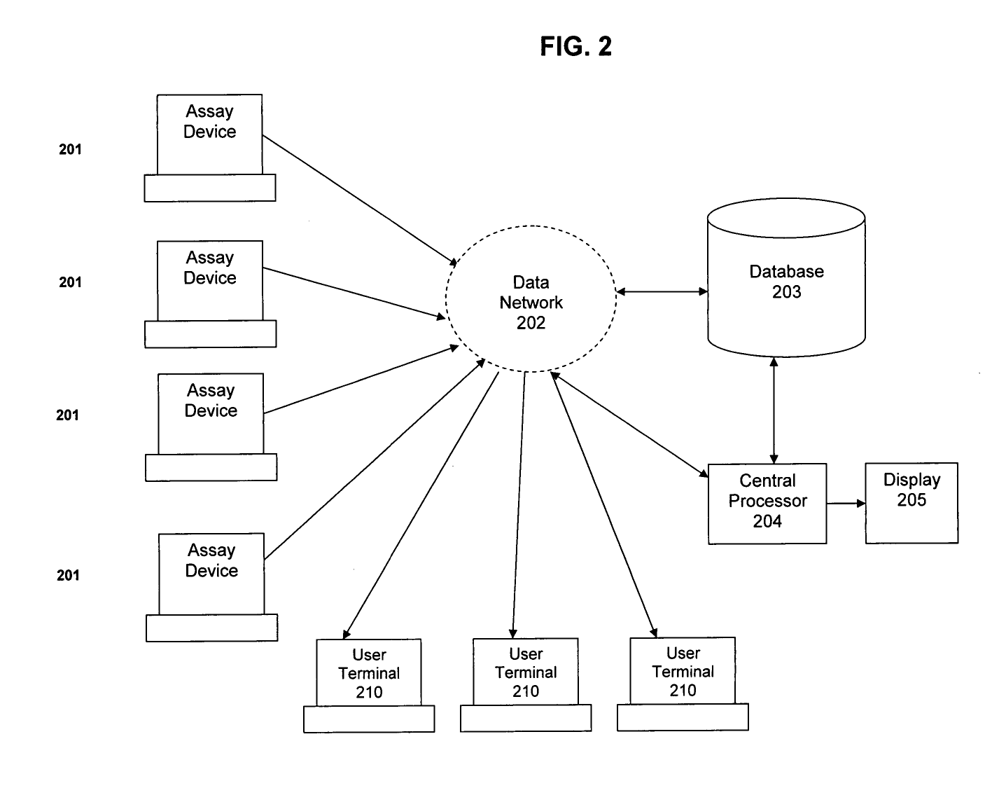

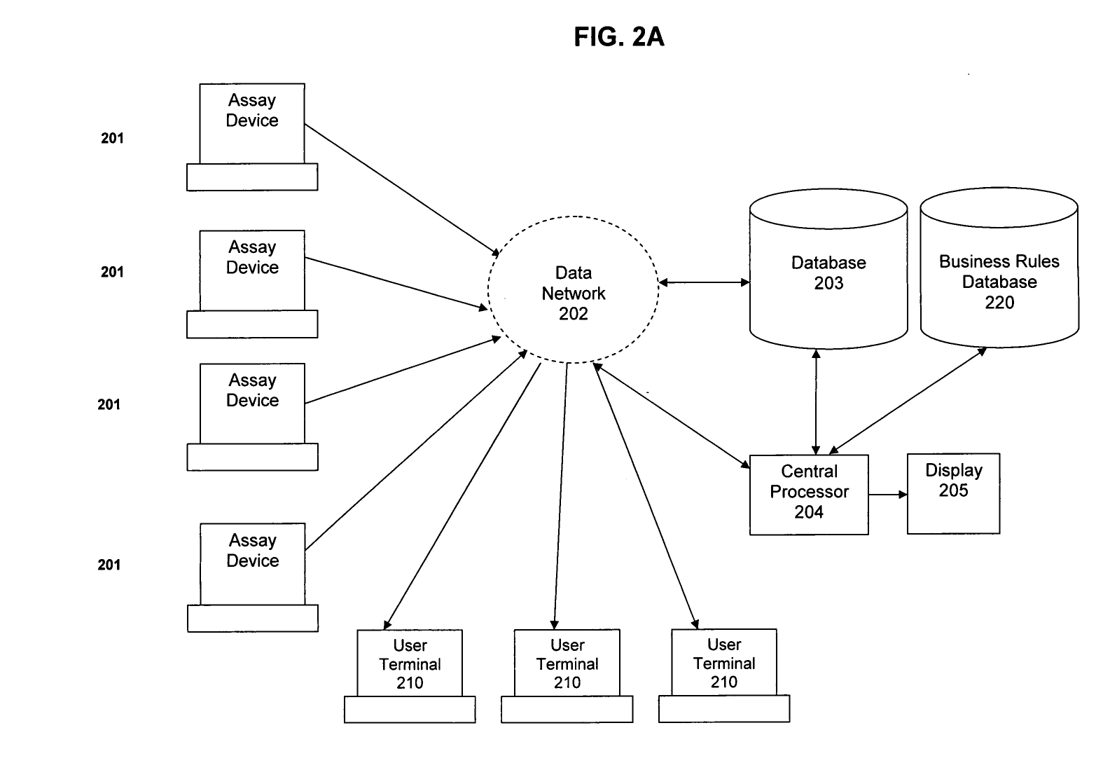

Before describing each of these three areas in detail, a brief overview of a generalized method and system according to exemplary embodiments of the present invention is presented with reference to FIGS. 1, 2 and 2A.

FIG. 1 depicts an exemplary process flow according to an exemplary embodiment of the present invention. Beginning at 101, an assay or panel of assays can be conducted on a biological sample, e.g., blood, urine, etc., which has been taken from an individual. Such individual can simple be an individual or he or she can be a member of a population or sub-population whose immunologic informatics are of use to some entity or enterprise. For example, the individual could be an insured of a health insurance company that is using the techniques of the present invention to efficiently manage the healthcare of its insureds so as to minimize costs. Or, alternatively, such an individual could be an immigrant whose vaccination history is unknown but whose immune status is of interest to his new country's immigration service. Such exemplary embodiments are described more fully below in Section III.

In FIG. 1, at 102 the results of the assay or assays conducted at 101 can be obtained, and at 103 there can be an optional step of analyzing the assay results locally. In exemplary embodiments of the present invention assays can be conducted and read in a variety of assay reading devices. There are many assays available using known techniques. Some of them are more sophisticated and some less sophisticated. In exemplary embodiments of the present invention, an assay reading device can, for example, obtain results at 102, store those results and analyze them locally, for example, in a processor communicably connected to the assay reading device. Alternatively, if only raw assay results are obtained from a less sophisticated technology, those results can, for example, be sent over a data network and stored in a database record. This is illustrated at 104. At 105, the results can be analyzed by accessing the particular record associated with the particular individual to whom the assay panel or panels were administered at a given time. Such analysis can involve a variety of algorithms ranging from a simplistic look at quantity of antibodies per defined unit of blood or other bodily fluid, or it can also, for example, include a complex analysis where a variety of assay results are input and combined in linear and non-linear ways to produce some metric of immunologic significance. Such algorithms are described more fully below in Section II. Finally, at 106, based on the results of the above described analysis, recommendations can be generated. Such recommendations can include, for example, that the individual obtain one or more vaccines, that the individual be administered prophylactic therapies to boost his or her immune system, or that the individual be administered gene therapy to correct the genetic defect which places him or her at risk of communicating a certain disease or condition, to name a few.

In general, in many exemplary embodiments according to the present invention process flow will be equivalent to or substantially similar to the process flow depicted in FIG. 1. In each of those exemplary embodiments, one or more panels of assays can be conducted with respect to one or more individuals. Results can be obtained, stored and analyzed, and based on such analysis, recommendations for action (or inaction, such as, for example, in cases of over-vaccination, as described above) can be recommended.

FIG. 2 is an exemplary generalized system diagram which correlates to the generalized method depicted in FIG. 1. With reference to FIG. 2, there can be seen a number of assay devices 201. These assay devices include one or more assay panels which have been conducted with respect to an individual or individuals and for which results have been obtained. The results obtained from the assay devices can, as described in connection with the generalized method in FIG. 1, be locally analyzed at each assay device, provided that such assay device has a data processor and memory and the results can be stored locally at the assay device. Alternatively, the assay device results can, for example, be communicated over a data network 202 to a central processor 204 and stored in a central database 203. The central processor 204 can access the records which it has received and analyze them by implementing a number of analytic algorithms as described more fully below.

Central processor 204, based on its analysis, can generate recommendations based on decision trees and criteria embedded in the various analytic algorithms it implements. These recommendations can be displayed locally at the central processor at display 205 and can there be printed in a tangible medium for distribution to interested persons. Alternatively, the central processor 204 can, for example, send the results of its analysis over a data network to various users who can access the results at user terminals 210.

FIG. 2A presents an alternative generalized system diagram similar to FIG. 2. However, as can be seen in FIG. 2A, there is an additional database, the business rules database 220, communicably connected to central processor 204. In such an exemplary system the central processor can implement algorithms to operate on stored assay data which can, for example, also take as inputs various business rules in generating a decision regarding a recommendation. For example, as described more fully below in Section III, an exemplary embodiment of the present invention can be utilized to help a health insurance underwriter manage its population of insureds. There can, for example, be an annual or semi-annual requirement of all insureds to have assays for various immunological components conducted on their blood or other bodily fluids. After analysis of the results of such assays, an insurance company can determine whether a particular insured is susceptible to one or more given diseases or other ailments which would result in increased expenditures for medical treatment. The insurance company could then decide if it was not more economical to require the insured to undergo certain prophylactic treatments, such as, for example, vaccines or immune system boosting therapies, etc., where the cost of such prophylactic therapies is less than, as determined by some user determined factor, the expected exposure for medical care if the insured contracts one or more of the diseases or ailments to which he or she is susceptible.

In such context, there would need to be a number of business rules where such user defined quantities, threshold levels, cost functions or metrics, figures of merit, expected risks, etc., can be input and articulated or incorporated in a number of rules. Such rules can then be taken into account by the central processor in implementing algorithms which take as inputs data from business rules database 220 as well as a primary ImmunoScore database 203.

Given the generalized exemplary method of FIG. 1 and the generalized exemplary systems of FIGS. 2 and 2A, what is next described are a number of exemplary assay panels which can be administered to an individual or members of a population according to exemplary embodiments of the present invention. The scientific background behind the various exemplary assay panel, as well as which segments of the general population such panels are best administered to, are also described in detail.

I. Exemplary Assay Panels

The present invention is generally directed towards assessing the “protective immune status” or “immunologic status” of an individual or population. A “protective immune status” is understood to be represented by an array of detectable components (phenotypic and/or genotypic) of an immune system (adaptive and/or innate) that comprise its protective capacity against harmful substances and/or cells (such as, for example, microorganisms or cancer). Such components can, for example, consist of genes as well as gene products. Genes can include, for example, those which encode immunologic receptors (such as, for example, toll-like receptors (“TLR”s) and chemoattractant receptors) as well as effector molecules (such as, for example, cytokines and chemokines) which may also, for example, exist as genetic polymorphisms capable of deleterious and/or beneficial effects. Gene products can include, for example, antibodies, complements, cytokines, chemokines, chemoattractant receptors, TLRs, lectins, and other immune-related ligands. Harmful substances can consist of, for example, chemicals and/or toxins originating from the environment, microorganisms, or one's self.

Once diagnostic information is acquired from an individual regarding his or her immune status, this information can be, for example, added to a system database. Such a database can contain, for example, not only the results of ImmunoScore diagnostic testing but demographic data and patient history information as well. Such a system database can also be used to record adverse events occurring coincident with immunizations. The information gathered in the database can be invaluable to, for example, the ACIP for making recommendations regarding immunization scheduling, as well as help discover unsuspected patterns and correlations relevant to immune status and immune response.

ImmunoScore diagnostic testing can be, for example, tailored to meet an individual's specific immunization status needs. In addition, each individual can, for example, receive their own personal ImmunoScore card that they could carry with them to office visits, and the database information can be easily transferable in the ever-increasingly likely event that they change physicians or other primary health care providers. Additionally, ImmunoScore data, analysis of data and relevant database information can be stored as part of a person's totality of health information and medical records, in electronic formats such as, for example, entries in electronic health information databases, or computer chips embedded in, for example, “smart” cards or “smart driver's licenses,” as was recently proposed by a panel of Virginia legislators.

For economy of description, the references cited herein are provided in full citation in Appendix A hereto. Throughout the text citations are made to author and year of publication alone.

One component of ImmunoScore data can be, for example, the raw as well as processed results of diagnostic tests or assays relating to immune status, as described below. ImmunoScore diagnostic testing is envisioned to be done on a small assay device or testing instrument that can be located, for example, in a doctor's office. The testing can be done, for example, with a sample of an individual's whole blood, plasma, serum, saliva, milk, semen, tears, or urine. In the case of blood, for example, the sample can be obtained by a finger prick, heel stick, ear stick, other skin prick, capillary draw, venous draw, or an arterial draw. The instrument can take assay panels and the patient sample. Patient information can also be input. The resulting information can be, for example, displayed to a user, printed, stored in a removal medium, stored in the instrument, and/or transmitted (wired or wireless) to other devices such as via an intranet or the Internet.

Numerous methods and systems have been developed for the detection and quantitation of analytes of interest in biochemical and biological substances that can be used, for example, in such an instrument. Such methods and systems which are capable of measuring trace amounts of microorganisms, pharmaceuticals, hormones, viruses, antibodies, nucleic acids and other proteins can be of great value to researchers and clinicians.

A substantial body of art has been developed based upon well known binding reactions, such as, for example, antigen-antibody reactions, nucleic acid hybridization techniques, and protein-ligand systems. The high degree of specificity in many biochemical and biological binding systems has led to many assay methods and systems of value in research and diagnostics. Typically, the existence of an analyte of interest is indicated by the presence or absence of an observable “label” attached to one or more of the binding materials. Of particular interest are labels which can be made to luminesce through photochemical, chemical, and/or electrochemical means. “Photoluminescence” is the process whereby a material is induced to luminesce when it absorbs electromagnetic radiation. Fluorescence and phosphorescence are types of photoluminescence. “Chemiluminescent” processes entail the creation of luminescent species by chemical transfer of energy. “Electrochemiluminescence” entails creation of luminescent species electrochemically.

Electrochemiluminescent (ECL) assay techniques are an improvement over chemiluminescent techniques. They can, for example, provide a sensitive and precise measurement of the presence and concentration of an analyte of interest. In such techniques, the incubated sample is exposed to a voltammetric working electrode in order to trigger luminescence. In the proper chemical environment, such electrochemiluminescence is triggered by a voltage impressed on the working electrode at a particular time and in a particular manner. The light produced by the label is measured and indicates the presence or quantity of the analyte. For a fuller description of such ECL techniques, exemplary reference is made to U.S. Pat. Nos. 5,221,605; 5,705,402; 6,140,138; 6,325,973; and 6,451,225. The disclosures of the aforesaid patents are hereby incorporated herein by reference.

Amplification techniques for nucleic acids may be combined with the above assay techniques. For example, U.S. Pat. No. 6,048,687 discloses how NASBA can be combined with an ECL technique; and U.S. Pat. No. 6,174,709 discloses how PCR can be combined with an ECL technique. The disclosures of the aforesaid patents are also hereby incorporated herein by reference.

The assay instrument can, for example, be, or be similar to, the BioVeris Corporation M1R or M1M instruments with an added sample processing front end. Aspects of these instruments are disclosed in pending U.S. patent application Ser. Nos. 10/600,165 and 10/841,569, each under common assignment herewith. The disclosures of these patent applications are hereby incorporated herein by reference. Additionally, for illustrative purposes, attached hereto as Appendix C is some descriptive data on the BioVeris M1M Analyzer, which, as noted, can be used in exemplary embodiments of the present invention to run the various panels of assays.

In exemplary embodiments of the present invention, an assay instrument can include, for example, amplification techniques such as PCR or NASBA. In exemplary embodiments, the instrument can use fluorescence, chemiluminescence, or ECL assay techniques. In exemplary embodiments, multiple measurements can be done simultaneously; in other exemplary embodiments, multiple measurements can be done sequentially. In exemplary embodiments of the present invention, an assay instrument can, for example, contain self-test and/or self-calibration components.

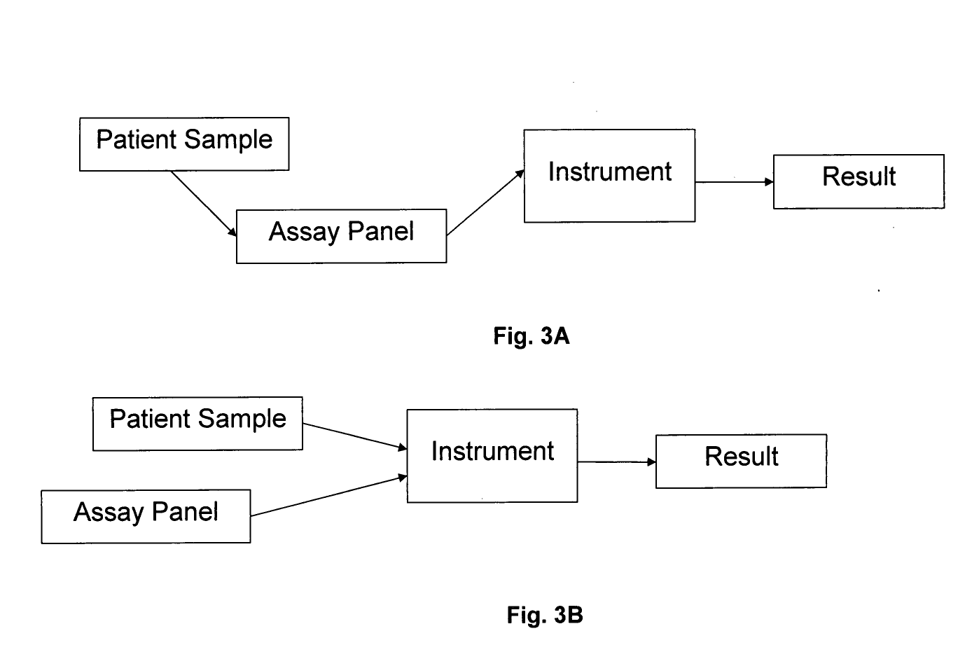

In exemplary embodiments of the present invention, a sample can be added to an assay panel, and the combination then inserted into the test instrument, as shown in FIG. 3A. In alternate exemplary embodiments, the sample and assay panel can be separately inserted into the test instrument, as shown, for example, in FIG. 3B.

As described below, entries to a master ImmunoScore database can be, for example, coded so as to protect patient confidentiality. A patient could, however, be able to learn from their physician in real time, for example, which vaccines he or she might need to ensure protection from vaccine-preventable illnesses. The physician can, for example, offer the vaccine during the same visit, or shortly thereafter. Any possible adverse effects from any delivered vaccinations could be subsequently entered into an ImmunoScore database and that information could be shared with the ACIP or other agencies or bodies, as described more fully below.

The actual assays can be performed, for example, based upon the needs of the individual or individuals being examined. Age, occupation, travel plans, immigration status, military status, and previous health status can all be considered prior to initiation of ImmunoScore diagnostic analyses in exemplary embodiments. In exemplary embodiments of the present invention, the following exemplary broad categories can, for example, be utilized as focal points for test panels:
1. Entry to primary school.
2. College entry.
3. Age 19-49 years.
4. Age 50-64 years.
5. Age>65 years.
6. Health-care professionals.
7. Military personnel:
   recruits and officer accessions;
   alert forces;
   individualized according to occupational or personal needs; and
   veterans.
8. Travelers.
9. Immigrants.
10. Individuals with identifiable health risks (not necessarily exclusively):
   a. Complement-deficient individuals (e.g. meningococcal disease susceptibility);
   b. Genetically identified (e.g. HLA haplotype, sepsis susceptibility) disease-susceptible individuals;
   c. Mannose-binding lectin-deficient individuals;
   d. Hepatitis B vaccine poor/non-responders; and
   e. Ethnic groups and others known to respond poorly to polysaccharide, conjugate, or other vaccines.

Although the health effects may be just as great, coverage levels for immunizations in adults are not as high as those achieved in children. Barriers to adult immunization can include, for example, not knowing that immunizations are needed, misconceptions about vaccines, and lack of recommendations from health care providers. Adding an ImmunoScore vaccine diagnostic component to routine physical examinations in adults could easily point out where immunizations are needed. Just as importantly, it could, for example, point out exactly which individuals would not need to be unnecessarily boosted if their serum antibody levels proved to be sufficient for any particular vaccine. The development and acceptance of an ImmunoScore vaccine diagnostic surveillance system can not only aid in increasing vaccine coverage, but can also, for example, add increased surveillance of the level of immune response and duration of protection thereof for a wide variety of recommended vaccines.

There are still great risks posed to the population by vaccine-preventable diseases. The risks posed by failure to immunize were vividly illustrated by the measles outbreak that began in 1989, which led to 43,000 cases and over 100 deaths, mainly among children in the United States. Despite better efforts at record-keeping for immunizations, for example, the development of the Adult and Adolescent Clinic Assessment Software Application (ACASA) to facilitate obtaining immunization data on adults and adolescents, adult compliance with vaccination protocols generally remains unsatisfactory. One way to demonstrate the need for vaccination in adults is to demonstrate a low antibody titer in an individual and present the need to boost the antibody titer using assigned correlates of protection from vaccine-preventable diseases. If an antibody titer to a specific agent is determined to not meet the recognized level of a correlate of protection by an exemplary ImmunoScore analysis, and that titer is easily boosted by vaccination, then that individual would likely be more easily convinced of the need for protective immunization. Not only would this diagnostic tool prove extremely beneficial to the individual, but data added to an ImmunoScore database can be collected regarding immune correlates of protection for very large populations. Demographic assessments can also be compiled from the database, leading to, for example, new discoveries regarding possible age-related or ethnic responses to immunizations and/or other immune status issues.

In what follows, targeted panels of immune status assays according to exemplary embodiments of the present invention are presented. The exemplary targeted panels fall into two broad groups, college students and adults.

For college students, three exemplary panels are presented: (a) a Meningococcal diagnostic panel, (b) a panel designed to measure the residual immunity induced by childhood vaccines, and (c) a panel directed to measuring immunity from common sexually transmitted diseases.

For the general adult population, exemplary panels directed to the following groups or categories are presented: (a) military personnel, (b) travelers, (c) adults—general immune status, (d) health care workers, (e) bioterrorism, (f) chronic disease, (g) Th-1-Th2 diagnostic panel, and (h) Immigrants and Internationally Adopted Children.

Further specific considerations can be made as adults age. Thus, as the idea of a 50 year-old check up has gained ground of late, this could be an opportune time to check an individual's overall immune status using the system and methods according to exemplary embodiments of the present invention. Also, individuals over the age of 65 rate special consideration as they are at increased risk for influenza and pneumococcal infections. It would thus particularly behoove these individuals to be aware of their immune status so that their later years need not be unnecessarily troubled by infection with a vaccine-preventable illness.

Using these basic panels of assays as building blocks, in exemplary embodiments of the present invention, aggregations of one or more panels, with variations thereto as may be desired, can be defined for various purposes. These can be referred to, for example, as ImmunoScore superpanels. For example, a primary school panel can be defined, to be administered upon a child's entry into grammar school. Such a panel could include, for example, a persistent immunity to childhood vaccines assay panel. Similarly, a middle school student superpanel can be defined as well. Such a superpanel could include, for example, a persistent immunity to childhood vaccines panel and a sexually transmitted disease panel. Another examplary superpanel could be defined for women of childbearing years, as described below. Such an exemplary superpanel can include, for example, a newly defined women of childbearing years panel, a persistent immunity induced by childhood vaccines panel, and a sexually transmitted disease panel.

A. College Student Diagnostic Panels

1. Meningococcal Diagnostic Panel

Infection with the intracellular Gram negative diplococcus Neisseria meningitidis remains a world-wide concern for public health care, because of the infection's ability to cause rapid and potentially fatal invasive disease, particularly in children and young adults. The spectrum of disease varies from a common cold to life-threatening disorders including meningitis and/or a fulminant septic shock. Meningococcal sepsis is characterized by a sudden onset of fever and a petechial or purpuric rash, which can be followed by hypotension and multiple organ failure. Mortality rates can be as high as 40% (Rosenstein, et al. 2001). Meningococcal disease also causes substantial morbidity: 11-19% of survivors have sequelae; neurologic disability, hearing loss, skin necrosis, or loss of limbs (Vermont, et al. 2002).

Nasopharyngeal carriage of meningococci is common: carriage rates are estimated at ˜10% in Europe, and they rise to >50% in closed or semi closed institutions, such as military recruit camps and universities (Williams, et al. 2003). Despite a high carriage rate, progression to invasive disease rarely occurs. There are several risk factors associated with contracting meningococcal disease. They include close contact with a patient with primary invasive disease, recent viral respiratory illness (e.g. influenza), smoking or exposure to secondary smoke, and host susceptibility. It is host susceptibility, in particular, that the vaccine immunodiagnostic panel will focus upon.

Two questions arise from the patterns of meningococcal disease. Why do some patients die within hours despite intensive treatment? Why is meningococcal bacteremia in other patients a self-limiting disorder? The explanation likely lies in the variability of host genetic factors. These factors include (but are not limited to) FcγRIIa receptor polymorphism, late component complement deficiency (LCCD), properdin deficiency, mannose-binding lectin (MBL) genetic polymorphisms and levels of expression, and genetic variability in expression and allotypes of interleukin-1 (IL-1) and IL-1R, IL-6, and IL-10.

The quadrivalent A, C, Y, W-135 meningococcal polysaccharide vaccine is the formulation currently available in the United States. Each dose consists of 50 μg of the four purified bacterial capsular polysaccharides. The immunogenicity and clinical efficacy of the serogroups A and C meningococcal vaccines have been well established. The serogroup A polysaccharide induces antibody in some children as young as 3 months of age. The serogroup C component is poorly immunogenic in recipients aged<18-24 months. The serogroups A and C vaccines have demonstrated clinical efficacies>85% in school-aged children and adults and are useful in controlling outbreaks. Serogroups Y and W-135 polysaccharides are safe and immunogenic in adults and in children aged>2 years; although clinical protection has not been documented, vaccination with these polysaccharides induces bactericidal antibody. The antibody responses to each of the four polysaccharides in the quadrivalent vaccine are serogroup-specific and independent. Although the polysaccharide vaccines have proven effective in the short term, concerns have been raised about the induction of immunologic hyporesponsiveness to C polysaccharide. This has been clearly demonstrated in infants and toddlers (Leach, et al. 1997). Meningococcal C polysaccharide vaccines are effective in preventing meningococcal disease in older children and adults in the short term. Vaccinating persons in high-risk situations (e.g. outbreaks) with polysaccharide vaccine provides protection until functional antibody levels decline. The demonstration of subsequent hyporesponsiveness to meningococcal C PS vaccine means that vaccinating low-risk persons (e.g. primary schoolchildren) may reduce the effectiveness of revaccination in a high-risk situation. Furthermore, it raises the theoretical concern that such persons may be unable to mount a protective antibody response if later exposed to serogroup C organisms and may be at increased risk of developing meningococcal disease (Granoff, et al. 1998). In November 1999, the United Kingdom introduced conjugate vaccines against meningococcal serogroup C disease into its immunization schedule for infants. The group C meningococcal PS conjugate vaccine was shown to be able to overcome the hyporesponsiveness induced by previous polysaccharide vaccine in adults (Richmond, et al. 2000). Use of the conjugate vaccine should be considered for long-term protection against serogroup C disease (Jokhdar, et al. 2004).

Evaluation of the meningococcal vaccination requires the use of quantitative and functional antibody determinations by enzyme-linked immunosorbant assay (ELISA) and serum bactericidal assay (SBA) activity, respectively. Evaluation of the need for meningococcal vaccination will require measurement of serum immunoglobulin (Ig) specific for the capsular polysaccharides from serogroups A, C, Y, and W-135. In addition, an assay component should be considered to measure serum IgM specific for the capsular polysaccharide from serogroup B. As yet, levels of serum Ig considered to be protective for meningococcal disease have not been agreed upon. The protective levels of antibody seem to vary with the pathogen. For example, the estimated protective serum Ig level is 0.15 μg/mL for invasive Haemophilus influenzae type b disease (Robbins, et al. 1973). The protective Ig level for protection from pneumococcal disease is estimated to be 1.0 μg/mL from studies analyzing immune response to conjugate vaccine (Jódar, et al. 2003). The maternal level of anti-GBS_{III} Ig estimated to be protective for infants is ≧10.0 μg/mL (Lin, et al. 2004). From studies examining immune response to Group A polysaccharide and Group C conjugate vaccines, it has been suggested that serum Ig≧2.0 μg/mL would be sufficient to protect from infection with microorganisms of these serogroups (Peltola, et al. 1977 and Burrage, et al. 2002, respectively). Importantly, the putative protective concentration of anti-meningococcal polysaccharide antibodies in subjects with complement deficiencies is not known and may be different from that needed for complement-sufficient individuals, because only the opsonizing antibody function is available in the former. It has been suggested that an Ig level of ≧5.0 μg/mL for the A, C, Y, and W-135 capsular polysaccharides be used for individuals with complement deficiencies (Platonov, et al. 2003). As yet, there is no vaccine available for Group B meningococcal disease. IgM directed to the capsular polysaccharide would be the primary analyte of choice, but a recommended protective level has not yet been ascertained. It is possible that serum antibodies to protein antigens as yet undetermined may prove useful in analyses for the immune response to vaccines for serogroup B.

In exemplary embodiments of the present invention, in order to make an appropriate recommendation for meningococcal immunization, it will also be necessary to measure, diagnostically, other immune system components. Included in an exemplary meningococcal diagnostic panel can thus be, for example, an assay to measure the allotypic expression of FcγRIIa receptor. There are two known allotypic forms of this receptor consisting of one amino acid substitution—FcγRIIa-H131 and FcγRIIa-R131. Homozygous R131 individuals are much less effective in binding IgG2 and are therefore less effective in the phagocytosis of Neisseria meningitidis, Haemophilus influenzae, and Staphylococcus aureus. In all age groups, a correlation has been found between FcγRIIa allotype and severity of meningococcal disease, indicated by a longer duration of hospitalization and a higher percentage of complications for patients homozygous for R131. R/H131 heterozygous individuals show intermediate levels of phagocytosis, resulting in intermediate associations with susceptibility to and severity of meningococcal disease (Platonov, et al. 1998).

Immune protection against N. meningitidis is complex. Although bactericidal antibodies directed against the organism can confer protection against it, defects in the complement system can make some individuals particularly susceptible to infection. The complement system is a series of 19 plasma and 9 membrane proteins. Activation of the complement cascade leads to the formation of the membrane attack complex (MAC), which results in the lysis and cell death of the bacterium. Terminal component and alternative pathway deficiencies seem to have a large effect on susceptibility to, and severity of, meningococcal disease. Individuals with LCCD (C5 to C9) have a 7,000-10,000 fold higher risk of symptomatic meningococcal infections. If a complement deficiency is revealed by immunodiagnostic assays, antibiotic prophylaxis can be considered, and special attention can be given to immunization against encapsulated organisms such as pneumococcus, Haemophilus influenzae, and Neisseria (Hoare, et al. 2002).

Properdin is a soluble glycoprotein which has a unique role as a positive regulator of the alternative complement pathway (Fearon and Austen, 1975). Type I properdin deficiency is characterized by the absence of detectable protein in plasma, type II by the presence of low but detectable plasma properdin (<10% of normal) and the rare type III by a normal serum concentration of a dysfunctional variant of properdin (Linton and Morgan, 1999). All three variants of properdin deficiency are clinically characterized by a significant risk of meningococcal disease (Figueroa and Densen, 1991). Genetic deficiency in humans of mannose-binding lectin (MBL) was shown to be associated with increased susceptibility to meningococcal disease (Hibbard, et al. 1999). The MBL gene determines the amount of MBL present in plasma. A large study in children with meningococcal disease revealed that the rates of homozygous as well as heterozygous MBL variant genotypes were much higher in patients than in controls (Hibbard, et al. 1999).

Additionally, unusually high levels of interleukin-1 (IL-1) and its endogenous antagonist IL-1 receptor antagonist (IL-1ra) can be detected in patients with meningococcal disease, particularly in those with severe manifestations (van Deuren, et al. 1997). The IL-1 gene cluster contains three related genes (IL1A, IL1B, and IL1RN) which encode IL-1α, IL-1β, and IL-1ra (Dinarello, 1996). Allelic variation at the IL-1 gene cluster influences many inflammatory and infective conditions. While a study found no difference in allelic variation between patients with meningococcal disease and a cohort of controls derived from a population of blood donors, patients carrying the common allele at IL-1B (−511) were more likely to survive meningococcal disease. This same group also found that both increasing age and infection with serogroup C influence (poorly) the likelihood of death (Read, et al. 2003).

Patients with meningococcal disease have increased plasma levels of pro-inflammatory cytokines IL-6, IL-1β, and TNF-α, with higher levels associated with fatal outcome. One study found that the IL-6-174 G/G and IL-10-1082 A/A genotypes to be more frequent in nonsurvivors compared to survivors of meningococcal disease (Balding, et al. 2003).

Given the above-described immunological background, in exemplary embodiments of the present invention, the following tests can be included in a meningococcal diagnostic panel:

1. Antibody (Ig) to (4 tests):
Group A Meningococcal Polysaccharide (GAMP)
Group C Meningococcal Polysaccharide (GCMP)
Group Y Meningococcal Polysaccharide (GYMP)
Group W-135 Meningococcal Polysaccharide (GWMP)

2. Antibody (IgM) to Group B Meningococcal Polysaccharide (GBMP) (1 test)

3. Serum levels of complement components (7 tests):
C6
C7
C8
C9
Properdin
MBL

4. Measurement of genetic polymorphisms (5 tests):
FcγRIIa receptor
IL-1
IL-1R
IL-6
IL-10

In exemplary embodiments of the present invention, results from an exemplary meningococcal diagnostic panel can be analyzed as follows:
Serum Ig levels for vaccine-preventable serogroups (A, C, Y, and W-135) of N. meningitidis can be assessed. An Ig level exceeding 2.0 μg/mL of all four serogroups would be presumptive of protection in an otherwise healthy individual. There would be no immediate recommendation for meningococcal vaccination in these individuals.
If deficiencies were to be revealed in any of an individual's complement components assayed, or if any unfavorable genetic polymorphisms were shown to exist, then an Ig level of ≧5.0 μg/mL for the vaccine-preventable serogroups could be desirable in these individuals. If these individuals had Ig levels exceeding 5.0 μg/mL for all four serogroups, no vaccination would be recommended. If, however, the level of antibody to any of the four serogroups were to be below 5.0 μg/mL, then a vaccination could be recommended.
Once individuals were shown to have complement deficiencies, or unfavorable genetic polymorphisms, they could be, for example, “flagged” for future monitoring. These are individuals at greatest risk for meningococcal infections, so serum antibody levels are very important in this group. Initially, they could be monitored every 3-5 years for serum Ig to the vaccine-preventable meningococcal serogroup capsular antigens.
As yet there is limited information available regarding the persistence of serum Ig to the N. meningitidis group polysaccharides. As the system database grows, more information regarding antibody persistence can become available and analysts can, for example, have a better idea as to when to recommend retesting and, perhaps, revaccination of individuals more susceptible to meningococcal disease.
It has previously been demonstrated that repeated vaccination with the capsular polysaccharide from Group C organisms promotes immune hyporesponsiveness (Richmond, et al. 2000, Jokhdar, et al. 2004). This is a red flag to overuse of the vaccination protocol. Currently, immunocompromised individuals are recommended for repeated immunizations every 3-5 years. In exemplary embodiments of the present invention, the ImmunoScore meningococcal diagnostic panel can prevent over-immunization with the polysaccharide formulation by first measuring immune status vis-à-vis menigococcal disease prior to simply vaccinating following a standard schedule. Immunization with Group C conjugate vaccine overcomes the hyporesponsiveness, but is not yet approved in the United States.

In exemplary embodiments of the present invention, the above described analysis can be, for example, expressed as a series of rules or processes, and implemented as a program or software on a data processor. Such a program can, for example, generate recommendations and/or conclusions, and write ImmunoScore data and such processing output to a system database. Such recommendations, can, for example, include scheduling of vaccinations and retesting, as appropriate.

2. Sexually Transmitted Diseases Assay Panel

Sexually transmitted diseases (STDs) are caused by organisms that infect the mucosal surfaces of the genitourinary tract. In spite of its public health importance, current STD vaccine research lags behind work against pathogens that target another mucosal region, the respiratory tract. In the latter case, live-attenuated viral vaccines, killed whole-cell bacterial vaccines, subunit/protein bacterial vaccines, and bacterial polysaccharide vaccines have been enormously successful. To move STD vaccine research forward, complex issues must be resolved. Particular scientific problems have delayed STD vaccine development, like incomplete attenuation, accentuated immunopathology, poor immunogenicity, and broad antigenic heterogeneity (Fletcher, 2002). ImmunoScore diagnostics will be an invaluable tool to assist in diagnoses of STDs and vaccine design.

Chlamydial species cause widespread infections in humans. Chlamydia trachomatis serovars D to K are considered the world's most common sexually transmitted pathogens (Gerbase, et al. 1998), and following vertical transmission through an infected birth canal, cause neonatal conjunctivitis and pneumonia. Respiratory infection with C. pneumoniae occurs in almost everyone during their lifetime (Hogan, et al. 2004). C. pneumoniae is estimated to cause an average of 10% of community-acquired pneumonia and 5% of bronchitis and sinusitis cases (Kuo, et al. 1995). In addition, avian strains of C. psittaci have long been known to cause psittacosis in humans.

In addition to these acute chlamydial infections, chlamydiae are associated with a range of chronic diseases that are characterized by inflammation and scarring and result in significant damage to the host (Hogan, et al. 2004). The World Health Organization estimates that 146 million people have trachoma due to ocular infection by C. trachomatis serovars A to C and that 4.9 million of these are totally blind (Whitcher, et al. 2001). Ascending infection by serovars D to K of the female upper genital tract, known as pelvic inflammatory disease, causes salpingitis, which in turn leads to fibrosis and scarring of the fallopian tubes, and eventual complications of ectopic pregnancy and tubal infertility (Cates and Wasserheit, 1991). C. trachomatis originating from the genital tract is also associated with reactive arthritis, which develops in 1-3% of patients after genital chlamydial infection (Wollenhaupt and Zeidler, 1990). C. pneumoniae, which can also disseminate from the site of the initial infection, has been associated with cardiovascular disease (Kuo, et al. 1993) and late-onset Alzheimer's disease (Balin, et al. 1998). In addition, unresolved respiratory C. pneumoniae infection may contribute to the pathogenesis of chronic inflammatory lung diseases, such as asthma (Hahn, et al. 1991) and chronic obstructive pulmonary disease (Blasi, et al. 1993).

The chlamydiae are an evolutionarily distinct group of eubacteria sharing an obligate intracellular lifestyle and a unique developmental cycle that has been well characterized. The cycle begins when infectious, metabolically inert elementary bodies (EB) attach to and stimulate uptake by the host cell. The internalized EB remains within a host-derived vacuole and differentiates to a larger, metabolically active reticulate body (RB). The RB multiplies by binary fission, and after 8 to 12 rounds of multiplication, the RB differentiate to EB (Moulder 1991). EB progeny are then released from the host cell to initiate another cycle (Wolf, et al. 2000).

Recurrent chlamydial disease my result from either repeated infections or persistence of the organism after unresolved infections. Chlamydial infections are particularly insidious and difficult to control because they can have two phases: an initial phase which frequently results in only mild symptoms or no symptoms at all and a secondary phase that may occur months or years later and result in infertility and/or debilitating disease.

Chronic infection with C. pneumoniae has been documented to be common in school children and the immune response to C. pneumoniae was associated with frequency of asthma exacerbations (Cunningham, et al. 1998). Recent infection with C. pneumoniae has also been claimed to be of importance for the development of asthma in previously healthy individuals. It had been postulated that acute C. pneumoniae infection of the respiratory tract in nonasthmatic individuals could lead to development of bronchial hyperresponsiveness. Therefore, patients with a new diagnosis of asthma should be evaluated for possible C. pneumoniae infection (Laurila, et al. 1997). By definition, a current infection with C. pneumoniae is characterized as a positive IgM titer value≧1:20. A past chlamydial infection is indicated by IgG titer≧1:32 and ≦1:256. Chronic infection with C. pneumoniae is characterized by an IgG titer≧1:512 combined with an IgA titer≧1:40 (Gencay, et al. 2001).

In exemplary embodiments of the present invention, ImmunoScore diagnostic testing for chlamydial infections can, for example, encompass serum IgG, IgM, and IgA levels for C. pneumoniae and C. trachomatis. Such testing could, for example, reveal chronic and past infections by these organisms, thereby revealing a need for treatment, or monitoring for future breakout infections by either of these pathogens. The information provided to the database regarding chlamydial infections and many associated chronic conditions (heart disease, arthritis, asthma, PID, Alzheimer's disease, etc.) could also be invaluable.

Intracellular microbial pathogens, like Chlamydia, cause a plethora of diseases that pose a huge public health challenge. Efficacious prophylactic vaccines are needed to protect the population from these infectious diseases. The research goal for an efficacious human chlamydial vaccine has faced key challenges to define the elements of protective immunity to facilitate vaccine evaluation, the judicious selection of appropriate vaccine candidates that possess stable antigenic and immunologic properties and the development of effective delivery vehicles and adjuvants to boost immune effectors to achieve long-term protective immunity (Igietseme, et al. 2003). Progress in the functional immunobiology of Chlamydia has established the essential immunologic paradigms for vaccine selection and evaluation, including the obligatory requirement for a vaccine to induce T-helper Type 1 immune response that controls chlamydiae. The most appropriate adjuvant for a given antigen will depend, to a large extent, on the type of immune response that is required for protective immunity. The type of adjuvant selected might orchestrate the type of immune response induced (Th1 or Th2), which in turn may have a significant impact on the protective efficacy of a vaccine. Consequently, induction of an inappropriate response type could, unfortunately, increase susceptibility to infection (Bandholtz, et al. 2002). ImmunoScore diagnostics can thus be invaluable to evaluate the immune response to chlamydial vaccines under development. The immune response to the immunogen, as well as Th1/Th2 ratios can, for example, be incorporated into the database analyses.

Infections with Herpes Simplex Virus (HSV) types 1 and 2 are ubiquitous, although there is marked regional variation in prevalence throughout the world. Among various populations, between 60-95% are infected with HSV-1, and between 6-50% with HSV-2 (Stanberry, et al. 2000). HSV-2 infection is common in the United States, affecting 21.9% of the population over 12 years of age (Fleming, et al. 1997). In that same study, only 9% of seropositive individuals reported that they had “ever had genital herpes.” Skin or mucosal infection is usually followed by transmission via sensory nerves to the spinal or trigeminal ganglia where life-long latent infection occurs. Subsequent frequent intermittent reactivation from ganglionic neurons results in viral shedding into the oral cavity in the case of HSV-1 or, in the case of HSV-2, into the vagina, perianal, or penile skin (Wald, et al. 2000).

Infection is accompanied by the acquisition of type-specific antibody, and this acts as a marker for infection. Most initial and recurrent infections are asymptomatic or unrecognized. One of the key questions concerns the difference between the genetic makeup and immune responses of patients with asymptomatic infections versus those with clinical disease, give that the viral gene sequences predicting disease as opposed to asymptomatic shedding have not been identified (Cunningham and Mikloska, 2001).

Studies of animal models have shown that neutralizing antibody is inefficient in preventing the axonal spread of the virus. Innate immunity (natural killer cells, interferon, and macrophages) is an important component of protection from HSV infection, but T cells, primarily CD 8⁺ cytotoxic T cells, are a major determinant of protective immunity (Aurelian, 1990). This is a particular challenge for the immune system, because HSV has developed various immune evasion mechanisms.

Unsuccessful attempts to produce an effective vaccine against HSV-2 or HSV-1 have extended for more than four decades (Stanberry, et al. 2000). The impediments to development of a successful prophylactic vaccine are the identification both of the principal immune mediators of protection (e.g. T cells or antibodies) and of the key immunogenic proteins, among the 80 or so produced by these viruses. The ability of the virus to enter nerve endings in the skin is also a major hurdle for a prophylactic vaccine (Milligan, et al. 1998).

HSV thus presents a unique ImmunoScore diagnostic opportunity. Current infections can be, for example, detected by measuring IgG antibody to HSV-2 and HSV-1. In addition, immune response of individuals to vaccine components could also be measured as vaccines entered the public domain. Concomitant with the measurement of antibody levels would be measurement of relevant innate and acquired cellular immune responses to the vaccine. The compiled ImmunoScore database can again provide a valuable tool for assessing the immune response to an HSV vaccine, as well as the status of the immune system relative to an ongoing infection.

Human papillomavirus (HPV) is one of the most common causes of sexually transmitted disease in both men and women worldwide and is thought to be the most common sexually transmitted viral disease in the United States. Genital HPV infection is not a reportable disease, so actual incidence and prevalence figures are not known; however, it is estimated that the incidence in the United States ranges from 1 million to 5.5 million per year, and the prevalence is estimated to be as high as 20 million (Cates et al. 1999).

Papillomaviruses are ubiquitous and have been detected in a wide variety of animals as well as in humans and are specific for their respective hosts. More than 200 types of HPV have been recognized on the basis of DNA sequence data showing genomic differences. HPVs can infect basal epithelial cells of the skin or inner lining of tissues and are categorized as cutaneous types or mucosal types. Cutaneous types of HPV are epidermitrophic and target the skin of the hands and feet. Mucosal types infect the lining of the mouth, throat, respiratory tract, or anogenital epithelium. Based on their association with cervical cancer and precursor lesions, HPVs can also be grouped to high-risk and low-risk HPV types (Burd 2003). Low-risk HPV types include types 6, 11, 42, 43, and 44. High-risk HPV types include types 16, 18, 31, 33, 34, 35, 39, 45, 51, 52, 56, 58, 59, 66, 68, and 70.

HPV is associated with a variety of clinical conditions that range from innocuous lesions to cancer. Most HPV infections are benign. HPV was first recognized as the cause of cutaneous warts. Strains that target the face make skin cancer more likely. Other strains, which grow primarily in the lining of the mouth, produce small elevated nodules that can develop into fatal squamous cell cancers.

Cervical cancer is the second most common cancer in women worldwide (Ries, et al. 2001). The magnitude of the association between HPV and cervical squamous cell carcinoma is higher than that for the association between smoking and lung cancer (Franco, 1995). Scientists have identified about 30 HPV types that are spread through sexual contact and infect primarily the cervix, vagina, vulva, penis, and anus. Of these, four are most often found within the malignant cells of cervical cancer, with type 16 accounting for about half the cases in the U.S. and Europe and types 18, 31, and 45 accounting for an additional 25-30% of cases (Harro, et al. 2001). HPV has been implicated in 99.7% of cervical squamous cell cancer cases worldwide (Walboomers, et al. 1999).

Transmission of papillomavirus may be prevented by the generation of antibodies to capsid proteins L1 and L2 that neutralize viral infection. However, because the capsid proteins are not expressed at detectable levels by infected basal keratinocytes or in HPV-transformed cells, therapeutic vaccines generally target nonstructural early viral antigens. Two HPV oncogenic proteins, E6 and E7 may provide the best option for controlling HPV-associated malignancies. Various candidate therapeutic HPV vaccines are currently being tested whereby E6 and/or E7 are administered in live vectors, as peptides or protein, in nucleic acid form, as components of chimeric virus-like particles, or in cell-based vaccines. Encouraging results from experimental vaccination systems in animal models have led to several prophylactic and therapeutic vaccine clinical trials. If these preventative and therapeutic HPV vaccines prove successful in patients, as they have in animal models, then oncogenic HPV infection and its associated malignancies may be controllable by vaccination (Roden, et al. 2004).

In exemplary embodiments of the present invention, an exemplary ImmunoScore diagnostic application can be very much like that for HSV. Current infections, as well as risk for cervical cancer can be diagnosed by screening. Immune response to vaccines, either prophylactic or therapeutic, can be monitored by incorporating the appropriate antigens into the diagnostic screening protocols. Also, information regarding the immune system response to infection and/or vaccination, as well as chronic carriage, can be monitored and tracked by the ImmunoScore database software.

Neisseria gonorrhoeae is an exclusively human pathogen transmitted most often by sexual contact. For the majority of patients, antibiotic treatment is effective and there are few sequelae. In some women, however, N. gonorrhoeae may infect the upper genital tract and cause pelvic inflammatory disease (PID) with serious consequences including sterility. Another consequence of gonococcal infection is its potential to enhance the risk of acquiring other sexually transmitted diseases, including human immunodeficiency virus infection. These important health concerns have sparked continuing interest into the development of vaccines against gonorrhea (Hedges, et al. 1999).

Several prototype gonococcal vaccines have shown limited or no protection against re-infection with N. gonorrhoeae despite the generation of serum antibody responses against the vaccine antigens (Boslego, et al. 1991). The results from vaccine trials parallel observations regarding natural gonococcal infections, where local and systemic antibodies have been detected by immunofluorescence in secretions and serum from infected patients, yet there is a high rate of recidivism of gonococcal infections among patients attending STD clinics. The concept that previous exposure to an antigen results in increased levels of protective antibodies upon re-exposure is central to the development of vaccines against infectious organisms. Repeated infections with N. gonorrhoeae are common. One study found that a history of gonorrhea did not significantly alter levels of any antibodies in male patients, and only serum IgA1 were elevated in female patients (Hedges, et al. 1999).

Offering of clinical (diagnostic and treatment) services has historically been a key strategy for STD control and prevention (Rietmeijer, et al. 2004). The opportunity for the ImmunoScore diagnostic panel is primarily for detection of infected individuals. As vaccines are developed, the ImmunoScore could then be applied to monitoring response to those vaccines in a vaccinated susceptible population of individuals.

Syphilis is a sexually transmitted disease caused by the spirochete Treponema pallidum. Syphilis facilitates the transmission of HIV and may be important in contributing to HIV transmission in those parts of the country where, and in those populations in which, rates of both infections are high. Untreated early syphilis during pregnancy results in perinatal death in up to 40% of cases and, if acquired during the four years preceding pregnancy, may lead to infection of the fetus in over 70% of cases (Syphilis Surveillance Report, 2004).

The rate of primary and secondary syphilis reported in the United States decreased during the 1990s and in 2000 was the lowest since reporting began in 1941 (CDC 2001). However, the number of cases of primary and secondary syphilis increased 2.1% between 2000 and 2001 and increased 12.4% between 2001 and 2002. Increases in cases during 2000-2002 were observed only among men. Increases in syphilis cases among men are associated with reports in several cities of syphilis outbreaks among men who have sex with men, and these outbreaks have been characterized by high rates of human immunodeficiency virus co-infection and high-risk sexual behavior (Syphilis Surveillance Report, 2004).

Despite the fact that humans develop robust immune responses against T. pallidum, they can be infected multiple times. Individuals infected with T. pallidum develop specific immune responses that are able to clear millions of treponemes from sites of primary and secondary syphilis (Engelkens, et al. 1993). The response is a T-cell mediated delayed type hypersensitivity response in which T cells infiltrate syphilitic lesions and activate macrophages to phagocytose antibody-opsonized treponemes (Baker-Zander and Sell, 1980; Lukehart, et al. 1980). Data seem to indicate that both antibodies and T cells play a role in protection but neither alone prevents infection (Morgan, et al. 2003).

Currently, syphilis is diagnosed in the United States by a nontreponemal screening test followed by a treponemal confirmatory test with a turn-around time of one week or more (Sutton, et al. 2004). T. pallidum-specific tests use either whole organisms as the antigens, or sonicates of the pathogen (Schmidt, 2004). Preparing a reproducible antigen is a difficult task because the bacterium cannot be cultured continuously in vitro and must be maintained in rabbits (Cox, 1994). The use of recombinant antigens in place of a poorly defined mixture of antigens from wild-type T. pallidum has the potential for improving the specificity of serologic tests (Schmidt, 2004). Recombinant antigens can be produced in large quantities to ensure reproducibility and cost-effectiveness. As with N. gonorrhea, the opportunity for the ImmunoScore diagnostic panel for T. pallidum is primarily for detection of infected individuals. As vaccines are developed, the ImmunoScore database could then be applied to monitoring responses to those vaccines in a vaccinated population of individuals.

The human immunodeficiency virus (HIV) kills the body's CD4⁺ cells. HIV is spread by sexual contact with an infected individual, by sharing needles and/or syringes with an infected individual, or rarely, through transfusions of infected blood or blood clotting factors. Babies born to HIV-infected mothers may become infected before or during birth or through breast-feeding after birth (Atkinson, 2002).

As HIV vaccines are developed, there is a need for screening assays that will distinguish uninfected HIV vaccine recipients from HIV-infected individuals. A number of problems may arise if HIV vaccines induce seropositivity, as measured by prevailing licensed assays, in large numbers of high-risk individuals:
vaccine efficacy may be more difficult to determine, as seroconversion will become an unreliable marker of infection,
recruitment of volunteers may be impeded if their informed consent requires their knowledge that subsequent screening will indicate a seropositive status,
implementing long-term follow-up and secondary confirmatory assays in seropositive vaccine trial participants returning to the general high-risk population after protocol completion could cause financial, social, and logistical problems (Schwartz et al. 1995).

Because HIV is a persistent infection rather than an acute self-limiting one, clinical endpoints demonstrating lack of disease (i.e. progression to AIDS) would take many years to assess. Therefore, if an HIV vaccine cannot prevent infection, it will be critical to determine the most expeditious methods for evaluating the efficacy of HIV candidate vaccines. Prevention of infection may not be achieved and there are, therefore, other outcomes of immunization that could be beneficial to the host and could also have a positive impact on the AIDS epidemic. It was recognized that it is important to establish alternate virologic, immunologic, and clinical endpoints that could be applied to HIV vaccine efficacy trials (AIDS Vaccine Trials: Considerations for Phase III Trial Design and Endpoints, 2001).

Considerable progress has been made over the past several years in the development of an HIV vaccine. As a result, a growing number of vaccine modalities are being investigated in preclinical and phase I/II clinical trials. However, a number of major scientific challenges still remain. It is widely believed that the ideal vaccine should elicit both neutralizing and cytotoxic T lymphocytes against diverse isolates of HIV, but the precise correlates of immunity have not been identified (Lemckert, et al. 2004). In addition, the patterns of innate cytokine production have been postulated to shift from Th1 cytokines, associated with cell-mediated immune responses to Th2 type cytokines, known to enhance humoral immune response with the progression of HIV-associated disease. One study of HIV-infected adolescents concluded that early in the course of HIV-associated disease in adolescents, there are no detectable shifts from Th1 to Th2 cytokine production (Douglas, et al. 2003). The ImmunoScore diagnostic database would be an invaluable tool for enabling research into HIV infection, and following the relative success of vaccine candidates for HIV. Humoral and cellular immune responses would be measured and demographic data would assist in arriving at conclusions regarding alternate endpoints to clinical trials.

Group B Streptococcus (GBS) is an opportunistic pathogen of humans. In the United States, GBS is the leading cause of serious bacterial infections in newborns (Schuchat, 1999). Other at risk populations include peripartum women, diabetics, and the elderly with underlying illnesses (Paoletti and Kasper, 2003). One-third of the cases of invasive GBS disease now occur in adults>65 years old (Palazzi, et al. 2004). Adherence of GBS to a mucosal surface is the first event in colonization and invasion.

Improved vaccines against GBS have been developed by covalent coupling of variably immunogenic capsular polysaccharide antigens to immunogenic carrier proteins (Paoletti, et al. 2000). Phase I and II clinical trials in healthy, non-pregnant adults have shown that capsular types Ia, Ib, II, III, and V GBS conjugate Vaccines are well tolerated and have superior immunogenicity to uncoupled, homologous polysaccharides (Baker, et al. 1999, Baker, et al. 2000, Kasper, et al. 1996). A GBS type III-TT conjugate was also used in a clinical trial in pregnant women. This particular study concluded that maternal immunization with GBS CPS-TT conjugates could prevent maternal, neonatal, and young infant GBS disease (Baker, et al. 2003).

As vaccines are developed for adults and women of child-bearing age, ImmunoScore diagnostic analyses could be performed on the vaccines. Antibody response to the immunogens would be measured, as well as all-around immune status measurements. Should a GBS vaccine, or vaccines for other sexually transmitted diseases be recommended for children, the diagnostic application would, of course, shift to the ImmunoScore measurement of immunity to childhood vaccines.

As mentioned above, several vaccines for sexually transmitted diseases are presently in development and the eventual availability of such vaccines is expected to result in the prevention of a significant number of burdensome conditions. Young adolescents are presumed to be the likely targets for these vaccines since adolescents' risk for STDs increases as they age and become sexually active. It is unclear, however, to what extent parents will agree to have adolescents receive vaccines for STDs. Inasmuch as acceptance is the foundation for effective immunization programs, an understanding of parental perspectives about this issue is required to inform future STD vaccine program strategies (Mays, et al. 2004). In this respect, the ImmunoScore diagnostic panel must be able to accommodate changes in recommendations of ages and status of individuals being immunized.

Accordingly, in exemplary embodiments of the present invention, the following tests can be used for ImmunoScore measurement of immunity to STDs:

Antibodies to Chlamydia—IgG, IgA, and IgM (3)

Antibodies to HSV—IgG to HSV-1 and HSV-2 (2)

DNA analyses of HPV types—particular emphasis on high-risk

Antibody to N. gonorrhoeae (1)

Antibody to T. pallidum (1)

T-cell related response to T. pallidum

Antibody to HIV

T-cell related response to HIV

Antibodies to GBS serotypes (at least 3)

Measurement of Th1/Th2 cytokines (many as current evolving definitions)
Currently, there are no vaccines available for any of these STDs. Until this situation is ameliorated, the objective of an ImmunoScore STD diagnostic panel would thus be to recommend treatment. The ImmunoScore database can generate correlate of protection information for all diseases. As vaccines are developed, ImmunoScore diagnoses can be designed to examine antibody and other related immune responses to vaccine components.
   3. Persistent Immunity Induced by Childhood Vaccines

Benefits and risks are associated with using all immunobiologics. No vaccine is completely safe or 100% effective. Benefits of vaccination include partial or complete protection against the consequences of infection for the vaccinated person, as well as overall benefits to the society as a whole. Vaccination risks range from common, minor, and local adverse effects to rare, severe, and life-threatening conditions. Therefore, recommendations for immunization practices balance scientific evidence of benefits for each person and to society against the potential costs and risks of vaccination programs.

Standards for child and adolescent immunization practices have been published to assist with implementing vaccination programs and maximizing their benefits (MMWR, 2002). The recommended childhood vaccination schedule is revised annually and is published each January. ACIP currently recommends over 23 immunizations covering 11 different pathogenic microorganisms with up to 19 distinct serogroups/types for children under 18 years of age (not including newly recommended yearly flu immunizations). Table 1 below depicts a current such schedule. The persistence of the duration of the immune response for many of these vaccines is not currently known. Vaccination rates are frequently considered a surrogate measure of protection from disease. Serum levels of protective antibody would prove a more objective measure of protection.

TABLE 1
Recommended Childhood and Adolescent Immunization Schedule

Age

| | | 1 | 2 | 4 |
|---|---|---|---|---|
| Vaccine | Birth | no | no | no |
| Hepatitis B | HepB #1 | ++++++ | ++++++ | |
| | ******* | | | |

HepB#2
******************************************************

| | | |
|---|---|---|
| Diphtheria Tetanus, Perussis¹ | DTaP | DTaP |
| Hemophilus Influenzee type b² | Hib | Hib |
| Inactivated Poliovirus | IPV | IPV |
| Measles, Mumps, Rubela³ | | |
| Varicella | | |
| Pseunococcal³ | PCV | PCV |
| Influenza⁴ | | |
| Hepatitis A⁵ | | |

Age

| | | | | |
|---|---|---|---|---|
| | 6 | 12 | 15 | 18 |
| Vaccine | no | no | no | no |

| | |
|---|---|
| Hepatitis B | HepB #3 |
| | ********************************************************************** |

| | | |
|---|---|---|
| Diphtheria Tetanus, Perussis¹ | DTaP | DTaP |
| | | ************************* |

| | | |
|---|---|---|
| Hemophilus Influenzee type b² | Hib | Hib |
| | | ******************************** |

| | |
|---|---|
| Inactivated Poliovirus | IPV |
| | ********************************************************************** |

| Measles, Mumps, Rubela³ | | | MMR #1 |
|---|---|---|---|
| | | | ******************************** |
| Varicella | | Varicella | |
| | | ****************************************************** | |
| Pseunococcal³ | PCV | | PCV |
| | | | ******************************** |

| | |
|---|---|
| Influenza⁴ | Influenza (Yearly) |
| | ********************************************************************** |

Hepatitis A⁵

| | Age | | | |
|---|---|---|---|---|
| | 24 | | | |
| Vaccine | no | 4-6 Y | 11-12 Y | 13-18 Y |
| Hepatitis B | | | $$$$$$$ | |

HepB Series
///////////////////////////////////////////////////////////////////////////////////////////////////

| | | | |
|---|---|---|---|
| | | $$$$$$$ | |
| Diphtheria Tetanus, Perussis¹ | DTaP | $$$$$$$ | |
| | | Td | Td |
| | | ************ | //////////// |
| | | $$$$$$$ | |
| Hemophilus Influenzee type b² | | $$$$$$$ | |
| Inactivated Poliovirus | IPV | $$$$$$$ | |
| Measles, Mumps, Rubela³ | MMR #2 | $$$$$$$ | |

| | MMR #2 |
|---|---|
| | //////////////////////////////////////////////// |
| | $$$$$$$ |
| Varicella | $$$$$$$ |

Varicella
//////////////////////////////////////////////////////////////////////////////////////////////////////

| | |
|---|---|
| | $$$$$$$ |
| Pseunococcal³ | $$$$$$$ |

PCV PPV
/////////////// ************************************************************************

| | |
|---|---|
| | $$$$$$$ |
| Influenza⁴ | $$$$$$$ |

Influenza (Yearly)
************************************************************************

| | |
|---|---|
| | $$$$$$$ |
| Hepatitis A⁵ | $$$$$$$ |

Hepatitis A Series
************************************************************************

$$$$$$$
This schedule indicates the recommended ages for routine administration of currently licensed childhood vaccines, as of Apr. 1, 2004, for children through age 18 years. Any dose not given at the recommended age should be given at any subsequent visit when indicated and feasible. ////// indicates age groups that warrant special effort to administer those vaccines not previously given. Additional vaccines may be licensed and recommended during the year. Licensed combination vaccines
# may be used whenever any components of the combination are indicated and the vaccine's other components are not contraindicated. Providers should consult the manufacturers' package inserts for detailed recommendations. Clinically significant adverse events that follow immunization should be reported to the Vaccine Adverse Event Reporting System (VAERS). Guidance about how to obtain and complete a VAERS form can be found on the internet: www.vaers.org or by called 800-822-7967,

******* Range of recommended ages
$$$$$ Preadolescent assessment
+++++ Only if another HBsAG(−)
///////// Catch-up immunization

Hepatitis B virus (HBV) is the most common known cause of chronic viremia, with an estimated 200 to 350 million carriers worldwide (Edlich, et al 2003). HBV infection is an established cause of acute and chronic hepatitis and cirrhosis. It is the cause of up to 80% of hepatocelluar carcinomas, and is second only to tobacco among known human carcinogens. More than 250,000 persons die worldwide each year of hepatitis B-associated acute and chronic liver disease (CDC, 2004).

Diagnosis of HBV disease is based on clinical, laboratory, and epidemiological findings. HBV infection can not be differentiated on the basis of clinical symptoms alone, and definitive diagnosis depends on the results of serologic testing. Serologic markers of HBV infection vary depending on whether the infection is acute or chronic. HBsAg (surface antigen) is the most commonly used test for diagnosing acute HBV infections or detecting carriers. The presence of HBsAg indicates that a person is infectious, regardless of whether the infection is acute or chronic. Anti-HBc (antibody to core antigen) develops in all HBV infections, appears shortly after HBsAg in acute disease, and indicates HBV infection at some undefined time in the past. Anti-HBc occurs only after HBV infection, and does not develop in persons whose immunity to HBV is vaccine-induced. Anti-HBc generally persists for life and is not a serologic marker for acute infection. IgM anti-HBc appears in persons with acute disease about the time of illness onset and indicates recent infection with HBV. IgM anti-HBc is the best serologic marker of acute HBV infection. Anti-HBs (surface antigen) is a protective, neutralizing antibody. The presence of anti-HBs following acute HBV infection generally indicates recovery and immunity from re-infection. Anti-HBs can also be acquired as an immune response to hepatitis B vaccine or passively transferred by administration of HBIG. Ten milli-International Units/mL (mIU/mL) is considered to indicate a protective level of immunity (CDC, 2004). Table 2 below summarizes possible results and their interpretations.

TABLE 2
Interpreting the Hepatitis B Panel

| Tests | Results | Interpretation |
|---|---|---|
| HBsAg | negative | |
| anti-HBc | negative | susceptible |
| anti-HBs | negative | |
| HBsAg | negative | |
| anti-HBc | negative | immune due to vaccination |
| anti-HBs | positive with >10 mIU/mL | |
| HBsAg | negative | |
| anti-HBc | positive | immune due to natural |
| anti-HBs | positive | infection |
| HBsAg | positive | |
| anti-HBc | positive | acutely infected |
| IgM anti-HBc | positive | |
| anti-HBs | negative | |
| HBsAg | positive | |
| anti-HBc | positive | chronically infected |
| IgM anti-HBc | negative | |
| anti-HBs | negative | |
| HBsAg | negative | |
| anti-HBc | positive | four possible |
| anti-HBs | negative | interpretations^{#} |

^{#}1. May be recovering from acute HBV infection
2. May be distantly immune and the test is not sensitive enough to detect a very low level of anti-HBs in serum.
3. May be susceptible with a false positive anti-HBc.
4. May be chronically infected and have an undetectable level of HBsAg present in the serum.

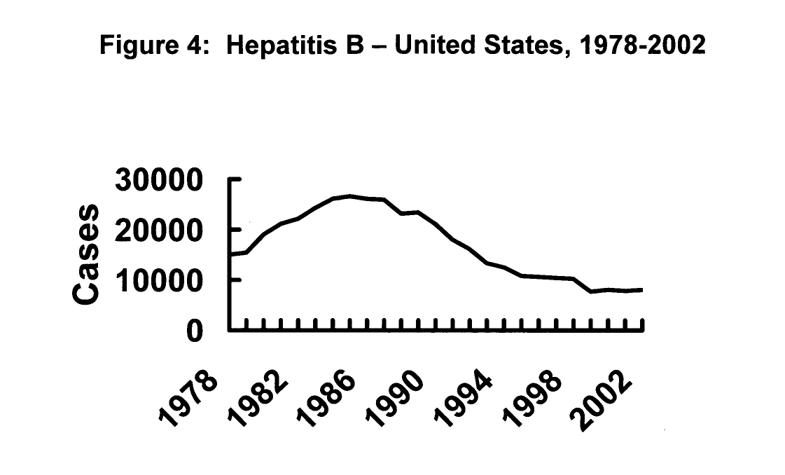

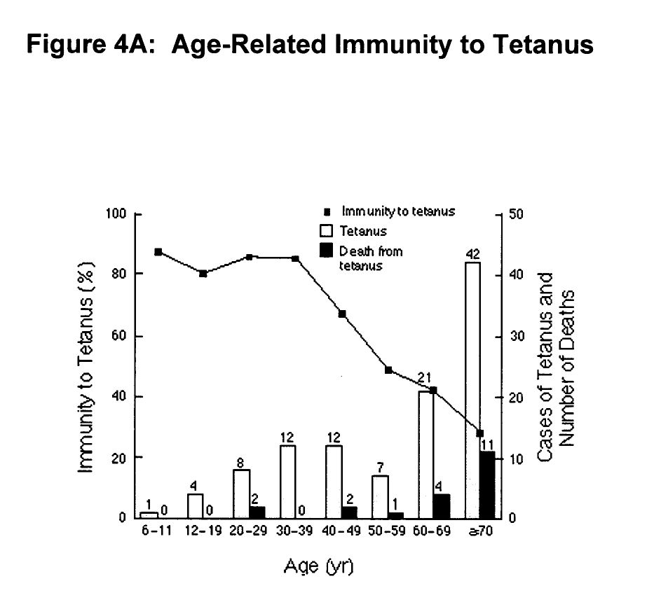

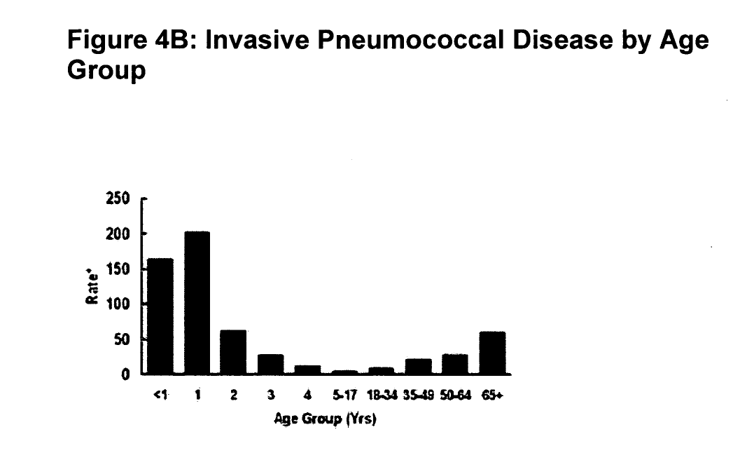

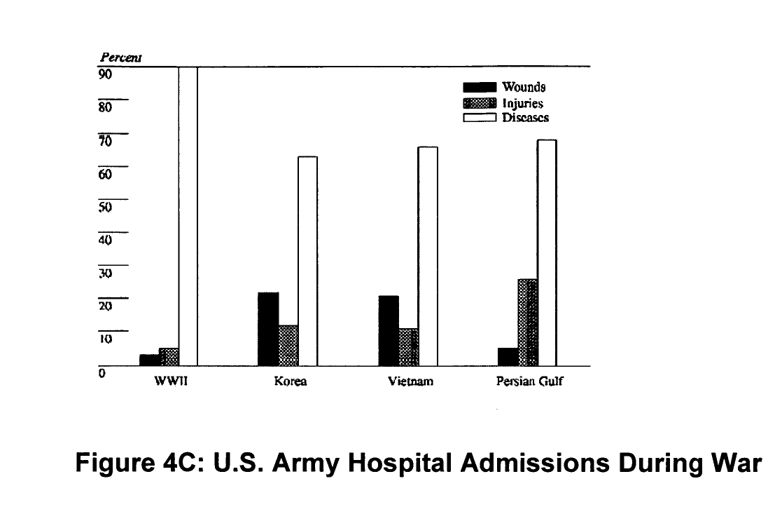

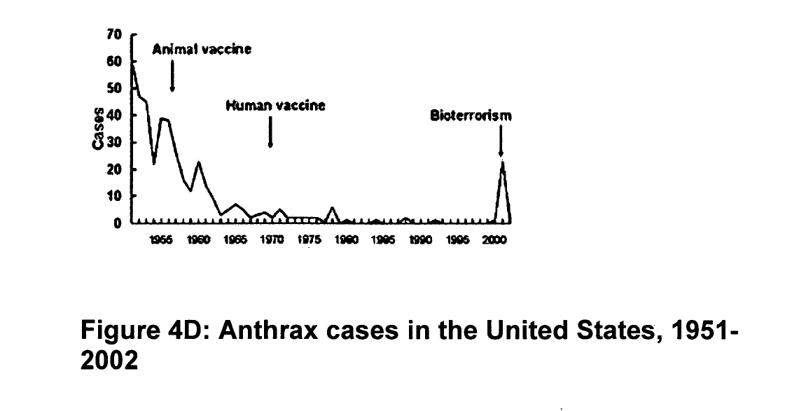

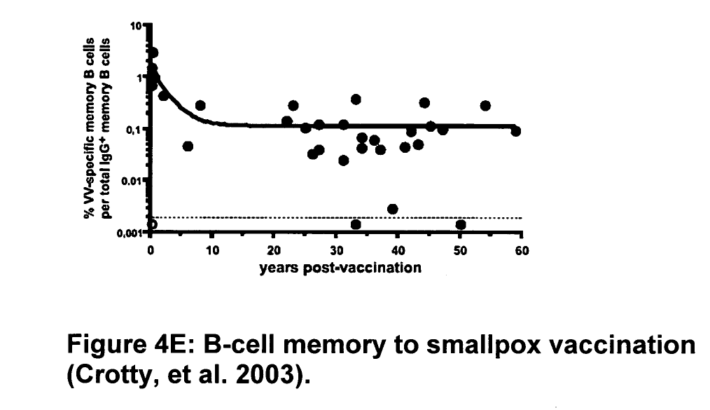

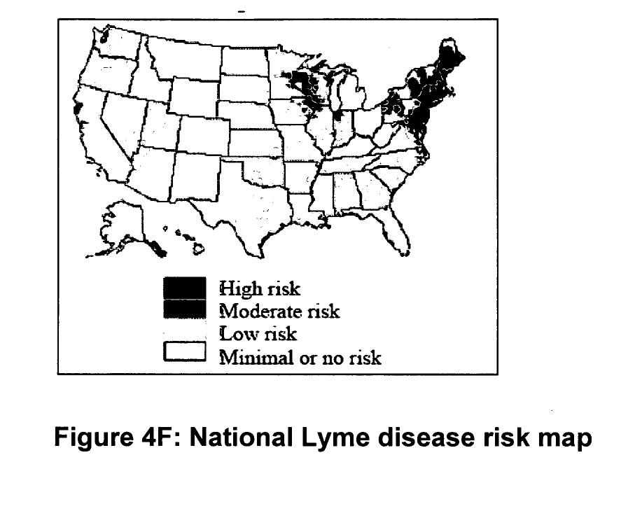

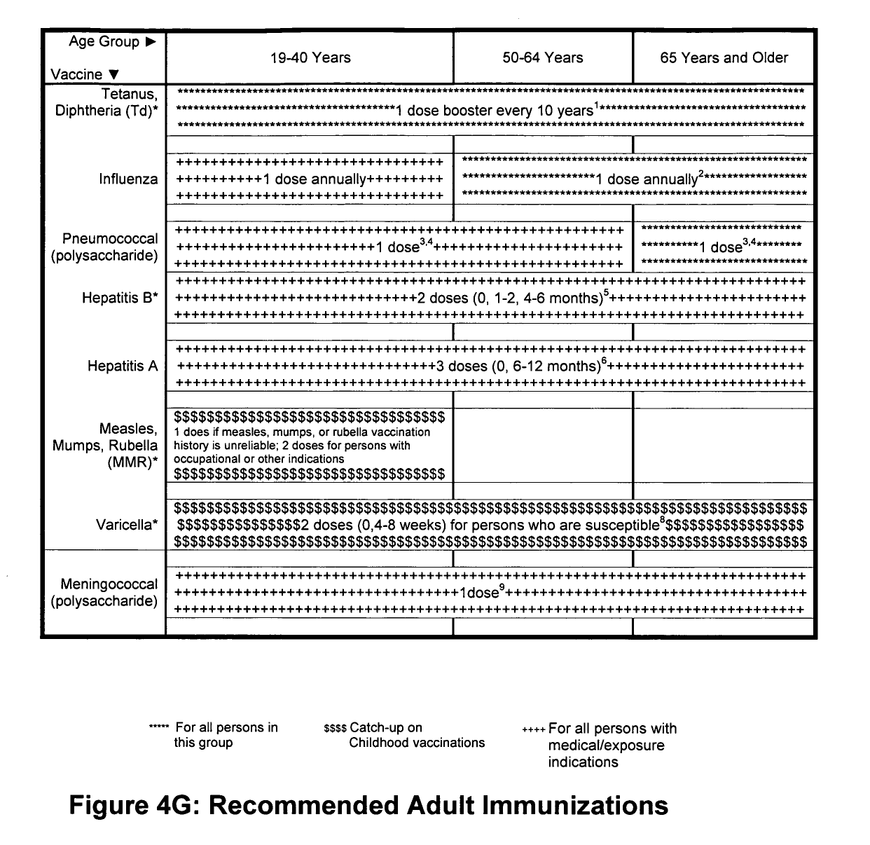

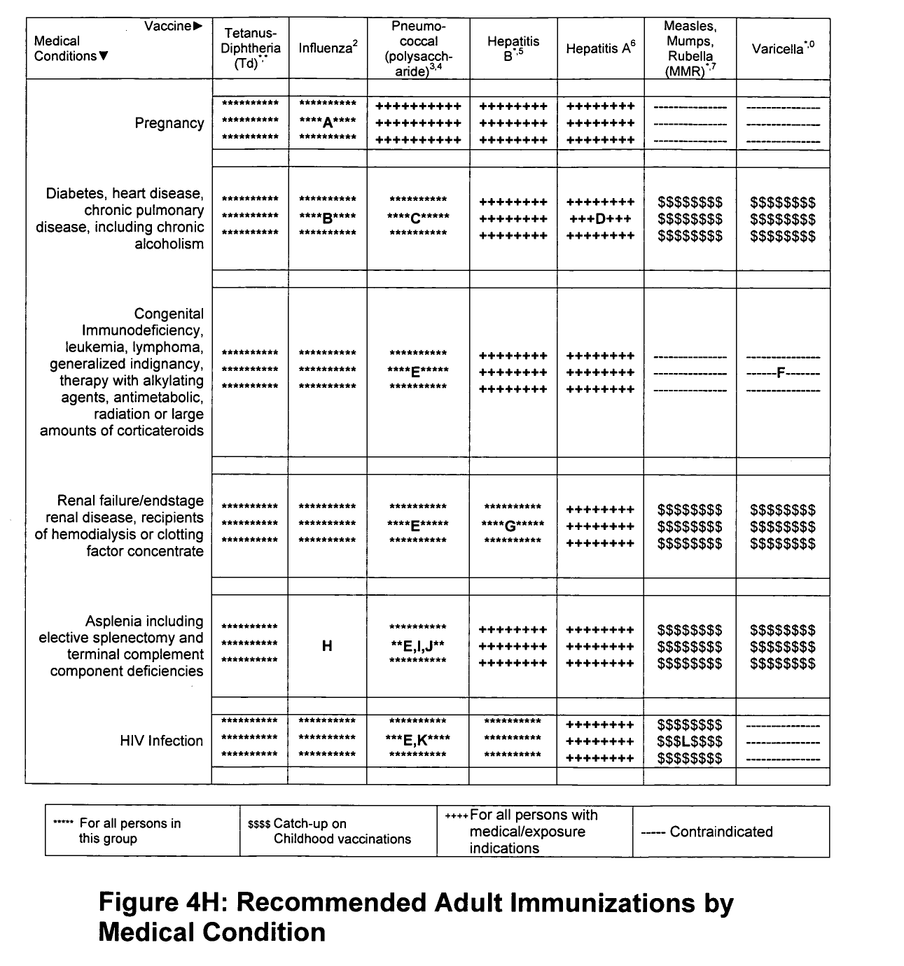

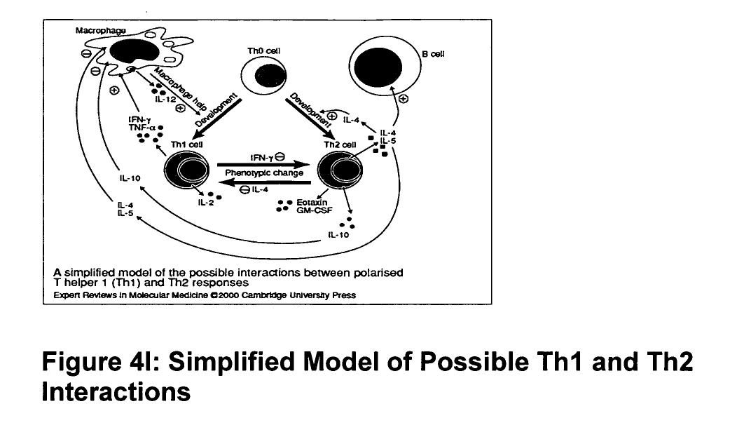

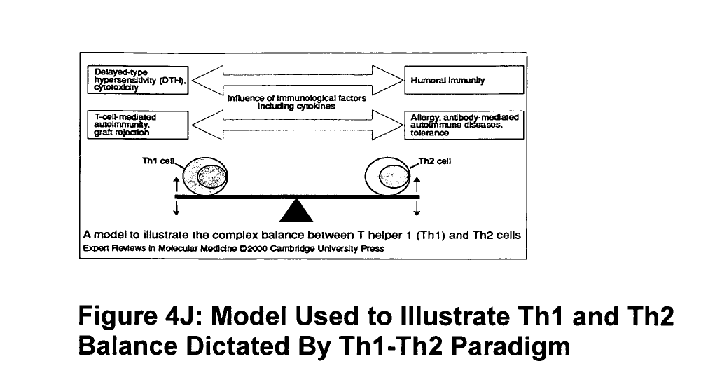

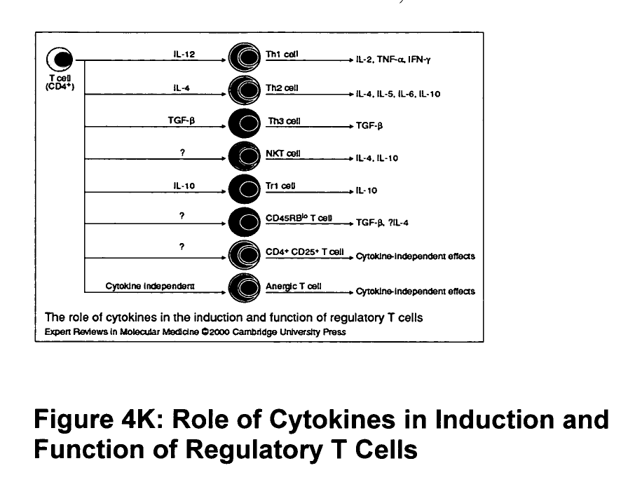

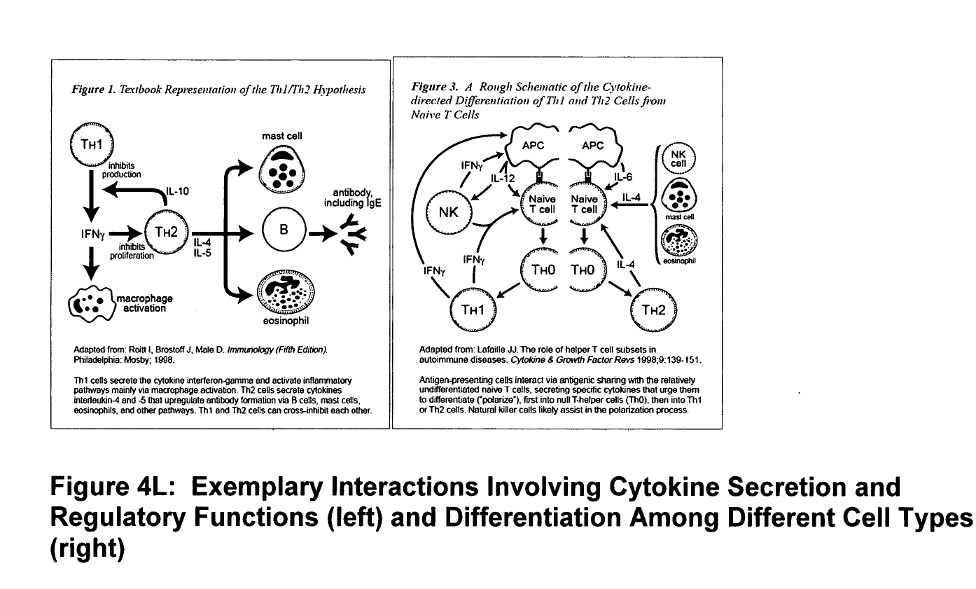

Hepatitis B vaccines have been available in the United States since 1981. However, the impact of the vaccine on HBV disease has been less than optimal, and the incidence of reported hepatitis B cases is now only slightly less than as it was before the vaccine was licensed, as shown in FIG. 4.

The apparent lack of impact from the vaccine can be attributed to several factors. From 1981 until 1991, vaccination was targeted to people in groups at high risk of HBV infection. A large proportion of persons with HBV infection (25-30%) deny any risk factors for the disease. These persons would not be identified by a targeted risk factor screening approach. The three major risk groups (heterosexuals with contact with infected persons or multiple partners, injection drug users, and men who have sex with men), are not reached effectively by targeted programs. Deterrents to immunization of these groups include lack of awareness of the risk of disease and its consequences, lack of effective public or private sector programs, and vaccination cost. Difficulty in gaining access to these populations is also a problem. Further, there has been limited success in providing vaccine to persons in high risk groups, due to rapid acquisition of infection after beginning high risk behaviors, low initial vaccine acceptance, and low completion rates.

A comprehensive strategy to eliminate hepatitis B virus transmission was recommended in 1991, and includes:
prenatal testing of pregnant women for HBsAg to identify newborns who require immunoprophylaxis for the prevention of perinatal infection and to identify household contacts who should be vaccinated,
routine vaccination of infants,
vaccination of adolescents, and
vaccination of adults at high risk of infection.

Prevaccination serologic testing is not indicated before routine vaccination of infants or children. Prevaccination serologic testing may be considered when vaccinating adolescents in group with high rates of HBV infection including Alaskan natives, Pacific islanders, children of immigrants from endemic countries, and family members of HBV carriers. Post vaccination serologic testing is not routinely recommended following vaccination of infants, children, adolescents, or most adults, but is recommended for infants born to HBsAg⁺ women, dialysis patients, immunodeficient persons, and certain healthcare workers.

An expert panel has declared that booster immunizations are not needed for lifelong immunity to hepatitis B (European Consensus Group 2000). The evidence for maintenance of immunity in teenagers after vaccination in infancy is slender. Also, the risk of hepatitis B virus infection is increased by sexual exposure. A study of children in Gambia (an area of high endemic disease) found that teenagers vaccinated in infancy have low concentrations of antibody to hepatitis B surface antigen (Whittle, et al. 2002). They found that breakthrough infections and chronic carriage were clearly and strongly related to peak antibody concentrations. Half of the children who failed to produce detectable concentrations of antibody became infected, most within the first five years after vaccination and of those infected nearly half became chronic carriers. In a study of children vaccinated as infants in Taiwan, it was found that a routine booster vaccination may not be required to provide protection against chronic HBV infection before 15 years of age (Lin, et al. 2003).

From the results of the studies mentioned above, ImmunoScore testing of college age students in the United States for the hepatitis B panel is imperative. In exemplary embodiments of the present invention, an exemplary hepatitis B panel can, for example, consist of testing serum samples for the presence of anti-HBs antibody, with further testing indicated if those results were to be positive.

Diphtheria is an acute, toxin-mediated disease caused by Corynebacterium diphtheriae. C. diphtheriae is an aerobic gram-positive bacillus. Toxin production occurs only when the bacillus is itself infected by a specific bacteriophage carrying the genetic information for the toxin. Only toxigenic strains can cause severe disease.

Because immunity to diphtheria wanes over time after vaccination and because many adults do not receive the recommended tetanus-diphtheria (Td) boosters every 10 years, half of the adults in the United States are estimated to have levels of diphtheria toxin antibodies below the level considered to be the lower limit of protection (≧0.10 IU/mL). Currently, few laboratories have the capability to accurately test antibody levels to diphtheria toxin. Thus, in exemplary embodiments of the present invention, an ImmunoScore test for anti-diphtheria toxin IgG can thus be a part of an exemplary childhood vaccine induced immunity immunodiagnostic panel.

Tetanus is an acute, often fatal, disease caused by an exotoxin produced by Clostridium tetani. Despite the fact that effective vaccines against tetanus have been available since the 1940s, many Americans do not have immunity to tetanus, particularly among the elderly. There is excellent immunity to tetanus among six year olds; however, antibody levels decline over time and one-fifth of older children (aged 10-16 years) do not have protective (>0.15 IU/mL) antibody levels, as shown in FIG. 4A (Gergen, et al. 1995). Diphtheria-tetanus boosters are called for among college-aged students. An ImmunoScore diagnostic assessment of these individuals can, for example, thus be helpful prior to vaccination, to monitor for possible effects of immune suppression among hyperimmunized individuals.

Pertussis, or whooping cough, is an acute infectious disease caused by the bacterium Bordetella pertussis. Pertussis is a highly contagious respiratory disease. Infected adolescents and adults with mild illness are the source of potentially life-threatening illness in infants and young children (Scott, et al. 1997). Whooping cough is currently one of the ten most common causes of death from infectious disease worldwide. The incidence of pertussis has increased substantially in some developed countries due to decreased pertussis vaccine use and waning post-vaccination immunity in the elderly population (Hewlett 2000). In one study, it was estimated that as many as 20 to 30% of adults with prolonged cough may have pertussis (Robbins 1999). The laboratory diagnosis of pertussis in adults, even those who have only mild symptoms, may be important, as they may transmit the disease to infants, who are more susceptible to serious complications (Cockerill and Smith 2004).

The relative value of antibodies and/or T-cell immune responses to Bordetella pertussis antigens in the immunity induced by acellular pertussis (aP) vaccines is still an open issue, probably due to the incomplete knowledge on the mechanisms of protective immunity to pertussis (Ausiello, et al. 2003). Little is known about the capacity of different pertussis vaccines to elicit cell-mediated immunity (CMI) in infants or whether these responses correlate with antibody titers against B. pertussis antigens and/or disease protection. Even less is known about the duration of immunity and about differences in the types of protection induced by immunization and by natural infection. The long-term evaluation of antibody profiles and CMI may therefore contribute to our understanding of the immunological characteristics of pertussis-specific protection and provide important clues as to the best vaccination schedule (Esposito, et al. 2001). Ideally, serum antibody response to pertussis antigens pertactin (PRN), pertussis toxin (PT), filamentous hemagglutinin (FHA) and fimbriae would be measured by the ImmunoScore Vaccine diagnostic panel.

Haemophilus influenzae is a gram-negative coccobacillus. The outermost structure of H. influenzae is composed of polyribosylribitol phosphate (PRP), a polysaccharide, which is responsible for virulence and immunity. The most striking feature of Hib disease is age-dependent susceptibility. Passive protection of some infants is provided by transplacentally acquired maternal IgG antibodies and breast feeding during the first six months of life. Peak attack rates occur at 6-7 months of age, declining thereafter. Hib disease is uncommon beyond five years of age. (CDC, 2004).

Concentrations of serum anti-Hib capsular polysaccharide (PRP)≧0.15 and ≧0.10 μg/mL are widely used as surrogates for protection against invasive Hib disease. However, the relationship between serum anti-Hib PS following immunization and protection against colonization is not known, making it difficult to evaluate new Hib or combination vaccines. A measurement of the amount of anti-PRP antibody would be an invaluable diagnostic tool at several stages in an individual's life. It is not certain that a low anti-PRP IgG level would be indicative of a recommendation for a booster at this time, but the information would be valuable to the construction of the immunization registry database.

Poliovirus is a member of the enterovirus subgroup. Enteroviruses are transient inhabitants of the gastrointestinal tract, and are stable at low pH. There are three poliovirus serotypes (P1, P2, and P3). There is minimal heterotypic immunity between the three serotypes.

The inactivated poliovirus vaccine (IPV) contains three serotypes of virus. This vaccine is highly effective in producing immunity to poliovirus. There is ≧90% efficacy after 2 doses and ≧99% efficacy following three doses (CDC, 2004). However, the duration of the immunity is not known with certainty, although antibody persists for at least four years (Duffy, et al. 1993). Thus, the immune response to inactivated poliovirus vaccine can be measured by ImmunoScore diagnoses. If Ig levels are shown to be sub-optimal, it has been demonstrated that booster response to a single dose of IPV is excellent, even if the priming doses were of oral polio vaccine, which is not currently recommended (General Recommendations on Immunization, 2002).

Measles is an acute viral infectious disease. Measles is the greatest vaccine-preventable killer of children in the world today and the eighth leading cause of death among persons of all ages worldwide (Murray and Lopez 1997). Prior to the license of the first vaccine in 1963, 400,000 cases were reported in the United States each year. More likely approximately 3.5 million (or the entire birth cohort) were infected annually. By the early 1980s, state requirements for a single dose of measles vaccine before school entry were instrumental in reducing the number of reported measles cases to record low levels (Wood and Brunell 1995). However, outbreaks of measles continued to occur, mainly among unvaccinated preschool-aged children, and vaccinated adolescents. Primary vaccine failure (the lack of an effective immune response) contributed to the high proportion of cases among vaccinated children (Frank, et al. 1985). Other surges through the 1990s (primarily from immigration) call for increased surveillance for outbreaks (Measles, Mumps, and Rubella—Vaccine Use and Strategies for Elimination 1998).

ACIP recommends that combined measles-mumps-rubella vaccine be used when any of the individual components is indicated. Measles vaccine produces an inapparent or mild, noncommunicable infection. Measles antibodies develop in approximately 95% of children vaccinated at 12 months of age and 98% of children vaccinated at 15 months of age. MMR vaccine failure may occur because of passive antibody in the vaccine recipient, damaged vaccine, incorrect records, and possibly other reasons. Most individuals who fail to respond to the first dose will respond to a second dose (CDC, 2004).

Mumps is an acute viral illness. Until relatively recently, mumps was viewed primarily as an illness that affected armies during times of mobilization. Mumps was a frequent cause of outbreaks among military personnel in the pre-vaccine era, and was one of the most common causes of aseptic meningitis and sensorineural deafness in childhood. Outbreaks of mumps have been reported among military personnel as recently as 1986 (CDC, 2004).

Mumps vaccine is available as a single antigen preparation, combined with rubella vaccine, or combined with measles and rubella vaccines. Mumps vaccine produces an inapparent, or mild, non-communicable infection. More than 97% of recipients of a single-dose develop measurable antibody. The duration of antibody is believed to be longer than 25 years and is probably life-long. As previously stated, the ACIP recommends that combined measles-mumps-rubella (MMR) vaccine be used when any of the individual components is indicated (CDC, 2004). All persons born in or after 1957 should have documentation of at least one dose of MMR vaccine.

Periodic outbreaks of mumps still occur, even in highly vaccinated populations. A “highly vaccinated” population is a population in which more than 95% of the individuals in that population have been vaccinated. In a study cohort of 318 students in the United States in 1990, clinical mumps developed in 54 students (18% attack rate), 53 of whom had been vaccinated (Cheek, et al. 1995). The authors stated that the overall attack rate was the highest reported to date for a population demonstrating virtually complete vaccine coverage. They concluded that even verified documentation of vaccination may not be an accurate indicator of an individual's protection against mumps. Therefore, despite the assumption that protection from mumps infection is likely to be life-long, there is no current measure in place for the assessment of an individual's immune status regarding mumps infection. This is another candidate for assessment by ImmunoScore diagnostics, and addition to the database.

Rubella virus is most closely related to group A arboviruses, such as Eastern and Western Equine Encephalitis viruses. Prevention of Congenital Rubella Syndrome (CRS) is the main objective of rubella vaccination programs in the United States. Infection with rubella virus can be disastrous in early gestation. The virus may affect all organs and cause a variety of congenital defects. Infection may lead to fetal death, spontaneous abortion, or premature delivery (CDC, 2004).

RA 27/3 rubella vaccine is safe and more immunogenic than previously used rubella viruses. In clinical trials, 95% or more of vaccinated individuals aged 12 months and older developed serologic evidence of rubella immunity after a single dose (Pink Book 2004).

Recent data indicate that the rate of rubella susceptibility and risk are highest among young adults. During 1992-94, approximately 8% of persons aged 15-29 years were estimated to lack serological evidence of immunity to rubella (CDC, unpublished). Thus, an ImmunoScore diagnostic panel would be very effective monitoring college aged students for immunity to rubella infection. This cohort would be fast approaching the age where rubella infection is most devastating, and diagnosis prior to tragedy would be most welcome.

Varicella (Chickenpox) is an acute, contagious disease caused by varicella zoster virus (VZV). Following acute varicella infection, VZV persist in latent form in sensory-nerve ganglia without clinical manifestation. Approximately 15% of the population will experience herpes zoster (shingles) at some point during their lifetimes (MMWR 1996). The immunological mechanism that controls latency of VZV is not well understood. However, factors associated with recurrent disease include aging, immunosuppression, intrauterine exposure to VZV, and varicella at a young age (<18 months). In immunocompromised individuals, zoster may disseminate, causing generalized skin lesions, and central nervous system, pulmonary, and hepatic involvement (Pink Book 2004).

Varicella zoster vaccine is a live attenuated viral vaccine. Among healthy adolescents and adults, an average of 78% develop antibody after one dose, and 99% develop antibody after a second dose given 4-8 weeks later. Studies on the persistence of antibody and clinical efficacy in both children and adults are ongoing (CDC, 2004).

Immunity to VZV appears to be long-lasting, but approximately 1% of vaccinated individuals each year develop breakthrough infections (CDC, 2004). One study investigated a chickenpox outbreak in a well immunized school in Oregon in 2001. In this study, 18 of 152 (12%) of vaccinated students developed chickenpox, compared with 3 of 7 (43%) of the unvaccinated students. The calculated vaccine effectiveness was 72%. Students vaccinated>5 years before the outbreak were 6.7 times more likely to develop breakthrough disease as those vaccinated≦5 years before the outbreak (Lee, et al. 2004). They concluded that a booster vaccination may deserve additional consideration. Again, this is likely an ideal candidate vaccine for monitoring with an ImmunoScore diagnostic panel.

Disease caused by Streptococcus pneumoniae results in wide-spread illness and death throughout the United States each year. Some pneumococcal bacteria are encapsulated, their surfaces composed of complex polysaccharides. Encapsulated organisms are pathogenic for humans and experimental animals, whereas organisms without capsular polysaccharides are not. Capsular polysaccharides are the primary basis for the pathogenicity of the organism. They are antigenic and form the basis for classifying pneumococci by serotypes (CDC, 2004).

More than 40,000 cases and more than 5,500 deaths from invasive pneumococcal disease (bacteremia and meningitis) occurred in the United States in 2002. In addition, there are thousands of cases of non-bacteremic pneumonia, and millions of cases of otitis media which are considered non-invasive infections. The highest rates of invasive pneumococcal disease occur in young children, especially those <two years of age (FIG. 4B).

There are two pneumococcal vaccines currently distributed in the United States. There is a 23-valent polysaccharide vaccine for use in adults aged 65 years and older, individuals≧2 years of age with a chronic illness, and immunocompromised individuals≧2 years of age, and a 7-valent polysaccharide conjugate vaccine intended for infant use. More than 80% of healthy adults develop antibodies against the serotypes contained in the polysaccharide vaccine. Elevated antibody levels persist for at least five years in healthy adults, but fall more quickly in individuals with underlying illnesses. After four doses of the pneumococcal conjugate vaccine, >90% of healthy infants develop antibody to all seven serotypes contained in the vaccine (with presumptive protective Ig levels≧1.0 μg/mL of type-specific antibody). The amount of antibody required to reduce either pneumococcal carriage or disease is unknown. Quantitative measurement of total Ig might not correlate with functional immune system response (MMWR 2000).

ImmunoScore diagnostic measurement of Ig against conjugate vaccine serotypes can, for example, be a valuable determination in students of college age. Individuals of this age category are not particularly susceptible to pneumococcal disease, but a determination of antibody levels would be informative as these individuals continue to age.

Thus, in exemplary embodiments according to the present invention, the following tests for ImmunoScore measurement of immunity to childhood vaccines can be included in an exemplary panel directed to college students, or in other exemplary embodiments, to adults in general:

Antibody to HBs (1)

Antibody to diphtheria toxin (1)

Antibody to tetanus toxin (1)

Pertusis antibodies (4):
Antibody to pertussis toxin (PT)
Antibody to pertactin (PRN)
Antibody to filamentous hemagglutinin (FHA)
Antibody to fimbriae

Antibody to PRP (Hib) (1)

Antibodies to poliovirus serotypes P1, P2, and P3 (3)

Antibody to measles (1)

Antibody to mumps (1)

Antibody to rubella (1)

Antibody to varicella (1)

Antibody to pneumococcal serotypes (7)

Given the above-described tests for persistent immunity induced by childhood vaccines, in exemplary embodiments the following exemplary analysis and recommendations can be made:
For Hepatitis B, post-vaccination titers of anti-HBs IgG of 10 mIU or greater correlate with the induction of T cell helper responses that mediate the memory of B cells (Plotkin, 2001). An antibody titer below 10 mIU would indicate need for vaccination—one booster dose if previously vaccinated, or a course of three doses if unvaccinated.
The current indication for partial protection from diphtheria disease is an anti-diphtheria toxin antibody concentration between 0.01 and 0.1 IU/mL. Protection is considered to be complete above 0.1 IU/mL (Plotkin, 2002). In exemplary embodiments ImmunoScore diagnostics can recommend a booster dose if antibody concentration were to fall below 0.1 IU/mL. The ImmunoScore database can shed further light in the future as to the true protective level of anti-diphtheria toxin antibody.
The current indication for partial protection from tetanus disease is an anti-tetanus toxin antibody concentration between 0.01 and 0.1 IU/mL. Protection is considered to be complete above 0.1 IU/mL (Plotkin, 2002). ImmunoScore diagnostics can recommend a booster dose if antibody concentration were to fall below 0.1 IU/mL. The ImmunoScore database can shed further light in the future as to the true protective level of anti-tetanus toxin antibody.
One of the most controversial areas within the subject of correlates of immunity is pertussis vaccine. Two separate trials conducted in Sweden indicate that pertussis toxin can protect on its own (Trollfors, et al. 1995). One trial suggests that the addition of filamentous hemagglutinin (FHA) is helpful, two trials suggest that pertactin augments the efficacy of PT and one trial suggests that agglutinogens add efficacy beyond those of PT, FHA, and pertactin (Plotkin, et al. 1997). The problem is that the vaccines do not resemble each other in quantity of antigens, and reliance can be placed only on demonstrated efficacy in the field. The role of ImmunoScore diagnostics for this population can, for example, best be served in data acquisition and correlation to incidence of disease. There is not yet an adult pertussis vaccine, but development proceeds along those lines. The ImmunoScore diagnostic application can be beneficial in exemplary embodiments to ACIP for vaccine recommendations. Testing four components for pertussis disease would lend weight to the accumulated data.
Individuals vaccinated with Hib conjugate vaccines are considered to be protected with an IgG level>0.15 μg/mL (Goldblatt, et al. 1999). Booster vaccination can be recommended if an individual's antibody titer fell below that level.
Individual that receive oral polio vaccine (OPV) are protected by both serum and secretory antibodies. Inactivated polio vaccine (IPV) recipients are dependent primarily on serum antibody for protection against infection and disease (Onorato, et al. 1991). Neutralizing antibody assays are currently used to assess protective Ig levels (titer≧1:8). The format of these assays would necessarily need to be updated to be included in the ImmunoScore analyses. Currently, the duration of protection from IPV vaccination is not known, and ImmunoScore database analyses could, for example, lend strength to the current knowledge levels.
Serum antibody levels>120 mIU are considered to be completely protective against measles infection (Plotkin, 2001). Vaccination can be considered for individuals whose antibody titers fall below this level.
Protection against mumps disease is currently assessed with neutralization assays. Like the polio vaccine, the assay format would need updating for ImmunoScore diagnosis.
Protection against rubella disease is currently assessed with neutralization assays. Like the polio and measles vaccine, the assay format would need updating for ImmunoScore diagnosis.
Protection against varicella disease is currently assessed with neutralization assays. The assay format would need updating for ImmunoScore diagnosis.
Little is known about the correlate of protection for pnuemococcal anti-capsular polysaccharide antibodies. It is likely that the protective IgG range would fall between 0.15 and 1.0 μg/mL, except for serotype 14, against which more antibody is necessary (Plotkin, 2001). An ImmunoScore diagnostic recommendation can, for example, initially be for a boost if antibody levels fell below 1.0 μg/mL, and then the database analyses can be able to shape future recommendations.
   B. Adult Diagnostic Panels
   1. Measurement of Immunity Induced By Vaccines for Military Personnel

Despite the tremendous strides that have been made in public health, the control of infectious diseases, and preventive medicine during the past century, infectious agents remain a substantial threat to the operational capacity of military forces at the onset of the new millennium for three distinct reasons:
new recruits are trained in groups under crowded conditions, increasing the risk of spread of infectious agents;
warfighters, as a result of deployments, may come into contact with pathogens with which they have no prior experience and, therefore, no immunity; and
warfighters, along with others, may face the intentional use of weaponized infectious agents.

Even in recent years, U.S. troops have been deployed to geographic regions where there exist endemic infectious disease agents against which the U.S. military does not have immediately available either suitable, safe, and effective vaccines or appropriate chemoprophylactic agents. Infectious diseases continue to contribute substantially to morbidity during deployments, as shown in FIG. 4C.

Immunization has long served as a key mode of prevention of infections in military populations. General George Washington ordered the first systematic immunization effort among American forces when he directed the variolation of Revolutionary War soldiers serving in the Continental Army to protect them from smallpox (Bayne-Jones, 1968). Military scientists and epidemiologists have historically played a key role not only in keeping soldiers healthy, but also in contributing to improvements in the general public health.

U.S. troops must be prepared to be deployed anywhere in the world, often on very short notice, whether it is for actual combat, for a training exercise, or to serve as peacekeepers. Given the political instabilities in many parts of the world, U.S. warfighters must be ready to be deployed into environments where the risk of exposure to infectious diseases may be significant. Deployments occur in areas with widely different climates and very different ecological and demographic settings, including, within just the past dozen years, the Caribbean, the Middle East, South-Central Asia, and the Western Pacific. Predicting the nature and magnitude of infectious disease risks in advance of deployments may not always be possible, but maintaining a high degree of awareness is mandatory, given the lessons of history and the clear benefit-to-cost ratio.

The U.S. Army Medical Research and Materiel Command (USAMRMC), a subordinate command of the U.S. Army Medical Command (MEDCOM), is charged with solving problems and providing the armed forces with solutions to these problems in the form of medical products; among these solutions are vaccines. USAMRMC's primary goal is to protect and sustain the health of the warfighter. To accomplish this goal, USAMRMC is “responsible for medical research, product development, technology assessment and rapid prototyping, medical logistics management, health facility planning, and medical information management and technology.

As part of its medical research and development charge, USAMRMC has the responsibility for managing research as well as product development related to, among other things, vaccines and therapeutic agents aimed at preventing and controlling naturally occurring infectious diseases that are perceived to threaten the operational effectiveness of the armed forces, However, USAMRMC does not manage the advanced development of vaccines against biological agents that may be weaponized; the Department of Defense (DoD) assigns that mission to the Joint Vaccine Acquisition Program (JVAP).

The DoD administers 17 different vaccines, as outlined in the Joint Instruction on Immunizations and Chemoprophylaxis, for the prevention of infectious diseases among military personnel, where appropriate. The vaccines are administered to military personnel on the basis of military occupation, the location of the deployment, and mission requirements.

In 1990, at the request of the DoD, FDA published an interim rule addressing the DoD's concerns about the use of products with IND status in combat situations. The interim rule allowed the FDA commissioner to waive the informed consent requirements when such a waiver was requested by the Assistant Secretary for Health Affairs. Application of the rule was restricted to the “use of an investigational drug (including an antibiotic or biological product) in a specific protocol under an investigational new drug application” and was “limited to a specific military operation involving combat or the immediate threat of combat.” The rule was applied during the Gulf War, allowing the use of pyridostigmine bromide and a botulinum toxoid vaccine to protect against the potential use of weaponized biological or chemical agents (Rettig, 1999).

When service members returned from the Gulf War deployment and reported medically unexplained symptoms, many questioned the safety and efficacy of the vaccine and drug products used during the war and the wisdom of DoD's use of the interim rule. These perceptions, which may have been different had there been credible evidence of the actual use of chemical or biological weapons by forces opposing U.S. and allied personnel, sparked changes in the government's use of the interim rule during the Gulf War, the U.S. Congress passed an amendment to the Defense Authorization Act for FY 1999 that vests solely with the president the authority to waive the informed consent requirement.

DoD was again criticized for administering a product with IND status without close adherence to the FDA guidelines when it used tick-borne encephalitis (TBE) vaccine in the Bosnian conflict. For many years, the military had administered the TBE vaccine to U.S. personnel who inspected military sites in the Soviet Union, where TBE is endemic. The vaccine, developed by scientists from Austria and the United Kingdom, had been widely used in Europe, but had not been licensed for use in the United States. In 1996, the Assistant Secretary of Defense for Health Affairs outlined, based on input provided by USAMRMC and the surgeons general, DoD policy regarding the use of a vaccine against TBE. The policy instructed that the TBE vaccine should be offered to “personnel at very high risk of tick exposure” and that it should not be used to routinely immunize all DoD personnel. DoD offered the TBE vaccine to soldiers deployed to areas in Bosnia known to be affected by TBE. To receive the vaccine, however, individuals had to volunteer to participate in a study of the IND product and, accordingly, to provide written informed consent.

An investigation by the General Accounting Office into the Army's record-keeping practices during the Bosnian conflict (GAO, 1997) found that nearly one-fourth of the immunizations against TBE in Bosnia were not properly documented. FDA, also, found “significant deviation” from the guidelines related to the use of a product with IND status in DoD's use of the TBE vaccine in Bosnia (FDA, 1997). Although DoD officials “acknowledged faulty recordkeeping,” they maintained that IND guidelines were followed (Gilbert, 1998). The TBE vaccine is no longer available to U.S. military personnel as a product with FDA IND status.

The sequence of events outlined above highlights the difficulties inherent in complying with FDA rules related to an IND product and conducting well-documented clinical trials of investigational vaccines among military personnel engaged in combat or participating in peacekeeping duties under hazardous conditions. They also point out the difficulties that commanders face when they must confront the rules and regulatory practices that are in place when they are deploying forces into situations that are likely to expose those forces to infectious disease threats for which licensed vaccines may not be available.

Currently, there are vaccines for six infectious diseases that are exclusive to military personnel. Those vaccines are for adenovirus serotypes 4 and 7, anthrax, cholera, Plague, smallpox, and Lyme disease. In addition, at any time, the military may be willing to use vaccines not yet approved by the FDA. The ImmunoScore diagnostic panel will be able to add analytes from these infectious agents for its military application.

Many of the special-use vaccines that were once licensed or used by the military as products with IND status are no longer available. This situation arises as a result of any of a variety of obstacles. For most vaccines that are products with IND status, there was simply insufficient funding for advanced development. For other products, it was deemed difficult, if not impossible, to demonstrate their effectiveness and safety in humans, thus preventing the possibility of their licensure. Market factors, such as inadequate sustained demand, are obstacles as are a lack of interest or monetary incentive for industry to participate in the development or scale-up of the production process, the lack of an adequate physical infrastructure to meet the regulatory requirements for manufacture of the vaccine, or the inability of manufacturers to meet other regulatory requirements.

Despite historic successes, in recent years DoD vaccine acquisition efforts have at times been troubled. This is best exemplified by the loss of the availability of adenovirus, plague, and anthrax vaccines. Although the circumstances contributing to the loss of the availability of each vaccine differ, each case illustrates the vulnerabilities inherent in the vaccine acquisition system.

In the 1960s and 1970s widespread adenovirus infections, especially those due to serotypes 4 and 7, plagued the armed forces basic training facilities throughout each winter-spring respiratory virus season, resulting in major morbidity and some mortality, overtaxed and overcrowded hospital facilities, and the loss of significant amounts of time from basic training as a result of recurrent explosive outbreaks. As a result, military research efforts were directed toward the development of serotype-specific vaccines. These vaccines were shown to be highly effective in clinical trials in the 1960s and early 1970s (Edmonson, et al. 1966; Top, et al. 1971) and became licensed in 1980. Administration of these oral, live encapsulated adenovirus types 4 and 7 vaccines to recruits on the first day of their arrival at a base rendered the outbreaks a thing of the past. After 25 years of successful use, discussions between DoD and the manufacturer failed to produce an agreement concerning improvements to the manufacturing facility that were required by the FDA. The sole manufacturer of the adenovirus vaccines stopped producing them in 1996, and the stock was totally depleted by mid-1999. Subsequently, adenovirus illness re-emerged as a major cause of illness and hospitalization among new trainees (Gray, et al. 1999; McNeil, et al. 1999; Sanchez, et al. 2001). Virus studies in 1999 and 2000 revealed that 82 percent of the infections were again due to types 4 and 7. Thousands of trainees have been affected, and as a result, many recruits must repeat their training because of time lost due to illness (Gray, et al. 2000). Three basic training facilities found their infirmary and hospital facilities overwhelmed and were forced to seek other accommodations for trainees requiring inpatient care. The deaths of at least two previously healthy recruits have been attributed to vaccine-preventable adenovirus infection (CDC, 2001).

The availability of the plague vaccine has also been interrupted. Plague vaccine, first manufactured in the United States in 1942, has mostly military, but some commercial applications. In a Sep. 22, 1997 warning letter to the manufacturer, the FDA outlined several significant deviations from FDA production guidelines in the manufacture of the plague vaccine. The manufacturer discontinued the vaccine in 1998 because “FDA requirements for further testing and validation of the product could not be financially justified, and the DoD was not able to fund further studies” (Greer Laboratories, 2001). Currently, plague vaccine is not available to protect U.S. armed forces.

Regardless of the current availability of vaccines, the ImmunoScore diagnostic panel for the military must include assays for vaccines specific for military personnel. It must be assumed that with the renewed emphasis on weapons of bioterror, and the need for rapid mobilization of the armed forces that facilitated approval for these vaccines will become a reality.

Anthrax presents in three clinical forms: cutaneous, gastrointestinal, and inhalation. The cutaneous form is the most common in natural exposure situations, with the lowest case fatality rates. The case fatality rates without antibiotic treatment ranges from 5-20%, and the case fatality rate in individuals treated with antibiotics is approximately 1%. Incidence of gastrointestinal anthrax is rare, with a case fatality rate estimated between 20 and 65%. Inhalation anthrax is the most deadly, and a cause for concern regarding bioterrorism, with case fatality rates of 75% (treated with antibiotics) and 86-97% (untreated). FIG. 4D shows the spike in reported anthrax cases in the United States following an act of bioterrorism, and reveals the need for increased surveillance.

The principle antigen responsible for producing immunity is protective antigen (PA). Approximately 95% of vaccinees seroconvert with a four fold rise in anti-PA IgG titers following three doses of vaccine. The correlation between antibody titer and protection against infection is not known with any degree of certainty. Certainly the ImmunoScore diagnostic database could help shed light onto the correlates of protection.

Cholera vaccine is administered to military personnel only upon travel or deployment to countries requiring cholera vaccination as a condition for entry, or upon the recommendation of the appropriate Surgeon General. At the present time, the manufacture and sale of the only licensed cholera vaccine in the United States (Wyeth) has been discontinued.

Plague is an acute, often fatal, and potentially epidemic disease caused by infection with Yersinia pestis. Plague vaccine (when it is available) is administered to personnel who are likely to be assigned to areas where the risk of endemic transmission or other exposure is high. The vaccine may not be effective in the prevention of airborne infection (CDC, 1996). The current licensed vaccine is a whole-cell vaccine. There are sub-unit vaccines under development (Williamson, et al. 2000).

Smallpox is caused by the variola virus. Variola virus infects only humans in nature, although primates and other animals have been infected in the laboratory. Vaccinia, cowpox, and monkeypox can infect both humans and other animals in nature. Some persons infected with variola virus have particularly severe illnesses. This suggests that there could be differences in the virulence of strains of the virus. However, no laboratory test has been devised that correlates with virulence in humans. Physiologic factors in the host are probably the more important determinant of severity of the illness. This is yet another opportunity to possibly apply ImmunoScore diagnostic applications toward further understanding of immune response to vaccine antigens. As the database grows, more and more detailed immunological information will be gleaned.

Smallpox vaccine contains vaccinia virus, not variola virus. Vaccinia is rarely isolated from animals outside the laboratory. There are multiple strains of vaccinia virus that have different levels of virulence for humans and animals. Vaccinia virus can also be genetically engineered to accept DNA and express other antigens, and has been used as a vector in laboratory experiments. Two clinical forms of smallpox have been described. While both forms are caused by the variola virus, they are caused by different strains of the virus distinguishable by specific biological properties. Variola major is the severe form of smallpox, with a more extensive rash, higher fever, and a greater degree of prostration. Variola major has a case fatality rate of 30%, or more. The last case of variola major occurred in Bangladesh in 1975. Variola minor was first described in South Africa and the United States in the late 19^{th} century. Variola minor is a much less severe disease, with a case fatality rate of 1%, or less. Variola minor was endemic in some countries of Europe and of North and South America, and in many parts of Africa. The last case of variola minor occurred in Somalia in October 1977, and was the last case of indigenous smallpox on earth.

The smallpox vaccine currently available in the United States is a live virus preparation of infectious vaccinia virus. Smallpox vaccine does not contain variola virus. The current vaccine was prepared in the early 1980s from calf lymph. Approximately 15 million doses of vaccine are available now in the United States. Testing has shown that existing supplies of vaccine could be diluted 1:5 and still remain effective and safe as full-strength vaccine. An additional 200 million additional doses of vaccine are being produced using cell culture methods to be available in case of an introduction of smallpox.

Neutralizing antibodies induced by vaccinia vaccine are genus-specific and cross-protective for other Orthopoxviruses (e.g. monkeypox, cowpox, and variola viruses). Neutralizing antibodies are detectable 10 days after primary vaccination, and 7 days after revaccination. Although the level of antibody that protects against smallpox infection is unknown, after percutaneous administration of a standard dose of vaccinia vaccine, >95% of primary vaccinees (i.e. persons receiving their first dose of vaccine) will develop neutralizing or hemagglutination inhibition antibody at a titer of >1:10. Neutralizing antibody titers of >1:10 persist among 75% of individuals for 10 years after receiving second doses and up to 30 years after receiving three doses of vaccine.

The efficacy of smallpox vaccine has never been measured precisely in clinical trials. However, protection has been determined in studies of people exposed to a smallpox patient in their household. These studies indicated a 91-97% reduction in smallpox among contacts with a vaccination scar compared to contacts without a scar. However, these studies did not always consider the time since vaccination or potency of vaccine, so may underestimate protection.

Epidemiologic studies demonstrated that a high level of protection (nearly 100%) against smallpox persists for up to 5 years after primary vaccination and substantial but waning immunity for ten years or more. Antibody levels after revaccination can remain high longer, conferring a greater period of immunity than occurs after primary vaccination alone. Although smallpox vaccination in the remote past may not completely protect against smallpox, vaccinated people appear to have less severe disease. Thus, in exemplary embodiments of the present invention, an ImmunoScore diagnostic panel could be able to evaluate levels of neutralizing antibody present in immunized individuals and an ImmunoScore database could add to the information regarding correlation of that antibody level to protection from disease. A recent study indicated persistence of a low level of B-cell response to smallpox in vaccinated individuals, as depicted in FIG. 4E (Crotty, et al. 2003). In addition, the longevity of antibody persistence could be measured in larger population samples.

Currently, the risk for smallpox occurring as a result of a deliberate release by terrorists is considered low. Therefore, pre-exposure vaccination is not recommended for any group other than laboratory or medical personnel. If an intentional release of smallpox virus does occur, vaccinia vaccine will be recommended for certain groups (CDC, 2001).

Lyme disease is a tick-borne zoonosis caused by infection with the spirochete Borrelia burgdorferi. These bacteria are transmitted to humans by the bite of infected deer ticks and caused more than 23,000 infections in the United States in 2002. In the United States, Lyme disease is mostly localized to states in the northeastern, mid-Atlantic, and upper north-central regions, and to several counties in northwestern California, as seen in FIG. 4F. Individuals who live or work in residential areas surrounded by tick-infested woods or overgrown brush are at risk of getting Lyme disease. Persons who work or play in their yard, participate in recreational activities away from home such as hiking, camping, fishing and hunting, or engage in outdoor occupations, such as landscaping, brush clearing, forestry, and wildlife and parks management in endemic areas may also be at risk of getting Lyme disease.

Lyme disease is a multisystem, multistage, inflammatory illness. In its early stages, Lyme disease can be treated successfully with oral antibiotics; however, untreated or inadequately treated infection can progress to late-stage complications requiring more intensive therapy. The first line of defense against Lyme disease and other tick-borne illnesses is avoidance of tick-infested habitats, use of personal protective measure, and checking for and removing attached ticks. Early diagnosis and treatment are effective in preventing late-stage complications.

The licensed Lyme disease vaccine is made from lipidated rOspA of B. burgdorferi, expressed in E. coli and purified. The vaccine was demonstrated to be safe and effective; however, anecdotal complaints of the vaccine's safety, in particular related to treatment-resistant arthritic disorders, were abundant. The vaccine was withdrawn from the market by the manufacturer in February 2002 because of low sales and is no longer commercially available.

The application of ImmunoScore diagnostic analyses to the immune status of individuals in the military can be of great benefit to both the military personnel and administration, and also to the expansion of an ImmunoScore system database. The flexibility built into diagnostic panels according to the present invention will be crucial to military applications. Vaccine requirements vary from service to service and also depend on the area of deployment. In times of rapid deployment, an ImmunoScore immune status diagnosis can, for example, give real time information regarding an individual soldier's immune status to a vast array of possibly infectious diseases. In addition, the possibly detrimental redundancy of vaccination can be eliminated in many of the military personnel, providing optimum coverage without fear of immunosuppression due to hyperimmunization.

In exemplary embodiments of the present invention military personnel can be administered the following diagnostic panels:

1. College Student ImmunoScore Panels consisting of:
Meningococcal Diagnostic Panel;
Persistent Immunity Induced by Childhood Vaccine Diagnostic Panel; and
Sexually Transmitted Disease Diagnostic Panel,
   as described above; and

2. In addition, military personnel can have specific vaccination needs as outlined in Table 3 below depending on their assignments and type of deployment. Specific branches of the service may also have specific vaccination needs and permutations of the basic diagnostic panels. Thus, in exemplary embodiments, military personnel can be administered one or more of the following tests:

Vaccine Diagnostic Panels Exclusive to the Military:

| Vaccine | Antibody Marker |
|---|---|
| Adenovirus 4 & 7 | Neutralizing antibody |
| Anthrax | PA |
| Cholera | LPS IgG |
| Plague | Fraction I Capsular Antigen |
| Smallpox | Neutralizing antibody |
| Lyme disease | OspA |

In addition to the analytes listed above as exclusive to the military, an ImmunoScore diagnostic panel can be extremely flexible at adding new diagnostic tests for vaccines under development.

Analysis of results/recommendations for use of ImmunoScore diagnostic panel data for analytes for specific military applications can, in exemplary embodiments of the present invention, include the following:
Adenovirus vaccine is not currently given to military recruits, but infection with adenovirus remains a concern. Development and use of adenovirus vaccines are likely in the future and an exemplary ImmunoScore diagnostic application can require an updated assay format over the currently accepted neutralizing antibody assay.
Currently, serological correlates of protection to inhalation anthrax are being developed in animal models (Little, et al. 2004). ImmunoScore diagnostics can, for example, measure level of serum IgG to protective antigen (PA) and the ImmunoScore database can, for example, thus build serologic correlates of protection in humans.
Immunity to cholera is currently not completely understood (Cohen, et al. 2002). ImmunoScore diagnostics can focus first on levels of anti-LPS IgG, and further attempt to build meaning into the database correlates of protection.
The need for a new vaccine for pneumonic plague is evident given the limited efficacy of the current cellular vaccines, which consist of either the killed virulent 195/P or live EV76 strains (Titball and Williamson, 2001). While an efficient and safe live cellular vaccine has not been identified yet, there is an effort to develop alternative subunit vaccines based on various antigens, including the F1 and V antigens (Flashner, et al. 2004). ImmunoScore diagnostics can, for example, monitor serum antibody levels to current plague vaccine components and be able to adapt to any new vaccine configurations. ImmunoScore database can compile immune response data and be correlate the relevant antibody levels to levels of protection.
Immune memory after smallpox vaccination is a valuable benchmark for understanding the kinetics and longevity of B cell memory in the absence of re-exposure to antigen, since immunization of the U.S. population was stopped in 1972 and smallpox disease was declared eradicated worldwide in 1980 (Fenner, et al. 1988). Immune memory to smallpox is a useful benchmark both for understanding the longevity and the stability of immune memory in the absence of re-stimulation. Circulating antibody persists for over 50 years (Crotty, et al. 2003). ImmunoScore diagnostics can, in exemplary embodiments, measure antibody to smallpox. Correlates of protection can be generated from analyses of the ImmunoScore database.

The human protective response to vaccination against Lyme disease is purely a serum-mediated antibody response. Individuals are considered protective with antibody levels against OspA greater than 1100 IU. Subjects with less antibody titers less than 1100 would, in exemplary embodiments of the present invention, be recommended to have a booster vaccination.

TABLE 3
Vaccines Typically Administered to U.S. Military Personnel
(U.S. Army, U.S. Navy, U.S. Marine Corps,
U.S. Air Force, U.S. Coast Guard)

| | | Routine |
|---|---|---|
| | | Schedule for |
| Timing | Vaccine | Basic Immunity** |
| Recruits and officer | Adenovirus 4 & 7*** | Single dose |
| accessions | Diphtheria | Every 10 years |
| | Hepatitis A* | Two doses |
| | Influenza | Annual |
| | Measles | Single dose |
| | Meningococcal disease* | Single dose |
| | Mumps* | Single dose |
| | Poliovirus | Single dose |
| | Rubella | Single dose |
| | Tetanus | Every 10 years |
| | Varicella* | Two doses |
| | Yellow fever* | One dose |
| Routine during career | Diphtheria | Every 10 years |
| (active duty and reserve | Influenza | Annual |
| component) | Tetanus | Every 10 years |
| Alert forces and forces | Anthrax | Six-dose series |
| deploying or traveling to | Cholera*** | Two doses |
| high risk areas* | Hepatitis A | Two doses |
| | Hepatitis B* | Three doses |
| | Japanese encephalitis | Three doses |
| | Meningococcal disease | Single dose |
| | Plague*** | Three doses |
| | Poliovirus | One dose |
| | Rabies* | Three doses |
| | Smallpox | Every 10 years |
| | Typhoid | One to two doses |
| | Yellow fever | Single dose |
| Individual according to | Hib | Single dose |
| occupational or personal | Hepatitis B | Three doses |
| needs | Lyme disease | Three doses |
| | Meningococcal disease | Single dose |
| | Pneumococcal disease | Single dose |
| | Rabies | Three doses |
| | Varicella | Two doses |

*Vaccination policy varies among Military Services.
**Booster doses may be required at annual or other intervals to sustain immunity.
***Vaccine seldom used and/or supply is limited.
Primary Source: Air Force Joint Instruction 48-110/United States Army Regulation 40-562/Navy Bureau of Medicine Instruction 6230.15/Coast Guard Commandant Instruction M6230.4E. Immunizations and Chemoprophylaxis. Washington, D.C. Nov. 1, 1995.

2. ImmunoScore Measurement of Immunity Induced By Vaccines for Travelers

The Centers for Disease Control and Prevention (CDC) has a National Center for Infectious Disease dedicated to travelers' health. This center has designated 17 specific destination regions as shown in Table 4. A different regimen of vaccines is recommended by the CDC depending on the region to be visited. Foreign countries might also have vaccination requirements for visitors. The ImmunoScore Diagnostic panel can be used, for example, to screen travelers prior to their departure. Travelers could be assured of their immune status regarding required immunizations, and at the same time, if their antibody levels were adequate, would not be required to undergo unnecessary re-immunizations. Knowledge of the persistence of humoral response acquired through the database would also be invaluable to researchers and members of the ACIP.

Hepatitis A is a viral infection of the liver caused by the hepatitis A virus (HAV). The clinical manifestations of HAV infection range n clinical severity from no symptoms to a mild illness lasting 1-2 weeks to a severely disabling disease lasting several months. Clinical manifestations of hepatitis A often include fever, malaise, anorexia, nausea, and abdominal discomfort, followed within a few days by jaundice.

The risk of acquiring HAV infection for U.S. residents traveling abroad varies with living conditions, length of stay, and the incidence of HAV infection in the area visited. Travelers to North America (except Mexico), Japan, Australia, New Zealand, and developed countries in Europe are at no greater risk for infection than in the United States. For travelers to low-income countries, risk for infection increases with duration of travel and is highest for those who live in or visit rural areas, trek in the back country areas, or frequently eat or drink in settings of poor sanitation. Nevertheless, many cases of travel-related hepatitis A occur in travelers to developing countries with “standard” tourist accommodations, and food consumption behaviors.

Hepatitis A is one of the most common vaccine-preventable diseases in travelers. Hepatitis A vaccine, immune globulin (IG), or both, are recommended for all susceptible individuals traveling to or working in countries with an intermediate or high endemicity of HAV infection. Two monovalent hepatitis A vaccines are currently licensed in the United States for persons≧2 years of age.

It is recommended that the first dose of hepatitis A vaccine should be administered as soon as travel to countries with high or intermediate endemicity is considered. One month after receiving the first dose of monovalent hepatitis A vaccine, 94-100% of adults and children will have protective concentrations of antibody. The final dose in the hepatitis A vaccine series is necessary to promote long-term protection.

Although vaccination of an immune traveler is not currently contraindicated and does not increase the risk of adverse effects, screening for total anti-HAV before travel can be useful in some circumstances to determine susceptibility and eliminate unnecessary vaccination or IG prophylaxis of immune travelers. The CDC states that such serologic screening for susceptibility might be indicated for adult travelers who are likely to have had prior HAV infection if the cost of screening (laboratory and office visit) is less than the cost of the vaccination or IG prophylaxis and if testing will not delay vaccination and interfere with timely receipt of vaccine or IG prior to travel.

In adults and children, who have completed the vaccine series, anti-HAV has been shown to persist for at least 5-8 years after vaccination. Results of mathematical models indicate that after completion of the vaccination series, anti-HAV will likely persist for 20 years or more. For children and adults who complete the primary series, booster doses of vaccine are not recommended.

Hepatitis B is caused by the hepatitis B virus (HBV). The clinical manifestations of HBV infection range in severity from no symptoms to fulminant hepatitis. Signs and symptoms of hepatitis B may include fever, malaise, anorexia, nausea, and abdominal discomfort, followed within a few days by jaundice. HBV is transmitted through activities that involve contact with blood or blood-derived fluids.

The prevalence of chronic HBV infection is low (<2%) in the general population in Northern and Western Europe, North America, Australia, New Zealand, Mexico, and southern South America. In the United States and many other developed countries, children and adolescents are routinely vaccinated against hepatitis B. The highest incidence of disease is in younger adults, and most HBV infections are acquired through unprotected sex with HBV-infected partners or through illicit injection drug use. The prevalence of chronic HBV infection is intermediate (2-7%) in South Central and Southwest Asia, Israel, Japan, Eastern and Southern Europe, Russia, most areas surrounding the Amazon River basin, Honduras, and Guatemala. The prevalence of chronic HBV infection is high (>8%) in all socioeconomic groups in certain areas: all of Africa; Southeast Asia, including China, Korea, Indonesia, and the Philippines; the Middle East, except Israel; south and Western Pacific islands; the interior Amazon River basin; and certain parts of the Caribbean (Haiti and the Dominican Republic).

The risk of HBV infection for international travelers is generally low, except for certain travelers in countries where the prevalence of chronic HBV infection is high or intermediate. Factors to consider in assessing risk include 1) the prevalence of chronic HBV infection in the local population, 2) the extent of direct contact with blood or secretions, or of sexual contact with potentially infected individuals, and 3) the duration of travel.

Hepatitis B vaccination should be administered to travelers to areas with intermediate to high levels of endemic HBV transmission and who will have close contact with the local population.

In particular, travelers who anticipate sexual contact or who will have daily physical contact with the local population; or who are likely to seek medical, dental, or other treatment in local facilities; or any combination of these activities during their stay should be advised to receive the vaccine. Hepatitis B vaccination is currently recommended for all United States residents who work in health-care fields (medical, dental, laboratory, or other) that entail exposure to human blood. Two monovalent vaccines are currently licensed in the U.S.

Ideally, vaccination should begin at least 6 months prior to travel so the full vaccine series can be completed before departure. Because some protection is provided by one or two doses, the vaccine series should be initiated, if indicated, even if it cannot be completed before departure. Optimal protection, however, is not conferred until after the final vaccine dose. There is no interference between hepatitis B vaccine and other simultaneously administered vaccine(s) or with IG.

Japanese encephalitis (JE) is a common mosquito-borne viral encephalitis found in Asia. Most infections are asymptomatic, but if clinical illness develops, the case-fatality rate can be as high as 30%. Neuropsychiatric sequelae are reported in 50% of survivors. In endemic areas, children are at the greatest risk of infection; however, multiple factors such as occupation, recreational exposure, gender, previous vaccination, and naturally acquired immunity alter the potential for infection and illness. A higher case-fatality rate is reported in the elderly, but serious sequelae are more frequent in the very young, possibly because they are more likely to survive a serious infection.

JE is transmitted chiefly by the bites of mosquitoes in the Culex vishnui complex; the vector species depends on the specific geographic area. This species feeds outdoors beginning at dusk and during evening hours until dawn; it has a wide host range, including domestic animals, birds, and humans. Larvae are found in rice fields, marshes, and small stable collections of water around cultivated fields. In temperate zones, the vectors are present in greatest numbers from June through September and are inactive during winter months. Swine and certain species of wild birds function as viremic amplifying hosts in the transmission cycle. Habitats supporting the transmission cycle of JE virus are principally in rural, agricultural locations. In many areas of Asia, however, the appropriate ecologic conditions for virus transmission occur near or occasionally within urban centers. Transmission is seasonal and occurs in the summer and autumn in the temperate regions of China, Japan, Korea, and eastern areas of Russia. The risk to short-term travelers and those who confine their travel to urban centers is very low. Expatriates and travelers living for prolonged periods in rural areas where JE is endemic or epidemic are at greatest risk. Travelers with extensive unprotected outdoor, evening, and nighttime exposure in rural areas, such as might be experienced while bicycling, camping, or engaging in certain occupational activities, might be at high risk even if their trip is brief.

The recommended primary immunization series is three doses, administered over a 30 day schedule. The full duration of protection is unknown; however, preliminary data indicate that the neutralizing antibodies persist for at least two years after primary immunization. Vaccination should be considered only by persons who plan to live in areas where JE is endemic or epidemic and by travelers whose activities include trips into rural farming areas. Evaluation of an individual traveler's risk should take into account his or her itinerary and the current level of JE activity in the country. ImmunoScore diagnostic evaluation would be valuable to JE immunization considerations in several respects. The most obvious is the measurement of protective serum antibody level. The other is to evaluate an individual's travel plan and include up-to-date information regarding current risk of JE infection in many areas around the globe. The CDC keeps up-to-date information regarding risk areas and those data could be an integral part of ImmunoScore diagnostic recommendations for travelers.

Meningococcal disease is an acute bacterial disease characterized by sudden onset of fever; intense headache; nausea and often vomiting; stiff neck; and, frequently, a petechial rash with pick macules. Up to 10% of populations in countries with endemic disease carry the bacteria asymptomatically in their nose and throat.

In sub-Saharan Africa, peaks of serogroup A meningococcal disease occur regularly during the dry season (December through June). In addition, major epidemics occur every 8-12 years, particularly in the savannah areas extending from Mali eastward to Ethiopia, known as the “meningitis belt.” Travelers to sub-Saharan Africa may be at risk for meningococcal disease. Because of the lack of established surveillance and timely reporting from many of these countries, travelers to the meningitis belt during the dry season should be advised to receive meningococcal vaccine, especially if prolonged contact with the local population is likely.

Vaccination against meningococcal disease is not a requirement for entry into any country, except Saudi Arabia, for travelers to Mecca during the annual Hajj. Vaccination is indicated for travelers to countries recognized as having epidemic meningococcal disease caused by a vaccine-preventable serogroup during the dry season. Advisories for travelers to other countries will be issued when epidemics of meningococcal disease caused by vaccine-preventable serogroups are recognized. These advisories are posted at the CDC Traveler's Health website at http://www.cdc.gov/travel/outbreaks.htm. The expanded ImmunoScore meningococcal diagnostic panel would be very useful in identifying individual travelers at increased risk due to genetic immunodeficiencies.

Rabies, an acute, fatal encephalomyelitis caused by neurotropic viruses, is almost always transmitted by an animal bite that inoculates the virus into wounds. Very rarely, rabies has been transmitted by non-bite exposures that introduce the virus into open wounds or mucous membranes. All mammals are believed to be susceptible, but reservoirs are carnivores and bats.

Although dogs are the main reservoir in resource-poor countries, the epidemiology of the disease differs sufficiently from one region or country to another to warrant the medical evaluation of all mammal bites.

Travelers to rabies-endemic countries should be warned about the risk of acquiring rabies, although rabies vaccination is not a requirement for entry into any country. Travelers with extensive unprotected outdoor, evening, and nighttime exposure in rural areas, such as might be experienced while bicycling, camping, or engaging in certain occupational activities, might be at high risk even if their trip is brief.

Pre-exposure vaccination with human diploid cell rabies vaccine (HDCV), purified chick embryo cell (PCEC) vaccine, or rabies vaccine adsorbed (RVA) may be recommended for international travelers based on the local incidence of rabies in the country to be visited, the availability of appropriate anti-rabies biologicals, and the intended activity and duration of stay of the traveler. Pre-exposure vaccination may be recommended for veterinarians, animal handlers, field biologists, spelunkers, missionaries, and certain laboratory workers. Pre-exposure vaccination does not eliminate the need for additional medical attention after rabies exposure, but simplifies post-exposure prophylaxis in populations at risk by eliminating the need for rabies immune globulin (RIG) and by decreasing the number of doses of vaccine required. Pre-exposure vaccination is of particular importance for travelers at risk of exposure to rabies in countries where locally available rabies vaccines might carry a high risk of adverse reactions. Pre-exposure vaccination can also provide protection when there is an unapparent or unrecognized exposure to rabies and when post-exposure prophylaxis might be delayed.

Routine serologic testing is not necessary for travelers who receive the recommended pre-exposure or post-exposure regiment with HDCV, PCEC, or RVA vaccines. Serologic testing is still recommended for travelers whose immune response might be diminished by drug therapy or by diseases. Approximately 6% of persons receiving booster vaccinations with HDCV can experience an immune complex like reaction characterized by urticaria, pruritus and malaise. Although antibody levels do not define an individual's immune status, they are a marker of continuing immune response. To ensure the continuity of an immune response, titers should be checked periodically, with booster doses administered as needed. Two years after primary pre-exposure vaccination, a 1:5 serum dilution will neutralize challenge virus completely among 93-98% of individuals who received the three-dose pre-exposure series. If the titer falls below the minimum acceptable antibody level, a pre-exposure booster dose of vaccine is recommended for an individual at continuous or frequent risk for exposure to rabies (Tables 5-6). Currently, state or local health departments can provide the names and addresses of laboratories performing rabies serologic testing. The ImmunoScore diagnostic application can, for example, provide serologic testing at many sites and expand the database regarding duration of antibody protection following vaccination.

Typhoid fever is an acute, life-threatening febrile disease caused by the bacterium Salmonella enterica Typhi. An estimated 16 million cases of typhoid fever and 600,000 related deaths occur worldwide each year. Typhoid vaccination is not required for international travel, but it is recommended for travelers to areas where there is a recognized risk of exposure to S. Typhi. Risk is greatest for travelers to the Indian subcontinent and other low-income countries (in Asia, Africa, and Central and South America) who will have a prolonged exposure to potentially contaminated food and drink. Vaccination if particularly recommended for those who will be traveling in smaller cities, villages, and rural areas off the usual tourist itineraries. Travelers should be cautioned that typhoid vaccination is not 100% effective and is not a substitute for careful selection of food and drink. The hallmark of infection is persistent high fevers. Other common symptoms and signs include headache, malaise, anorexia, splenomegaly, and relative bradycardia. Many mild and atypical infections occur.

Two typhoid vaccines are currently available for use in the United States: an oral, live, attenuated vaccine and a Vi capsular polysaccharide vaccine. Both vaccines have been shown to protect 50-80% of recipients. Live, attenuated vaccine should not be given go immunocompromised travelers, including those infected with human immunodeficiency virus (HIV). The capsular polysaccharide vaccine presents theoretically safer alternatives for this group. Theoretical concerns have been raised about the immunogenicity of live, attenuated vaccine in individuals concurrently receiving antibiotics, immune globulin, or viral vaccines. ImmunoScore diagnostics would be a valuable tool to assess immune response to either vaccine as well as duration of protective antibody.

Yellow fever is a mosquito-borne viral disease. Illness ranges in severity from an influenza-like syndrome to severe hepatitis and hemorrhagic fever. Yellow fever is caused by a zoonotic virus that is maintained in nature by transmission between non-human primates and mosquito vectors. In some situations, humans may serve as the primary host in the transmission cycle (“urban yellow fever”). The disease occurs only in sub-Saharan Africa and tropical South America where it is endemic and intermittently epidemic. In Africa, a variety of vectors are responsible for the disease, and it is in Africa where most of the cases are reported. The case fatality rate is >20%, and infants and children are at the greatest risk for infection. In South America, cases occur most frequently in young men who have occupational exposure to mosquito vectors in forested or transitional areas of Bolivia, Brazil, Columbia, Ecuador, Venezuela, and Peru.

The risk of a traveler acquiring yellow fever is determined by immunization status, geographic location, season, and duration of exposure, occupational and recreational activities while traveling, and the rate of yellow fever virus transmission at the time. Although reported cases of human disease are the principle guide to the level of transmission, they may be absent (because of a high level of immunity in the population) or not detected as a result of poor surveillance. Only a small proportion of yellow fever cases are officially noted, because of the occurrence of the disease in remote areas and lack of specific diagnostic facilities. Indeed, the majority of cases during outbreaks in Africa are missed despite an extraordinarily high incidence of infection and disease. During interepidemic periods, the incidence of overt disease is below the threshold of detection by existing surveillance. Such interepidemic conditions may last years or even decades in certain countries or regions. This “epidemic silence” may provide a false sense of security and lead to travel without the benefit of vaccination. The incidence of yellow fever in South America is lower than that in Africa because virus transmission between monkeys and mosquitoes occurs in the canopy of the forest, isolated from human contact, and because immunity in the indigenous human population is high. In West Africa, the most dangerous time of year is during the late rainy and early dry seasons (July-October). Virus transmission is highest during the rainy season (January-March) in Brazil.

The low incidence of yellow fever, generally a few hundred cases per year, has led to complacency among travelers. In addition to vaccination, travelers should be advised to take precautions against exposure to mosquitoes when traveling in areas with yellow fever transmission.

Yellow fever is preventable by a relatively safe, effective vaccine. International regulations require proof of vaccination for travel to and from certain countries. For purposes of international travel, vaccines produced by different manufacturers must be approved by the World Health Organization and administered at an approved yellow fever vaccination center. State and territorial health departments have authority to designate non-federal vaccination centers; these can be identified by contacting state or local health departments. Vaccinees should receive a completed International Certificate of Vaccination, signed and validated with the center's stamp where the vaccine was given. This certificate is valid 10 days after vaccination and for a subsequent period of 10 years. Rather than rely on the International Certificate, the ImmunoScore diagnostic system could definitively quantitate the amount anti-yellow fever antibody and thereby reduce the risk of unnecessary vaccination. Also, if someone were to misplace their Certificate during a 10 year period, an ImmunoScore analysis can be used to certify that individual's immunity and can thus be used instead of the Certificate.

A new serious adverse reaction syndrome has recently been described among recipients of yellow fever vaccines. This syndrome was previously reported as febrile multiple organ system failure and is now called yellow fever vaccine-associated viscerotropic disease. The reports of this disease described patients with severe multiple organ system failure. Yellow fever vaccines must be considered as a possible, but rare, cause of yellow fever vaccine-associated viscerotropic disease that is similar to fulminant yellow fever caused by wild-type yellow fever virus.

Because of recent reports of deaths from yellow fever among unvaccinated travelers to areas endemic for yellow fever and of these reports of vaccine-associated viscerotropic disease, yellow fever vaccination of travelers to high-risk areas should be encouraged as a key prevention strategy, however, physicians should be careful to administer yellow fever vaccine only to persons truly at risk for exposure to yellow fever. Additional surveillance to better monitor and quantify yellow fever vaccine-specific adverse outcomes should be established. This is another excellent example of where the ImmunoScore diagnostic analysis can be of tremendous importance.

Infection with yellow fever virus poses a theoretical risk for travelers with immunosuppression in association with AIDS or other manifestations of HIV infection; leukemia, lymphoma, or generalized malignancy; or with the administration of corticosteroids, alkylating drugs, antimetabolites, or radiation. Such patients should not be vaccinated. It is possible that an ImmunoScore diagnostic analysis of an individual's personal immune status may reveal other individuals that should not be vaccinated, or conversely, individuals that need be vaccinated with greater regularity.

In general, the ImmunoScore system and methodologies allow persons to be vaccinated when and only when they truly need it. This is a significant advance over simply following generalized schedules which may address a populations needs as a whole, but can at best be only an approximation for each individual in the population.

Diphtheria is an acute bacterial disease involving primarily the tonsils, pharynx, larynx, nose, skin, and occasionally other mucous membranes. The characteristic lesion is marked by a patch or patches of an adherent grayish membrane with a surrounding inflammation.

Diphtheria remains a serious disease throughout much of the world. In particular, large outbreaks of diphtheria occurred in the 1990s throughout Russia and the independent countries of the former Soviet Union. Most life-threatening cases occurred in unvaccinated or inadequately immunized individuals. Travelers to disease-endemic areas may be at risk for exposure to toxigenic strains of Corynebacterium diphtheriae when travel is for extended periods, when there is contact with children, or when conditions are crowded or foster sharing of respiratory secretions.

Tetanus is an acute disease characterized by muscle rigidity and painful spasms, often starting in the muscles of the jaw and neck. The disease is caused by neurotoxin produced by anaerobic tetanus bacilli growing in contaminated wounds. Lesions that are considered “tetanus prone” are wounds contaminated with dirt, feces, or saliva, deep wounds, or those with necrotic tissue. However, tetanus has been associated with apparently clean superficial wounds, surgical procedures, insect bites, dental infections, chronic sores and infections, and intravenous drug use. In 5-10% of reported cases in the United States, no antecedent wound was identified.

Tetanus is a global health problem since Clostridium Tetani spores are ubiquitous. The disease occurs almost exclusively in individuals who are unvaccinated or inadequately immunized.

Pertussis is an acute bacterial disease involving the respiratory tract, characterized by prolonged paroxysmal coughing. Individuals in all age groups can be infected. Complications and deaths from pertussis are most common among infants.

Pertussis is severe primarily in children; it is often associated with complications and has a relatively high case-fatality ration in unvaccinated infants. Pertussis can also occur in adolescents and adults after immunity from vaccines has waned. Pertussis is highly communicable and is common, particularly in countries where vaccination is not generally provided.

It is assumed that protective levels of antibody for protection against diphtheria, tetanus, and pertussis would be present in individuals with a full series of DTP vaccinations for these diseases. In fact, immunity may wane for all three of these conditions, and to assess travelers for immunity to these diseases can be an important ImmunoScore function. Lacking an immunization recommendation, it would also be an ideal time to assess a great number of individuals (travelers) to build upon the database as well. There is a wide range of ages of individuals that travel. Information gathered amongst traveling individuals to the ImmunoScore database can draw from this wide array of ages.

Measles is an acute, highly communicable viral disease with prodromal fever, conjunctivitis, coryza, cough, and Koplik spots on the buccal mucosa. A characteristic red blotchy rash appears around the third day of the illness, beginning on the face and becoming generalized. Measles is frequently complicated by middle ear infection or diarrhea. The disease can be severe, with bronchopneumonia or brain inflammation leading to death in approximately 2 of every 1,000 cases. The risk of exposure to measles outside the United States could be high. Measles remains a common disease in many countries of the world, including some developed countries in Europe and Asia.

Measles vaccine contains live, attenuated measles virus. It is available as a single-antigen preparation or combined with live, attenuated mumps or rubella vaccines, or both. Although vaccination against measles, mumps, or rubella is not a requirement for entry into any country (including the United States), individuals traveling abroad should ensure that they are immune to all three diseases. In general, travelers can be considered immune to measles if they have documentation of physician-diagnosed measles, laboratory evidence of measles immunity, or proof of receipt of two doses of live measles vaccine on or after their first birthday. Most individuals born before 1957 are likely to have had measles disease and generally need not be considered susceptible.

Replication of vaccine viruses can be prolonged in persons who are immunosuppressed or immunodeficient for any reason (e.g. who have congenital immunodeficiency, HIV infection, leukemia, lymphoma, or generalized malignancy, or who are receiving therapy with alkylating agents, antimetabolites, radiation, or large doses of corticosteroids). Evidence based on case reports has linked infection with measles vaccine virus to subsequent death in six severely immunosuppressed individuals. For this reason, persons who are severely immunocompromised for any reason should not be given MMR vaccine. Healthy, susceptible close contacts of severely immunosuppressed persons may be vaccinated. Individuals receiving large daily doses of corticosteroids for 14 days or more should not receive MMR vaccine because of concern about vaccine safety. MMR and its component vaccines should be avoided for at least one month after cessation of high-dose therapy.

Measles disease can be severe in individuals with HIV infection. Available data indicate that vaccination with MMR has not been associated with severe or unusual adverse events in HIV-infected individuals without evidence of severe immunosuppression, although antibody responses have been variable. A theoretical risk of an increase in HIV viral load after MMR vaccination exists because such an effect has been observed with other vaccines. The clinical significance of such an increase is not known. MMR and other measles-containing vaccines are not recommended for HIV-infected persons with evidence of severe immunosuppression (e.g. a very low CD4⁺ T lymphocyte count), primarily because of the report of a case of measles pneumonitis in a measles vaccine recipient who had an advanced case of AIDS.

In exemplary embodiments of the present invention, ImmunoScore diagnostic analyses can reasonably be applied to all components of the MMR vaccine for travelers as previously described above in the Persistent Immunity to Childhood Vaccines section of the College Student panel. This can be most useful for the measles component of this vaccine. Since the MMR components are available as monovalent vaccines, it is reasonable to boost only for those diseases in which protection was not achieved.

Poliomyelitis is an acute infection that involves the gastrointestinal tract and, occasionally, the central nervous system. It is acquired by fecal-oral transmission. In the pre-vaccine era, infection with poliovirus was common, with epidemics occurring in the summer and fall in temperate areas. The incidence of poliomyelitis declined rapidly after the licensure of inactivated polio vaccine in 1955 and oral polio vaccine in 1960. The last cases of indigenously acquired polio in the United States occurred in 1979. The global polio eradication initiative has reduced the number of reported polio cases worldwide by >99% since the mid-1980s and the worldwide eradication of the disease appears feasible in the near future.

Travelers to countries where polio is epidemic or still endemic should be fully immunized.

Because of polio eradication, the number of countries where travelers are at risk for polio has decreased dramatically. Concurrent with the decline in polio incidence, the number of polio-endemic countries decreased from >120 in 1988 to approximately 10 in 2001. Most of the world's remaining poliovirus transmission is in five countries: Afghanistan, India, Pakistan, Nigeria, and Niger.

Clinical manifestations of poliovirus infections range from asymptomatic (the majority of infections) to symptomatic, including acute flaccid paralysis of a single limb to quadriplegia, respiratory failure, and rarely, death. An individual is considered to be fully immunized if he or she has received a primary series of inactivated poliovirus vaccine (IPV), live oral poliovirus (OPV), or four doses of any combination of IPV and OPV. To eliminate the risk of vaccine-associated paralytic poliomyelitis, OPV is no longer recommended for routine immunizations in the United States as of Jan. 1, 2000.

Administration of IPV to immunodeficient travelers is safe, and IPV is the only polio vaccine recommended for use in immunodeficient travelers and their household contacts. Although a protective immune response cannot be ensured, IPV might confer some protection to the immunodeficient individual. Individuals with primary immunodeficiency should avoid contact with excreted polio vaccine virus (e.g. as may occur with a child vaccinated with OPV within the previous six weeks).

The CDC recommends that adults who are traveling to polio-endemic areas and are unvaccinated or whose vaccination status is unknown should receive IPV. The duration of IPV protection against poliomyelitis is not currently known. The ImmunoScore diagnostic panel would not only be able to inform traveling individuals of their immune status regarding poliovirus, but would also be able to compile information to the database regarding the duration of protection provided by an IPV schedule that excluded OPV immunizations.

In exemplary embodiments of the present invention, an ImmunoScore Travelers Diagnostic Panel can comprise tests for all diseases of particular risk to travelers. The tests drawn from the panel can rely heavily on the itinerary of the individual traveler. If the traveler were interested as to his or her immune status for any other of the diseases in particular that he or she has been vaccinated for, those assays could also be included in a more comprehensive analysis. The traveler component to the ImmunoScore diagnostic database could potentially be a large addition. It can also be a highly valuable one to use as an overall assessment of the immune status of a growing number of entrants to the database.

The CDC regularly updates the vaccine recommendations for travelers depending on the endemic and epidemic disease conditions in travel destinations. A necessary coordination must exist between testing sites and the updated CDC list of recommendations. An ImmunoScore Diagnostic system can well provide a necessary means to update physicians and health care providers as to the most up-to-date recommendations of the CDC regarding travelers and vaccinations required by them.

Thus, in exemplary embodiments of the present invention, tests for ImmunoScore measurement of traveler's immunity can, for example, include the following:

Antibody to HAV (1)

Antibody to HBs (1)

Antibody to Japanese Encephalitis (1)

Antibody to rabies (1)
other rabies related cytokine assays (as necessary)

Antibody to Typhoid fever (1)

Antibody to yellow fever (1)

Antibody to diphtheria toxin (1)

Antibody to tetanus toxin (1)

Pertusis antibodies (4):
Antibody to pertussis toxin (PT)
Antibody to pertactin (PRN)
Antibody to filamentous hemagglutinin (FHA)
Antibody to fimbriae

Antibodies to poliovirus serotypes P1, P2, and P3 (3)

Antibody to measles (1)

Antibody to mumps (1)

Antibody to rubella (1)

In exemplary embodiments of the present invention, recommendations for use of a ImmunoScore diagnostic panel for analytes specific to travelers can include all of the uses of the results of the Meningococcal Diagnostic Panel tests, as described above. Additionally, the following recommendations/conclusions can be implemented:
The protective level of antibody to hepatitis A has been established to be approximately 10 mIU/mL if that concentration is maintained for a two month period, although some individuals may be protected at much lower concentrations (Conrad and Lemon, 1987). An individual that had less than 10 mIU/mL of antibody to hepatitis A can be recommended for vaccination.
Current analyses of antibody levels to Japanese Encephalitis consist of neutralization assays. These assays would need to be refined for ImmunoScore diagnostic applications. In exemplary embodiments of the present invention, an ImmunoScore database can catalog antibody levels in anticipation of establishing future serologic correlates of protection.
The most important immune response to rabies vaccines is antibody to the G envelope protein (Wicktor, et al. 1973), and passively administered antibody is part of standard treatment to neutralize cell-free virus before it attaches to the axon of a neuron (Plotkin, 2000). Because passive antibody alone is poorly effective unless supplemented by active vaccination, CD4+ and CD8+ cell responses are probably also important to protection, but whether these critical responses relate to cytotoxic T lymphocyte function, interferon synthesis or other cytokines is unknown (Hemachudha, et al. 1999). ImmunoScore diagnostic assays can include at the very least antibody levels to G envelope protein and can also measure relevant cytokines to assess serological correlates of protection.
Protection against typhoid fever might be best achieved by a vaccine that stimulates IgG antibody to Vi capsular polysaccharide in serum, IgG antibody to 0 antigen in serum, and cell-mediated immune responses (Tackett, et al. 2004). ImmunoScore diagnostic analysis can thus, in exemplary embodiments of the present invention, focus on antibody to Vi capsular polysaccharide. ImmunoScore data analyses can create necessary correlates of protection against typhoid fever disease.

Protection against yellow fever appears to correlate with antibody titers above 0.7 IU. ImmunoScore diagnostic analysis can recommend that an individual with antibody titer below 0.7 IU be boosted.

TABLE 4
CDC Travelers' Health Destinations - Regions

Africa, Central
Africa, East
Africa, North
Africa, Southern
Africa, East
Asia, East
Asia, Southeast
Australia and the South Pacific
Caribbean
Central America and Mexico
Eastern Europe and the Newly Independent States
of the former Soviet Union
Europe, Western
Indian Subcontinent
Middle East
North America
South America, Temperate
South America, Tropical

TABLE 5
Traveler's Vaccine Recommendations

| Vaccine | Number of Regions Recommending |
|---|---|
| Hepatitis A | 16 |
| Hepatitis B | 14 |
| Japanese Encephalitis | 3 |
| Meningococcal | 4 |
| Rabies | 15 |
| Typhoid | 15 |
| Yellow Fever | 7 |
| Tetanus | 17 |
| Diphtheria | 17 |
| Measles | 17 |
| Polio | 2 |

TABLE 6
Rabies Pre-exposure Prophylaxis Guide - United States 1999

| | | | Pre-exposure |
|---|---|---|---|
| Risk Category | Nature of Risk | Typical Populations | Recommendations |
| Continuous | Virus present | Rabies research lab | Primary course. |
| | continuously in high | workers, rabies | Serologic testing |
| | concentrations. | biologics production | every 6 months. |
| | Specific exposures | workers. | Booster vaccination |
| | likely to go unrecognized. | | if AB titer is below |
| | | | acceptable level. |
| Frequent | Exposure usually | Rabies diagnostic | Primary course. |
| | episodic, with | lab workers, | Serologic testing |
| | source recognized, | spelunkers, | every 2 years. |
| | but exposure might | veterinarians, and | Booster vaccination |
| | also be | animal control and | if AB titer is below |
| | unrecognized. | wildlife workers in | acceptable level. |
| | | rabies enzootic | |
| | | areas. | |
| Infrequent (greater | Exposure nearly | Veterinarians and | Primary course. No |
| than population at | always episodic | animal control and | serologic testing or |
| large) | with source | wildlife workers in | booster vaccination. |
| | recognized. | areas with low | |
| | | rabies rates. | |
| | | Veterinary students. | |
| | | Travelers visiting | |
| | | areas where rabies is | |
| | | enzootic. | |
| Rare (population at | Exposure always | U.S. population at | No vaccination |
| large) | episodic with source | large, including | necessary. |
| | recognized. | persons in rabies | |
| | | enzootic areas. | |

3. ImmunoScore Measurement of Immune Status in Adults

Infectious diseases remain major causes of illness, disability, and death. Moreover, new infectious agents and diseases are being detected, and some diseases considered under control have re-emerged in recent years. In addition, antimicrobial resistance is evolving rapidly in a variety of hospital- and community-acquired infections. These trends suggest that many challenges still exist in the prevention and control of infectious diseases.

Between 1980 and 1992, the number of deaths from infectious diseases rose 58% in the United States (Hoyert, et al. 1999). Considered as a group, three infectious diseases—pneumonia, influenza, and HIV infection constituted the fifth leading cause of death in the United States in 1997.

The direct and indirect costs of infectious diseases are significant. Every hospital-acquired infection adds an average of $2,100 to a hospital bill. Bloodstream infections result in an average of $3,517 in additional hospital charges per infected patient because the patient stay averages an additional 7 days. A typical case of Lyme disease diagnosed in the early stages incurs about $174 in direct medical treatment costs. Delayed diagnosis and treatment, however, can result in complications that cost from $2,228 to 6,724 per patient in direct medical costs in the first year alone (Meltzer, et al. 1999).

Vaccines can prevent the debilitating and, in some cases, fatal effects of infectious diseases. Vaccines help to eliminate the illness and disability of polio, measles, and rubella. However, the organisms that cause these diseases have not disappeared. Rather, they have receded and will reemerge if the vaccination coverage drops. The serious health burden of vaccine-preventable diseases is evident from the measles resurgence of 1989 to 1991, resulting in more than 55,000 cases, 11,000 hospitalizations, 120 deaths, and $100 million in direct medical costs (Atkinson, et al. 2000; Gindler, et al. 1992).

Vaccines protect more than the vaccinated individual. They also protect society. When vaccination levels in a community are high, the few who can not be vaccinated—such as young children and individuals with contraindications to vaccination—are often indirectly protected because of group (or “herd”) immunity, because they live among vaccinated persons who may offer protection from exposure to the disease. Vaccines provide significant cost benefits. Three childhood vaccines—diphtheria, tetanus toxoids, and acellular pertussis (DTaP); measles, mumps, and rubella (MMR); and Haemophilus influenzae type b (Hib) vaccine—result in substantial direct medical savings for each dollar spent to vaccinate children against these diseases. Consideration of indirect savings—prevention of work loss by parents to care for ill children and prevention of death and therefore lost earnings from disability—show that vaccines routinely recommended for children are highly cost saving. Since 1989, vaccination requirements have been expanded for schools and day care settings. Similarly, all States and the District of Columbia now require vaccination for children in day care (CDC, 1999).

In addition to very young children, many adults are at increased risk for vaccine-preventable diseases. Vaccination against pneumococcal infections and influenza among individuals aged 65 years and older has increased slightly for African Americans and Hispanics. The coverage in these groups, however, remains substantially below the general population. For example, influenza vaccination rates for whites were 66 percent in 1997, while for African Americans and Hispanics, rates were only 45 and 53 percent, respectively. In September 1997, the U.S. Department of Health and Human Services approved a plan to improve adult vaccination rates and reduce disparities among racial and ethnic groups (CDC, 1999). The elimination of disparities, however, may require further interventions in particular geographic, cultural, and racial and ethnic populations.

A coordinated strategy is necessary to understand, detect, control, and prevent infectious diseases. Such a strategy will protect the gains achieved in life expectancy in the 20^{th} century from control and prevention of infectious diseases and ensure further improvements in the 21^{st} century.

In the United States, most vaccine-preventable diseases occur among adults. Pneumococcal disease and influenza account for more than 30,000 deaths annually, most of which occur in elderly persons. Studies have consistently shown that focusing efforts to improve coverage on health care providers, as well as adult health care systems, is the most effective means of raising vaccine coverage in adults. Healthy People 2010 recommends that all health care providers should assess routinely the vaccination status of their patients. Only record keeping is currently available to assess the vaccination status of patients. In exemplary embodiments of the present invention, an ImmunoScore diagnostic panel cab truly reveal an individual's immune status with regard to vaccine-preventable diseases. Likewise, health plans should develop mechanisms for assessing the vaccination status of their participants. Also, nursing home facilities and hospitals should ensure that policies exist to promote vaccination.

Vaccine safety research and monitoring are also necessary to identify and minimize vaccine-related injuries and illnesses. As programs continue to reduce the new cases of vaccine-preventable diseases, concerns about vaccine adverse events have emerged, posing a threat to public acceptance of vaccines. Knowing the safety profile of vaccines is essential to assess accurately the risks and benefits, to formulate appropriate vaccine recommendations, and to address public concerns. Once firmly entrenched, the ImmunoScore diagnostic database can, for example, compile information regarding safety profiles of vaccines as administered.

FIG. 4G displays the current recommended immunizations for adults from the ACIP. The ACIP uses the following age categories to classify adults regarding immunization priorities:
19-49 years
50-64 years
65 years and older

The ACIP also has made recommendations for adult immunizations based upon medical conditions, as shown in FIG. 4H. These recommendations vary by category and in exemplary embodiments the placement of ImmunoScore diagnostic analyses at health-care settings for these specific medical conditions will ensure that patients are properly immunized and not hyperimmunized. In addition, as the ImmunoScore database is constructed and enlarged, the ACIP can fine-tune recommendations for immunization using ImmunoScore data.

The ACIP makes broad recommendations for immunization of persons aged 19-49 years for:
Tetanus, Diphtheria (Td)
   1 dose if last dose was 10 years ago, or longer
Influenza
   1 dose annually for persons with medical or occupational conditions
Pneumococcal disease
   1 dose for persons with medical or other conditions
Hepatitis B
   3 doses at 0, 1-2, and 4-6 months, for persons with medical, behavioral, occupational, or other indications
Hepatitis A
   2 doses at 0 and 6-12 months, for persons with medical, behavioral, occupational, or other indications
Measles, Mumps, Rubella (MMR)
   1 dose if history is unreliable, 2 doses for persons with occupational, geographic, or other indications
Varicella
   2 doses at 0 and 4-8 weeks, for persons who are susceptible
Meningococcal disease
   1 dose for persons with medical or other indications

The 19-49 year old age group is somewhat problematic regarding their pattern of visits to physicians. Women in this age category are much more likely to see a physician where vaccine recommendations can be made. This is one reason the college admission physical is important as an attempt to capture adults as they enter this age classification.

The ACIP makes broad recommendations for immunization of persons aged 50-64 years for:
Tetanus, Diphtheria (Td)
   1 booster dose every 10 years
Influenza
   1 dose annually for all persons, particularly health-care workers and individuals likely to transmit influenza to persons at high risk
Pneumococcal disease
   1 dose for individuals with medical or other conditions
Hepatitis B
   3 doses for persons with medical, behavioral, occupational, or other indications
Hepatitis A
   2 doses for persons with medical, behavioral, occupational, or other indications
Varicella
   2 doses for individuals who are susceptible
Meningococcal disease
   1 dose for persons with medical or other indications

There is building support for a 50 year old check up. This would be an ideal time to introduce this segment of the population to one or more appropriate ImmunoScore diagnostic panels and assess each 50 year old's immune status. Immune status and antibody levels to vaccine-preventable disease can, for example, easily be monitored at this point.

West Nile virus (WNV) was first identified in 1937 in a febrile person in the West Nile district of Uganda. Prior to 1999, the virus was found only in the Eastern hemisphere, with wide distribution in Africa, Asia, the Middle East, and Europe. There were infrequent reports of human outbreaks, mainly associated with mild febrile illnesses.

In late summer 1999, the first domestically acquired human cases of West Nile encephalitis were documented in the United States. By the end of 2002, WNV activity had been identified in 44 states and the District of Columbia. The 2002 WNV epidemic and epizootic resulted in reports of 4,156 reported human cases of West Nile disease (including 2,942 meningoencephalitis cases and 284 deaths), 16,741 dead birds, 6,604 infected mosquito pools, and 14,571 equine cases. In 2002, four novel routes of WNV transmission to humans were documented for the first time: 1) blood transfusion, 2) organ transplantation, 3) transplacental transfer, and 4) breast-feeding.

Since the mid-1990s, the frequency and apparent clinical severity of WNV outbreaks have increased. Outbreaks in Romania (1996), Russia (1999), and Israel (2000) involved hundreds of persons with severe neurological disease. It is unclear if this apparent change in disease severity and frequency is due to differences in the circulating virus's virulence or to changes in the age structure, background immunity, or prevalence of other predisposing chronic conditions in the affected populations.

The CDC has set goals of surveillance for human cases: To 1) assess the local, state and national public health impact of WNV disease and monitor national trends; 2) demonstrate the need for public health intervention programs; 3) allocate resources; 4) identify risk factors for infection and determine high-risk populations; 5) identify geographic areas in need of targeted interventions; and 6) identify geographic areas in which it may be appropriate to conduct analytical studies of important public health issues.

WNV infection can be suspected in a person based on clinical symptoms and patient history. Laboratory testing is required for confirmed diagnosis. Currently, the most efficient diagnostic method is detection of IgM antibody to WNV is serum collected within 8 to 14 days of illness onset or CSF collected within 8 days of illness onset using the IgM antibody-capture ELISA (MAC-ELISA). Since IgM antibody does not cross the blood-brain barrier, presence of IgM in cerebrospinal fluid strongly suggests central nervous system infection. Patients who have been recently vaccinated against or recently infected with related flaviviruses (e.g., yellow fever, Japanese encephalitis, dengue) may have positive WNV MAC-ELISA results, although vaccination or non-CNS infections should not give CSF IgM, and killed vaccines (e.g., JE-VAX) should not produce IgM at all. Alternatively, IgA antibody to WNV can be detected using the techniques as described in the WNV Provisional patent application, referenced above. These techniques allow a quicker response time, as decsribed therein, relative to current techniques based on IgM detection.

The diagnosis of WNV infection relies on a high index of clinical suspicion and on results of specific laboratory tests. WNV or other arboviral diseases, such as St. Louis encephalitis, should be seriously considered in adults 50 years of age or older who have onset of unexplained encephalitis or meningitis in late summer or early fall. Severe neurologic disease due to WNV infection has occurred in persons of all ages, and because year-round transmission is possible in southern states, WNV should always be considered in persons with unexplained encephalitis and meningitis.

Thus, ImmunoScore diagnostic analysis of antibody to WNV would be a valuable component in the examination of individuals over 50 years of age. Tracking of WNV infection in the ImmunoScore database would shed more light onto the population of individuals most likely to be susceptible to WNV infection. If infection by WNV continues to expand, other groups would fall under consideration, including possibly travelers and inhabitants of regions where the virus is more likely to be endemic.

The ACIP makes broad recommendations for immunization of persons aged 65 years and older for:
Tetanus, Diphtheria (Td)
   1 booster dose every 10 years
Influenza
   1 dose annually for all persons, particularly health-care workers and persons likely to transmit influenza to persons at high risk
Pneumococcal disease
   1 dose for all unvaccinated persons
Hepatitis B
   3 doses for persons with medical, behavioral, occupational, or other indications
Hepatitis A
   2 doses for persons with medical, behavioral, occupational, or other indications
Varicella
   2 doses for individuals who are susceptible
Meningococcal disease
   1 dose for persons with medical or other indications

The ACIP makes specific recommendations for individual adults with medical conditions. The following are categories are considered:
Pregnancy
Diabetes, heart disease, chronic pulmonary disease, chronic liver disease (including chronic alcoholism)
Congenital immunodeficiency, leukemia, lymphoma, generalized malignancy, therapy with alkylating agents, antimetabolites, radiation, or large amounts of corticosteroids
Renal failure, end stage renal disease, recipients of hemodialysis
Asplenia
HIV infection

The specific recommendations for pregnant adults are as follows:
For all persons in this group:
   Tetanus, Diphtheria (Td)
   Influenza, if pregnancy is at second or third trimester during influenza season
For persons with medical/exposure indications:
   Pneumococcal polysaccharide
   Hepatitis B
   Hepatitis A
Vaccines that are contraindicated:
   Measles, Mumps, Rubella (MMR)
   Varicella

The specific vaccine recommendations by the ACIP for adult individuals with diabetes, heart disease, chronic pulmonary disease, and chronic liver disease (including chronic alcoholism) are the following:
For all persons in this group:
   Tetanus, Diphtheria (Td)
   Influenza
   Pneumococcal polysaccharide
For all persons with medical/exposure indications:
   Hepatitis B
   Hepatitis A (all persons with chronic liver disease)
Catch up on childhood vaccinations:
   Measles, Mumps, Rubella (MMR)
   Varicella

The specific recommendations by the ACIP for adult individuals with congenital immunodeficiency, leukemia, lymphoma, generalized malignancy, therapy with alkylating agents, antimetabolites, radiation, or large amounts of corticosteroids are as follows:
For all persons in this group:
   Tetanus, Diphtheria (Td)
   Influenza
   Pneumococcal polysaccharide
For persons with medical/exposure indications:
   Hepatitis B
   Hepatitis A
Contraindicated:
   Measles, Mumps, Rubella (MMR)
   Varicella (persons with impaired humoral, but not cellular immunity may be vaccinated)

The specific recommendations by the ACIP for adult individual with renal failure, end stage renal disease, recipients of hemodialyis or clotting factor concentrates are as follows:
For all persons in this group:
   Tetanus, Diphtheria (Td)
   Influenza
   Pneumococcal polysaccharide
   Hepatitis B (with annual assessment of anti-HBs antibody)
For persons with medical/exposure indications:
   Hepatitis A
Catch-up on childhood vaccinations:
   Measles, Mumps, Rubella (MMR)
   Varicella

The ACIP makes the following recommendations for asplenic individuals, including those individuals with a splenectomy and those with terminal complement component deficiencies:
For all persons in this group:
   Tetanus, Diphtheria (Td)
   Influenza
   Meningococcal polysaccharide
   Pneumococcal polysaccharide
For persons with medical/exposure indications:
   Hepatitis B
   Hepatitis A
Catch-up on childhood vaccinations:
   Measles, Mumps, Rubella (MMR)
   Varicella

For adults with HIV infections, the ACIP recommends the following:
For all persons in this group:
   Tetanus, Diphtheria (Td)
   Influenza
   Hepatitis B
   Pneumococcal polysaccharide
For persons with medical/exposure indications:
   Hepatitis A
Catch-up on childhood immunizations:
   Measles, Mumps, Rubella (MMR)— withhold MMR or other measles containing vaccines from HIV-infected persons with evidence of severe immunosuppression
Contraindicated:
   Varicella

The application of ImmunoScore diagnoses for adults with medical conditions is a natural “fit” in health-care settings. These individuals are generally being treated and seen by physicians on a somewhat regular basis. The ImmunoScore database could, for example, easily track these individuals and get regular measurements of serum antibodies to vaccine-preventable diseases, as well as overall immune health.

Additionally, there are other diseases which are or could be vaccine preventable, where an approved vaccine does not yet, but may soon, exist. In such cases ImmunoScore diagnostic panels can incorporate tests for the presence of, or for immunity to, such diseases in anticipation of a vaccine becoming available.

For example, there is no vaccine for West Nile Virus for humans in the United States. Nonetheless, researchers at the National Institute for Allergy and Infectious Diseases Vaccine Research Center are developing a vaccine for West Nile virus. This vaccine is made from a short circular piece of West Nile DNA modified to produce West Nile proteins. Researchers are evaluating the vaccine in animal models. If successful, it could proceed to early phase human clinical trials by 2005, pending Food and Drug Administration approval (reported on an NIAID October 2004 Fact Sheet on West Nile Virus, available on the Internet at http://www.niaid.nih.gov/factsheets/westnile.htm). NIAID is also funding a chimeric virus vaccine for the disease being developed by Acambis.

When a West Nile Virus (“WNV”) vaccine does exist for approved use in humans, it is primarily the adult population over 50 years old who will benefit, as noted above, inasmuch as most patients with WN encephalitis or meningitis (WNME) are older adults, generally over 50. Centers for Disease Control and Prevention, Epidemic/Epizootic West Nile Virus in the United States: Guidelines for Surveillance, Prevention, and Control (CDC, National Center for Infectious Diseases, Division of Vector-Borne Infectious Diseases, Fort Collins, Colo., 3rd Revision 2003). Thus, as noted above, in exemplary embodiments of the present invention, there can be various contexts in which a WNV test would be desirably included in an ImmunoScore diagnostic assay panel directed to adults, and the results of such a test can be, for example, used to recommend vaccination. Such a WNV assay could be, for example, one or more assays as described in the WNV Provisional patent application, described (and incorporated herein by reference) above.

Moreover, because WNV is seasonal, and also varies with geographic location, a WNV assay could be, for example, also included in other ImmunoScore diagnostic panels in exemplary embodiments of the present invention, such as, for example, panels directed to health care workers, travelers, or to the general population in areas of high risk of human infection, both for purposes of tracking infection, as noted above, and for potential reccommendations for vaccination.

4. ImmunoScore Measurement of Immunity in Health Care Workers

Because of their contact with patients or infective material from patients, many health-care workers (e.g. physicians, nurses, emergency medical personnel, dental professionals and students, medical and nursing students, laboratory technicians, hospital volunteers, and administrative staff) are at risk for exposure to and possible transmission of vaccine-preventable diseases. Maintenance of immunity is therefore an essential part of prevention and infection control programs for health-care workers. Optimal use of immunizing agents safeguards the health of workers and protects patients from becoming infected through exposure to infected workers. Consistent immunization programs could substantially reduce both the number of susceptible health-care workers in hospitals and health departments and the attendant risks for transmission of vaccine-preventable diseases to other workers and patients. In exemplary embodiments of the present invention, the judicious application of ImmunoScore diagnostics to the needs of health-care workers can assure that these individuals will be appropriately immunized and protected from both becoming infected and spreading infection. The Centers for Disease Control (CDC) has recommended immunizing agents for health-care workers (Table 7; CDC, 1997).

Any medical facility or health department that provides direct patient care is encouraged to formulate a comprehensive immunization policy for all health-care workers. The American Hospital Association has endorsed the concept of immunization programs for both hospital personnel and patients (AHA, 1992). The use of ImmunoScore diagnostic capability coupled with rigorous immunization programs can assist in the decline of nosocomial infections.

There are diseases for which the CDC strongly recommends vaccination for health-care workers. These include Hepatitis B, influenza, measles, mumps, rubella, varicella-zoster, and tuberculosis. There are other diseases that vaccination may be indicated for; these include Hepatitis A, meningococcal disease, Typhoid, and smallpox. Finally, for some health-care workers, there may be a recommendation for tetanus, diphtheria, pertussis, and pneumococcal disease.

Hepatitis B virus (HBV) infection is the major infectious health hazard for health-care personnel. Data indicate that 5-10% of HBV-infected workers become chronically infected. Individuals with chronic HBV infection are at risk for chronic liver disease and are potentially infectious throughout their lifetimes. The risk of acquiring HBV infection from occupational exposures is dependent on the frequency of percutaneous and permucosal exposures to blood or body fluids containing blood. Depending on the tasks he or she performs, any health-care or public safety worker may be at high risk for HBV exposure. Workers performing tasks involving exposure to blood or blood-containing body fluids should be vaccinated. For public safety workers whose exposure to blood is infrequent, timely post-exposure prophylaxis may be considered, rather than routine pre-exposure vaccination.

Pre-vaccination serologic screening for prior infection is not currently indicated for persons being vaccinated because of occupational risk. Post-vaccination testing for antibody to hepatitis B surface antigen response is indicated for health-care workers who have blood or patient contact and are at ongoing risk for injuries with sharp instruments or needlesticks. Knowledge of antibody response aids in determining appropriate post-exposure prophylaxis.

Vaccine-induced antibodies to HBV decline gradually over time, and <60% of persons who initially respond to vaccination will lose detectable antibodies over 12 years (Stevens, et al. 1992). Studies among adults have demonstrated that, despite declining serum levels of antibody, vaccine-induced immunity continues to prevent clinical disease or detectable viremic HBV infection (Hadler, et al. 1992). Therefore, booster doses are not considered necessary. Periodic serologic testing to monitor antibody concentrations after completion of the three-dose series is currently not recommended. An obvious advantage of the ImmunoScore diagnostic panel would be that periodic monitoring of immune status could be correlated with any outbreak of disease in health-care workers. The availability of an ImmunoScore database can be exceedingly beneficial to the health-care workers in the care settings. The possible need for booster doses can be assessed as additional data become available.

Asymptomatic HBV infections have been detected in vaccinated individuals by means of serologic testing for antibody to hepatitis B core antigen. However, these infections also provide lasting immunity and are not associated with HBV-related chronic liver disease.

During community influenza outbreaks, admitting patients infected with influenza to hospitals has led to nosocomial transmission of the disease (Balkovic, et al. 1980), including transmission from staff to patients. Transmission of influenza among medical staff causes absenteeism and considerable disruption of health care. In addition, influenza outbreaks have caused morbidity and mortality in nursing homes. Because there is a recommendation for an annual influenza vaccination for health-care workers, it is unlikely that there would be an ImmunoScore diagnostic application for flu. The only potential here would be to correlate vaccination and protection in a multitude of individuals working in the health-care field.

Nosocomial measles transmission has been documented in the offices of private physicians, in emergency rooms, and on hospital wards. Although only 3.5% of all cases of measles reported during 1985-1989 occurred in medical settings, the risk for measles infection in medical personnel is estimated to be thirteen fold that for the general population (Watkins, et al. 1987; Atkinson, et al. 1991). Of the 3,659 measles cases reported during 1992-1995, the setting of transmission was known for 2,735; 385 (13.9%) of these cases occurred in medical settings (CDC, 1997). Although birth before 1957 is considered acceptable evidence of measles immunity, serologic studies of hospital workers indicate that 5-9% of those born before 1957 are not immune to measles (Smith, et al. 1990). During 1985-1992, 27% of all measles cases among health-care workers occurred in individuals born before 1957 (CDC, 1997). Measles vaccination is contraindicated in pregnant and immunocompromised individuals, including HIV-infected persons who have evidence of severe immunosuppression. Measles is also contraindicated following recent administration of immune globulin.

In recent years, a substantial proportion of reported mumps has occurred among unvaccinated adolescents and young adults on college campuses and in the workplace (Cochi, et al. 1988; Kaplan, et al. 1988). Outbreaks of mumps in highly vaccinated populations have been attributed to primary vaccine failure. During recent years, the overall incidence of mumps has fluctuated only minimally, but an increasing proportion of cases have been reported in individuals aged>15 years (CDC, 1995). The CDC states that programs to ensure that medical personnel are immune to mumps are prudent and are easily liked with measles and rubella control programs (CDC, 1997). Mumps vaccination is contraindicated in pregnant and immunocompromised individuals.

Nosocomial rubella outbreaks involving both health-care workers and patients have been reported (Greaves, et al. 1982). Although vaccination has decreased the overall risk for rubella transmission in all age groups in the United States by ≧95%, the potential for transmission in hospital and similar settings persists because 10-15% of young adults are still susceptible (Bart, et al. 1985). Although not as infectious as measles, rubella can be transmitted effectively by both males and females. Transmission can occur whenever many susceptible individuals congregate in one place. Aggressive rubella vaccination of susceptible men and women with trivalent MMR vaccine can eliminate rubella transmission. Persons born before 1957 are generally considered to be immune to rubella. However, findings of seroepidemiologic studies indicate that about 6% of health-care workers (including individuals born before 1957) do not have detectable rubella antibody (CDC, 1997). Rubella vaccination is contraindicated in pregnant and immunocompromised individuals.

For all the infectious disease above covered by the MMR vaccination, an ImmunoScore diagnostic panel would be a useful application for health-care workers. These vaccines are available as monovalent or trivalent combinations. Lack of protective levels of antibody to any one of the components could be ameliorated by vaccination. In addition, health-care workers would again provide a large population to add an exemplary ImmunoScore database.

Nosocomial transmission of varicella zoster virus (VZV) is well recognized. Sources for nosocomial exposure of patients and staff have included patients, hospital staff, and visitors who are infected with either varicella (chickenpox) or zoster (shingles). In hospitals, airborne transmission of VZV from persons who had varicella or zoster to susceptible persons who had no direct contact with the index case patient has occurred. Although all susceptible hospitalized adults are at risk: pregnant women, premature infants born to susceptible mothers, infants born at <28 weeks' gestation or who weigh<1,000 grams regardless of maternal immune status, and immunocompromised persons of all ages (including persons who are undergoing immunosuppressive therapy, have malignant disease, or are immunodeficient).

Strategies for managing clusters of VZV infections in hospitals include:
isolating patients who have varicella and other susceptible patients who are exposed to VZV;
controlling air flow;
using rapid serologic testing to determine susceptibility;
furloughing exposed susceptible personnel or screening these persons daily for skin lesions, fever, and systemic symptoms; and
temporarily reassigning varicella-susceptible personnel to locations remote from patient-care areas.

A reliable history of chickenpox is a valid measure of VZV immunity. Serologic tests have been used to assess the accuracy of reported histories of chickenpox. Among adults, 97-99% of individuals with a positive history of varicella are seropositive. In addition, the majority of adults with negative or uncertain histories are seropositive (range 71-93%). Persons who do not have a history of varicella, or whose history is uncertain can be considered susceptible, and can be tested serologically by ImmunoScore diagnostic methodology to determine their immune status. In health-care institutions, serologic screening of personnel who have a negative or uncertain history of varicella is likely to be cost effective (CDC, 1996).

If susceptible health-care workers are exposed to varicella, they are potentially infective 10-21 days after exposure. They must be furloughed during this period, usually at substantial cost. Administration of varicella zoster immune globulin (VZIG) after exposure can be costly. VZIG does not necessarily prevent varicella, and may prolong the incubation period by a week or more, thus extending the time during which personnel should not work.

Varicella virus vaccine protects approximately 70-90% of recipients against infection and 95% of recipients against severe disease for at least 7-10 years after vaccination. Significant protection is long-lasting. Breakthrough infections have occurred among vaccinees after exposure to natural varicella virus. Estimates of vaccine efficacy and persistence in vaccinees are based on research conducted before widespread use of varicella vaccine began to influence the prevalence of natural VZV infection. Therefore, the extent to which boosting from exposure to natural virus increases the protection provided by vaccination remains unclear. Whether longer term immunity may wane as the circulation of natural VZV decreases also is unknown.

The CDC recommends that vaccination should be considered for unvaccinated health-care workers who lack documented immunity if they are exposed to varicella. However, because the effectiveness of post-exposure vaccination is unknown, individuals vaccinated after an exposure should be managed in the manner recommended for unvaccinated persons. Here, again, the ImmunoScore diagnostic assay for varicella would be a valuable assessment tool prior to initiation of vaccination of individuals uncertain of their immune status or disease history.

In the United States, Bacille Calmette-Guérin (BCG) vaccine has not been recommended for general uses because the population risk for infection with Mycobacterium tuberculosis (TB) is low and the protective efficacy of BCG vaccine uncertain. The immune response to BCG vaccine also interferes with the use of the tuberculin skin test to detect M. tuberculosis infection (CDC, 1996). TB prevention and control efforts are focused on interrupting transmission from patients who have active infectious TB, skin testing those at high risk for TB, and administering preventive therapy when appropriate. However, in certain situations, BCG vaccination may contribute to the prevention and control of TB when other strategies are inadequate. The fundamental strategies for the prevention and control of TB include:
Early detection and effective treatment of patients with active communicable TB.
Preventative therapy for infected persons. Identifying and treating persons who are infected with M. tuberculosis can prevent the progression of latent infection to active infectious disease.
Prevention of institutional transmission. The transmission of TB is a recognized risk in health-care settings and is of particular concern in settings where HIV infected individuals work, volunteer, visit, or receive care. Effective TB infection-control programs should be implemented in health-care facilities and other institutional settings (e.g. shelters for homeless persons and correctional facilities).

In a few geographic areas of the United States, increased risks for TB transmission in health-care facilities (compared with risks observed in health-care facilities in other parts of the United States) occur together with an elevated prevalence among TB patients of M. tuberculosis strains that are resistant to both isoniazid and rifampin. Even in such situations, comprehensive application of infection control practices should be the primary strategy used to protect health-care workers from infection with M tuberculosis. BCG vaccination of health-care workers should not be used as a primary TB control strategy because:
the protective efficacy of the vaccine in health-care workers is uncertain;
even if BCG vaccination is effective for a particular health-care worker, other persons in the health-care facility (e.g. patients, visitors and other health-care workers) are not protected against possible exposure to and infection with drug-resistant strains of M. tuberculosis; and
BCG vaccination may complicate preventive therapy because of difficulties in distinguishing tuberculin skin test responses caused by infection with M. tuberculosis from those caused by the immune response to vaccination.

Hepatitis C virus (HCV) is the etiologic agent in most cases of parenterally transmitted non-A, non-B hepatitis in the United States. CDC estimates that the annual number of newly acquired HCV infections has ranged from 180,000 in 1984 to 28,000 in 1995. Of these, and estimated 2-4% occurred among health-care personnel who were occupationally exposed to bleed. At least 85% of individuals who contract HCV infection become chronically infected, and chronic hepatitis develops in an average of 70% of all HCV-infected individuals (Shakil, et al. 1995).

Serologic enzyme immunoassays licensed for the detection of antibody to HVD have evolved since their introduction in 1990 and a third version is now available which detects anti-HCV in >95% of patients with HCV infection. These assays do not detect anti-HCV in all infected individuals and do not distinguish among acute, chronic, or resolved infection. In 80-90% of HCV-infected individuals, seroconversion occurs an average of 10-12 weeks after exposure to HCV. These screening assays also yield a high proportion (up to 50%) of falsely positive tests when they are used in populations with a low prevalence of HCV infection (CDC, 1991).

Based on seroepidemiological surveys, the core and the non-structural 3 (NS3) proteins of hepatitis C virus are thought to be two of the most immunogenic proteins of HCV (Chiba, et al. 1991). A majority of HCV-infected immuno-competent individuals develop HCV core antibodies (Chen, et al. 1995). However, there seems to be low or no HCV antibodies early in acute infection (Chen, et al. 1995). The major IgG subclasses of antibodies to HCV core are IgG1 and IgG3 (Sällberg, et al. 1992).

In the absence of effective prophylaxis, individuals who have been exposed to HCV may benefit from knowing their infection status so they can seek evaluation for chronic liver disease and treatment. IG or antiviral agents are not recommended for post-exposure prophylaxis of hepatitis C. No vaccine against hepatitis C is available. Health-care institutions should consider implementing policies and procedures to monitor health-care workers for HCV infection after percutaneous or permucosal exposures to blood. The CDC recommends, at minimum, that such policies should include:
For the source, baseline serologic testing for anti-HCV;
For the person exposed to an anti-HCV positive source, baseline and follow-up (e.g. six months) serologic testing for anti-HCV and alanine aminotransferase activity;
Confirmation by supplemental anti-HCV testing of all anti-HCV results reported as repeatedly reactive by EIA;
Education of health-care workers about the risk for and prevention of occupational transmission of all blood borne pathogens, including hepatitis C, using up-to-date and accurate information (CDC, 1997).

There are other diseases for which immunizations of health-care workers are or may be indicated. Diseases are included in this category for one of the following reasons:
Nosocomial transmission occurs, but health-care workers are not at increased risk as a result of occupational exposure (e.g. hepatitis A),
Occupational risk may be high, but protection via active or passive immunization is not available (i.e. pertussis), or
Vaccines are available but are not routinely recommended for all health-care workers or are recommended only in certain situations (i.e. vaccinia and meningococcal vaccines).

Occupational exposure generally does not increase health-care worker's risk for hepatitis A virus (HAV) infection. When proper infection control practices are followed, nosocomial HAV transmission is rare. Outbreaks caused by transmission of HAV to neonatal intensive care unit staff by infants infected through transfused blood have occasionally been observed (Rosenblum, et al. 1991). Transmission of HAV from adult patients to health-care workers is usually associated with fecal incontinence in the patients. However, most patients hospitalized with hepatitis A are admitted after onset of jaundice, when they are beyond the point of peak infectivity (Goodman, 1985). Serologic surveys among many types of health-care workers have not identified an elevated prevalence of HAV infection compared with other occupational populations.

Two specific prophylactic measures are available for protection against hepatitis A—administration of immune globulin (IG) and hepatitis A vaccine. When administered within two weeks after an exposure, IG is >85% effective in preventing hepatitis. There are two inactivated hepatitis A vaccines currently available in the United States. The duration of clinical protection has not yet been established. An ImmunoScore database built from surveillance of health-care workers can thus be instrumental in the determination of the duration of clinical protection of each of these vaccines.

Nosocomial transmission of Neisseria meningitidis is uncommon. In rare instances, direct contact with respiratory secretions of infected persons (e.g. during mouth to mouth resuscitation) has resulted in transmission from patients with meningococcemia or meningococcal meningitis to health-care workers. Although meningococcal respiratory infections are rare, health-care workers may be at increased risk for meningococcal infection if exposed to N. meningitidis-infected patients with active productive coughs. Health-care workers can decrease the risk for infection by adhering to precautions to prevent exposure to respiratory droplets.

Post-exposure prophylaxis is advised for individuals who have had intensive, unprotected contact with infected patients (e.g. intubating, resuscitating, or closely examining the oropharynx of patients). Antimicrobial prophylaxis can eradicate carriage of N. Meningitidis and prevent infections in individuals who have unprotected exposure to patients with meningococcal infections.

Although useful for controlling outbreaks of serogroup C meningococcal disease, administration of quadrivalent A, C, Y, W-135 meningococcal polysaccharide vaccines is of little benefit for post-exposure prophylaxis. The serogroups A and C vaccines, which have demonstrated estimated efficacies of 85-100% in older children and adults, are useful for control of epidemics (CDC, 1997). The decision to implement mass vaccination to prevent serogroup C meningococcal disease depends on whether the occurrence of more than one case of the disease represents an outbreak or an unusual clustering of endemic meningococcal disease. Surveillance for serogroup C disease and calculation of attack rates can be used to identify outbreaks and determine whether use of meningococcal vaccine is warranted. The meningococcal diagnostic panel of the ImmunoScore diagnostic application would be a useful tool to monitor health-care workers, and to also identify health-care workers at increased risk for meningococcal disease.

Pertussis is highly contagious. Secondary attack rates among susceptible household contacts exceed 80% (Mortimer, 1990). Transmission occurs by direct contact with respiratory secretions or large aerosol droplets from the respiratory tract of infected persons. The incubation period is generally 7-10 days. The period of communicability starts with the onset of the catarrhal stage and extends into the paroxysmal stage. Vaccinated adolescents and adults, whose immunity wanes 5-10 years after the last dose of vaccine (usually administered at age 4-6 years), are an important source of pertussis infection for susceptible infants. The disease can be transmitted from adult patients to close contacts, especially unvaccinated children. Such transmission may occur in households and hospitals.

Transmission of pertussis in hospital settings has been documented in several reports (Christie, et al. 1995; Kurt, et al. 1972; Valenti, et al. 1980). Transmission has occurred from a hospital visitor, from hospital staff to patients, and from patients to hospital staff. Although of limited size, documented outbreaks were costly and disruptive. In each outbreak, larger numbers of staff were evaluated for cough illness and required nasopharyngeal cultures, serologic tests, prophylactic antibiotics, and exclusion from work.

During outbreaks that occur in hospitals, the risk for contracting pertussis among patients or staff is often difficult to quantify because exposure is not well defined. Serologic studies conducted among hospital staff during two outbreaks indicate that exposure to pertussis is much more frequent than the attack rates of clinical disease indicate (Mortimer, 1990; Christie, et al. 1995; Kurt, et al. 1972; Valenti, et al. 1980). Seroprevalence of pertussis agglutinating antibodies correlated with the degree of patient contact and was highest among pediatric house staff (82%) and ward nurses (71%), and lowest among nurses with administrative responsibilities (35%) (Linnemann, et al. 1975).

Prevention of pertussis transmission in health-care settings involves diagnosis and early treatment of clinical cases, respiratory isolation of infectious patients who are hospitalized, exclusion from work of staff who are infectious, and post-exposure prophylaxis. Early diagnosis of pertussis, before secondary transmission occurs, is difficult because the disease is highly communicable during the catarrhal stage, when symptoms are still non-specific. Pertussis should be one of the differential diagnoses for any patient with an acute cough illness of >7 days duration without another apparent cause, particularly if characterized by paroxysms of coughing, post-tussive vomiting, whoop, or apnea (CDC, 1997). Health-care settings would be the ideal placement for ImmunoScore diagnostic assays for pertussis. Periodic measurement of the level of pertussis antibody in health-care workers could become part of routine screening to protect both the health-care worker and the patient populations.

One acellular pertussis vaccine is immunogenic in adults, but does not increase risk for adverse events when administered with tetanus and diphtheria (Td) toxoids, as compared with the administration of Td alone (Edwards, et al. 1993). If acellular pertussis vaccines are licensed for use in adults in the future, booster doses of adult formulations of acellular pertussis vaccines may be recommended to prevent the occurrence and spread of the disease in adults, including health-care workers. However, acellular pertussis vaccines combined with diphtheria and tetanus toxoids (DTaP) will need to be reformulated for use in adults, because all infant formulations contain more diphtheria toxoid than is recommended for individuals aged≧7 years. Recommendations regarding routine vaccination of adults will require additional studies (e.g. studies of the incidence, severity, and cost of pertussis among adults; studies of the efficacy and safety of adult formulations of DTaP; and studies of the effectiveness and cost-effectiveness of a strategy of adult vaccination, particularly for health-care workers). Even prior to such a recommendation by the ACIP, an ImmunoScore diagnostic assay for pertussis and the patient database can be a valuable tool for evaluating the need and the effectiveness of the vaccine application.

The incidence of typhoid fever declined steadily in the United States from 1900 to 1960 and has remained at a low level. During 1985-1994, the average number of cases reported annually was 441 (CDC, 1997). Nearly three quarters of patients infected with Salmonella typhi reported foreign travel during the 30 days before onset of symptoms. During this ten year period, several cases of laboratory acquired typhoid fever were reported among microbiology laboratory workers, only one of whom had been vaccinated. S. typhi and other enteric pathogens may be nosocomially transmitted via the hands of personnel who are infected. Generally, personal hygiene, particularly hand washing before and after all patient contacts, will minimize risk for transmitting enteric pathogens to patients. If health-care workers contract an acute diarrheal illness accompanied by fever, cramps, or bloody stools, they are likely to be excreting large numbers of infective organisms in their feces. Excluding these workers from care of patients until the illness has been evaluated and treated will prevent transmission. Workers in microbiology laboratories who frequently work with S. typhi should be vaccinated with any one of the three typhoid vaccines distributed in the United States. ImmunoScore diagnostics would be able to monitor the immune status of vaccinated individuals.

Smallpox is a highly contagious infection caused by the DNA virus variola, a member of the genus Orthopoxvirus. As recently as 1967, millions of smallpox cases per year were reported in Asia and Africa. Smallpox is spread most efficiently in droplets or aerosols from the oropharynx of infected individuals. Smallpox also can be spread by direct contact with infected lesions or with clothing or bed linens contaminated with the virus. After the incubation period of 7 to 17 days, the period of infectivity begins as an exanthema and rash characterized by maculae progressing to papules, vesicles, and pustules all in the same stage, developing first on the face and extremities. Patients remain contagious until the scabs have been shed. Most patients are sick enough during the prodromal period to be confined to bed by the time the rash develops. For this reason, household contacts, hospital workers, and other health-care professionals are the most likely individuals to develop secondary cases.

Case fatality rates of 30% or higher were observed during epidemics of smallpox. In the absence of pre-existing immunity, a favorable prognosis is less likely for infants, the elderly and pregnant women. Immunodeficiency, whether from immunosuppressive therapy or from human immunodeficiency virus (HIV) infection, is likely to have a negative impact on prognosis. Secondary bacterial infections of the skin, eyes and respiratory tract can develop and lead to septicemia and disseminated bacterial disease. Laryngeal lesions can lead to edema and airway obstruction. Encephalitis also may complicate smallpox.

After an extensive worldwide eradication program, the last non-laboratory case of smallpox occurred in 1977 in Somalia. In 1972, routine smallpox immunization was discontinued in the United States, and since 1983, vaccine production has been halted. Stockpiled vaccine has been used only for laboratory researchers working on orthopoxviruses. In recent years, there has been a concern that smallpox virus stocks may be in the hands of bioterrorists, and this concern has been heightened by the terrorist attack on the World Trade Center and the Pentagon on Sep. 11, 2001. Because most of the population is considered to be non-immune, there is debate as to whether smallpox immunization should be resumed.

Protection from infection was provided in the past by immunizing all children beginning at one year of age. An individual's concentration of neutralizing antibodies declines significantly over a 5 to 10 year period, and people who were immunized as infants or children before 1972 are unlikely to remain fully protected against disease, but protection against death afforded by antibodies and cell-mediated immunity may persist for 30 years (AAC, 2003).

There are current concerns regarding smallpox. Stocks of smallpox virus were retained in government run laboratories of the United States and former Soviet Union. There are reports that, before the dissolution of the Soviet Union, smallpox was being developed as a weapon of biological warfare (Henderson, et al. 1999). In addition, decreasing financial support for Russian government laboratories in recent years led to concern that the virus and the expertise to propagate a large amount of smallpox virus may have fallen into non-Russian hands. The rapidity with which smallpox could spread in the U.S. population has led to concern that this agent would present a particularly potent threat if it were to be used as an agent of bioterrorism (O'Toole, 1999; Meltzer, et al. 2001).

Immunization causes a local infection that is pruritic and uncomfortable. Fever, malaise, and regional lymphadenitis often occur about a week after immunization. The site of immunization develops a papule that matures into a pustule and then a scab that separates by about the third week after immunization. Re-immunization typically causes a milder lesion that develops more quickly. Occasionally, satellite or distant pustules develop when a vaccine recipient scratches the pustule and auto inoculates the virus at another site.

A major reason not to initiate universal immunization in the absence of actual cases of smallpox besides the limited availability of vaccine is the risk of serious complications of immunization.

Severe complications of immunization include death, post-vaccinal encephalitis, progressive vaccinia, eczema vaccinatum, generalized rash, and accidental inoculation to the face, eye or other sites. Smallpox vaccine has been known for decades to produce significant adverse effects, especially with immunocompromised individuals. In patients with chronic skin conditions, smallpox vaccine can cause a severe, sometimes fatal dermatologic involvement termed eczema vaccinatum. The list of conditions that place patients at risk of eczema vaccinatum is long and includes most disorders that disrupt epidermal integrity. Atopic dermatitis is the most common disorder associated with severe eczema vaccinatum, and people with this disorder may be susceptible even if the skin disorder is in remission. Even unimmunized susceptible individuals can have such reactions if the virus spreads to them from those who have been immunized.

Smallpox vaccine is not recommended for people with eczema or other exfoliative skin disorders, for pregnant women, or for people with immunodeficiencies, whether primary or secondary. Atopic dermatitis, a genetically based immune abnormality, occurs within the first five years of life and affects 15% of the population.

Before its discontinuation, universal smallpox immunization was recommended in the United States for children 1 to 2 years of age. Re-immunization was recommended every 5 years and annually for people working in endemic areas. The current recommendation for those individuals at high risk because of occupational exposure is immunization every 3 years. People with multiple immunizations during childhood probably have longer-lasting immunity, but the degree of protection for those immunized before 1972 is unknown.

In the event of a known bioterrorist release of the smallpox virus, vaccine would be administered to exposed individuals. If vaccine is given within 3 to 4 days of exposure, immunity can develop before the disease occurs, and this would be expected to prevent or ameliorated the severity of the disease. Post-exposure immunization is recommended for persons who have had face-to-face, household contact with or have been in proximity to a person who has active smallpox skin lesions, persons who have been involved in the care of such an individual, and persons exposed in any way to laboratory specimens or bedding from an infected patient. Such a plan (referred to as a “ring vaccination” program) would allow the most effective use of available stocks of vaccine while exposing a minimal number of individuals to the risks of immunization.

Variola virus as an agent of bioterrorism has been discussed widely, but the difficulty of introducing the virus into the population and the limited effects of doing so have persuaded most public health authorities that the chances of a smallpox outbreak are very small. Because of the known adverse effects of smallpox immunization, the large number of immunocompromised people in the population, and the currently limited supplies of vaccine and IG, all stockpiled vaccine is considered an investigational agent and is available for use by public health authorities only.

The major proposed strategies for smallpox immunization in the face of a bioterrorism threat include mass immunization, voluntary immunization, and ring vaccination or “surveillance and containment.” The proponents of mass immunization claim it to be the strategy that would most effectively prevent spread of disease. They also postulate that a bioterrorist would be unlikely to introduce variola into a well immunized population. Those who favor voluntary immunization feel that each individual should be allowed to weigh the pros and cons of immunization and act according to his or her own analysis (Bicknell, 2002). Unfortunately, much of the population is not familiar with the problems and complications of vaccinia immunization. The ring vaccination strategy is supported by the American Academy of Pediatrics (AAP), which considers this option to be the best approach at present (AAP, 2002).

The AAP supports the current CDC recommendation of the strategy known as ring vaccination, also referred to as surveillance and containment. Using this approach, if smallpox were introduced in an act of terrorism, infected patients would be isolated. Contacts of infected individuals as well as their contacts would then be identified and immunized by specially trained teams of health-care professionals. This strategy can control a localized outbreak with minimal exposure of vulnerable populations to the complications of immunization. The ring strategy is based on the knowledge that vaccination can prevent or ameliorate disease severity if given within 3 to 4 days of initial exposure and can decrease symptoms if given within the first week of exposure.

Immunizing and monitoring a ring of people around each infected individual and his or her contacts would help protect those at the greatest risk of contracting the disease and form a buffer of immune individuals to prevent the spread of disease. The AAP supports the opinion of the ACIP that it is desirable to have patients with smallpox cared for by persons who have been immunized. Thus, national, state-based, and local teams of health-care professionals who already have been immunized will be trained in all aspects of smallpox investigation and care and will be available to go immediately to the site of a suspected or proven smallpox case. With teams available in every state, approximately 10,000 to 20,000 carefully screened individuals will receive smallpox vaccination. For health-care workers, it will be necessary to monitor their immune status with regards to smallpox immunizations. This can be a function of the ImmunoScore diagnostic panel. Health-care professionals and military personnel can provide a sound basis for the accumulated database regarding the persistence of immunity to smallpox infection.

Health-care workers are not at greater risk for diphtheria, tetanus, and pneumococcal disease than the general population. ACIP recommends that all adults be protected against diphtheria and tetanus, and recommends pneumococcal vaccination of all persons aged≧65 years and of younger individuals who have certain medical conditions. Thus, in exemplary embodiments of the present invention, an ImmunoScore diagnostic evaluation can be used to assess an individual health-care worker's immune status with regards to these, or other, as may be appropriate, infectious diseases.

Immunocompromised Health-Care Workers

A physician must assess the degree to which an individual health-care worker is immunocompromised. Severe immunosuppression can be the result of congenital immunodeficiency; HIV infection; leukemia; lymphoma; generalized malignancy; or therapy with alkylating reagents, antimetabolites, radiation, or large amounts of corticosteroids. All persons affected by some of these conditions are severely immunocompromised, whereas for other conditions (e.g. HIV infection), disease progression or treatment stage determines the degree of immunocompromise. A determination that a health-care worker is severely immunocompromised ultimately must be made by his or her physician. Immunocompromised health-care workers and their physicians should consider the risk for exposure to a vaccine-preventable disease together with the risks and benefits of vaccination.

The exact amount of systemically absorbed corticosteroids and the duration of administration needed to suppress the immune system of an otherwise healthy individual are not well defined. Most experts agree that steroid therapy usually does not contraindicate administration of live virus vaccines such as MMR and its component vaccines when therapy is a) short term (≦14 days) low to moderate dose; b) low to moderate dose administered daily or on alternate days; c) long-term alternate day treatment with short-acting preparations; d) maintenance physiologic doses (replacement therapy); or e) administered topically (skin or eyes) by aerosol, or by intraarticular, bursal, or tendon injection. Although the immunosuppressive effects of steroid treatment vary, many clinicians consider a steroid dose that is equivalent to or greater than a prednisone dose of 20 mg per day sufficiently immunosuppressive to cause concern about the safety of administering live virus vaccines. Persons who have received systemic corticosteroids in excess of this dose daily or on alternate days for an interval for ≧14 days should avoid vaccination with MMR and its component vaccines for at least one month after cessation of steroid therapy. Individuals who have received prolonged or extensive topical, aerosol, or other local corticosteroid therapy should not receive MMR or its component vaccines, and varicella vaccine for at least one month after one month after cessation of therapy. Persons who have a disease that, in itself, suppresses the immune response and who are also receiving either systemic or locally administered corticosteroids generally should not receive MMR, its component vaccines, or varicella vaccine. The use of ImmunoScore diagnostic analyses and database for immunocompromised health-care workers can be used for assessing these workers and in monitoring them following corticosteroid therapy for levels of immune response.

In general, symptomatic HIV-infected individuals have suboptimal immunologic responses to vaccines. The response to both live and killed antigens may decrease as the disease progresses (Vardinon, et al. 1990). Administration of higher doses of vaccine or more frequent boosters to HIV-infected persons may be considered. However, because neither the initial immune response to higher doses of vaccine nor the persistence of antibody in HIV-infected patients has been systematically evaluated, recommendations cannot be made at this time (CDC, 1997).

Limited studies of MMR immunization in both asymptomatic and symptomatic HIV-infected patients who did not have evidence of severe immunosuppression documented no serious or unusual adverse events after vaccination (Onorato, et al. 1992). HIV-infected persons are at increased risk for severe complications if infected with measles. Therefore, MMR vaccine is recommended for all asymptomatic HIV-infected health-care workers who do not have evidence of severe immunosuppression. Administration of MMR to HIV-infected health-care professionals who are asymptomatic but do not have evidence of severe immunosuppression because of a) a case of progressive measles pneumonia has been reported after administration of MMR vaccine to a person with AIDS and severe immunosuppression, b) the incidence of measles in the United States is currently very low, c) vaccination-related morbidity has been reported in severely immunocompromised persons who were not HIV-infected, and d) a diminished antibody response to measles vaccination occurs among severely immunocompromised HIV-infected individuals.

Recommendations of the CDC (1997)

Recommendations for administration of vaccines and other immunobiological agents to health-care professionals are organized in three broad disease categories:
those for which active immunization is strongly recommended because of special risks for health-care workers (i.e. hepatitis B, influenza, measles, mumps, rubella, and varicella);
those for which active and/or passive immunizations of health-care workers may be indicated in certain circumstances (i.e. tuberculosis, hepatitis A meningococcal disease, typhoid fever, and vaccinia) or in the future (i.e. pertussis); and
those for which the immunization of all adults is recommended (i.e. tetanus, diphtheria, and pneumococcal disease).

Immunization is strongly recommended for hepatitis B, influenza, measles, mumps, rubella, and varicella. In exemplary embodiments of the present invention, an ImmunoScore diagnostic panel can be in place in health-care settings for the routine monitoring of health-care professionals. Such an ImmunoScore database can combine information obtained from immune status of health-care workers with that of other segments of the population. The panel can be a valuable tool for the health-care industry and hopefully reduce the burden of vaccine-preventable nosocomial illnesses. There are other diseases for which vaccination should be considered; those include tuberculosis (pretty much as a last resort), hepatitis A, pertussis, meningococcal disease, typhoid fever, and vaccinia. Other vaccine-preventable diseases for which protection should be maintained include tetanus, diphtheria, and pneumococcal disease. Levels of antibodies can be monitored periodically by ImmunoScore diagnostic immune status assays. In addition, the overall immune health could be measured initially using the meningococcal diagnostic panel. A typhoid fever antibody assay could also be developed for health-care professionals. In addition, a hepatitis C antibody assay can also need to be established. There is as yet no vaccine for hepatitis C, but an HCV infection presents a risk of nosocomial infections.

TABLE 7
Recommended Immunizing Agents for Health Care Workers

| Vaccine | Schedule |
|---|---|
| Hepatitis B | 3 doses |
| Influenza | Annual |
| Measles, Mumps, Rubella | 1 dose |
| Varicella | 2 doses |
| Tuberculosis (BCG) | 1 does (in high risk settings) |
| Hepatitis A | 2 doses |
| Meningococcal disease | 1 dose |
| Pertussis | 1 dose (needs reformulation) |
| Typhoid | 1 dose, boost 2 yrs. |
| Vaccinia | 1 dose, boost 10 yrs. |
| Tetanus, diphtheria (Td) | 1 dose, boost 10 yrs. |
| Pneumococcal polysaccharide | 1 dose, boost >5 yrs. in high risk settings |

The only analyte specific for health care workers is tuberculosis. There is no easily measurable correlate of immunity to tuberculosis. Delayed type hypersensitivity, as measured by the tuberculin test, is not a measure of resistance, because both reactivation and superinfection may occur in tuberculin-positive subjects (Plotkin, 2001). Although current experimental vaccinology is investigating the potential of proteins and capsule of Mycobacterium tuberculosis, the majority opinion is still that antibodies are irrelevant to protection (McAdam, 1997). Thus, in exemplary embodiments of the present invention, ImmunoScore diagnostic analysis can include measurement of as yet undetermined cellular components important to controlling TB infection.
   5. ImmunoScore Analyses and Bioterrorism

Before the anthrax attacks of 2001, the threat of bioterrorism was based primarily on the events surrounding the sarin nerve agent attacks in Japan in 1995 and the biological weapons production and stockpiling programs of the former Soviet Union and Iraq. Now that terrorists have used the United States Postal Service to disseminate anthrax spores contained in letters, the threat of lethal bioterrorism has become a reality (Darling, et al. 2002).

In June of 1999, the Centers for Disease Control and Prevention (CDC) and a multidisciplinary panel of experts formed a strategic workgroup to outline steps to strengthen the US public health infrastructure and health-care capacity to protect against bioterrorism (CDC, 2000). They stated that the public health infrastructure must be prepared to prevent illness and injury that would result from biological and chemical terrorism, especially a covert terrorist attack.

In the past, most planning for emergency response to terrorism has been concerned with overt attacks (e.g. bombings). Chemical terrorism acts are likely to be overt because the effects of chemical agents absorbed through inhalation or by adsorption through the skin or mucous membranes are usually immediate and obvious. Such attacks elicit immediate response from police, fire, and EMS personnel.

In contrast, attacks with biological agents are more likely to be covert. They present different challenges and require an additional dimension of emergency planning that involves the public health infrastructure. Covert dissemination of a biological agent in a public place will not have an immediate impact because of the delay between exposure and the onset of illness.

Consequently, the first casualties of a covert attack probably will be identified by physicians or other primary health-care providers.

Potential biological and chemical agents are numerous, and the public health infrastructure must be equipped to quickly resolve crises that would arise from a biological or chemical attack. However, to best protect the public, the preparedness efforts must be focused on agents that might have the greatest impact on U.S. health and security, especially agents that are highly contagious or that can be engineered for widespread dissemination via small-particle aerosols. Early detection requires increased biological and chemical terrorism awareness among front-line health-care providers because they are in the best position to report suspicious illnesses and injuries. Also, early detection will require improved communication systems between those providers and public health officials. In addition, state and local health-care agencies must have enhanced capacity to investigate unusual events and unexplained illnesses, and diagnostic laboratories must be equipped to identify biological and chemical agents that are rarely seen in the United States. Fundamental to these efforts is comprehensive, integrated training designed to ensure core competency in public health preparedness and the highest levels of scientific expertise among local, state, and federal partners. ImmunoScore diagnostic analyses can be an integral part of preparation of events of bioterrorism.

The CDC has outlined the following steps for preparation for terrorist attacks using biological agents:
Enhance epidemiologic capacity to detect and respond to biological attacks.
Supply diagnostic reagents to state and local public health agencies.
Establish communication programs to ensure delivery of accurate information.
Enhance bioterrorism-related education and training for health-care professionals.
Prepare educational materials that will inform and reassure the public during and after a biological attack.
Stockpile appropriate vaccines and drugs.
Establish molecular surveillance for microbial strains, including unusual or drug-resistant strains.
Support the development of diagnostic tests.
Encourage research on antiviral drugs and vaccines.

The planning group assembled by the CDC categorized biological agents according to their perceived level of threat. The first of these are Category A agents. These high-priority agents include organisms that pose a risk to national security because they:
can be easily disseminated or transmitted person-to-person;
cause high mortality, with potential for major public health impact;
might cause public panic and social disruption; and
require special action for public health preparedness.

Category A agents include:
Variola major (smallpox)
Bacillus anthracis (anthrax)
Yersinia pestis (plague)
Clostridium botulinum toxin (botulism)
Francisella tularensis (tularaemia)
filoviruses:
   Ebola hemorrhagic fever
   Marburg hemorrhagic fever
arenaviruses:
   Lassa (Lassa fever)
   Junin (Argentine hemorrhagic fever) and related viruses

It would be difficult to create a more “perfect” biological weapon than Bacillus anthracis, the causative agent of anthrax. Infection, usually by spores, is introduced through scratches or abrasions of the skin, inhalation, eating insufficiently cooked infected meat, or by the bites of flies. Anthrax spores may remain stable for decades or can be produced, weaponized, and delivered as a wet or dry aerosol cloud.

The bioterrorism related inhalational anthrax cases that occurred during the fall of 2001 presented in a predictable manner with a few exceptions. Nearly all patients initially developed fatigue and malaise followed by minimal or non-productive cough. They soon developed fever, chills, nausea, vomiting, and drenching sweats. This progressed to chest pain and dyspnea.

Bacillus anthracis is detectable by Gram stain of the blood, blood culture on routine media, and by ELISA, but often not until later in the course of the illness. Approximately 50% of the cases are accompanied by hemorrhagic meningitis, and therefore organisms may also be identified in cerebrospinal fluid (Bush, et al. 2001). Only vegetative encapsulated bacilli are present during infection. Spores are not found within the body unless the bacilli are exposed to ambient air. Toxin production parallels the appearance of bacilli in the blood, and tests are available to rapidly detect the toxin. With the appearance of symptoms, the white blood cell count becomes elevated and remains so until death. The primary cause of morbidity and mortality is believed to be the extreme toxin load generated by the organism.

More than 30,000 patients have been taking ciprofloxacin or doxycycline as post-exposure prophylaxis since the bioterrorism incidents of October 2001 (CDC, 2001). If confirmed that anthrax has been used as a biological weapon, antibiotics should be continued for at least 60 days in all exposed individuals, and patients should be closely followed after antibiotics are discontinued. Military doctrine also requires that service members begin active immunization with anthrax vaccine while taking post-exposure antibiotics. Anthrax vaccine is not currently available for use by the general public. In response to the bioterrorism events of 2001, however, CDC offered anthrax vaccine as part of an investigational new drug (IND) protocol. This was necessary because anthrax vaccine was never licensed for use as a post-exposure treatment (Michigan Dept. Public Health, 1978). The CDC had no recommendation as to whether patients should or should not receive the vaccine; this led to considerable confusion and consternation among the public. Consequently, few patients chose to receive the vaccine.

On discontinuation of antibiotics, patients should be closely observed. If clinical signs of anthrax develop, empiric therapy for anthrax is indicated, pending etiologic diagnosis. Optimally, patients should have medical care available from a fixed facility with intensive care capabilities and readily available access to infectious disease consultants. ImmunoScore diagnostic assays could become integral to diagnosis and treatment of anthrax patients. The database information would be valuable at helping to determine the serological correlate of protection for anthrax vaccine.

Smallpox is caused by the Variola virus. There are no non-human reservoirs for smallpox and no human carriers. The disease has survived throughout history through continual person-to-person transmission. Smallpox was probably responsible for more than 100 million deaths during the 20^{th} century alone.

Smallpox is perhaps the most feared of potential biological warfare agents. Researchers estimate that vaccinated individuals retain immunity for approximately 10 years, although in selected populations this may continue past 20 years (Henderson and Moss, 1999). Therefore, most of the population of the United States is probably susceptible to smallpox. Vaccines are in short supply; however, the Federal government has entered into contracts to rectify this. Finally, because few physicians are familiar with the clinical presentation of smallpox, recognizing an outbreak may be problematic.

The smallpox virion is readily transmitted from person to person by way of respiratory particles. Virions can also remain viable on fomites for up to one week. The virus initially replicates in respiratory tract epithelial cells then migrates to regional lymph nodes. From there, a massive asymptomatic viremia ensues three to four days later and may result in focal infections involving lymphoid tissues, skin, intestines, lungs, kidneys, or brain (Henderson, et al. 1999). Initial symptoms resemble an acute viral illness. Following an incubation period of approximately 12 days, a second viremia, lasting two to five days, results in high fevers, malaise, headache, backache, rigors, and vomiting. The patient may develop delirium. A rash typically develops within 48 hours, beginning in the mouth, and heralds the onset of viral shedding. The rash rapidly spreads to the hands and forearms followed by the legs and trunk. The rash becomes distinctive when the lesions become pustular. Viral shedding and secondary infection cases may occur from the onset of rash until scabs have separated (Henderson, et al. 1999). Death usually occurs late in the first week or during the second week of the illness and is caused by the toxemia induced by the overwhelming viremia.

During the vesicular stage, the rash may resemble chickenpox. There are two important distinctions, however. First, the rash of smallpox develops synchronously, in contrast to the asynchronous development observed with varicella. Second, the rash of smallpox is concentrated on the face and extremities, as opposed to on the trunk as occurs in chickenpox (Henderson, 1999)

Initial diagnosis will likely be clinical, based on the characteristic rash. Diseases with similar skin manifestations must be considered in the differential diagnosis, including cutaneous lues (syphilis), meningococcemia, acute leukemia, or drug toxicity. Laboratory confirmation is extremely important, as a single case of smallpox must be treated as an international public health emergency. Smallpox can be confirmed through clinical presentation and identification of the virion particles on electron microscopy of vesicular fluid, although this only confirms presence of an orthopox virus. Further classification of the orthopox virus requires cell culture or growth on chorioallantoic egg membrane. ImmunoScore diagnostic analysis can be used to identify levels of smallpox antibody in sera of individuals. In addition, ImmunoScore database analyses could be performed on larger numbers of individuals to track the longevity of serum antibody to smallpox.

Yersinia pestis, a gram-negative bacillus, has tormented mankind throughout history. The Byzantine Empire recorded a sixth century pandemic, and the Black Death killed millions of people throughout 14^{th} century Europe. The most recent pandemic originated in China and spread worldwide at the turn of the 20^{th} century (McGovern, et al. 1989).

Plague is a zoonosis with a rodent host and a flea vector. The vector is not essential, however, and direct host-to-host transmission can occur by way of an infectious aerosol. A bite from an infected flea causes an infection in the lymphatic system leading to the bubonic form of the disease. Inhalation of aerosolized bacillus, preferred for deliberate dissemination, results in a primary pulmonary infection, known as pneumonic plague. The disease is rapidly fatal in the absence of prompt antibiotic treatment and may result in secondary contagion spread.

Modern efforts to weaponize Y pestis were begun by the Japanese during World War II, but dissemination attempts were met with limited success. Infected fleas were bred by the billions and then released over northern Chinese cities that had not previously recorded plague casualties. Epidemics subsequently occurred and plague has remained endemic in the region since (Williams and Wallace, 1989). The United States dismissed plague as a potential weapon because of its persistence in the environment and friendly casualties after an attack. The former Soviet Union's extensive biological warfare program, however, reportedly included, dry, antibiotic-resistant, environmentally stable forms of the plague organism that could be disseminated as an aerosol (Alibek, 1999).

Skin penetration or direct ingestion of fewer than ten Y. pestis organisms can induce an infection in humans. The clinical course will vary substantially with the route of exposure. If plague were used as a biological weapon, the most likely exposure would be via inhalation. Pneumonic plague presents without buboes and may progress rapidly if vegetative organisms with previously developed antiphagocytic capsules and Yersinia outer-membrane protein (Yop) antigens have been inhaled as an aerosol (Poland, 1989). Most patients develop a productive cough with blood-tinged sputum within 24 hours of the onset of symptoms. This is an important diagnostic clue that should lead one to consider bioterrorism if many previously well patients present with this sign (Cavanaugh, et al. 1982).

Serum antibody to Fraction I capsular antigen, as measured by the passive hemagglutination (PHA) test, is correlated with resistance to Y. pestis infection in experimental animals. A comparable correlation between PHA titer and immunity probably occurs in humans (CDC, 1982). Plague vaccine that was protective against bubonic plague is no longer available. At any rate, it did not protect against aerosol infection in test models (Ehrenkranz and Meyer, 1955). A vaccine for pneumonic plague is under development, but that is not a guarantee of success. ImmunoScore diagnostic analysis should be a willing partner for analyses of any vaccines under development for combating plague.

The causative agent of tularemia, Francisella tularensis, is a small aerobic, non-motile, gram-negative, cocco-bacillus. Tularemia is a zoonotic disease that humans may acquire after skin or mucous membrane contact with tissues or body fluids of infected animals, or from bites of infected ticks, deerflies, or mosquitoes. Less commonly, inhalation of contaminated dusts or ingestion of contaminated foods or water may produce clinical disease. Respiratory exposure by aerosol would typically cause typhoidal or pneumonic tularemia. F. tularensis remains viable for weeks in water, soil, carcasses, hides, and for years in frozen meat. Resistant for months to temperatures of freezing and below, it is easily killed by heat and disinfectants (Evans and Friedlander, 1997).

Francisella tularensis was weaponized by the United States in the 1950s and 1960s during the U.S. offensive biowarfare program. Other countries are suspected to have weaponized this agent. This organism could potentially be stabilized for weaponization by an adversary and produced in a wet or dried form for delivery against U.S. forces or as a weapon of terror.

Onset of disease is usually acute and occurs after an incubation period that ranges from 1 to 21 days. In humans, as few as 10 to 50 organisms may cause disease if inhaled or injected intradermally (McCrumb, et al. 1957). All ages are susceptible, and recovery is generally followed by permanent immunity.

Typhoidal tularemia occurs mainly after inhalation of infectious aerosols, but can also occur after intradermal or gastrointestinal challenge. F. tularensis would most likely be delivered as an aerosol if used as a weapon and would primarily cause typhoidal tularemia that manifests as fever, prostration, and weight loss. Pneumonia may be severe and fulminant. Respiratory symptoms and a cough (productive or non-productive) may also be present. Case fatality rates may be greater than the 1-3% seen with appropriately treated natural disease. Case fatality rates are approximately 35% in untreated naturally acquired typhoidal cases (Darling, et al. 2002). Similar to many bacterial and viral diseases, early symptoms of exposure to F. tularensis are fairly generic and nonspecific, making differential diagnosis difficult.

At present, a live vaccine strain (LVS) tularemia vaccine is under IND status in a protocol at the U.S. Army Medical Research Institute of Infectious Diseases (USAMRIID), and is available only for at-risk U.S. military personnel. It is administered by scarification. Despite the increased risk of a bioterror threat felt after September 11, further vaccine development for tularemia remains slow. The projected date of a new licensed vaccine in the United States is not until 2009 (Nierengarten and Lutwick, 2002). There is some confusion over which arm of the immune system should be targeted. New lots of LVS produced in the United States show immunogenicity in human volunteers, producing both brisk cell-mediated and humoral immune responses. ImmunoScore diagnostic analysis can be applied in this setting to monitor the response to vaccines in clinical trials and follow the duration of the immune response. In addition, cellular components of the immune system can also be tracked, for example, through compilation of information added to an ImmunoScore database.

The viral hemorrhagic fevers are caused by a diverse group of RNA viruses in four separate families: Arenaviridae, Bunyaviridae, Filoviridae, and Flaviviridae. All have lipid envelopes, limited geographic ranges, are highly infectious by way of the aerosol route (except Dengue), and are believed to have animal reservoirs with arthropod vectors. Terrorist groups have attempted to weaponize agents from this class (Carus, 2001). Each disease is characterized by its own unique characteristics, but all have a final common pathway of diffuse hemorrhage and bleeding diathesis.

Yellow fever and dengue (Flaviviridae) are probably the archetypical diseases of this group, but are not considered significant biological warfare threat agents. Hantavirus (Bunyaviridae) is enzootic in rodents. West Africa's Lassa fever and Argentine, Bolivian, Brazilian, and Venezuelan hemorrhagic fevers (Arenaviridae) are also enzootic in rodents within their respective areas. The most publicized viral hemorrhagic fevers are the Ebola and Marburg (Filoviridae) viruses. These viruses produce grotesquely lethal diseases. The reservoir and natural transmission of Ebola and Marburg are unknown but they are readily transmissible by infected blood and tissue. Aerosols may be formed naturally when infectious body fluids are expelled or in the case of hantavirus when rodent feces and urine are resuspended by movement in the area. Laboratory cultures can yield sufficient concentrations of organisms to provide a credible terrorist weapon if disseminated as an aerosol (Darling, et al. 2002).

In a bioterrorism scenario, aerosol dissemination would result in many patients who shared a common location approximately three to eight days before presentation. Specific disease identification currently requires ELISA detection of antiviral IgM antibodies or direct culture of the viral agent from blood or tissue samples. During the clinical course of each of the diseases, hepatocellular enzymes are often elevated. Appropriate precautions should be observed in collection, handling, and processing of diagnostic samples, which should be sent to a Level D laboratory that currently exist only at the CDC or USAMRIID. The only likely application of ImmunoScore for hemorrhagic fever viruses would be in analyses of vaccines in development and the possible placement with the CDC or USAMRIID for diagnoses.

Clostridium botulinum is a gram-positive, spore-forming anaerobic bacillus found in soil around the world. Botulism is the syndrome caused by botulinum toxin produced by this bacterium. Cases have historically been categorized according to transmission as food-borne illness (from ingestion of the toxin in home-canned goods, poorly heated vegetables, or meats), wound botulism (secondary to soil-contaminated wounds, drug abuse, and C-section deliveries), and infantile illness (from ingestion of spores) (Amon, et al. 2001).

Botulinum toxin is one of the most toxic substances known (Middlebrook and Franz, 1998). Seven distinct types of toxin exist, identified by antigenicity and referred to as types A-G. Botulinum toxin could be used to sabotage food supplies, although a more likely scenario would involve dissemination as an aerosol. During the Gulf War, Iraq produced 20,000 L of botulinum toxin, 12,000 L of which were used in field testing and to fill warheads (Zilinskas, 1997). Despite the efforts to produce an effective botulinum toxin weapon, most authorities agree that it is unlikely this toxin could ever be effectively deployed as a weapon of mass destruction. Aerosol delivery over a battlefield or a defined geographic region populated by civilians would require a precisely orchestrated effort. Large quantities of toxin would have to be delivered to the area at the optimum time because botulinum toxin quickly degrades in the environment. Even municipal water reservoirs are most likely safe from contamination by terrorist actions because literally ton quantities would be necessary because of the effects of dilution.

In the emergency setting the diagnosis of botulism intoxication will be clinical. An influx of patients with descending muscle paralysis and bulbar findings may herald a bioterrorist event or a natural outbreak of food-borne botulism. No routine laboratory tests will aid in the diagnosis. The toxin may be detected by assays of serum or gastric contents.

Three different antitoxin preparations are available in the United States. Antitoxin may prevent progression or shorten the course of the illness. A pentavalent toxoid of Clostridium botulinum toxin types A, B, C, D, and E is available as an IND product for pre-exposure prophylaxis. The currently recommended primary series of 0, 2, and 12 weeks, followed by a 1-year booster, induces immunity in greater than 90% of vaccinees after one year (Darling, et al. 2002). In exemplary embodiments, ImmunoScore analyses can be useful in examining response to the prophylactic vaccine as well as in following the duration of protection.

The Category B agents are the second highest priority and they include those agents that:
are moderately easy to disseminate;
cause moderate morbidity and low mortality; and
require specific enhancements of CDC's diagnostic capacity and enhanced disease surveillance

Category B agents include:
Coxiella burnetti (Q fever)
Brucella species
Burkholderia mallei
alphaviruses
ricin toxin from Ricinus communis (castor beans)
epsilon toxin of Clostridium perfringens
Staphylococcus enterotoxin B
Salmonella species
Shigella dysenteriae
Escherichia coli O157:H7
Vibrio cholerae
Cryptosporidium parvum

The Category C agents are the third highest priority and they include emerging pathogens that could be engineered for mass dissemination in the future because of:
availability;
ease of production and dissemination; and
potential for high morbidity and mortality and major health impact.

Category C agents include:
Nipah virus
hantaviruses
tickborne hemorrhagic fever viruses
tickborne encephalitis viruses
yellow fever
multidrug-resistant tuberculosis

Preparedness for Category C agents requires ongoing research to improve disease detection, diagnosis, treatment, and prevention. Knowing in advance which newly emergent pathogens might be employed by terrorists is not possible; therefore linking bioterrorism preparedness efforts with ongoing disease surveillance and outbreak response activities is imperative.

Although once considered unlikely, bioterrorism is now a reality in the United States since the anthrax cases began appearing in the fall of 2001. Intelligence sources indicate there are many countries and terrorist organizations that either possess biological weapons or are attempting to procure them. In the future it is likely that we will experience additional acts of bioterrorism. The CDC category A agents represent the greatest challenge because they have the potential to cause grave harm to the medical and public health systems of a given population. Thus, it is imperative that plans be developed now to deal with the consequences of an intentional release of any one or more of these pathogens (Darling, et al. 2002).

In exemplary embodiments of the present invention, an ImmunoScore diagnostic platform can be constructed so as to be able to grow with the needs of bioterror agent analyses. As new agents arise, diagnostic testing can available to test for immune responses to such agents as well as any vaccines that have been or will be developed.

6. ImmunoScore Analyses for Infection and Chronic Disease

Chronic diseases take a huge toll. In the United States, more than 70% of all deaths are due to one or more chronic diseases, and more than 90 million people suffer daily. Even diseases not typically associated with pathogens may have underlying infectious causes. Of the eight million new cases of cancer in the world each year, one million are attributable to a known infectious agent.

The infectious origins of some chronic diseases have been known for decades. These include tuberculosis, syphilis, leprosy, and a number of parasitic diseases. Only more recently has it been realized that coronary artery disease, diabetes mellitus, cancer, and neurological disorders can have an infectious etiology, either as a cause or a co-factor.

Koch's postulates for distinguishing a pathogenic from an adventitious microbe were formulated in 1884. Koch stated for an organism to be pathogenic, it needed to fulfill the following criteria:
the organism is regularly found in the lesions of the disease
the organism can be isolated in pure culture on artificial media
inoculation of this culture produces a similar disease in experimental animals
the organism can be recovered from the lesions of these newly infected animals

Koch's postulates are indeed relevant for acute infections, but there is a problem using them when considering chronic or long-term symptoms. With chronic illness, the requirements to culture microorganisms and demonstrate infectivity may have become obsolete. Microbes do not use one single strategy to disable their hosts for the long term. Several of the long-term strategies include:
induction of autoimmunity (Group A Streptococcus and heart valve disease)
persistent or repeated infection (HIV)
Non-obvious connection to chronic disease (Helicobacter pylori and gastric ulcers)

Helicobacter pylori is a gram-negative bacterium that causes a lifelong infection in over half of the world's human population. Without specific antimicrobial treatment, all infected individuals exhibit chronic gastric inflammation, and a small percentage will develop peptic ulcers and gastric adenocarcinoma or mucosa associated lymphoid tissue lymphoma. In response to infection, the host launches a vigorous immune response, including the mucosal infiltration of neutrophils, lymphocytes, and macrophages. This immune response is insufficient for clearance of the bacterium, suggesting the H. pylori is capable of evading host immune responses.

Infection with H. pylori induces apoptosis in macrophages, disrupts phagosome maturation, and disrupts cytokine signaling. Induction of macrophage apoptosis may represent a mechanism by which H. pylori usurps the host immune response to establish a chronic infection in humans.

Cardiovascular disease for all causes accounts for 29% of all deaths worldwide (behind only infectious and parasitic diseases). Deaths from cardiovascular disease are often premature, and millions of non-fatal events result in disability. Atherosclerosis, a major component of cardiovascular disease, has been considered a public health problem of industrialized countries.

Many individuals with atherosclerosis lack identifiable traditional risk factors (smoking, diet and exercise, hypercholesterolemia, hypertension, diabetes, and genetic factors). Atherogenic processes resemble many aspects of chronic inflammation, a response that may be promoted by microorganisms. Both Chlamydia pneumoniae and cytomegalovirus (CMV) are widely distributed, can infect blood vessel walls, and exhibit persistence, latency, and recurrence of infection.

There are several lines of evidence associating C. pneumoniae infection with atherosclerosis. These lines of evidence include:
seroepidemiologic studies
direct detection of bacterial components in atherosclerotic lesions
isolation of viable organisms from coronary and carotid atheromatous tissue
in vitro and animal experiments

Cross-sectional and prospective studies have correlated seroprevalence with myocardial infarction, chronic coronary heart disease, or stroke.

More than 38 studies have reported a positive association between antibodies to C. pneumoniae and atherosclerotic disease. However, more than 50% of adults have been infected with C. pneumoniae at least once. The strongest evidence associating C. pneumoniae and atherosclerotic cardiovascular disease has been detection of bacterial components in atherosclerotic lesions. Historic findings do not, however, establish a causal role for C. pneumoniae in atherogenesis.

Studies have linked cytomegalovirus to three arterial diseases—primary atherosclerosis, post-angioplasty restenosis, and post-transplantation arteriosclerosis. Seroepidemiology has relied on single measures of viral IgG antibodies which only indicate previous exposure. Like C. pneumoniae, the worldwide ubiquity of lifelong, latent CMV infections could mask or falsely highlight causality.

Other microbes may be associated with cardiovascular disease. Several reports have suggested a relationship between chronic oral infections (e.g. periodontitis) and cardiovascular disease. These oral pathogens include Porphyromonas gingivalis, Bacteroides forsythus, Campylobacter rectus, Fusobacterium nucleatum, Treponema spp., Prevotella spp., and Streptococcus sanguis. Raised H. pylori and Herpes Simplex Virus (HSV) antibody levels have also been associated with cardiovascular disease. Mycobacterial disease shares interesting connections to heart disease. Not only is tuberculosis the only microorganism to depend on cholesterol for its pathogenesis, but CDC maps for cardiovascular disease bear a striking similarity to those of State and regional tuberculosis cases. Present day markers suggested as indicators for heart disease susceptibility such as C-reactive protein (CRP), interleukin-6, and homocysteine are all similarly elevated in tuberculosis.

Group A streptococci are important human pathogens which cause a variety of pyrogenic infections that can be mild (e.g. pharyngitis, impetigo) to extremely severe (cellulites, necrotizing fasciitis, septicemia, pneumonia and streptococcal toxic shock syndrome). Molecular mimicry between streptococcal and heart components has been proposed as the triggering factor leading to autoimmunity in rheumatic heart disease.

Medically significant complications of Herpes Simplex Virus (HSV) are rare, but constitute a significant burden, given the high rates of HSV seropositivity in the population. HSV ocular infection is the leading cause of infectious corneal blindness in the United States. HSV-1 shedding is associated with reduced hospital survival in patients receiving assisted ventilation in intensive care units. Following productive infection by HSV at the site of inoculation, the virus spreads to and enters sensory neurons, where it establishes a latent infection. Latent infection forms a reservoir of virus for recurrent infection, disease, and transmission to other individuals. HSV-1 is usually associated with primary infections of the orofacial area and latent infections of the trigeminal ganglion. HSV-2 is usually associated with genital infections and latent infection in sacral ganglia.

Human papillomavirus (HPV) is one of the most common causes of sexually transmitted disease in both men and women world wide. HPV is associated with a variety of clinical conditions that range from innocuous lesions to cancer. Most HPV infections are benign—plantar and palmar warts, common warts, and flat warts. Strains that target the face make skin cancer more likely. Other strains that grow primarily in the lining of the mouth produce small elevated nodules that can develop into fatal squamous cell cancers. Cervical cancer is the third most common cancer in women in the United States. The magnitude of the association between HPV and cervical squamous cell carcinoma is higher than that for the association between smoking and lung cancer. HPV has been implicated in 99.7% of cervical squamous cell cancer cases world wide.

Pseudomonas aeruginosa is classified as an opportunistic pathogen, primarily infecting individuals who are immunocompromised, such as patients with cancer or AIDS. Cystic fibrosis (CF) almost always leads to chronic airway infection with P. aeruginosa. Despite advances in antibiotic therapy, after chronic infection, rapid deterioration in lung function occurs, increasing morbidity and mortality. Chronic P. aeruginosa airway infections remain the primary cause of morbidity and mortality in the CF population. Young children with CF may be infected as early as 6 months of age and P. aeruginosa becomes chronic in the first decade of life with pulmonary exacerbations increasing in frequency. A pulmonary infection with P. aeruginosa is characterized by a strong recruitment of neutrophils and significant inflammation in the lung parenchyma, which results in extensive damage to the lung tissue through the action of neutrophil enzymes and oxidants.

Tuberculosis has been declared a global emergency. Pulmonary TB is the second leading cause of mortality from infectious disease world wide, with 8 million new cases and 2 million deaths due to TB each year. There is an urgent need for rapid, cost-effective, and accurate methods for the diagnosis of TB. A serologic test is attractive because it would be relatively rapid and would not require sputum expectoration. Challenges for the development of effective serologic tests include:
the need to discriminate active disease from latent infection
to avoid cross-reactivity with M. bovis BCG or mycobacteria other than M. tuberculosis
to perform consistently with genetically and immunologically diverse populations

Lyme disease is a troubling chronic infection. Infection of humans by Borrelia burgodorferi results in a spectrum of clinical illnesses. Earliest symptoms may include a typical or atypical rash, followed by flu-like illness. As the disease progresses, other neurologic and musculoskeletal symptoms and signs may develop. The pathophysiology of the chronic symptoms is not well understood, with hypotheses ranging from persisting infection to autoimmunity to a combination of the two. The diagnosis of chronic Lyme disease has been made difficult because of several factors. The multi-symptom complex consisting of fatigue, musculoskeletal pains and neurocognitive dysfunction cannot be distinguished from disorders that have been termed fibromyalgia, chronic fatigue and Gulf War syndrome. Laboratory testing has not been reliable, including cultures, antibody studies (ELISA, Western blot) and PCR-DNA tests.

Malaria, which had been eliminated or effectively suppressed in many parts of the world, is undergoing a resurgence. Malaria is estimated to cause up to 400 million clinical cases and 2 million deaths each year. Many of the clinical manifestations of malaria (including acute febrile illness, anemia, cerebral malaria, and hypoglycemia) are mediated in part by overproduction of pro-inflammatory cytokines such as tumor necrosis factor (TNF-α), interleukin-1 (IL-1) and gamma interferon (IFN-γ).

Hepatitis C virus (HCV) is a small RNA virus that chronically infects 170-350 million people world wide. Of those acutely infected, only 15% recover, while the remaining 85% succumb to chronic hepatitis. Up to 20% of the individuals with chronic hepatitis C progress to cirrhosis and these patients are at greater risk of developing hepatocellular carcinoma. Extensive studies have been carried out in the past decade in order to find immunodominant HCV peptides and there are many peptides capable of inducing cellular immune responses. None of these, however, has proven to be clinically effective in preventing HCV disease. Although interferon and other agents are effective for eliminating HCV in certain patients, they are too expensive for the majority of HCV patients in most countries. There is an urgent need to determine immunodominant peptides useful for the development of effective and low-priced vaccines. In addition, there is also a need to develop a simple and low-priced diagnostic tool for HCV since the currently available kit is also expensive for the majority of people not living in developed countries.

Epstein-Barr Virus (EBV) is a B-lymphotropic human herpesvirus, and like other herpesviruses, establishes a lifelong presence in the host. The virus infects the vast majority of the world's adult population and is well known for its association with a broad spectrum of benign and malignant diseases including:
infectious mononucleosis
Burkitt's lymphoma
nasopharyngeal carcinoma
B-cell lymphoma in immunocompromised individuals.

Respiratory tract infections caused by viruses, Chlamydia, and Mycoplasma have been implicated in the pathogenesis of asthma. Viruses have been demonstrated to be associated with asthma epidemiologically in at least two ways. During infancy, certain viruses have been implicated as potentially being responsible for the inception of the asthmatic phenotype. In patients with established asthma, viral upper respiratory tract infections play a significant role in producing acute exacerbations of airway obstruction that may result in frequent outpatient visits or hospitalizations. Recent attention has focused on Chlamydia and Mycoplasma as potential contributors to both exacerbations and the severity of chronic asthma in terms of loss of lung function or medication requirements.

Various microorganisms are implicated in the initiation and/or progression of chronic illnesses. There are other effects of carriage of these microorganisms on the immune system (e.g. cytokines, cellular responses, effector molecules). Monitoring of antibody responses and plasma cytokine levels merit serious consideration for ImmunoScore diagnostic analyses in exemplary embodiments of the present invention.

7. Th1-Th2 Paradigm

Lymphocytes are the effector cells of acquired (or adaptive) immunity, originating as bone marrow stem cells that undergo hematopoiesis. A portion of these lymphocytes migrate to the thymus to undergo further differentiation and maturation to become T cells, which can be divided into subsets based on physical markers or surface receptors (e.g., CD4, CD8, and either αβ or γδ T cell receptor), representing a generally irreversible genetic commitment (for review, see Kidd P, 2003; Pier G B et al., 2004). Other subsets have been defined by functional properties that may be environmentally altered; e.g., expression of different cytokines, which are chemicals used for cell-to-cell communication. It was originally determined in mice that there are two T helper cell subsets, Th1 and Th2, based on two distinct cytokine profiles that resulted in the overall regulation of an immune response (Mosmann T R et al., 1986; Mosmann T R, Coffman R L, 1989). For example, FIG. 4I (from Harber M et al., 2000) shows some of the complex interactions between the polarized Th1 and Th2 responses.

It is clear from this Th1-Th2 paradigm that the cytokines secreted by the Th cells will feedback and reinforce the particular clonal phenotype from which they originated (e.g., IL-4 for Th1 vs. IFN-γ for Th2), as well as suppress the alternate phenotype, resulting in crossregulation.

The same Th1-Th2 paradigm from mice has been applied to humans (Romagnani S, 1991; Del Prete G F et al., 1991) to also explain the immunologic aspect of disease (Lucey D R et al., 1996; Romagnani S et al., 1997; Romagnani S, 1997). FIG. 4J (from Harber M et al., 2000) shows the complex balance between Th1 and Th2 cells as dictated by the Th1-Th2 paradigm regarding disease.

The Th1 cell (with its associated cytokines: INF-γ, TNF-α, IL-2, IL-12) is biased towards the cell-mediated side of immunity, effective against intracellular parasites, and its downregulation of Th2 can provide relief from allergic reactions due to IgE; but detrimental effects may result in autoimmunity and graft rejection. On the other hand, the Th2 cell (with its associated cytokines: IL-4, IL-5, IL-6, IL-10, IL-13) favors humoral (antibody) immunity, providing an effective correlate of protection for most vaccines, and its downregulation of Th1 can result in some benefit of tolerance to prevent cellular autoimmune reactions; but certain harmful characteristics related to IgE-based allergies and autoimmunity may result.

The simplicity of the mouse system, however, has not translated well to humans. The clear Th1-Th2 polarization in mice with discrete cytokine profiles has given way to a more flexible continuum of responses in humans, where the functionality of Th cells may be more variable and not necessarily locked into a single type for subsequent generations (Kelso A, Groves P, 1997; Kelso A et al., 1999; Doyle A G et al., 1999; Fitzpatrick D R at al., 1999). This flexibility in human Th subsets and complexity of Th cell interactions have led some researchers to question the Th1-Th2 paradigm and the difficulty to generalize for all situations (Kelso A, 1995; Kunzendorf U et al., 1998; Biaze M E et al., 2003; Sheikh A, Strachan D P, 2004; Chaouat G et al., 2004). Nonetheless, the Th1-Th2 paradigm has provided valuable insight into the nature and treatment/prevention of infectious diseases and immunologic disorders (e.g., allergies and autoimmunity).

FIG. 4K (from Harber M et al., 2000) shows a more comprehensive picture of immune regulation with additional cell types. From this figure, one can see additional T regulator cells which contribute to the paradigm by providing suppressor functions (e.g., NKT, CD45RB^{lo}, CD4⁺CD25⁺), including some that are antigen-specific (e.g., Th3, Tr1), thereby preventing autoimmune diseases. In addition, others have identified a nonpolarized effector T cell, T_{FH} (follicular helper T cell), that specifically provides help for the antibody-producing B lymphocytes (Mackay C R, 2000; Schaerli P et al., 2001; Chtanova T et al., 2004).

Cytokine secretion and regulatory functions are not restricted to just lymphocytes or lymphoid cells, but these activities are also provided by and impact myeloid cells (also originating from stem cells through hematopoiesis), including neutrophils, eosinophils, basophils, mast cells, dendritic cells, monocytes, and macrophages.

FIG. 4L (left and right) (from Kidd P, 2003) show more of these interactions (left panel) as well as differentiation among different cell types (right panel), including antibody-producing B cells, antigen presenting cells (APC), and natural killer cells (NK).

Studies have shown that macrophage activation may occur in two different states (classical vs. alternative) that operate in parallel to the Th1-Th2 paradigm, resulting in pro-vs. anti-inflammatory responses (Birk R W et al., 2001), as well as regulation of endocytosis/antigen uptake through decreased vs. increased mannose receptor expression (Montaner L J et al., 1999). During the effector phase of an immune response, T cells and other effector cells find their way into specific tissue where needed and interact with each other in spatial and temporal patterns by way of secreted chemokines (chemotactic cytokines) and chemokine receptors expressed on their surfaces. T cells interact with eosinophils, mast cells, and basophils during allergic reactions, or with macrophages and neutrophils for delayed-type hypersensitivity reactions (Sallusto F et al., 2000).

Specific disease states have been identified that are associated with, and possibly result from, an imbalance of the immune regulatory process already described. The predominance of a particular phenotype (Th1 vs. Th2), or polarization towards one extreme, may determine the presentation and/or severity of disease (for reviews, see Lucey D R et al., 1996; Harber M et al., 2000; Kidd P, 2003). Atopy (familial allergy) in humans was shown to be characterized by a Th2 profile in whole blood cell culture, where high levels of IL-4 and low levels of IFN-γ were observed for CD4⁺ T cells, but the Th2 deviation in atopic asthma showed high levels of IFN-γ for CD8⁺ T cells (Magnan A O et al., 2000). IL-4 was used therapeutically to ameliorate the clinical disease in mice that were experimentally given an autoimmune disease, allergic encephalomyelitis, switching the Th1 cells to Th2 cells (Racke M K et al., 1994). It is now clear that the application of exogenous cytokines can be used to push the Th status in either direction, enabling the development of potential therapeutic applications (Lucey D R et al., 1996; Harber M et al., 2000; Kidd P, 2003; Sun Q L, Ran W, 2004).

7.1 Th1-Th2 Based Diagnostic Panel

In order to diagnose or predict an immunologic disease and/or provide therapy or prophylaxis, the Th polarization status must be determined; this should also be applied to measure susceptibility to infectious and neoplastic diseases. Th status is measurable in terms of cytokine profiles (House R V, 1999; Harber M et al., 2000; House R V, 2001), chemokine/chemoattractant receptors (Sallusto et al., 1998; Syrbe U et al., 1999; Sallusto et al., 2000; Kaplan A P, 2001; Cosmi L et al., 2001), specific effector cell products (Venge P et al, 1999; Venge P, 2004), or gene expression profiles (Rogge L, 2002). Table 8 below shows how the cytokines and chemokine/chemoattractant receptors can, for example, be aligned within the Th1-Th2 paradigm for an exemplary diagnostic panel according to an exemplary embodiment of the present invention.

TABLE 8

| | |
|---|---|
| Th1 | Th2 |

| Cytokines | Receptors | Cytokines | Receptors |
|---|---|---|---|
| INF-γ | CCR5 | IL-4 | CCR3 |
| TNF-α | CXCR3 | IL-5 | CCR4 |
| IL-2 | CCR1 | IL-6 | CCR8 |
| IL-12 | | IL-10 | CRTh2 |
| | | IL-13 | |

There are 4 major ways to measure cytokine profiles (House R V, 2001): bioassays, immunoassays, molecular biological techniques, and flow cytometry. Bioassays require living material to induce proliferation, maintain viability, stimulate migration, induce a secondary function, or inhibit a function. Immunoassays are commonly the enzyme-linked immunosorbent assay (ELISA) or the radioimmunoassay (RIA); the ELISA is most often used, being a colorimetric antibody-based assay. Molecular biological methods usually employ the polymerase chain reaction (PCR), or reverse transcriptase PCR (RT-PCR) to measure the mRNA representing a particular cytokine. Flow cytometry is used to detect and quantify cells that are stained with fluorescent anti-cytokine antibodies. These methods are all compared in Table 9 following (from House RV, 2001):

TABLE 9
The relative advantages and disadvantages of cytokine assay techniques

| | |
|---|---|
| Bioassays | |
| Advantages | Detect only Functional molecules (especially important for discovering new molecules); exquisitely |
| | sensitive (pg/ml or less); can be used to assess production or activity in multiple species |
| Disadvantages | Often lack specificity; indicator cells sometimes ‘problematic’; requires cell culture; time- and labor- |
| | intensive; not very useful for mechanistic studies; generally impractical for assessment of chemokines |
| Immunoassays | |
| Advantages | Rapid; monospecific; no cell culture required; easy to perform; economical |
| Disadvantages | May not detect functional molecules (nonfunctional fragments, functional mutations); sensitivity may be |
| | less than bioassay; reagents not available for all species; limited utility in mechanistic evaluation |
| Molecular biology | |
| Advantages | Most specific method; can detect changes at single-cell level; earlier detection of cytokines (transcription) |
| | than other techniques |
| Disadvantages | Generally expensive and time-consuming; requires specialized equipment and techniques; message not |
| | necessarily translated into protein |
| Flow cytometry | |
| Advantages | Sensitive and extremely specific (single-cell); can be used to evaluate cytokine production and action at |
| | the single-cell level; ability to perform rapid analysis may limit artifacts from culture; several cytokines can |
| | be monitored simultaneously; other relevant molecules (e.g. CD4, CD8) can be examined concomitantly; |
| | excellent technique for mechanist studies |
| Disadvantages | Accurate and complete interpretation of results requires specialized skill |

### Table 9

In addition, combinations of these assays can used for improved results concerning a particular application (House R V, 2001); e.g., RT-PCR ELISA, where the RT-PCR amplifies the message and the ELISA detects the result; in situ hybridization, where genetic material is detected with labeled antibodies; ELISPOT assay, where cytokines are detected from single cells by ELISA and molecular methodology; and cytokine immunotrapping assay, a capture ELISA where cytokine antibodies are used to capture cytokines expressed from isolated cells for analysis. Over 60 chemokine receptors have been identified (Pier G B et al., 2004), but only a few are preferentially expressed by specific Th clones (Sallusto et al., 2000) as indicated in a previous table. These receptors may appear as cell surface-bound and in soluble forms. Bioassays and immunoassays can measure soluble receptors, but flow cytometry and in situ hybridization would be more appropriate for surface-bound receptors (House R V, 2001).

Effector cells, such as eosinophils, release different cytotoxic products upon activation during allergic inflammation (Venge P, 2004). Some products include eosinophil cationic protein (ECP), eosinophil peroxidase (EPO), and eosinophil protein X/eosinophil-derived neurotoxin (EPX/EDN). ECP and EPO are most cell-specific for eosinophils, while EPX/EDN is also produced by neutrophils. Table 10 following (from Venge P, 2004) shows examples of secretory products that can used as markers for other inflammatory cells:

TABLE 10
Inflamatory cells and some of their secretory products that may be
used as markers of their activity and turnover

```
Eosinophils
    Eosinophil cationic protein (ECP)*
    Eosinophil peroxidase (EPO)*
    Eosinophil protein X/eosinophil derived neutrotoxin (EPX/EDN)
Mast cells
    Tryptase*
Neutrophils
    Bastase
Human neutrophil lipocalin (HNL)*
Lactoferrin
Myeloperoxidase (MPO)
Manacytes/macrophages
    Lysozyme
    Interleukin (IL)-6
T-lymphocytes
SIL2r
Endothelial cells
    E-selectin*
Unique and cell-specific markers are marked with asterisk.
```

### Table 10

ECP may be measured in serum, plasma, sputum, or saliva as an indicator of eosinophil granulocyte activity and turnover in the allergic or asthmatic patient (Venge P et al., 1999; Bjork A et al., 2000; Venge P, 2004). EPX/EDN may be measured in urine as another noninvasive way of monitoring eosinophil-related allergic inflammation (Venge P, 2004). Elevated urine levels of EPX/EDN have been shown in atopic dermatitis (Breuer K et al., 2001) and have also been predictive of asthma development in children (Oymar K, 2001).

As an alternative to measuring cytokines, receptors, and other immunologic products, the gene expression of these substances can also be evaluated, deriving gene expression profiles to correlate with the Th1-Th2 paradigm (Rogge L, 2002). Oligonucleotide microarrays have been used to assess human gene expression with a transcript level display capacity of 6000 human genes. From purified and stimulated Th1 and Th2 cells, 215 genes were found to be differentially expressed at a 95% confidence level (Rogge L, 2002). These results were also confirmed by RT-PCR for 28 out of 29 genes.

7.2 Infectious and Neoplastic Diseases

In the event of a microbial or cancerous attack, the type of immune response will usually dictate the outcome. It is generally considered that a Th1 cell-mediated response would be desirable against viruses, intracellular bacteria, fungi, parasites, and cancer, while a Th2 humoral response might work better for most mucosal and extracellular bacterial infections; however, this is really an over-simplification for a complex area fraught with conflicting scenarios (for review, see Lucey D R, 1996). For example, humoral antibody responses are often established as measurements of potency or correlates of protection for vaccines, even against viruses, such as poliovirus (Fox J P, 1984; Salk J, 1984; Sutter R W et al., 1995), and intracellular bacteria, such as Salmonella typhi (Klugman K P et al., 1996; Tacket C O et al., 2004). It is clear that 2 distinct mechanisms of protection (humoral vs. cell-mediated) can occur against the same disease (Kaul D, Ogra P L, 1998; Tacket C O et al., 2004). Due to the complexity of pathogenesis, with different stages of infection and transmission, it is likely that a balance of Th1 and Th2 is required to enable either part to play a role as needed. Nonetheless, it appears that a simple Th1-Th2 paradigm does apply to certain organisms, such as Mycobacterium tuberculosis, during a natural infection (Kidd P, 2003). Epidemiological studies have shown that Th1-mediated (IFN-γ, IL-12) protection is essential for protection against tuberculosis, and Th2 predominance leads to severe disease that is often fatal (Newport M J et al., 1996; Lienhardt C et al., 2002). It is probable that people who are predisposed to only one side of the Th1-Th2 paradigm would be at a disadvantage in terms of options available in response to disease. For a detailed review of infectious and neoplastic diseases in relation to Th1 and Th2 profiles, see Lucey D R et al., 1996.

7.3 Th1-Th2 and Immunologic Diseases (Allergy/Atopy and Autoimmunity/Inflammatory Disease)

Early Innate Modulation of Th1 or Th2 Cells

The nature of the immune response is first influenced by the specific signals that are involved in the early recruitment of immune components to the site of inflammation (Cookson, 2004). As different pattern-recognition receptors can signal through different pathways, different pathogens or antigens can induce different immune responses (Palaniyar, et al. 2002). Second, the nature of the local immune response might also be strongly influenced by tissue-specific facors, and it has been suggested that the epithelial cells, in general, tend to initiate Th2 rather then Th 1-type responses (Matzinger, 2002). In addition, there is evidence that dendritic cells from airways encourage Th2-cell development by default (Stumbles, et al. 1998), and that the induction of Th2 or Th1 type responses by dendritic cells depends on the stimulus with which they are activated (Mazzoni and Segal, 2004).

The perception that specific early signals induced by different infections (or damage by different proteins or other entities) might modify the nature of the subsequent immune response has implications for the Th1-Th2 paradigm of atopic disease. One important issue is the timing of establishment of the Th2-cell bias: on the one hand, Th1- or Th2-cell responses to allergens might be fixed at the time of first exposure in early childhood, and the bias might be subsequently manipulated by bacterial and other adjuvants. On the other hand, Th1- or Th2-cell responses might develop as a consequence of activation of particular patter-recognition receptors by particular pattern-associated molecular patterns (PAMPs) that are present in allergens (Cookson, 2004).

Allergy/Atopy

Allergy or atopy (familial allergy) usually involves Th2 predominance, particularly related to IgE antibodies which attach to basophils and mast cells and cause the release of mediators such as histamine, leukotrienes, and prostaglandins (Kidd P, 2003). Injection of purified allergens (e.g., grass pollen) has been used successfully for immunotherapy towards allergies (Bousquet et al., 1991) by reducing Th2 (IL-4) cytokines (Secrist H et al., 1993) and increasing Th1 (IL-12) cytokines (Hamid Q A et al., 1997). The scientific evidence generally supports the idea that allergies correlate with detectable Th2-dominant conditions that can be treated with Th2-directed immunotherapy.

Any model of the immunology of asthma and atopic dermatitis (eczema) has to take into account the observation that both diseases have increased in prevalence during the past century. Asthma prevalence has been linked to increasing hygiene standards and the progressive westernization of lifestyles in many countries, and a protective effect against asthma of microbial exposure in early childhood has been suggested by the “hygiene hypothesis” (Strachan, 1989). This hypothesis argues that early childhood exposure to infections inhibit the tendency to develop allergic disease. As a consequence, children with westernized lifestyles, protected as they are from the infectious burdens of early life that are common in the developing world, suffer an increased risk of developing allergic disease. There is now strong evidence indicating that microbial exposure is important for protection against asthma, although the nature of the microbial protective effect is still unknown (Cookson, 2004).

Several theories have been put forward to explain the association between asthma and hygiene. The theory of immune deviation suggests that atopic asthma is initiated shortly after birth, when the naive immune system is first confronted with potentially allergenic airborne antigens (Holt, et al. 1999). It is suggested that the initial phase of allergen exposure results in compartmentalization of immunological memory into either Th1 or Th2 cell phenotypes in non-atopic and atopic individuals, respectively. Microbial exposure in infancy encourages a milieu in which initail allergen exposures produce benign Th1 cell responses. In the absence of such exposure, Th2 cell responses predominate, and can be followed by chronic Th2 cell driven inflammation in the airways (Holt, et al. 1999). This raises the possiblity that manipulation of the immune system in early life could result in persistent Th1 or Th2 type responses. If this is the case, vaccination to induce Th1 cell responses might be effective against asthma and other allergic disorders (Holt, 1994). As an alternative to the immune deviation theory, it has been proposed that lack of normal microbial exposure leads to reduced activity of regulatory T cells rather than Th2 cell deviation (Romagnani, 2004).

Asthma is an inflammatory condition, both atopic and nonatopic, that is generally Th2 (IL-4) dominant (Larche M, 2003). Asthma has now reached epidemic proportions, with more than 10% of children being affected in many westernized societies (Cookson, 2004). Allergen injections have been used effectively as immunotherapy in IgE-mediated disease (Abramson M J et al., 1995).

Studies of candidate genes have identified genes that might be involved in asthma susceptibility, many of which exert their effects in the mucosa. For example, IL-13 polymorphism influences mucus production as well as serum IgE levels through a receptor encoded by the polymorphic IL-4R (Ober, et al. 2000). FCERIB variants modify the activity of FcεRI on mast cells, possibly by modulating the level of expression of the receptor on the cell surface (Donnadieu, et al. 2003). A receptor expressed by T cells for the key mast cell signalling factor prostanoid DP has also been reported to be associated with asthma (Oguma, et al. 2004). These findings indicate that the role of mast cells in epithelial inflammation might also be a potential target in asthma therapy.

Other asthma susceptibility genes include the pattern-recognition receptors of the innate immune system, which are expressed by dendritic cells and other cells, and recognize specific microbial components and activate innate immune responses (Cookson, 2004). Polymorphism in CD14, Toll-like receptor 2 (TLR2), nucleotide-binding oligomerization domain 2 (NOD2, or alternatively CARD 15), and T-cell immunoglobulin domain and mucin domain 1 have all been shown to influence asthma susceptibility (Baldini, et al. 1999; Eder, et al. 2004; Kabesch, et al. 2003; McIntire, et al. 2003), indicating that these genes might be important in providing the link between microbial exposure and reduced susceptibility to asthma (Cookson, 2004). TLR10, which responds to an unknown ligand, has recently been associated with asthma (Lazarus, et al. 2004). However, none of these studies has tested for IgE responses to particular allergens, so systemic studies of pattern-recognition receptor activation in asthma are now needed (Cookson, 2004).

Other recognized effects are from tumor-necrosis factor (Moffatt and Cookson, 1997), which encodes a potent pro-inflammatory cytokine that is released by many cells, including airway epithelial cells and transforming growth factor-β (Pulleyn, et al. 2001), which is an important local regulator of epithelial inflammation.

Atopic dermatitis (eczema) can involve a mixture of Th1 and Th2 states, depending on the type or stage of disease. The acute disease is usually Th2 (IL-4), while the chronic disease may show more Th1 (IL-12) cytokines (Singh V K et al., 1999). Further studies indicate that the initial phase of disease is Th2, while Th1 may appear later (Bohm I, Bauer R, 1997).

For more detailed reviews and applications concerning Allergy/Atopy, see: Lucey D R et al., 1996; Kidd P, 2003.

Although the current emphasis in understanding asthma and atopic dermatitis is now moving from involvement of distant adaptive immune responses to local responses at epithelial-cell surfaces, it is probable that a full understanding of these diseases will also depend on studies that include commensal bacteria.

Current understanding of the hygiene hypothesis rests on the suggestion that microbial stimulation during early life is essential for the normal development of the immune system and to achieve the correct cytokine balance (Rook and Standford, 1998). However, the evidence described earlier indicated that damage to the epithelium is probably the initiating event in atopic disease, and the Th1- or Th2-cell bias of subsequent inflammation might be secondary to the nature of the damage (Cookson, 2004).

Alternative mechanisms for bacterial products to modify the risk of atopic diseases include the enhancement of an effective airway barrier by the induction of mucus production through IL-13 stimulation (Kuperman, et al. 2002), or the induction of sufficient polyclonal IgA or IgE to provide nonspecific protection against allergens. Additionally, a protective role by microorganisms might follow the acquisition of distinct commensal or symbiotic organisms. Once an individual's commensal microflora is established in the first year of life, it remains relatively stable (Hooper and Gordon, 2001). Substantial differences have been observed in the intestinal microflora between neighboring countires with a different prevalence of atopic disease (Sepp, et al. 1997), and between atopic and non-atopic children living in each of these countries (Bjorksten, et al. 1999). As commensal and symbiotic organisms actively manipulate host immunity and the activity of other bacteria, it should be considered that interactions among commensal bacteria, pathogens and the host might contribute to the increase and prevalence of asthma and atopic dermatitis (Cookson, 2004).

7.4 Autoimmunity/Inflammatory Disease

Rheumatoid arthritis (RA) is an autoimmune disease with apparent Th cell involvement. Activated T-helper cells are found in the inflammatory filtrates, and T cell-directed therapies have provided some clinical benefit (Schulze-Koops H, Kalden J R, 2001). It appears to be Th1-driven (IFN-γ), but there may be a Th2 (IL-4, IL-10) component at the early stages of disease (Gerli R et al., 2002). It is interesting to note that pregnancy, which seems to have a Th2 bias, appears to ameliorate the progression of RA, providing indirect evidence of the role of Th1 in RA (Da Silva J A, Spector T D, 1992). Schulze-Koops and Kalden suggest that several of the current anti-RA drugs work by altering Th1/Th2 balance. But evidence for this is indirect and comes mostly from non-clinical settings. Schulze-Koops and Kalden concede that it may be overly simplistic to remold the RA data to make it fit the Th1/Th2 hypothesis. They admit it is possible Th1 is subject to simple guilt-by-association with RA, rather than being a major mechanism driving the disease (Schulze-Koops and Kalden, 2001). ImmunoScore analyses would further the understanding of the relationship between RA and the Th1/Th2 paradigm.

Multiple sclerosis (MS) is an autoimmune disease that appears to be Th1-driven (IL-12, IFN-γ), with some conflicting data (Kidd P, 2003); this may be a complication of the role of regulatory T cells (Tr) secreting cytokines (IL-10) to normally downregulate the Th1 cells (Bettelli E et al., 1998). Defining the factors that initiate and perpetuate the ongoing pathogenesis, as well as designing treatment strategies for this disease, have been complicated by absence of an identifiable causative agent, diversity of co-existing CNS lesion stages (ie, acute, chronic active, chronic inactive, remyelinating, gliotic plaque), an unpredicatble relapsing-remitting clinical course early in the disease, lack of a direct correlation of clinical symptoms to the occurence of new white matter lesions, and the absence of a naturally occurring animal form of the disease (Jordan, et al. 1999). One group tried T cell receptor peptide therapy on MS patients. Of the less than 200 patients studied, 50-90 percent supposedly showed immunological response to vaccination and as much as 35 percent had some degree of favorable clinical response (Vandenbark, et al. 2001).

Type 1 diabetes is an autoimmune disease that may be Th1 dominant. Data available thus far in human diseases favor a prevalent Th1 lymphokine profile in target organs of patients with organ-specific autoimmunity. Adjuvant therapy with BCG injections seems to benefit patients and nonobese diabetic mice by raising Th2 (IL-4) cytokine levels (Singh V K et al., 1999). However, administration of Th2 cells to nonobese diabetic mice can worsen the disease, if the recipient mice are immunocompromised (Pakala, et al. 1997).

In summary, for three major autoimmune diseases—RA, MS, and type 1 diabetes—a Th1 dominance has not been well enough established to rationalize balancing intervention. On both pragmatic and theoretical grounds there is real possibility of making the patient sicker through efforts to intervene with Th@ cells or Th2 cytokines (Kidd, 2003). The ImmunoScore diagnostic panel would be invaluable in assessing the relationship of Th1/Th2 cytokine levels in relationship to these disease conditions.

Miscarriage might be the result of an autoimmune response to the fetus during pregnancy, where the normally Th2 (IL-3, IL-4, IL-10) dominance during pregnancy has shifted to a Th1 state (IL-2, IFN-γ, TNF-α), allowing the maternal cell-mediated response to be directed towards the paternal antigens of the fetus (Chaouat G et al., 2004). While the simplicity of the Th1-Th2 paradigm applied to pregnancy is being questioned, particularly in terms of potential therapy and the inability to generalize across all individuals, there may still be a Th2 bias for normal pregnancies (Chaouat G et al., 2004).

Systemic lupus erythematosus is a chronic, recurrent, potentially fatal multisystem inflammatory disorder that typically shows anti-nuclear and other autoantibodies, with elevated Th1 (IL-2, IFN-γ) and Th2 (IL-4) cytokines (Kidd P, 2003). Patients with arthritis have higher Th1 cytokine levels, while those with CNS involvement have higher Th2 cytokine levels (Chang D M et al., 2002).

It is possible that the association of genetic polymorphism (Chang D M et al., 2002), along with disease stage and presentation, all work together to affect the Th1-Th2 pattern. Complete complement components C4A and C4B deficiencies have been identified and studied clinically (Yang, et al. 2004a). All but one of the complete C4-deficient subjects experienced symptoms related to immune complex clearance disorders such as SLE, a lupus-like disease, or glomerulonephritis (Yu, et al. 2003). The human C4 locus is remarkably complex. Among different individuals in a population, two to seven (possibly eight) C4 genes may be present in a diploid genome, leading to a 3- to 5-fold variation in plasma C4 protein concentrations and the presence of multiple allotypes (Yang, et al. 2003). Considering the roles of C4A and C4B in immunoclearance, memory, and effector functions of the humoral immune response, it is not unexpected that a deficiency of C4A or C4B is frequently associated with infectious and/or autoimmune diseases (Yang, et al. 2004b). An elucidation of the molecular basis of complete C4A and C4B deficiencies may help in designing a comprehensive screening strategy to determine the prevalence of C4A and C4B mutations in autoimmune, infectious, and kidney diseases (Yang, et al. 2004b).

In humans, outside of major histocompatibility complex (MHC) class II, genetic polymorphisms or defects in genes involved in antigen uptake and/or process and in immune complex clearance such as complement, FCGR2A and FCGR3A have been identified to contribute to SLE susceptibility (Wakeland, et al. 2001). Recently, programmed cell death gene 1 (PDCD1) which regulates B cell activation has been identified as an autoimmunity candidate gene in the mouse (Nishimura, et al. 1999), and a single-nucleotide polymorphism (SNP) in a putative RUNX1 binding site in the promoter of human PDCD1 gene has been inplicated as a risk allele for SLE (Prokunina, et al. 2002).

Fibrotic disease, involving tissue fibrosis (scarring), is the result of a Th1-Th2 imbalance during wound healing in response to chronic inflammation, and is responsible for an estimated 45% of U.S. deaths (Wynn T A, 2004). In this case, the wound healing Th2 (IL-4, IL-5, IL-13) response, opposing the initial Th1 (IFN-γ, IL-12) regenerative inflammatory response, is continuous and leads to excessive tissue remodeling (permanent scar tissue). While the Th2 wound healing is necessary for long-term survival from an injury, persistent healing, in response to a chronic Th1 stimulus, might end in fibrotic tissue causing major organ failure and death (Wynn T A, 2004). For more detailed reviews and applications concerning Autoimmunity/Inflammatory Disease, see: Lucey D R et al., 1996; Kidd P, 2003.

8. ImmunoScore Analyses for Immigrants and Internationally Adopted Children

The current U.S. Immigration and Naturalization law has vaccination requirements for the following vaccine-preventable diseases:
Measles
Mumps
Rubella
Polio
Tetanus
Diphtheria
Pertussis
Haemophilus influenzae type B (Hib)
Hepatitis B
Varicella
Pneumococcal disease
Influenza
   Vaccination of Internationally Adopted Children

The ability of a clinician to determine that an individual is protected from vaccine-preventable disease on the basis of their country of origin and their personal medical records alone is limited. Currently, only written documentation should be accepted as evidence of prior vaccination. Although vaccines with inadequate potency have been produced in other countries, the majority of vaccines used worldwide are produced with adequate control standards and are potent. Data are inconclusive regarding the extent to which an internationally adopted child's immunization record reflects the level of the child's protection from vaccine-preventable diseases. For example, a record might indicate administration of Measles, Mumps, and Rubella (MMR) vaccine when only single antigen measles vaccine was administered. A study of children adopted from China, Russia, and Eastern Europe determined that only 39% of children with documentation of >3 doses of DTP had protective levels of diphtheria and tetanus antitoxin (Hostetter, et al. 1998). Rather than rely on records and memories that may be less than satisfactory, the ImmunoPrint diagnostic assay system would be a highly practical tool to test the specific antibody levels of individuals entering the country or children being adopted from other lands.

The CDC states that doses of measles-containing vaccine administered prior to the first birthday should not be counted as part of the series (CDC, 2002). They also state that serological testing for IgG antibody to MMR vaccine viruses can be considered if the individual lacks the appropriate paperwork. A child whose record indicates receipt of measles or measles-rubella vaccine at age≧1 year and who has protective antibody levels against measles and rubella should receive a single dose of MMR as age appropriate to ensure protection against mumps.

Regarding poliovirus vaccine, the CDC suggests that the “simplest approach” is to revaccinate immigrants with IPV according to the U.S. schedule (CDC, 2002). They also state that children appropriately vaccinated with three doses of oral polio vaccine (OPV) in economically developing countries might have suboptimal seroconversion. Currently, serologic testing for neutralizing antibody to poliovirus types 1, 2, and 3 can be obtained commercially and at certain state health department laboratories. Incorporation of poliovirus assays into ImmunoPrint diagnostics would enable immigration authorities to screen individuals for seroconversion to poliovirus types 1, 2, and 3. Recommended immunization boosters could then be followed through with in timely fashion.

Vaccination providers can re-vaccinate a child with DTaP vaccine without regard to recorded doses; however, one concern regarding this approach is that data indicate increased rates of local adverse reactions after the fourth and fifth doses of DTP or DTaP. If a re-vaccination approach is adopted and a severe local reaction occurs, serologic testing for specific IgG antibody to tetanus and diphtheria toxins can be measured by ImmunoPrint analyses before administering additional doses. Protective concentration indicates that further doses are unnecessary and subsequent vaccination should occur as age-appropriate. There is, as yet, no serologic correlate of protection for pertussis. The lack of a serologic correlate of protection is one area where application of the ImmunoPrint database would be of great value.

Because the number of vaccinations needed for protection from Haemophilus influenzae type B (Hib) disease decreases with age and adverse events are rare, age-appropriate vaccinations for immigrants should be provided (CDC, 2002). Hib vaccination is not routinely recommended for children over 5 years of age.

The ACIP recommends serologic testing for hepatitis B surface antigen (HBsAg) for international adoptees (CDC, 2002). Children determined to be HBsAg positive should be monitored for the development of liver disease. Household members of HBsAg-positive children should be vaccinated. The current recommendation from the ACIP states that a child whose records indicate receipt of ≧3 doses of vaccine can be considered protected and additional doses of vaccine are not needed if ≧1 doses were administered at >6 months of age. Those who have received <3 doses should complete the series at the recommended intervals (CDC, 2002). This rather complicated recommendation depending on accurate record-keeping could be replaced with ImmunoPrint diagnostic testing. A positive anti-HBsAg IgG antibody would be indicative of protection in these individuals.

Varicella vaccine is not administered in the majority of countries. The ACIP recommends that a child who lacks a reliable medical history regarding prior varicella disease should be vaccinated as age-appropriate (CDC, 1996). A well-timed ImmunoPrint diagnostic assay would remove speculation from the vaccination protocol.

Pneumococcal conjugate and pneumococcal polysaccharide vaccines are not administered in the majority of countries. The CDC recommends that vaccines should be administered as age-appropriate or as indicated by the presence of underlying medical conditions (CDC, 2002). ImmunoPrint diagnostic analysis could be used to point out the need for vaccination in immigrating individuals.

Each country may have needs for assessing the immune status of immigrants that may not necessarily coincide with the U.S. requirements as previously outlined. In addition, there may be other needs inside or outside the U.S., dictated by a particular investigation at a particular site. For example, Greenaway et al. (2004) have embarked on a mission to assess the immune status of immigrants in the Montreal area of Canada, with initial emphasis on 5 different infectious agents: hepatitis A, measles, mumps, rubella, and varicella (Greenaway C A, Boivin J F, Dongier P, Miller M A, Schwartzman K. Susceptibility to vaccine-preventable diseases in newly arrived immigrants. Abstract G-538, pp 254-5, In 44^{th} ICAAC Abstracts 2004 [Interscience Conference on Antimicrobial Agents and Chemotherapy, Washington, D.C., Oct. 30-Nov. 2, 2004]: American Society for Microbiology, Washington, D.C.). Subsequent studies may expand to include tetanus and diphtheria, as well as other agents listed for routine immunizations of Canadians, which are essentially identical to those listed for the U.S. Note that hepatitis A, in the above study, does not represent an infectious agent designated for routine immunization in Canada or the U.S., but it is listed for selective immunization where people have been identified to be at greater risk of disease. Likewise, diagnostic panels may be expanded where appropriate to represent additional infectious agents that are listed for selected immunization.

In exemplary embodiemtns of the present invention diagnostic subpanels can be developed to accommodate the needs for different researchers, such as Greenaway et al., where there is an interest to follow up on particular infectious agents in a region where there may be new or ongoing outbreaks of disease. The influx of immigrants that are unprotected against vaccine preventable diseases (VPD) has already been shown to contribute the increased incidence of disease. For example, varicella and rubella vaccines are not routinely administered in many countries, and this has therefore resulted in an over representation of immigrants in outbreaks of varicella and rubella in areas where these vaccines already exist. The Greenaway et al. (2004) studies have shown that in Canada, “immigrants are more likely to be susceptible to varicella, rubella, and mumps than North Americans.” In addition, they have stated, “Adult immigrants may benefit from targeted vaccination programs but given the geographic variation in susceptibility to VPD, this must be taken into consideration when developing these programs.” For this scenario, ImmunoScore diagnostic panels can prove invaluable to identify the target according to the individually assessed immune status.

Tuberculosis (TB) is another disease that may be of considerable importance to monitor, not necessarily for immune status, but for active infection, particularly in immigrant populations. It is estimated that one third of the global population is infected with TB. Due to improved laboratory services during the 1990s, there has been a resumption of an overall decline in U.S. cases of TB. Nonetheless, the CDC states, “TB continues to pose substantial social, public health, and economic costs.” (Centers for Disease Control and Prevention. National plan for reliable tuberculosis laboratory services using a systems approach: recommendations from CDC and the Association of Public Health Laboratories Task Force on Tuberculosis Laboratory Services. MMWR 2005; 54[No. RR-6]:1-12). This 2005 CDC report indicates that the U.S. spends nearly $1 billion annually on TB-related costs, with 9-14 million people having latent TB infections and 15,000 new cases reported in 2003. The CDC also states, “to eliminate TB in the United States, further improvements are needed in laboratory services to support TB treatment, prevention, and control.” As a result, “TB control is now entering a new phase in the United States, a transition from low incidence to elimination.” (Centers for Disease Control and Prevention. Progressing toward tuberculosis elimination in low-incidence areas of the United States: recommendations of the Advisory Council for the Elimination of Tuberculosis. MMWR 2002; 51 [No. RR-5]:1-16). An ImmunoScore diagnostic panel containing TB, for example, could be utilized in this regard in the U.S.

The BCG vaccine, currently licensed for TB, is not recommended for routine use in the U.S. because of questionable efficacy; however, there are other countries that routinely use this vaccine. The United Kingdom, in 2005, announced that, after 50 years, it is dropping its school TB vaccination program for young teenagers, in favor of targeting infants in ethnic populations that are at greater risk (Celia Hall, Medical Editor, Telegraph Group Limited, Jul. 7, 2005). For example, they have indicated that the case rate in whites is 3.6 per 100,000, while the rate in Africans is 279.8 per 100,000, and the rate in Indian, Pakistani, and Bangladeshi people is 126.7. New immigrants from countries with high TB incidence would also be targeted for vaccinations. It is possible that a diagnostic panel which includes TB would prove useful for screening these populations.

As demonstrated in the UK, there is a greater incidence of TB in immigrants from certain regions of the world. It would be therefore useful to add a TB diagnostic to immigrant panels previously described. For example, a TB diagnostic could be included in the subpanel proposed for Canada, or used as a separate diagnostic, as a follow-up to the Greenaway et al. study. It is possible to use specific antibody detection to distinguish active TB infections from non-active or non-TB (Tong M et al. 2005. J Immunol Methods. 301:154-63). In this case, specific TB antigens, particularly those of a carbohydrate nature, may be selected for inclusion in the proposed diagnostic panels to identify people with active TB infections in need of treatment.

C. Immunoscore Exemplary Superpanels

1. Middle School Student ImmunoPrint Super Diagnostic Panel

A study performed recently described the fact that little information is available about the effectiveness of school entry vaccination requirements at the middle school level (Fogarty, et al. 2004). This particular study examined coverage levels among students entering seventh grade in Florida following implementation of a school entry vaccination requirement in 1997. The authors concluded that the seventh grade vaccination entry requirement was associated with high levels of vaccination coverage and that passing a school entry vaccination requirement appeared to be sufficient to increase coverage, but that other strategies may be required to achieve full immunization of middle school students. Very shortly, there is likely to be a new recommendation for a pertussis booster immunization for middle school students and the possibility looms for an HPV immunization program. ImmunoScore diagnostics and database storage would track the success and help establish the need for such immunization programs.

In exemplary embodiments of the present invention, a middle school superpanel can comprise the following exemplary panels:

1.1 Persistent Immunity Induced by Childhood Vaccines

This panel is described above in section A3.

1.2 Sexually Transmitted Disease (STD) Diagnostic Panel

For children entering middle school (grades six through eight) a baseline determination for antibody levels to STDs is advisable. Recommended tests for ImmunoPrint measurement of immunity to STDs:
Antibodies to Chlamydia—IgG, IgA, and IgM (3)
Antibodies to HSV—IgG to HSV-1 and HSV-2 (2)
DNA analyses of HPV types—particular emphasis on high-risk
Antibody to N. gonorrhoeae (1)
Antibody to T. pallidum (1)
T-cell related response to T. pallidum
Antibody to HIV
T-cell related response to HIV
Antibodies to GBS serotypes (at least 3)
Measurement of Th1/Th2 cytokines (many as current evolving definitions)
Antibodies to organisms that cause Urinary Tract Infection (UTIs)
   Escherichia coli
   Staphylococcus saprophyticus
   Proteus mirabilis
   Klebsiella pneumoniae
   Enterococcus species
   Pseudomonas aeruginosa

Currently, there are no vaccines available for any of these STDs. Merck has had very successful Phase III clinical trial with their HPV vaccine. They are going to ask for approval from the FDA and could begin marketing in late 2006 (http://www.medicalnewstoday.com/medicalnews.php?newsid=31783). Until this situation is ameliorated, the objective of an ImmunoPrint STD diagnostic panel would thus be to recommend treatment. The ImmunoPrint database can generate correlate of protection information for all disease-causing organisms. As vaccines are developed, ImmunoPrint diagnoses could be designed to examine antibody and other related immune responses to vaccine components.

Currently, there are no vaccines available for any of these STDs. Merck has had very successful Phase III clinical trial with their HPV vaccine. They are going to ask for approval from the FDA and could begin marketing in late 2006 (http://www.medicalnewstoday.com/medicalnews.php?newsid=31783). Until this situation is ameliorated, the objective of an ImmunoPrint STD diagnostic panel would thus be to recommend treatment. The ImmunoPrint database can generate correlate of protection information for all disease-causing organisms. As vaccines are developed, ImmunoPrint diagnoses could be designed to examine antibody and other related immune responses to vaccine components.
Chlamydia trachomatis infection is the most commonly reported sexually transmitted disease in the United States, with the highest rates among adolescent females and young women. Because up to 70% of chlamydial infections in women are asymptomatic, routine screening and treatment of infected persons is essential to prevent pelvic inflammatory disease, infertility, ectopic pregnancy, and perinatal infections. The third U.S. Preventive Services Task Force (USPSTF) recommends that primary care physicians routinely screen all women whether or not they are pregnant if they:
   Are sexually active and aged 25 or younger.
   Have more than one sexual partner, regardless of age.
   Have had an STD in the past, regardless of age.
   Do not use condoms consistently and correctly, regardless of age.
According to studies reviewed by the third USPSTF:
   The cost of screening women who are not pregnant and who are at risk for chlamydial infection may be less than the cost of treating Chlamydia and its complications.
   Screening patients at greatest risk is more cost effective than screening all patients.
   DNA or RNA amplification tests are more sensitive than culture.
A low cost diagnostic test for Chlamydia infection or immune response to a Chlamydia vaccine would be a welcome addition to immune status determination by ImmunoPrint diagnostic testing.
Herpes simplex virus type 2 (HSV-2) is the primary cause of genital herpes, a common sexually transmitted disease with at least 40 to 60 million infected individuals in the U.S. Medically serious complications of HSV are rare but constitute a significant burden, given the high rates of HSV seropositivity in the population. Many prophylactic and therapeutic vaccination approaches have been explored for the prevention or treatment of HSV infection. Infection induces both humoral and T-cell immunity. Vaccine candidates for HSV-2 infection include subunit vaccines, killed and live attenuated virus vaccines, and viral DNA vaccines.
Human papillomaviruses (HPV) are small double-stranded DNA viruses that are responsible for pathological conditions ranging from benign skin warts to invasive cervical carcinomas. Cervical cancer is the second leading cause of cancer death among women worldwide, and more than 99% of cervical cancers contain HPV, particularly the high-risk HRP type 16 (HPV-16). Two HPV oncoproteins, E6 and E7, are consistently expressed in HPV-associated cancer cells and are responsible for their malignant transformation. These oncogenic proteins represent ideal target antigens for developing vaccines and immunotherapeutic strategies against HPV-associated neoplasms. More than 10,000 American women a year are diagnosed with cancer or precancerous cells caused by HPV, and 3,700 of them will die. Eighty times that number will die worldwide. An effective vaccine could prevent nearly all of those deaths. The CDC is currently considering an HPV vaccine for all children aged 12 years. A positive recommendation by the ACIP could start states thinking of requiring the vaccine for entry into middle school.
Neisseria gonorrhoeae, the causative agent or gonorrhea, is one of the most common sexually transmitted pathogens worldwide. Although a robust inflammatory response ensues during symptomatic infection, no apparent protective immunity is developed following infection, as shown in a male human challenge study and by the high incidence of recidivism among patients attending sexually transmitted disease clinics. The search for a vaccine against gonorrhea has been largely disappointing. In human vaccine trials, partially lysed gonococci, purified pilin, and purified porin were shown to be immunogenic, but all failed to elicit protection upon subsequent natural exposure. The lack of protective immunity is likely due, in part, to the capacity of many gonococcal surface antigens to undergo high-frequency phase and antigenic variation.
Individuals infected with Treponema pallidum subsp. pallidum develop specific immune responses that are able to clear millions of treponemes from sites of primary and secondary syphilis. Despite the fact that humans develop robust immune responses against T. pallidum, they can be infected multiple times. The response is a T-cell mediated delayed-type hypersensitivity response in which T cells infiltrate syphilitic lesions and activate macrophages to phagocytose antibody-opsonized treponemes. How treponemes from heterologous isolates can evade the recall response of a previously infected individual is unknown. Data from animal studies suggest that both antibodies and T cells play a role in protection but neither alone prevents infection. It is possible that antigenic diversity of T. pallidum accounts for the lack of heterologous protection. The T. pallidum repeat protein K (TprK) is a strong candidate for a treponemal factor involved in immune evasion. Epitope mapping studies revealed that, during experimental infection, T cells are directed to the conserved regions of TprK, while the antibodies are directed to the variable regions.
A safe, effective prophylactic human immunodeficiency virus (HIV) vaccine is urgently needed to curb the current AIDS epidemic. There are currently 40 million individuals in the world infected with HIV, and nearly 16,000 new infections occur worldwide each day. Effective HIV-1 vaccines must be capable of protecting immunized individuals from infection with a broad array of diverse viral variants. Attempts to develop a safe and effective AIDS vaccine have been slowed, in part, by the difficulty in clearly defining specific immune responses that can prevent infection and limit disease progression. This is in part due to the poor immunogenicity of the envelope glycoprotein, the tremendous variability of the virus, its ability to evade and impair the host's immune system, and its ability to persist by integrating into the host's immune system, and its ability to persist by integrating into the host's genome of a number of different cell types. It is generally believed that an effective HIV-1 vaccine must be capable of inducing neutralizing antibodies as well as strong cell-mediated immune responses in outbred populations.
Group B Streptococci (GBS) emerged dramatically in the 1970s as the leading cause of neonatal infection and as an important cause of maternal uterine infection. The burden from GBS disease in elderly persons has also increased. In 1996, the first national consensus guidelines were released. Since then, there has been a 70% reduction in early-onset neonatal GBS infection. In 2002, new national guidelines were released recommending:
   solely a screen-based prevention strategy
   a new algorithm for patients with penicillin allergy
   more specific practices in certain clinical scenarios
Yet clinical issues remain, including implementation of new diagnostic techniques, management of preterm rupture of membranes, use of alternative antibiotic approaches, improvement of compliance, prevention of low birth weight infants, emergence of resistant organisms, and vaccine development.
Urinary tract infections (UTIS) are a leading cause of morbidity and mortality and health care expenditures in persons of all ages. Sexually active young women are disproportionately affected, but several other populations, including elderly persons and those undergoing genitourinary instrumentation and catheterization, are also at risk. UTIs are the leading cause of gram-negative bacteremia (Orenstein and Wong, 1999).

Lymphocytes are the effector cells of acquired immunity. There are two T helper subsets, Th1 and Th2, based on two distinct cytokine profiles that resulted in the overall regulation of the immune response. The Th1 cell (with its associated cytokines: INF-γ, TNF-α, IL-2, IL-12) is biased towards the cell-mediated side of immunity, effective against intracellular parasites, and its down regulation of Th2 can provide relief from allergic reactions due to IgE; but detrimental effects may result in autoimmunity and graft rejection. On the other hand, the Th2 cell (with its associated cytokines IL-4, IL-5, IL-6, IL-10, IL-13) favors humoral immunity, providing an effective correlate of protection for most vaccines, and its down regulation of Th1 can result in some benefit of tolerance to prevent cellular autoimmune reactions; but certain harmful characteristics related to IgE-based allergies and autoimmunity may result. In order to diagnose or predict an immunologic disease and/or provide therapy or prophylaxis, the Th polarization status must be determined; this should also be applied to measure susceptibility to infectious and neoplastic diseases. Th status is measurable in terms of cytokine profiles, chemokine/chemoattractant receptors, specific effector cell products, or gene expression profiles. An exemplary diagnostic panel is described in the table below:

| | |
|---|---|
| Th1 | Th2 |

| Cytokines | Receptors | Cytokines | Receptors |
|---|---|---|---|
| INF-γ | CCR5 | IL-4 | CCR3 |
| TNF-α | CXCR3 | IL-5 | CCR4 |
| IL-2 | CCR1 | IL-6 | CCR8 |
| IL-12 | | IL-10 | CRTh2 |
| | | IL-13 | |

2. Women of Child-Bearing Years ImmunoScore Super Diagnostic Panel

Adult immunization rates have fallen short of national goals partly because of misconceptions about the safety and benefits of current vaccines. The danger of misconceptions is magnified during pregnancy when concerned physicians are hesitant to administer vaccines and patients are reluctant to receive them. Routine vaccines that are generally safe to administer during pregnancy include diphtheria, tetanus, influenza, and hepatitis B. Other vaccines, such as meningococcal and rabies, may be considered. Vaccines that are contraindicated, because of the theoretical risk of fetal transmission, include measles, mumps and rubella; varicella; and BCG. A number of other vaccines have not yet been adequately studied; therefore, theoretic risks of vaccination must be weighed against the risks of disease to mother and fetus.

The administration of vaccines during pregnancy poses a number of concerns to physicians and patients about the risk of transmitting a virus to a developing fetus. This risk is primarily theoretical. No evidence exists of risk from vaccinating pregnant women with inactivated virus or bacterial vaccines or toxoids (CDC, 2002). Physicians should consider vaccinating pregnant women on the basis of the risks of vaccination versus the benefits of protection in each particular situation, regardless of whether live or inactivated vaccines are used (Sur, et al. 2003). Generally, live-virus vaccines are contraindicated for pregnant women because of the theoretical risk of transmission of vaccine virus to the fetus. The following table summarizes recommendations for vaccines commonly administered and their indication for use during pregnancy.

TABLE 11
Immunizations During Pregnancy

| | CONTRAINDICATED | |
|---|---|---|
| CONSIDERED | DURING | |
| SAFE IF | PREGNANCY OR | SPECIAL |
| OTHERWISE | SAFETY | RECOMMENDATIONS |
| INDICATED | NOT ESTABLISHED | PERTAIN |
| Tetanus and | BCG* | Anthrax |
| diphtheria toxoids | | |
| (Td) | | |
| Hepatitis B | Measles* | Hepatitis A |
| Influenza | Mumps* | Japanese encephalitis |
| Meningococcal | Rubella* | Pneumococcal |
| Rabies | Varicella* | Polio (IPV) |
| | | Typhoid |
| | | Vaccinia* |
| | | Yellow fever* |

*= Live, attenuated vaccine

Women in their second and third trimesters of pregnancy have an increased risk of influenza-related complications including pneumonia and a four-fold risk of hospitalization (Neuzil, et al. 1998). The CDC has recommended that women who will be in the second or third trimester during influenza season and all pregnant women with additional high-risk medical conditions should receive vaccination in the fall. Despite publication of these guidelines, rates of vaccination among high-risk patients remain low (Silverman and Greif, 2001; Schrag, et al. 2003). Many possible explanations exist for this discrepancy, including vaccine unavailability, logistical concerns, poor reimbursement, fear of side effects, and lack of adequate patient or physician education (Wallis, et al. 2004).

A number of maternal conditions were perceived as potential contraindications to influenza vaccination during pregnancy. The most common of these were the first trimester, history of preterm labor, history of intrauterine fetal demise, and pregnancy induced hypertension; none of these are listed by the CDC as contraindications (Wallis, et al. 2004). According to this group, another potentially significant obstacle to influenza vaccination during pregnancy was physician reimbursement. Several responders remarked that reimbursement from insurance companies played a part in whether they stocked the vaccine in their offices and whether it was administered to pregnant patients. Although they acknowledged the indications for the vaccine, some obstetricians stated that insurance plans have refused reimbursement for vaccination because they were not the patient's primary care provider for this “preventive” service. Although patients may still be instructed to obtain vaccination elsewhere, this additional obstacle to recommended obstetrical care may result in lower immunization rates. These authors concluded by stating that further research is needed to determine effective methods of increasing vaccination rates in this high-risk population.

Cytomegalovirus (CMV) is found universally throughout all geographic locations and socioeconomic groups, and infects between 50-80% of adults in the United States by 40 years of age. CMV is also the virus most frequently transmitted to a developing child before birth. The incidence of primary CMV infection in pregnant women in the U.S. varies from 1-3%. Healthy pregnant women are not at special risk for disease from CMV infection. When infected with CMV, most women have no symptoms and very few have a disease resembling mononucleosis. It is their unborn babies that may be at risk for congenital CMV disease. CMV remains the most important cause of congenital viral infection in the U.S. For infants who are infected by their mothers before birth, two potential problems exist:
1. Generalized infection may occur in the infant, and symptoms may range from moderate enlargement of the liver and spleen (with jaundice) to fatal illness. With supportive treatment most infants with CMV disease usually survive. However, from 80-90% will have complications within the first few years of life that may include hearing loss, vision impairment, and varying degrees of mental retardation.
2. Another 5-10% of infants who are infected but without symptoms at birth will subsequently have varying degrees of hearing and mental or coordination problems.

However, these risks appear to be almost exclusively associated with women who previously have not been infected with CMV and who are having their first infection during pregnancy. There appears to be little risk of CMV-related complications for women who have been infected at least six months prior to conception. The current recommendations from the CDC for pregnant women with regard to CMV infection are:
1. Throughout the pregnancy, practice good personal hygiene, especially hand washing with soap and water, after contact with diapers or oral secretions (particularly with a child who is in day care).
2. Women who develop a mononucleosis-like illness during pregnancy should be evaluated for CMV infection and counseled about the possible risks to the unborn child.
3. Laboratory testing for antibody to CMV can be performed to determine if a woman already had a CMV infection.
4. Recovery of CMV from the cervix or urine of women at or before the time of delivery does not warrant a cesarean section.
5. The demonstrated benefits of breast-feeding outweigh the minimal risk of acquiring CMV infection from the breast-feeding mother.
6. There is no need to either screen for CMV or exclude CMV-excreting children from schools or institutions because the virus is frequently found in many healthy children and adults.

Recently, it was found that hyperimmune globulin therapy in pregnant women was associated with a significantly lower risk of congenital CMV disease (Nigro, et al. 2005). This group concluded that treatment of pregnant women with CMV-specific hyperimmune globulin is sage, and their findings suggested that it may be effective in the treatment and prevention of congenital CMV infection.

Specific ImmunoScore diagnostic panel recommendations must take into account the woman of child-bearing years status with regard to pregnancy. Ideally, an ImmunoScore screening of a young women prior to child-bearing years would give an appropriate “baseline” reading of that individual. In this instance, for example, a positive serologic test for CMV would be an indication that CMV-like illness during pregnancy would not be a cause of concern regarding transmission to that mother's infant during a pregnancy later in that woman's life.

Clearly, women of child-bearing years that are not pregnant, or not planning to get pregnant in the six months following ImmunoScore screening would have different recommendations than pregnant women. An ideal location and time for ImmunoScore diagnostic screening women of child-bearing years would be during their annual recommended visit to the OB/GYN. An early baseline could be achieved for each patient and the Specialist could make use of the specific recommendations without confusion as to which immunizations would be appropriate. It is very important to assure immunity to the components of the measles-mumps-rubella vaccine prior to pregnancy and the ImmunoScore service would enable that assurance.

Accordingly, in exemplary embodiments of the present invention a Women of Child-Bearing Years ImmunoScore superpanel can be defined as follows.

2.1 Recommended Tests for ImmunoScore Measurement of Immunity:

Antibody to Cytomegalovirus (1)
   History of CMV infection needs to be captured to complete ImmunoScore database and add relevance to pregnancy.
Pregnancy test (1)

A pregnancy test is critical to making the correct decisions regarding administration of vaccines to women of this age group. There are, of course, other considerations here, but the status of the woman in question regarding pregnancy must be resolved in order to make accurate therapeutic decisions. In addition to CMV antibody, the physician(s) of women of child bearing years need to be aware of the recommendations of the CDC regarding immunizing pregnant women and the risks of immunization vs. the risks of foregoing immunizations. In addition, physicians should be aware that following appropriate immunization protocols and assuring a competent immune status is extremely important for women of child-bearing years.

2.2 Persistent Immunity Induced by Childhood Vaccines Diagnostic Panel

Described above in section A3.

2.3 Sexually Transmitted Disease (STD) Diagnostic Panel

Described above in section C1.2.

II. Immunoscore System Database

A. General Overview

In exemplary embodiments of the present invention the results of assays of the immune status of an individual together with additional medical and demographic information which can be collected at the same time as, or derived from, the collected data, can be, for example, stored in a system database. Such a database can, for example, serve as an electronic record of the immune status data over a period of time, both for individuals and populations or sub-populations as described below.

For each run of an exemplary assay within an exemplary system, various categories of data can be collected. Data can, for example, be stored in an electronic database using standard techniques as are known in the art. An example of data which can be stored and the manner in which it can be stored is next described. It is understood that this example is not intended to preclude the storage of additional collected or derived data as may prove useful for the purposes of trending, data mining, evaluation or diagnostic improvement, as described more fully.

For each assay an exemplary system can record a unique assay ID, which can incorporate, among other information, an identifier for the assay instrument. This ID can be unique over the universe of instruments, ensuring that when data is aggregated into a central system no two assay result records will have the same identifier. A possible implementation of this functionality is given, for example, by Microsoft's use of the GUID (Globally Unique Identifier), a 16 byte identifier generated by a computer and guaranteed to be unique across all computers.

Each record can include the time and date that the assay was performed, stored to a time resolution of one second. There are a variety of standard means of storing time and date information in a database. One simple means is, for example, to record the number of seconds from an arbitrary start time, such as, for example, Jan. 1^{st}, 1900 at midnight.

Each record can, for example, also include an indication of the location where the sample was processed. This can include, for example, an identifier of the instrument used, as well as real-world location information, such as, for example, the name and address of the facility where the instrument has been installed.

The aforementioned exemplary fields comprise identification information which is important to maintain for all samples. In addition, information about the sample and patient can be stored in the database as well. Patient information can be, for example, stored in a form which is separate from the bulk of the data, and referenced by a data link. Patient information, which can include, for example, name, social security number, birth date or other information, can be maintained with emphasis on security standards are known in the art. The storage of identifiable individual patient information in a separate virtual location from the remaining data can help to maintain such a high level of security.

In exemplary embodiments of the present invention, a system can also store an identifier indicating exactly which assay was performed on the sample. This can indicate not only the analyte to be determined, but also information regarding the production of the reagents used in the assay. This information can be used to distinguish between, and compensate for, for example, lot-to-lot variations in assay manufacture.

The measurement of an immune response to a particular disease or other analyte can involve the collection of a large quantity of low level data generated by an instrument. For an ECL instrument, for example, an instrument can measure the light emitted from the electrochemiluminescence over some time period as well as other information such as voltages and currents used to induce the electrochemiluminescence and the temperature near the electrodes through which the electrical energy is delivered to drive the electrochemiluminescent reaction. From this “raw data” and possibly instrument calibration information, a single number, for example, can be computed to represent an ECL signal for that measurement. Additional information can be computed from the raw data and instrument calibration information that indicates the quality of the ECL signal, for example, whether the instrument was operating in an appropriate environmental condition, whether sample was present, or whether the instrument was operating as expected. The raw data and such derived data can, for example, be stored in an exemplary ImmunoScore system database. In general the size of the storage required for this raw data can vary depending upon the resolution at which the data is captured. It is possible that a finer-grained resolution, resulting in a larger data storage requirement, will yield more useful analysis for some assays rather than others. Storage of both the raw data and the derived values can be done, for example, using industry-standard methods for the persistence of floating point numbers. For example, four (4) bytes of storage, yielding approximately six (6) significant digits, can be used for each stored value.

The quantity of greatest interest in an assay is the concentration of the analyte under evaluation. This concentration can be determined by converting a computed ECL signal to a concentration. This conversion can be done, for example, by backfitting the ECL signal through a calibration curve that relates ECL signal to analyte concentration. In general, such a calibration curve can vary from assay to assay, and can change over time for a given assay as that assay is refined.

Calibration curves enable both interpolation and extrapolation of ECL signal measurements for samples with known analyte concentrations for ECL signal measurements of samples of unknown amounts of analyte. The form of the mathematical functions used in a curve fit can, for example, make assumptions regarding the continuity and/or smoothness of the underlying relation such as through interpolating the measurements with functions such as piecewise constant, piecewise linear, cubic spline, or for example, by throughfitting all the data with linear, quadratic, cubic, or quartic polynomials. For overconstrained systems, parameters can be computed by minimizing an error function such as, for example, least squares (e.g., Press et al. 1992) or total least squares (e.g., Van Huffel et al. 1991). The form of the mathematical function may make assumptions about the assay mechanism, such as a one site saturation, two site saturation, one site saturation with nonspecific binding, two site saturations with nonspecific binding, a sigmoidal dose response curve with or without a variable slope, one-site competition, two-site competition, or a four-parameter logistic. Generation of a calibration curve entails selecting the form of the mathematical function and then fitting the parameters of the function with measurements. The measurements can, for example, be done on the test instrument or can be done in whole or in part elsewhere (e.g., at the place the assay is manufactured). The measurements can either perfectly constrain or over-constrain the mathematical function. As noted, for overconstrained systems, model parameters can be computed by minimizing an error function such as least squares.

In exemplary embodiments of the present invention, for each analyte the form of the mathematical function or model (stored, for example, as an index into a table of known models), the computed model parameters, as well as the data used to compute the model parameters, can be associated with each measurement of the analyte. To reduce the amount of redundant information stored in the database, the association for each measurement can be a link to the calibration data rather than the calibration data itself. Instruments can be re-calibrated at any time, such as, for example, on a weekly basis or with every measurement. The quality of the calibration can also be assessed, for example, through the running of controls or by computing the residual error from an overconstrained curve fit.

Thus, a calculated concentration can be stored by the system. This can be, in exemplary embodiments of the present invention, the primary input to analysis recommendation algorithms employed by the remainder of the system. It is noted that not all assays will result in a quantitative concentration. For example, some assays, due to the shape of their calibration curve, may yield two different concentrations for the same measured signal. Such assays are said to “hook.” In such cases the most an exemplary system can store is an indicator that the measured concentration is above a certain level, the lower of the two returned calculated values. Other assays, for various reasons, may return only qualitative results rather than true quantitative results. In all cases, a system database can be capable of storing and retrieving the result. For this reason, in exemplary embodiments of the present invention, the result of an assay can be stored not as a simple floating point number, but as a complex object which can take into account the various scenarios described above. Such an object can have, for example, several fields of its own.

A compressed version of the database can, in exemplary embodiments of the present invention, consist of only the initial ID information, patient ID information, test ID information, and the calculated concentration of analyte. This is a minimal set of data which can prove productive for data mining and trending analysis, as detailed below. The additional data described herein can, for example, be used to enhance the value of this analysis.

Algorithms encoded or implemented or implemented in an exemplary system can be used, for example, to determine a recommendation for action. This recommendation can be based upon a calculated concentration of, for example, antibody response. Other information can also be considered, including, for example, the results of other assays upon the same sample within a given assay panel.

Regardless of the means of determining the recommended action, as described above, a final recommendation can be stored in the database. A system database can, for example, also store the “reasoning” behind the recommendation, allowing a human to later query the database to determine why a given course of action was recommended. Given that the number of recommended courses of action can be broad, these actions can be categorized and encoded. For example, a recommendation to administer a particular vaccination may be encoded with one byte to indicate “give vaccination” and two additional bytes to indicate the particular vaccination that is warranted. A field for comments can also be included, to allow the capture of the system's reasoning—in this case, an explanation of how algorithms and rules were applied to determine the stated conclusion.

A system database according to an exemplary embodiment of the present invention can be implemented, for example, as a shared resource spread over multiple computer platforms. For purposes of trending and analysis, it may be necessary to accumulate the data from a large number of systems into a central repository as depicted in database FIG. 2, or in the case of having only decentralized information, a mechanism to locate and query the distributed sources. The individual databases can therefore require the capability to link up with a defined central database and upload their contents. This may occur on a periodic basis, or as triggered by the user of the system. Additionally, there can be multiple central servers, so that a given enterprise may choose to aggregate their data at any level. The unique IDs associated with sample and panel records can serve to allow combination of data from disparate sources without data “collision”.

The linkage between local databases and a central database can be implemented, for example, across a local area network (LAN), a private data network, an intranet or across the Internet. It is also possible to link databases on a periodic basis using physical media, such as CD-ROMs.

Once data has been accumulated into a central repository, a separate system can be used to perform data mining and data trending analysis upon the stored data. There are many valuable sorts of analysis which can be performed on the accumulated data in an exemplary system according to the present invention.

Given that each data record can, for example, be identified with a particular patient and a particular time and date, it becomes possible to perform trending analysis of a patient's ImmunoScore profile over time. In many cases an individual's absolute measured value of an analyte is not as important as the trending of that value over a time. Some individuals may have naturally low or naturally high values which are not best measured against a statistical mean for their demographic population, but rather against that individual's own measured history.

As described above, each patient can, for example, may also be placed within certain demographic categories. It can be useful to compare a patient's measured ImmunoScore profile against the corresponding profile for the demographic groups to which he or she belongs. Deviation from the measured means for a demographic slice of the population can prove more meaningful than can a comparison to a total threshold. Thus, in exemplary embodiments of the present invention collected data can be used to continually to modify the demographic profile averages known to the system, taking care to not pollute the system with outlying data points. For example, it may prove useful to produce separate ImmunoScore demographic profiles for patients who are known to have experienced vaccinations versus those for whom there is no known immunization record. Alternatively, as is described below in Section III, such an immunization record can be inferred and reconstructed, as in the provision of ImmunoScore services to National Immigration Services.

Trending information in a demographic profile, for example, can also be useful. For example, tracking an indication of the typical person (e.g., mean, median, or mode), or an indication of the spread amongst people (e.g., standard deviation, interquartile range, or range) over time can enable a system to assess the relationship between immune status indicia and external factors, such as, for example, seasonal effects. Eating habits, sleeping habits, time aboard ship, etc. can be found to affect immune status in groups where these external factors are partially controllable (such as, for example, in military personnel). Comparing immune status indicators of differing demographic profiles can have important epidemiological significance.

Finally, it is expected that the collection of ImmunoScore data from a large number of individuals and/or populations can eventually lead to the improvement of diagnostic tests, thus forming a feedback loop. These improved diagnostic tests can then, for example, be deployed to field instruments, resulting in more accurate measurements and diagnoses. Such exemplary embodiments having feedback loops can be implemented, for example, with respect to particular populations or demographic groups, such as, for example, the military, college students, immigrants or any other group or combination thereof as described above.

B. Example Illustrative Database

1. Overall Description

To illustrate the systems and methods of the present invention, a database system was constructed to serve as a testbed for the exercise of the business models described below. Such an exemplary database system was used to demonstrate the tools and techniques that might be used in a full scale system according to the present invention. Accordingly, a large data set was constructed using statistical techniques. The data was produced according to match existing knowledge about the distribution of immune response values among the general population.

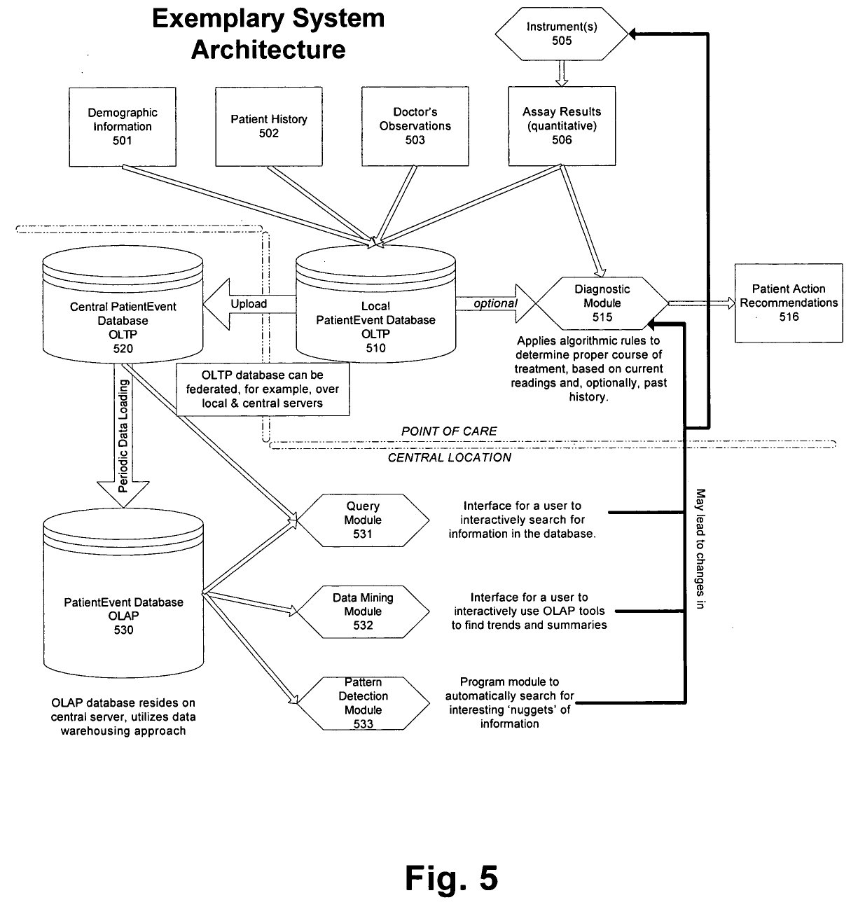

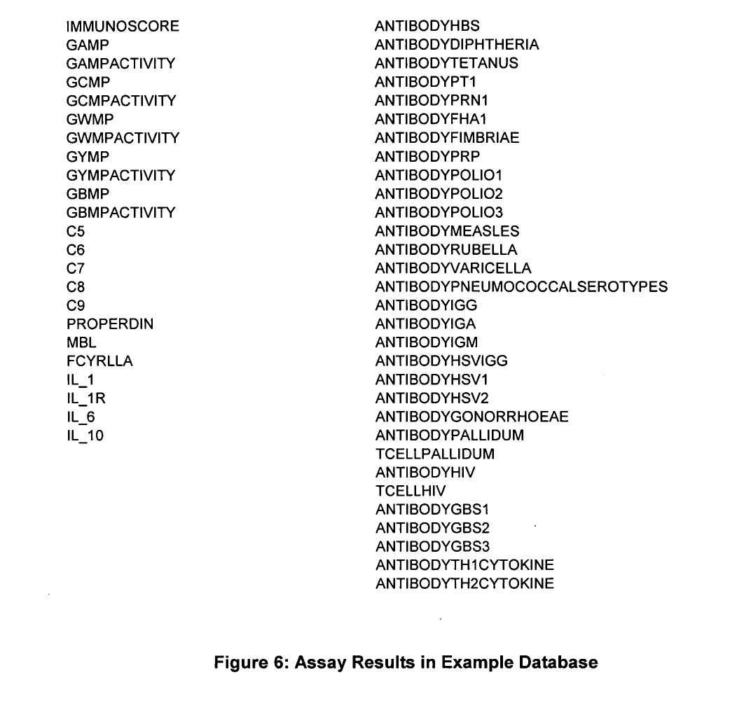

The exemplary database system has two primary components. These two components represent the algorithmically interesting sections that can be, for example, present in a full-scale operational system. Such a full system could, for example, contain other modules as well, along the lines of industry standard large scale database systems. Such an exemplary system is depicted in FIG. 5.

The following is a general description of an exemplary system architecture according to an exemplary embodiment of the present invention as depicted in FIG. 5. With reference to FIG. 5, an exemplary system architecture can be constructed. The exemplary system architecture can be, for example, divided into two sub-systems, one relatively local to “point of care” or locations where the individuals or patients whose immune status is to be analyzed are located. The other subsystem can be in a central location where complex data mining and analysis can occur. Thus, with reference to FIG. 5, the upper portion of the figure contains components which can be located at the point of care and a lower portion of the figure contains components which can be, for example, located at a system central location. The point of care is divided from the central location in the figure by a double dotted and dashed line for ease of identification.

With reference to the point of care sub-system, there can be seen one or more Instruments 505 which are devices which can read immunologic assays. Instruments 505 yield Assay Results 506. Assay Results 506, along with Doctor's Observations 503, Patient History 502 and Demographic Information 501 regarding the individual or patient can all be stored in Local PatientEvent Database 510. Database 510 can be, for example, an online transaction processing database. Because the point of care sub-system is directed to generating a recommendation in a relatively short time, there are two pathways to Diagnostic Module 515. Diagnostic Module 515 applies algorithmic rules to the assay results to determine proper course of treatment or action based on current readings and optionally on past history. Thus, there is a flow of information from Assay Results 506 to Diagnostic Module 515. Alternatively, Diagnostic Module 515 can implement algorithms having other inputs besides the current Assay Results 506, such as, for example, Demographic Information 501, Patient History 502, and Doctor's Observations 503 which are stored in the Local PatientEvent Database 510. As a result, there is an arrow labeled “optional” running from Local PatientEvent Database 510 to Diagnostic Module 515. Regardless of which source of information Diagnostic Module 515 draws upon, it can output the patient action recommendation 516 as indicated.

Returning now to the central location sub-system, a connection exists between the Local PatientEvent Database 510 and a Central PatientEvent Database 520. This connects the two sub-systems. It is contemplated that at regular intervals data from Local PatientEvent Database 510 will be uploaded to Central PatientEvent Database 520. Moreover, although the central location sub-system could be mirrored in a number of distributed central location subsystems, the point of care sub-system is contemplated to take data from numerous instruments and in fact have numerous local patient event databases in those locales. In short, the point of care sub-system is found wherever potential customers or patients are found. Therefore, there could be a great number of local patient event databases all of which feed into Central PatientEvent Database 520. None of these additional point of care sub-systems are shown in FIG. 5 for ease of illustration.

Returning again to Central PatientEvent Database 520, it is noted that this database is also an online transaction processing database or OLTP. It is contemplated that this database periodically loads data to an online analytic processing database, or OLA, P in the form of PatientEvent Database 530. It is PatientEvent Database 530 that is adapted to provide inputs to complicated algorithms dealing with data mining and pattern detection, as next described.

PatientEvent Database 530 can, for example, reside on a central server and utilize a data warehouse approach. There can be a variety of connections to PatientEvent Database 530 such as, for example, a Query Module 531, a Data Mining Module 532 and a Pattern Detection Module 533. Query Module 531 is an interface by which a user can interactively search for information in database 530. Query Module 531 can also access Central PatientEvent Database 520 and conduct a variety of searches there as well. Data Mining Module 532 is an interface by which a user can interactively use OLAP tools to finds trends and summaries in the stored data. Finally, Pattern Detection Module 533 is a program module which can be used to automatically search for patterns or other “hidden” correlations between various data points in the database. It is contemplated that in exemplary embodiments of the present invention the Pattern Detection Module 533 can regularly sort through all of the stored data looking for patterns using various algorithms. Some of such algorithms can articulate some hunch or a correlative assumption provided by a panel of immunological experts for which they do not have hard data. The Pattern Detection Module 533 is thus an important feature in exemplary embodiments of the present invention.

The exemplary system depicted in FIG. 5 will next be described in greater detail. A first module of interest is termed the Diagnostic Module 515. The function of this software module is to input a set of assay results 506 obtained through measurements by instruments 505, and to make one or more recommendations 516 based upon the analysis of assay results 506. Diagnostic Module 515 can be designed in such a way that additional assay panels can be slotted into an existing system as they are developed. Some exemplary algorithms used to make recommendations as a function of assay results are described in more detail below, including descriptions both of algorithms used in the exemplary database as well as additional algorithms that could be implemented in various exemplary embodiments of the present invention.

The Diagnostic Module 515 rests upon a Local Database 510 containing Assay Results 506 obtained from Instruments 505. These results are pertinent to an individual patient. Local Database 510 can also, for example, contain background medical history 502 for that patient, demographic information 501 pertinent to the patent, and a summary of other medical observations 503 made by medical professionals. Local Database 510 can also, for example, contain statistical information obtained from a larger central database, as described below.

A second exemplary module of interest is Data Mining Module 532. Whereas the diagnostic module 515 is intended for the analysis of a particular individual's data at a particular point in time, the data mining module 532 is intended to look at a broader range of data collected from many individuals over a long range of time. The intent is that through analysis of this collected data a system can be used to support various business methods and other applications by deducing trends and patterns within the immunological landscape. A particular result could be fed back into the diagnostic module algorithms, improving their effectiveness by providing additional specificity with regard to an individual's background, possibly in terms of demographic information such as, for example, gender, racial background, geographic origin or age.

As can be seen from FIG. 5 while the Diagnostic Module's functionalities are primarily local in nature and patient-specific, the Data Mining Module's functionalities are primarily central, and system-wide. This structure is reflected in the division of FIG. 5 into two zones, the “Point of Care” zone, shown at the top of the figure, and the “Central Location” zone, shown at the bottom of the figure.

The Data Mining module 532 depends upon the existence of a large central database containing records from a wide variety of individuals over a long span of time. Thus, the local databases described above can exist in a federated state with the central database, uploading their information on a regular basis, where this information can, for example, be integrated into the full system.

2. Impact of Data Mining

The impact of the results from data mining on the subsequent design of the database is estimated, particularly with respect to the Diagnostic Module. It is anticipated that patterns can be detected which are related to demographic information such as, for example, gender, age, ethnicity, etc. These patterns may not be obvious until large numbers of individuals are assessed, using a computer that can be by nature much more efficient, unbiased, and precise in pattern recognition.

From such patterns, new correlates can, for example, can be established, and old correlates can be changed. For example, it may be proposed, based on previous data, that a serum antibody concentration of 2 micrograms per ml should be used to represent a threshold of protection against meningococcal disease, so that anyone with less antibody would be recommended for immunization. Subsequent and continued analysis, however, may show that this threshold value should be reduced or raised, depending on, for example, age or ethnic background, or some other undefined parameter. In turn, an ethnicity evaluation could lead to the discovery of a specific biological or genetic marker. For example, the functional activity of Haemophilus influenzae type b (Hib) antibodies may vary with different individuals, where the same antibody concentration may not possess the same level of bacteriocidal activity due to differences in antibody avidity (Amir J et al., 1990, J Infect Dis 162:163-71; Amir J et al., 1990, Pediatr. Res. 27:358-64). For example, regarding age, Hib polysaccharides were shown to be poorly immunogenic in children less than 2 years of age (Granoff D M, 1985, J Pediatr 107:330-36). Similarly, regarding ethnicity it has been shown from previous studies that Eskimos and Apaches are more susceptible to Hib meningitis because they possess a less effective antibody repertoire to the Hib polysaccharide capsule, based on the presence or absence of certain variable region genes used in the production of the polysaccharide-specific antibodies (Ward J et al., 1990, N Engl J Med 323:1393-1401; Siber G R et al., 1990, N Engl J Med 323:1387-92; Lucas A H, Granoff D M, 1990, J Clin Invest 85:1158-66; Lucas A H et al., 1991, J Clin Invest 88:1811-18).

Additionally, variations in host factors can lead to significant differences in the immune response to vaccines, which can also be discerned by data mining. For example, late-stage complement deficiency may have no impact on antibody production, but would certainly reduce the effectiveness of those antibodies in killing bacteria, thereby lowering their activity. In such case, the antibody threshold for protection may need to be raised in order to achieve the same level of protection in this subpopulation.

As previously described for Hib, the capacity for protective antibody production is the direct result of variable region gene haplotypes. In this case, ethnic differences were first observed as a gross marker, but the presence of specific genes was later determined to be responsible. In a similar but different manner, HLA haplotypes have also been correlated with the susceptibility to certain infections, as well as the unresponsiveness to certain vaccines. For example, certain HLA antigens appear to be correlated with chronic hepatitis B virus (HBV) infections and HBV vaccine nonresponsiveness (Thio C L et al., 2003, J Virol 77:12083-87; De Silvestri A et al., 2001, 2:367-72; Leroux-Roels G et al., 2001, Acta Clin Belg 56:209-19). In such cases, in exemplary embodiments, of the present invention, subpopulations can be identified, initially by ethnicity, then later by genetics, to evolve a more specific and appropriate diagnostic outcome.

Another example of the influence of ethnicity on responsiveness to treatment is the case of NitroMed's BiDil™, which was approved by the U.S. FDA in 2005 for the treatment of heart failure in African Americans. BiDil™ is an orally administered, nitric oxide-enhancing drug that was shown to have clearly different effects on blacks versus whites in clinical trials, where the “differences may be related to environmental, social, lifestyle, or genetic factors or to interactions among all of these.” (http://www.fda.gov/fdac/features/2005/505_BiDil.html). In exemplary embodiments of the present invention, data mining can, for example, be used to observe and identify these kinds of effects and correlations, and then be later used to determine the specific underlying mechanisms.

Data mining can also be used, for example, to change or reverse previously held dogma(s) concerning long-term protection from vaccination. For example, immunity resulting from the smallpox vaccine, used extensively during the previous century, was originally thought to last for less than a decade. Recent analyses however, have shown that “more than 90% of volunteers vaccinated 25-75 years ago still maintain substantial humoral or cellular immunity (or both) against vaccinia, the virus used to vaccinate against smallpox.” (Hammarlund E et al., 2003, Nature Medicine 9:1131-37). This study further showed that, “Antiviral antibody responses remained stable between 1-75 years after vaccination, whereas antiviral T-cell responses declined slowly, with a half-life of 8-15 years.” While it is not clear what level and combination of responses is required for protection, the authors concluded that “the morbidity and mortality associated with an intentional smallpox outbreak would be substantially reduced because of pre-existing immunity in a large number of previously vaccinated individuals.” This is exactly the kind of information that could be obtained through data mining over time on large populations, as contemplated in exemplary embodiments of the present invention.

As noted above, an exemplary system similar to that of FIG. 5 was built using standard software development tools and packages. The algorithms were encoded using the XML data description language. The engine for executing the algorithms was built using the Java programming language. An Oracle database was used for data storage and data mining querying. Excel spreadsheets were used for data construction and analysis. Details of the construction are given below.

3. Diagnostic Module

3.1 Overview

Diagnostic Module 515 forms the heart of an exemplary ImmunoScore decision system. At a basic level, the diagnostic module exists to suggest courses of recommended action based upon an individual's immune status, as measured by instrumentation or obtained from elsewhere, in combination with other supporting data. There are many different ways that such a determination could be made. Next described are both the algorithms that used in the example system as well as other exemplary decision support algorithms which could be implemented using the same techniques.

An essential function of a diagnostic module is to assist a medical or other professional in making decisions regarding which actions to take with a specific individual, making use of data regarding that person's immune status. As noted, in exemplary embodiments of the present invention, an individual's immune status can be determined by conducting a panel of assays, each of which assays can produce an element of data. For purposes of the example database, information presumed to be obtainable through such assays is summarized in FIG. 6. It should be noted that in practice some of this information may not yet be obtainable, although it is expected that assays could be developed along the lines of existing tests in order to complete this spectrum.

In addition to immune status information obtained from assays, a diagnostic module can make use of other information specific to the patient being examined. This information falls into two principal categories: demographic information, such as, for example, age and gender, and patient medical history. Most demographic information can be simply expressed in a database. Patient medical history is more problematic, although there are many existing healthcare database systems which do this adequately. The difficulty with patient medical history, however, is in devising algorithms which can make use of this qualitative data. It is expected that particular care can be taken to use algorithmic techniques which have proven adept in dealing with inconsistent or unreliable data, such as, for example, neural networks, described in greater detail below. This is due to the inherent unreliability of self-reported medical history data, along with the historic problems found in the transfer of medical records. If a system with built-in reliability checks is implemented, then it can be possible to rely more strongly upon historical data.

Thus, the exemplary system described below can store both demographic and past medical history information for individual patients, but does not make use of these factors in performing diagnostic assessments or recommendations of courses of action. However, the algorithms implemented can easily be extended into these realms once more information becomes available.

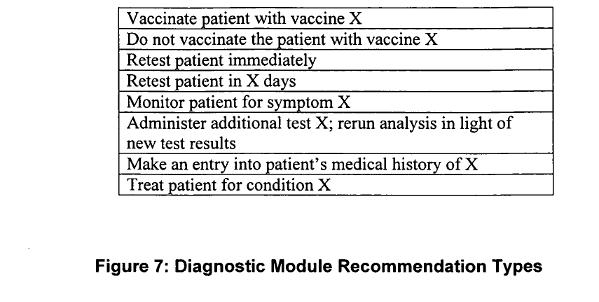

The output of Diagnostic Module 515 is a series of recommendations. A recommendation is simply defined as any discernible bit of data which might be of interest to a medical professional, health care or life insurer, researcher or other user of the present invention in determining a given course of action. In the case of a patient's immune status, a common recommendation could be, for example, to recommend a particular vaccination. Of equal importance is a recommendation to not vaccinate, as it is relevant to several business models to reduce the over-vaccination of the populace. A summary of the types of exemplary recommendations that can be offered by a Diagnostic Module are given in FIG. 7.

The Diagnostic Module is capable of producing a set of recommendations for each analysis. For example, it might recommend that both vaccine V be administered and that the individual be retested in three weeks to monitor his or her response to such vaccine. For each recommendation, the Diagnostic Module can, for example, also provide a confidence level, which is a measure of the system's support for any given conclusion. A user can take this confidence level into account when deciding upon a course of action. A course of action with a low confidence level but a high financial cost, for example, could be delayed until additional data could be gathered to more strongly support the course of action.

The Diagnostic Module can, for example, be constructed in a manner to allow the deployment of many different algorithms within its basic shell. For the exemplary system, an algorithmic approach based upon perceptrons used. This approach is detailed below. Additionally described are alternative algorithmic approaches, each of which has different strengths and weaknesses. It is noted that some of these approaches are realistically infeasible until such time as large-scale data collection of immune status informatics becomes available.

3.2 Perceptron Algorithms

A perceptron is a simple neural network, a computer science representation based upon an analogy with the operation of human neurons. Perceptrons were invented by Frank Rosenblatt in 1957, and have been used in artificial intelligence research since that time. A perceptron is simplistic, but adequate for the computation of algorithmic diagnostic results within the exemplary system of the invention. More importantly, there is a clear progression between perceptrons and more sophisticated artificial intelligence techniques, which may be of use in more complex embodiments of the invention.

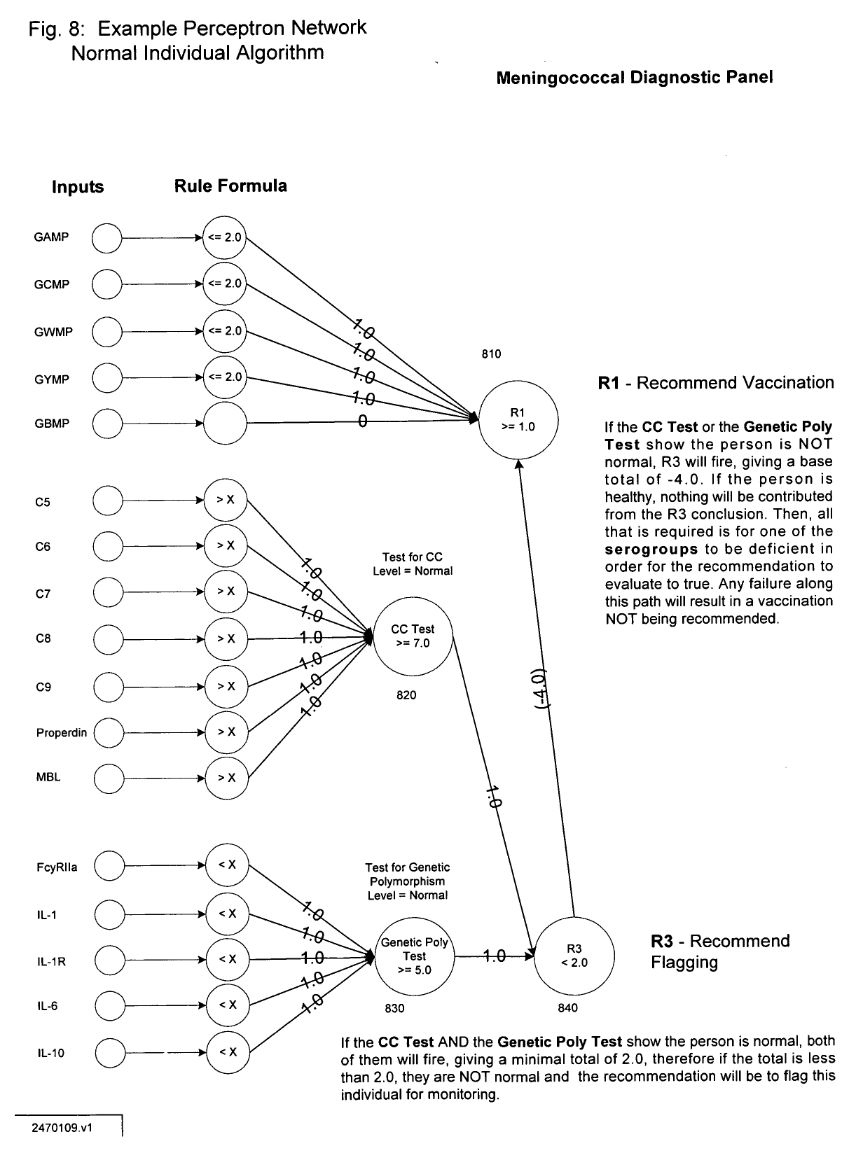

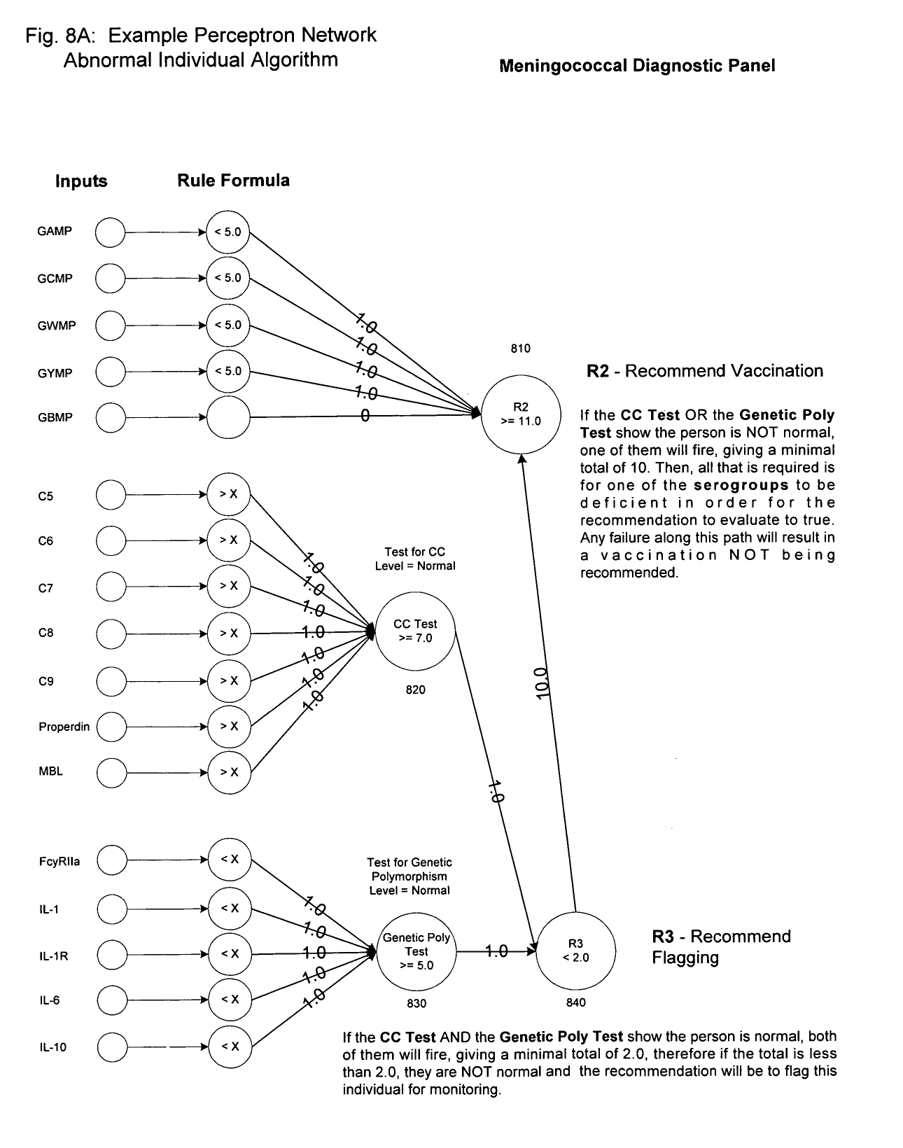

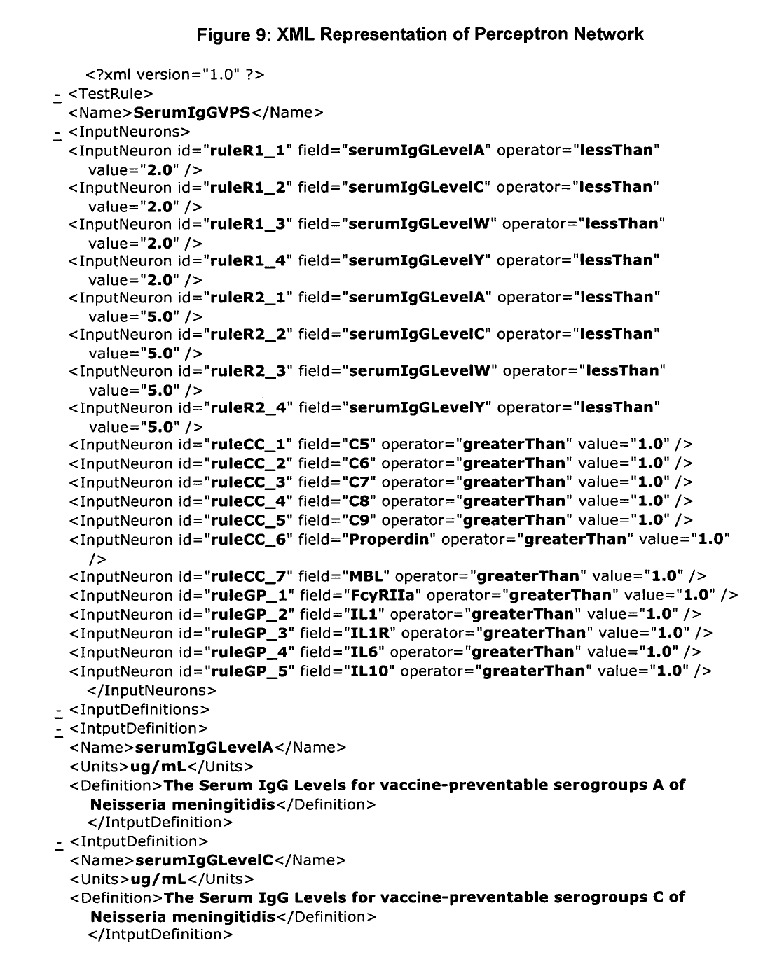

An example of a perceptron is given in FIGS. 8 and 8A. These networks encode the decision making process for the running of a Meningococcal Diagnostic Panel, as described above. There are seventeen inputs to the algorithm, one for each of the measurements that can be taken in an exemplary meningococcal assay panel. Five inputs are for the meningococcal serogroups, seven for the complement components, and five for the genetic poymorphisms. There are two output recommendations from this panel R1 810 (or in FIG. 8A, R2 810) and R3 840. R1/R2 is a recommendation to vaccinate an individual with a meningococcal vaccine. R3 840 is a recommendation to monitor the individual on a stricter interval schedule than normal, because the individual may be more susceptible to this condition than the average individual in the populace. FIGS. 8 and 8A depict the same prerceptron, with different values for the various nodes upon firing.

With reference to FIG. 8, serum IgG levels for vaccine-preventable serogroups (A, C, W-135, and Y) of Neisseria Meningitis can be assessed. As seen in the fifth input to R1, the panel also has a built-in facility to measure and consider serogroup B, but there is no currently available vaccine or clearly known threshold of protection for this serogroup, so it was left blank. A serum IgG level exceeding 2.0 ug/mL for all four serogroups would be presumptive of protection in an otherwise healthy individual, i.e., an individual (i) found not deficient in serum levels of measured complement components, and (ii) having no deleterious genetic polymorphisms as indicated in the CC Test 820 and Genetic Polymorphism Test 830. There would be no immediate recommendation for meningococcal vaccination for these individuals. The following is a description of rule execution flow for FIGS. 8 and 8A.

R1—Recommend Vaccination. With reference to FIG. 8, If the CC Test 820 and the Genetic Poly Test 830 show the person is normal, both of them will fire, giving a minimal total of 2.0 at R3. Then no contribution at R1 from R3, and if any of the serogourps is deficient, R1 will be at least =1.0 and R1 will fire. If the CC Test 820 or the Genetic Poly Test 830 show that the person is not normal, R3 840 will fire, giving a base total of −4.0. Nothing will be contributed from the R3 conclusion as even if the inputs to R1 810 from the four serogroup assays are all 1.0 (all deficient), this added to −4.0=0, which is <1.0, and R1 needs to be >=1.0 to fire. Thus FIG. 8 only operates as to normal individuals vis-à-vis the CC and Genetic Poly tests.

R3—Recommend Flagging. If the total at R3 840 is less than 2.0, the individual is not normal, R3 fires and the recommendation will be to flag this individual for monitoring.

FIG. 8A is similar to FIG. 8, applying a different recommend vaccination rule, R2 at 810, for a different immunological context. The perceptron is modified as to values, but the nodes are identical.

R2—Recommend Vaccination. With refernce to FIG. 8A, if deficiencies were to be revealed in any of an individual's complement components, or if any unfavorable genetic polymorphisms were shown to exist, then it is likely that a serum IgG level of >5.0 ug/mL (not the >2.0 UG level as in the rule of FIG. 8) for the vaccine-preventable serogroups would be desirable in these individuals. If these individuals had IgG levels exceeding 5.0 ug/mL for all four serogroups, no vaccination would be recommended. If the level of antibody to any of the four serogroups were to be below 5.0 ug/mL, then a vaccination would be recommended. If the CC Test or the Genetic Poly Test show the person is not normal, one of them will fire, giving a minimal total of 10 at R2. Then, all that is required is for one of the serogroups to be deficient (i.e., <5.0 ug/ml) in order for the recommendation at R2 to evaluate to true.

R3—Recommend Flagging. If the CC Test and the Genetic Poly Test show the person is normal, both of them will fire, giving a minimal total of 2.0. If the total is less than 2.0, R3 fires, they are not normal and the recommendation will be to flag this individual for monitoring.

Because al perceptrons operate on the data in parallel, an abnormal individual will be captured in the perceptron of FIG. 8A and will receive no vaccination recommendation from the perceptron of FIG. 8.

A perceptron operates through software by simulating the “firing” of nodes based upon numerical conditions being met. As each node fires, it can contribute to the firing of other nodes, in some cases positively and in some cases in an inhibitory fashion. The network as a whole has completed execution when the rightmost nodes, representing diagnostic recommendations, have either fired or have come to rest.

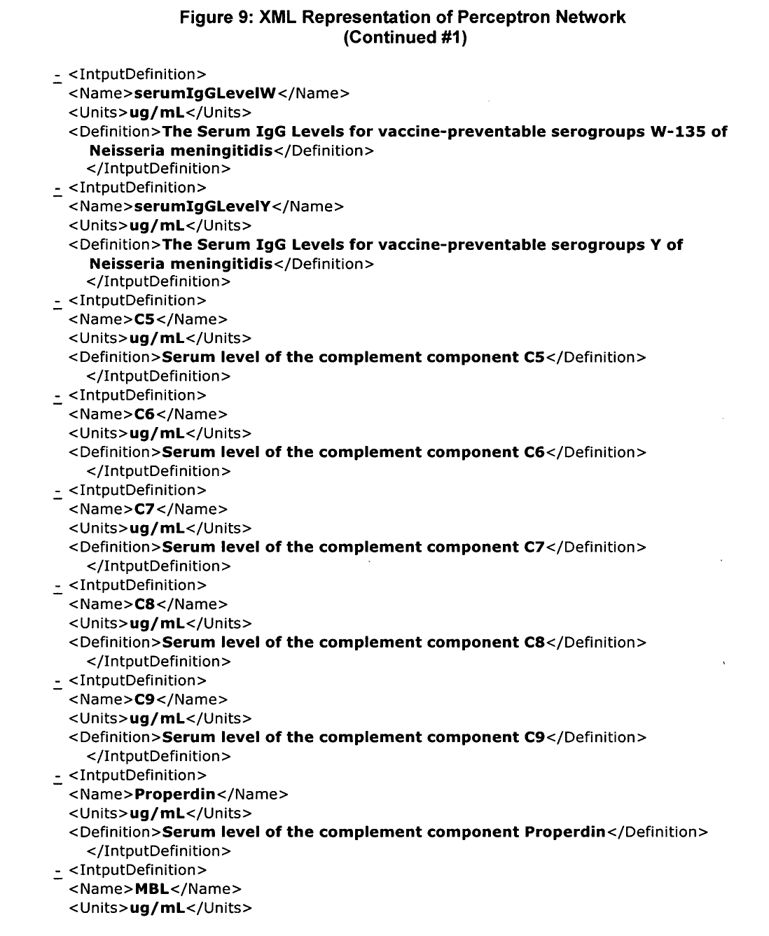

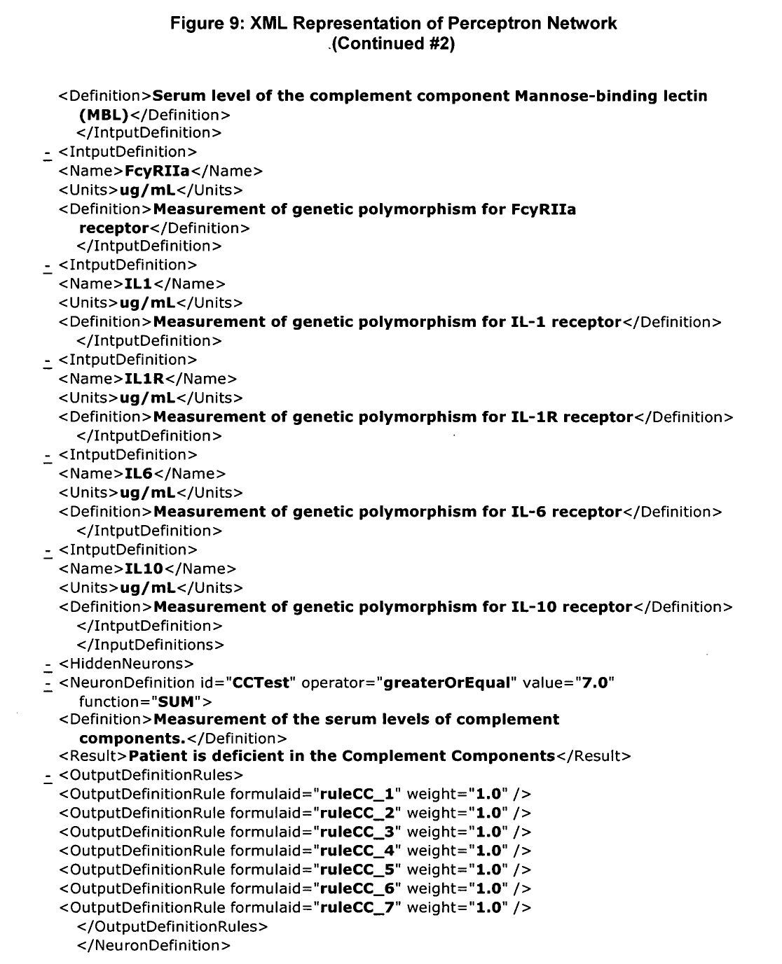

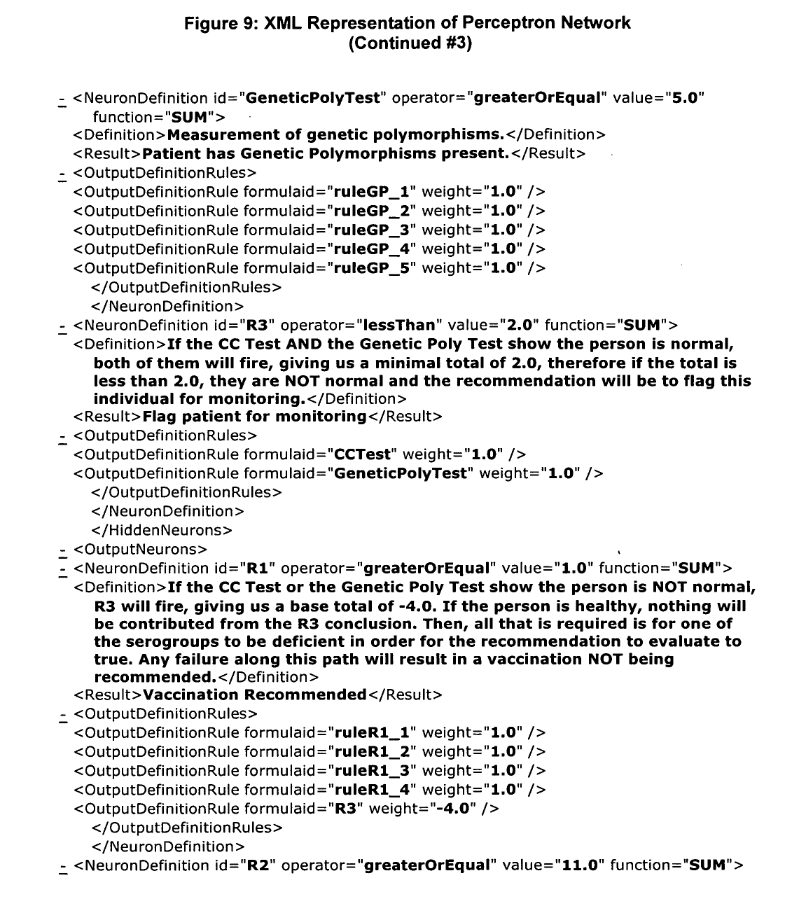

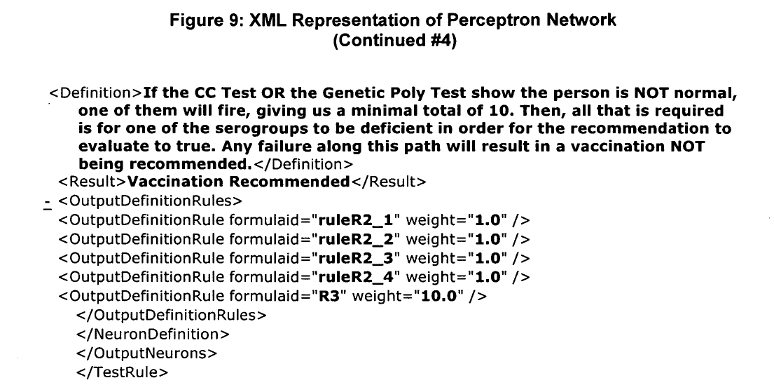

The perceptrons in the exemplary were encoded manually based upon existing knowledge of diagnostic recommendations in use today. Each perceptron can be represented either graphically, as in FIG. 8, or textually, as in FIG. 9. FIG. 9 is thus a textual representation of the perceptron network using a language called XML, or eXtensible Markup Language. In the exemplary these XML files can be deployed to the diagnostic module as discrete packets. A Diagnostic Module connected to an instrument, or bank of instruments, could, for example, be configured with only those perceptron algorithms required for that site.

In addition, updated versions of these algorithms could be deployed as the algorithms are improved over time. Thus, in exemplary embodiments of the present invention, it is anticipated that knowledge gained through use of the data mining module, detailed below, can be fed back into the individual diagnostic modules, thus improving the accuracy of the entire system. For example, it may be deduced through data mining of an exemplary database that the level of antibody activity which is a strong indication of the need for vaccination is lower in men than in women. A new perceptron algorithm could then be deployed, for example, including the gender of the patient as a new input node, with a link to the vaccination recommendation node.

More subtly, a perceptron can include within it a series of weights which can, for example, correspond to the importance of each bit of evidence to the recommendation procedure. Over time these weights can be adjusted and redeployed to reflect increased understanding of the role of each of the immunological factors being measured.

3.3 Alternate Algorithmic Approaches

There are a number of alternate algorithmic approaches which can be used within a Diagnostic Module. Each has varying strengths and weaknesses. An exemplary system can employ a combination of these approaches in order to come up with the most complete recommendation for a course of action.

The in the process of evaluating algorithmic approaches is a consideration of the goals which are to be met. A Diagnostic Module can, for example, be configured to optimize for any one of a number of different criteria. Possible goals can include, for example, optimizing the welfare of the patient, minimizing costs for the patient related to the disease in question, minimizing overall patient healthcare costs, and minimizing life insurance costs. The decision algorithm used in the diagnostic module can thus vary depending on how these goals are prioritized.

A key difference between a system according to the present invention and existing systems is the use of an individual's immune status information as an input to the decision procedure. This allows the system to provide more tailored and individualized recommendations instead of relying upon aggregate statistical measures. A second key difference is the introduction of historical patient immune status data. It is possible, for example, that a given individual's antibody level is below some computed norm, but is in fact high in relation to that individual's past results. This might conventionally be a contraindication for vaccination, a recommendation which would not be made if the individual's immune status were only to be compared to the population standards.

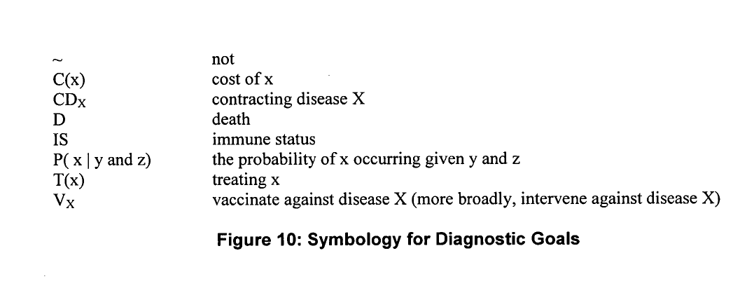

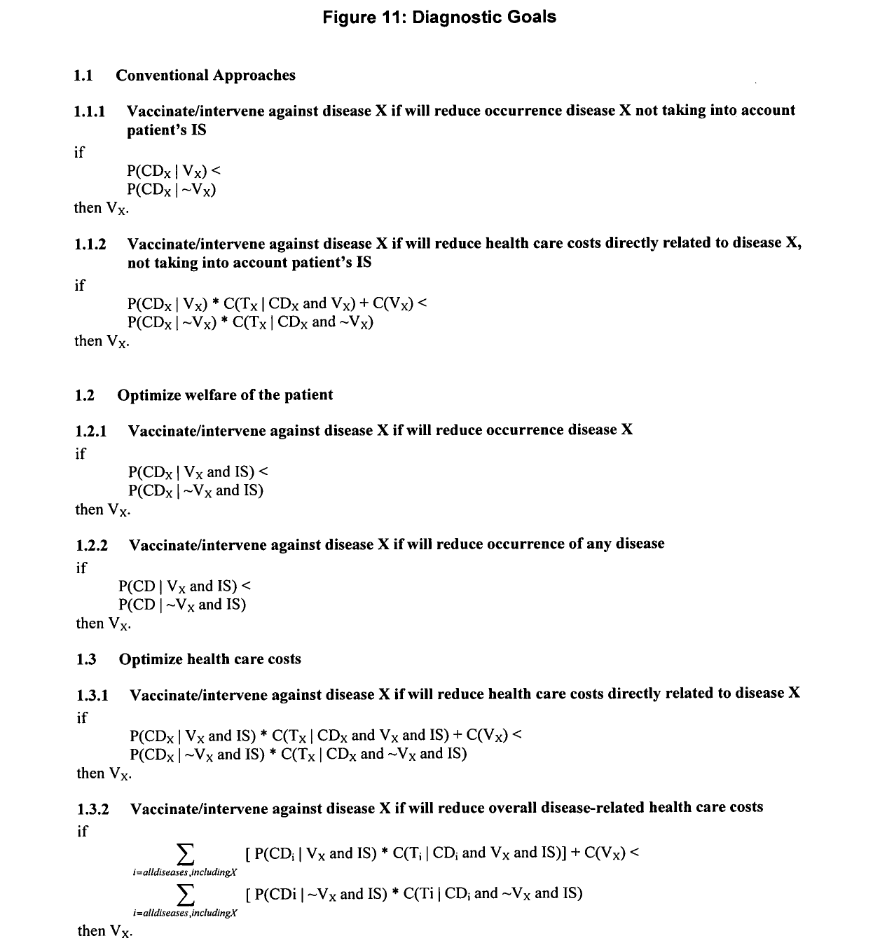

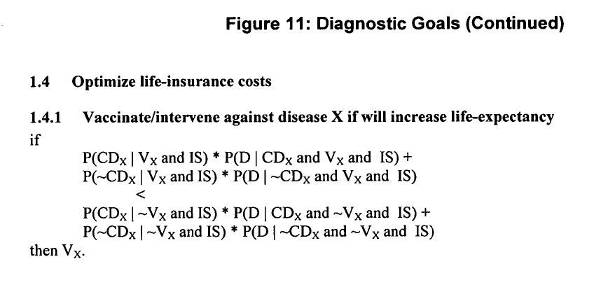

Using the exemplary symbology laid out in FIG. 10, various diagnostic goals as shown in FIG. 11 can be summarized.

3.3.1 Additional Input Data

This section describes additional data which could be incorporated into the diagnostic module.

As noted above, historical immune status information can be a useful addition. Basing a recommendation solely upon an individual's status at the current point in time is an adequate approach, but it risks making incorrect recommendations for those patients who do not fall within the average range of the population at large. A simple extension to the system would be to move away from absolute measures of, for example, antibody level and antibody activity level, and to substitute instead relative measures based upon the percent change in these values since the last historical measurement, or in comparison to the individual's historical averages. The same decision procedures could be applied, but retooled so that a decision rule such as “the level is greater than 30″ becomes “the level is greater than 15% above the patient's baseline”. In order for this to occur, an exemplary system can either maintain a central record of the patient's immune status over time, or provide means to allow the portable storage and transfer of this historical record, perhaps under the patient's control. Various forms of “smartcard” or electronic storage technologies as are known could be used for this purpose.

A second type of additional input data relates to demographic information. Current decision procedures do little to distinguish treatment recommendations based upon an individual's age, gender or racial background, although it is known that these factors have a considerable effect on the interpretation of immune status information. Thus, an exemplary system could make use of such demographic information, customizing the diagnostic algorithms to take into account observed patterns. Additional research would be required to deduce these patterns in the population as a whole in order to make reasonable modifications to the decision procedures.

3.3.2 Decision Rule Algorithms

A clear successor to the perceptron approach could be to extend the system to full neural networks. The distinction between perceptrons and more complex neural networks is the incorporation into the latter of feedback links from later nodes to earlier nodes in the network. This not only increases the complexity of the algorithms which can be implemented, but allows for algorithms which improve over time through a learning mechanism. Neural networks are a well-established domain of artificial research. The primary impediment to neural networks is that they are difficult to construct by hand. A typical neural network is instead evolved through the use of training algorithms. These training algorithms require as input a set of training data. In an exemplary embodiment of the present invention, the training data could consist of immune status data from a large population of people coupled with data about the eventual onset of diseases in that population. Were such a database to exist, neural networks could be constructed which could predict the onset of disease based upon features in an individual's immune status information. An advantage to using neural networks is that they could be a simple drop-in replacement to the current Diagnostic Module in terms of inputs and outputs.

4. Data Mining Module

4.1 Overview

The Data Mining Module is the large-scale component of exemplary systems, according to the present invention. As noted above, while the Diagnostic Module focuses upon obtaining results specific to a particular individual, the Data Mining Module is designed to examine trends in large data sets assembled for many individuals and with many readings per individual. This capability is necessary to support business models in which information is deduced about immune status patterns, as well as to improve the functionality of the Diagnostic Module over time.

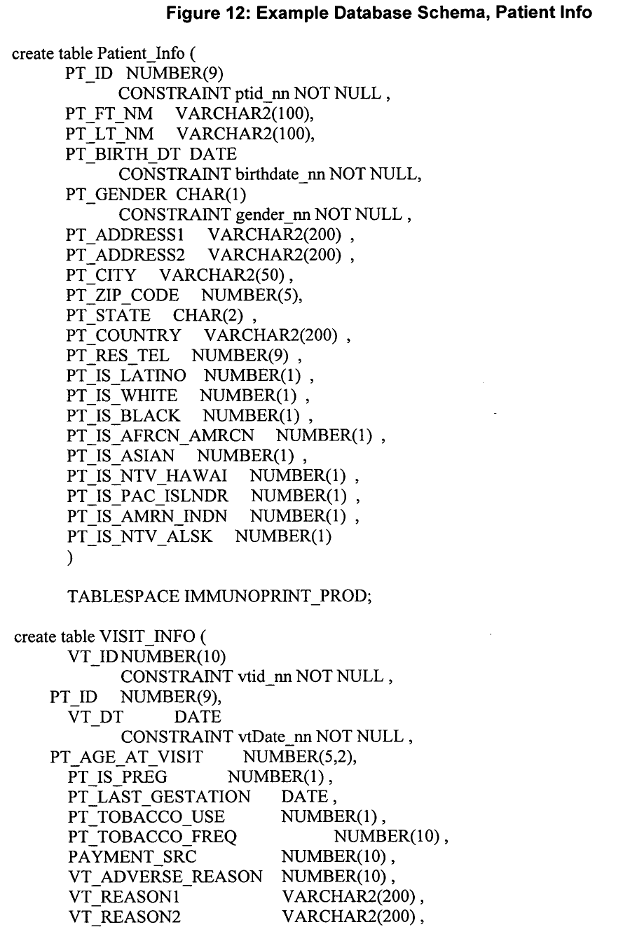

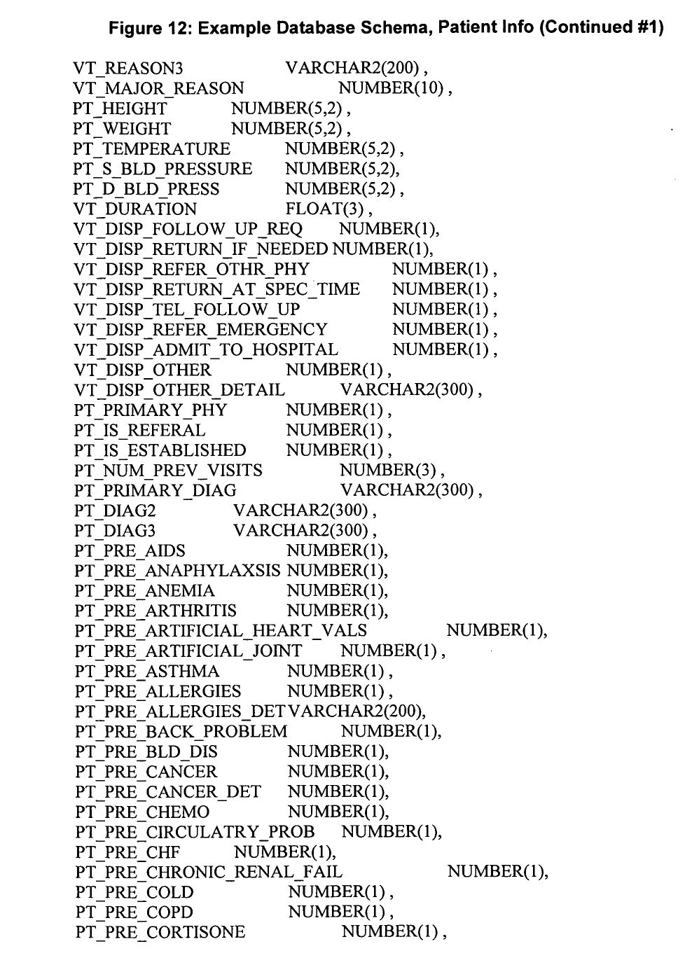

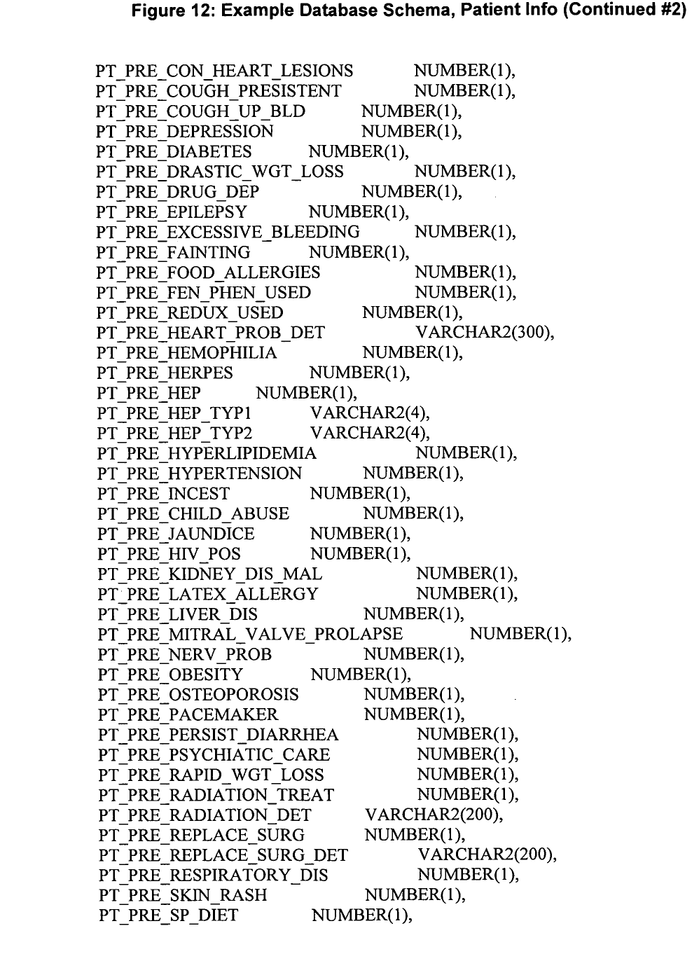

As noted, an exemplary system was constructed using an Oracle database server. The schema for the database system is given in FIGS. 12 through 14. The schema used is termed a ‘star schema’, which is a database layout optimized for online analytical processing. This is a standard concept in data mining. More information about the data storage is given below.

4.2 Sample Data

The sample database was intended to represent actual immune status information which could be collected from a large population over a large span of time. The test measurements contained within the database are randomly generated within the constraints detailed below.

The exemplary database contains three distinct sorts of information.

The first block of information is individual immune status information. As an example, the individual is assumed to be a patient in some healthcare context. The schema for the patient information table is given in FIG. 12. To summarize, the database contains information on the patient's birthdate, gender, racial background and geographic location. All of this information can potentially be used for data mining efforts related to immune status. The database also contains other information strictly for identification purposes, such as name and ID.

In the exemplary database, patient information was randomly generated. Gender was split evenly, and geographic placement was divided among four test cities. Racial backgrounds were assigned to match latest U.S. census figures available.

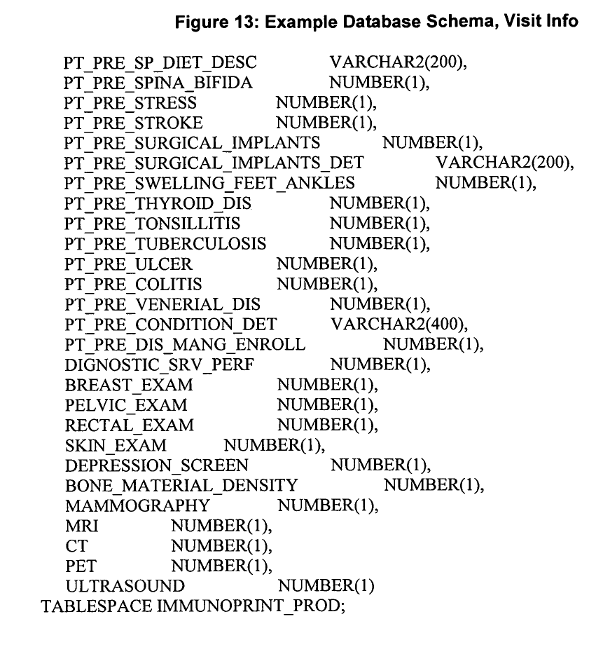

The second block of information is patient visit information. A schema for the patient visit information table is given in FIG. 13. To summarize, this information covers data that could, for example, be collected by a physician at the time of a patient's visit. There can be multiple visit information records for each patient. The majority of this information covers various symptoms present in the patient at the time of the visit. This information can be used within the Diagnostic Module, above, as part of an algorithm which takes into account diagnostic information other than the immune status assay results. This information can also be used in data mining to discover correlations between physical symptoms, immune status indicator levels, and subsequent onset of disease. The visit information section of the database is also used to store recommendations from the Diagnostic Module.

In the exemplary database, symptomatic information was assigned randomly. The example Diagnostic Module did not make use of symptomatic information.

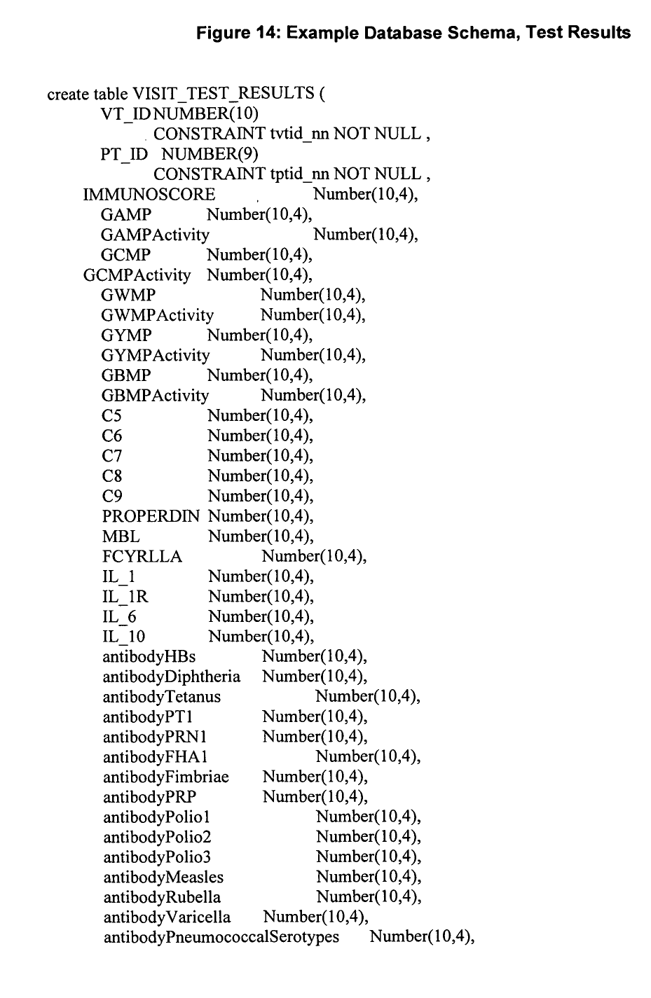

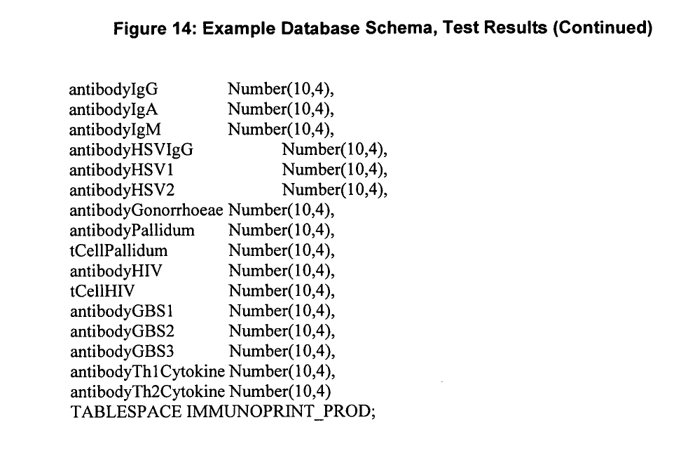

The third block of information is the actual results of immune status assays. In the exemplary database there are 48 distinct simulated measured quantities, although this can be expanded, for example, to any reasonable number in a straightforward manner. The schema for this data block is given in FIG. 14.

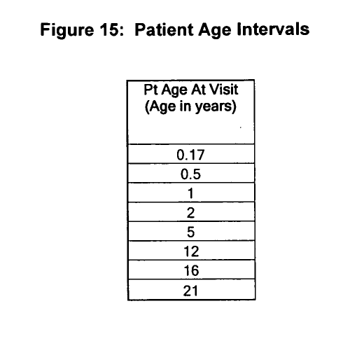

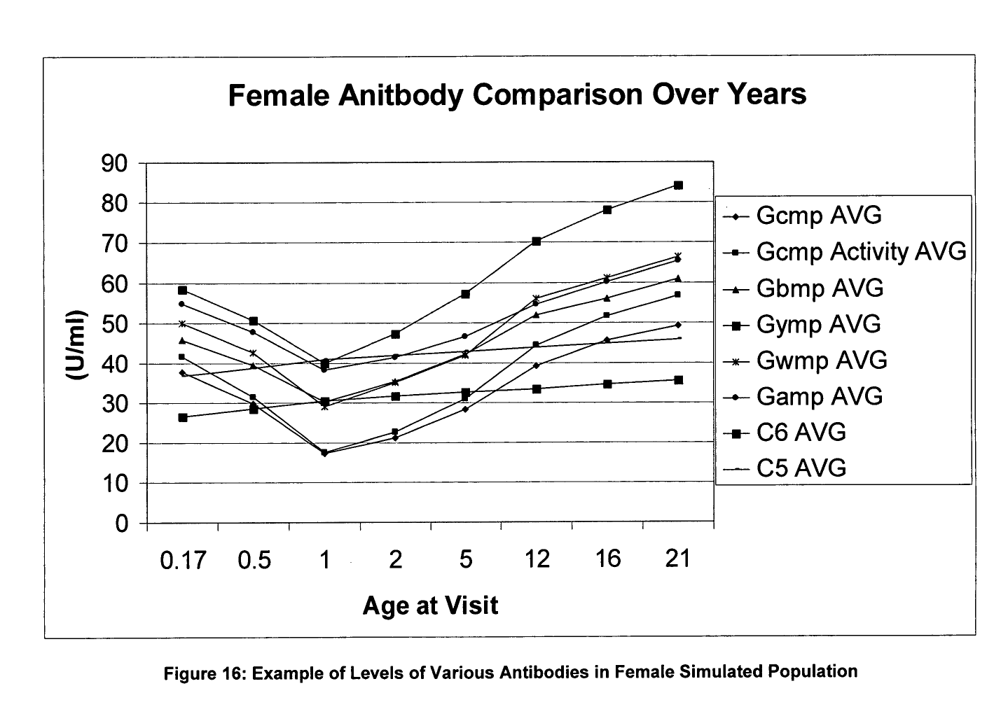

In the exemplary database, assay test results are generated with care. The distribution of antibody levels are randomly generated based upon a log-normal distribution with an average of 50 micrograms per milliliter, as is consistent with measured antibody levels in practice. These values are used as initial baseline levels for the patients in the database. New values are then entered to simulate readings taken at set time intervals in the exemplary patients' lives, as indicated in FIG. 15. At each age, the antibody levels were perturbed using a small normal distribution, to simulate variation in the population over time. Results are biased to match the observed behavior of antibody activity in populations as they age, as shown in FIG. 16. All data in FIG. 16 is from simulated vaccinated patients.

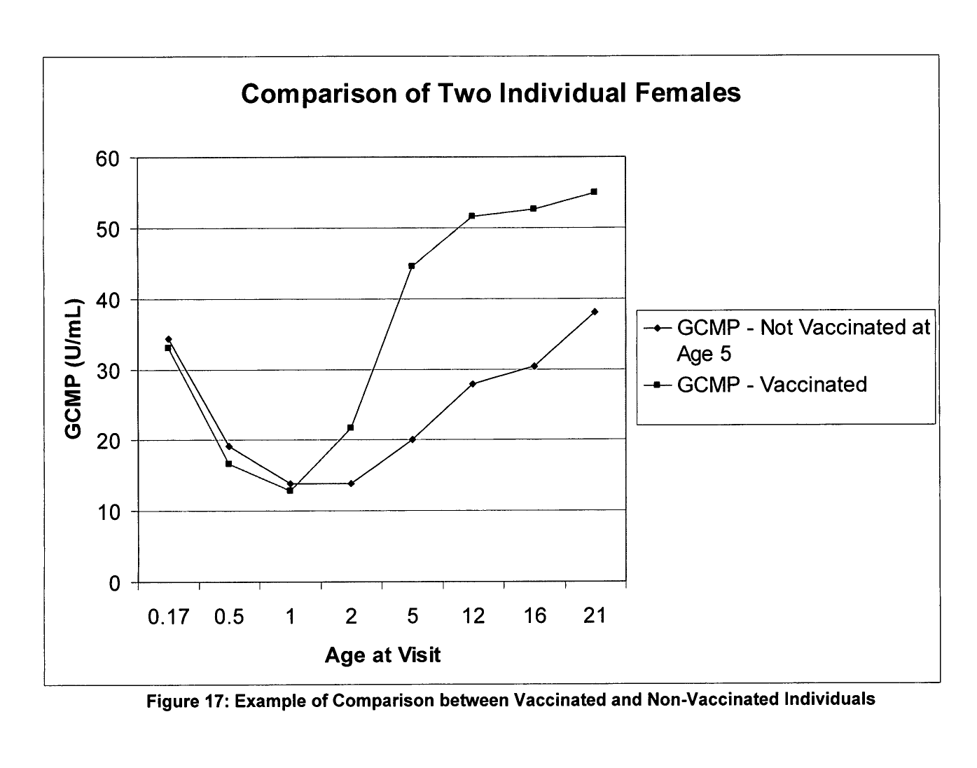

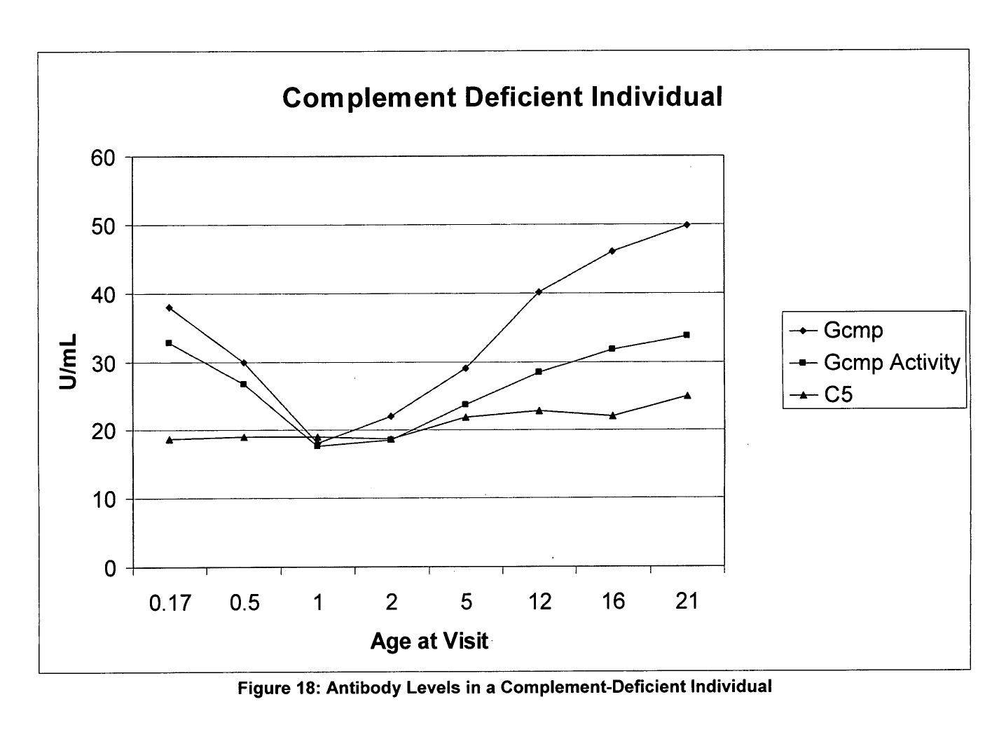

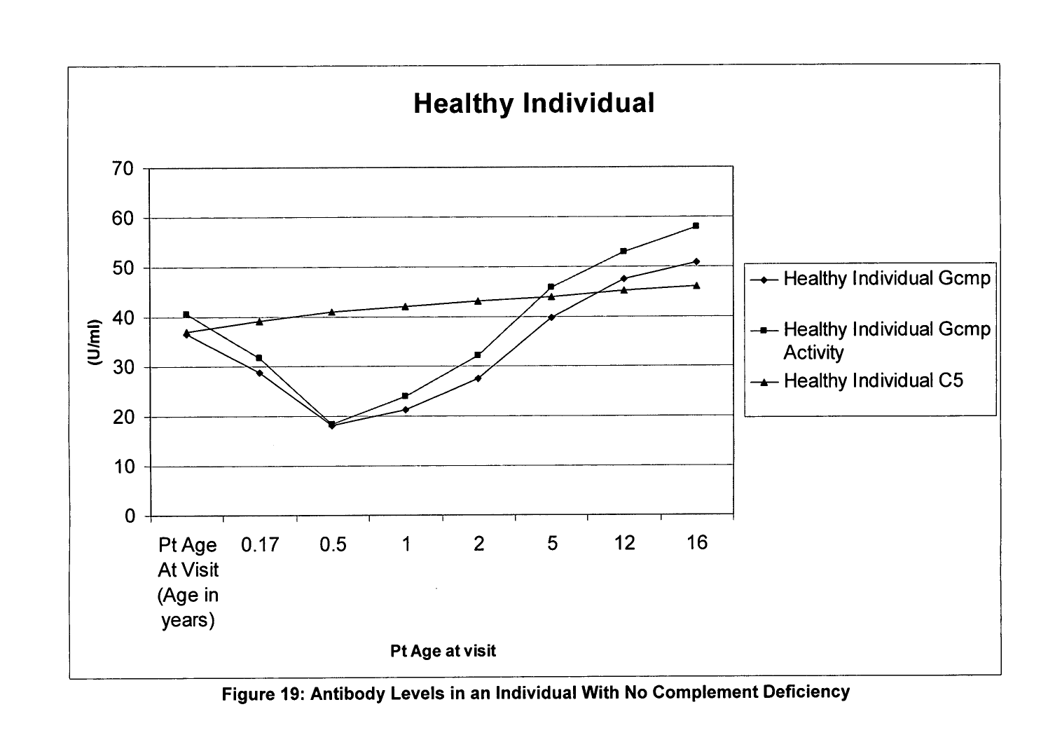

Half of the sample population was treated as if they had received a standard vaccination schedule at age 5; the other half was left untreated. Antibody levels were adjusted to suit, as shown in FIG. 17. In addition, a subset of patients were given artificially lowered complement levels and antibody activity levels with no change to the measured antibody levels, simulating the effect of complement-deficient patients on the data mining procedure. This is shown in FIGS. 18 and 19.

The intent behind this production of sample data was to produce a population with interesting characteristics that could be highlighted in the data mining module. Although the exact features used may not be strictly representative of the population as a whole, they represent the type of correlation that a system such as this could detect within real patient data. It could easily be imagined, for example, that individuals of a particular racial background might naturally have elevated levels of a particular antibody. The system being described could be used to deduce that fact, which may have implications for the immunological care that such individuals would receive.

It is noted that all assay results, such as antibody levels, such as, for example, “Gcmp AVG” in FIG. 16, may be measured and quantified as units (U) per volume (e.g., ml), where U may be defined as some arbitrary unit of a particular assay for the purpose of relative comparisons. In addition, U may be replaced by a more precise measurement of mass, such as micrograms, where possible and appropriate. Antibody activity, such as, for example, “Gcmp AVG” in FIG. 16, refers to the functional activity of an antibody, which may consist of, but not necessarily be restricted to, bactericidal or bacterial killing properties. In these specific examples, assay results from individuals may be processed for statistical purposes in the evaluation of a population, as in FIG. 16, where individuals may be averaged (AVG) by appropriate statistical formulas. Where statistical processing assumes a normal distribution, geometric means may be used to average the results from different individuals, thereby requiring a log transformation of data sets, since it is generally found that only the log values of immune responses will follow a normal distribution.

4.3. Exemplary Use of the Data Mining Module

In a full system, the database used for data mining may be accessed in three different modes, as indicated in FIG. 5.

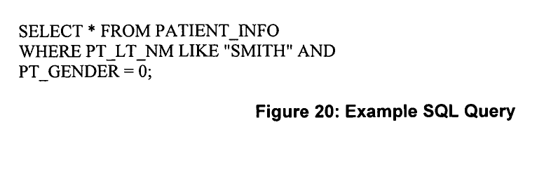

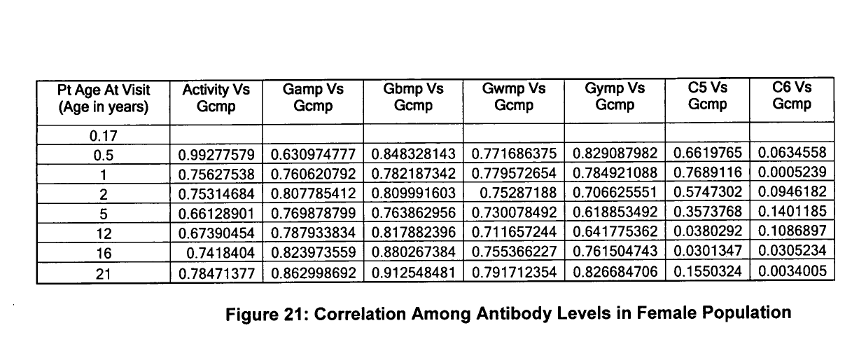

The first mode is, for example, an interactive query mode. A user can interactively search for results in the database. Typically queries might include the retrieval of a single individual's immune status over time, or the comparison of two such individuals, as shown in FIG. 21. Queries can be submitted, for example, using either a graphical query tool or through the use of Structured Query Language (SQL), a computer language for the querying of databases. An exemplary SQL query is shown in FIG. 20. Both of these methods of access are well-known in the industry. With reference to FIG. 5, a user can use the query mode via Query Module 531.

The second mode is the use of Online Analytical Processing tools, or OLAP tools, to find patterns within the database. A simple example of this is the production of aggregate statistics for subpopulations within the whole. In FIG. 21, for example, a query for correlation coefficients to GCMP levels is restricted to female patients. A similar query might look at only patients from a distinct geographical area or racial background. Correlation statistics can also be generated, to test hypotheses about possible causal links among measured antibodies, between antibody measurements and physical symptoms, or correlations between any of these and demographic information. The utility of such a tool depends directly on the quantity and quality of data that is input into the system. For the exemplary system, trends that were deliberately introduced into the sample data can be “discovered”, but other correlations are simply a function of random noise. In a real system, it is expected that more interesting patterns could be deduced. For the exemplary database, standard OLAP tools were used. With reference to FIG. 5, a user can use the data mining mode via Data Mining Module 532.

The third mode that is anticipated is the construction of a pattern detection module. This can, for example, comprise software programmed to sift through the accumulated immune status data and search for patterns that might not be evident to a human observer. It is possible that there are statistically significant patterns in the underlying data which are too subtle or too complex for simple detection schemes. Such an automated detection system can rely upon one or more of the artificial intelligence pattern recognition techniques as described above and in the standard literature. Both neural networks and genetic algorithms may prove well suited to this task. With reference to FIG. 5, a user can use the pattern detection mode via Pattern Detection Module 534.

III. Uses of Immunoscore Information in Various Commercial, Research and Governmental Contexts

In exemplary embodiments of the present invention, information obtained from one or more assay panels, processed in an exemplary system and stored in an exemplary database can be used in a variety of commercial, research and governmental contexts. These uses can range from optimizing the health care costs of a medical insurance underwriter to facilitating immunogenicity studies for a pharmaceutical manufacturer to tracking the incoming and subsequent immune status of immigrants. Following are descriptions of several exemplary business methods which leverage the use of immunological informatics.

A. Health Insurance Underwriting/Health Care Provision Optimization

The system and methods of the present invention can be used, for example, to optimize the business of health insurers as well as healthcare providers who are essentially self insurers. In general, health insurance underwriter or a health insurance provider has a population of individuals, generally called insureds or plan members, whose cost of medical care are reimbursed or paid for directly by the healthcare insurer or the healthcare plan. In such contexts, it is useful to monitor the health of the population of insureds or plan members, especially those who are older and in those years, generally, for example, starting at age 60, when individuals begin to encounter greater health and medical problems.

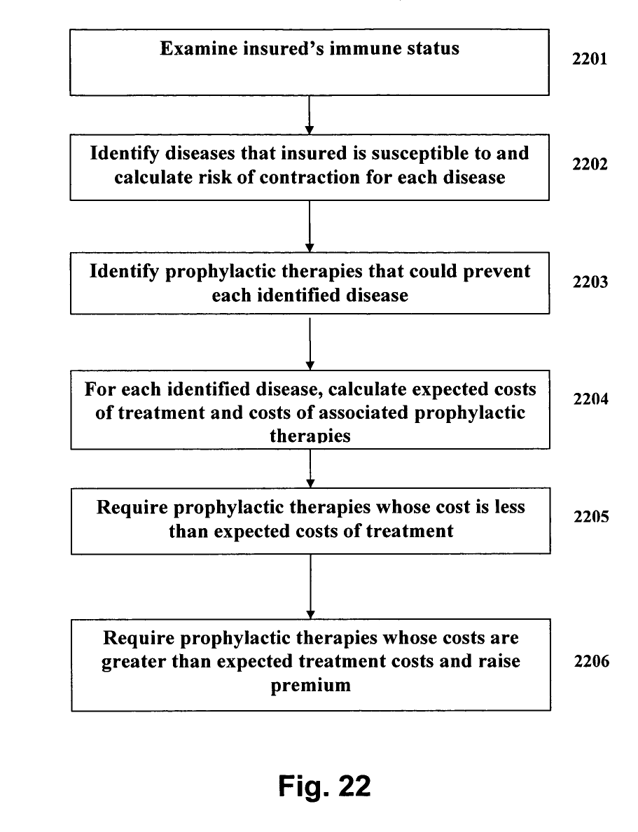

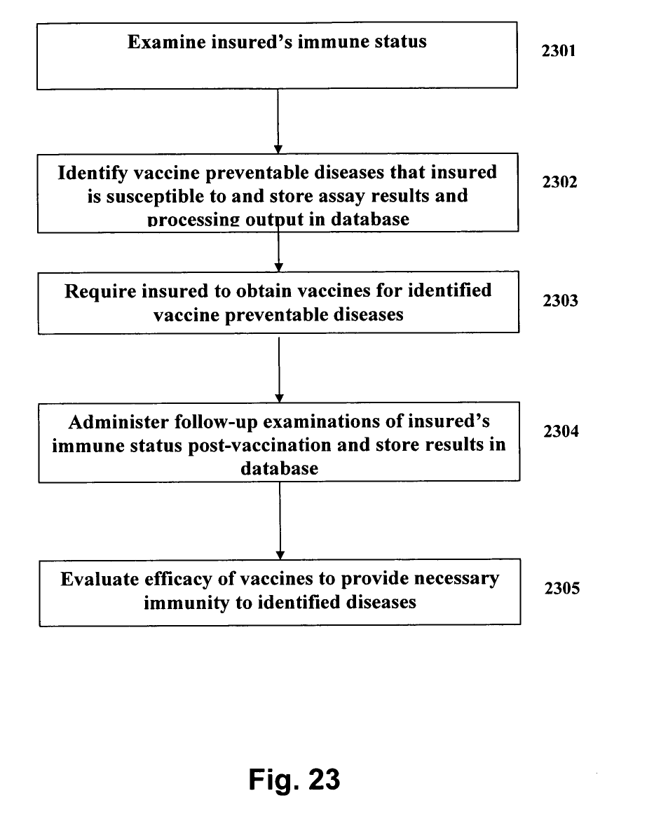

In exemplary embodiments of the present invention, each plan member or insured, or each plan member or insured above a certain age, can be assayed, and the results used to determine whether any prophylactic therapy should be administered to these individuals. Sometimes the decision is as simple as identifying vaccine preventable diseases for which the individual does not have sufficient levels of antibodies. In that case, the prophylactic therapy would be the adminsitration of the vaccine in question. More complicated decisions could include diseases that an insured or plan member is susceptible to that do not have a direct and economical prophylactic therapy. In that case, there could be a more complex algorithm which decides, given the results of the assays and the relative costs of assuming the risk that the insured will contract the disease or diseases and the costs of prophylactic therapies to prevent the disease or diseases implicated. Such algorithms could, for example, be implemented in a system such as depicted in FIG. 2A, where, for example, in addition to database 203 where the results of assays conducted on individuals are stored, there can also be a business rules database 220 which can also supply inputs to a central processor 204 which implements the algorithms. The inputs to such algorithms can then be, for example, not just assay results, medical history and demographic information, but also a set of business rules allowing a decision to be made or facilitated, taking into account the relative costs and benefits of administering prophylactic therapies. Such benefits to be considered, can for example, be those inuring to the individual as well as those inuring to the members of the health care plan as a whole. In exemplary embodiments of the present invention such a healthcare insurance optimization method could be implemented as is illustrated in process flow diagrams FIGS. 22 and 23.

With reference to FIG. 22, at 2201 an insured's immune status can be examined, for example by conducting one or more assays or panels of assays such as, for example, those that are described above. At 2202, the results of those assays can be used to identify diseases that the insured is susceptible to, and moreover, the risk of contraction of each disease for that individual can be calculated. At 2203, prophylactic therapies that could prevent each identified disease can be identified, and at 2204, for each identified disease a decision can be made by calculating the expected costs of treatment (such as, for example, by taking the known costs of treatment multiplied by the probability of contraction) and the costs of associated prophylactic therapies. Finally, at 2205, prophylactic therapies that cost less than the expected costs of treatment can be required for the insured as a condition of maintaining his or her insurance coverage or membership in the health plan, and at 2206, for those prophylactic therapies whose costs are greater than expected treatment costs, they can also be required and the insured's premium increased.

FIG. 23 depicts a particular subset of the process flow illustrated in FIG. 22 where the prophylactic therapies are simple and the ailments identified are vaccine preventable diseases. Beginning at 2301, an insured or plan member's immune status is examined by conducting one or more assays or panels of assays such as those described above. At 2302, vaccine preventable diseases that the insured is susceptible to are identified based on an analysis of the results of the immune status from 2301. At 2303, the insured can be, for example, required to obtain vaccines for the identified vaccine preventable diseases. At 2304, follow-up examinations of the insured's immune status post-vaccination can be made, again by conducting one or more assays or panels of assays, and these results can also be stored in the database. At 2305, the follow-up examination results can be used to evaluate the efficacy of any administered vaccines to provide the necessary immunity to the identified diseases for this individual. When extended to an entire population, such as the insureds a health insurance company or the members of a health plan, this can, for example, provide a means of evaluating the efficacy of vaccines in an aging population. This can also be very useful in the context of measuring and dealing with immunosenescense, as described below.

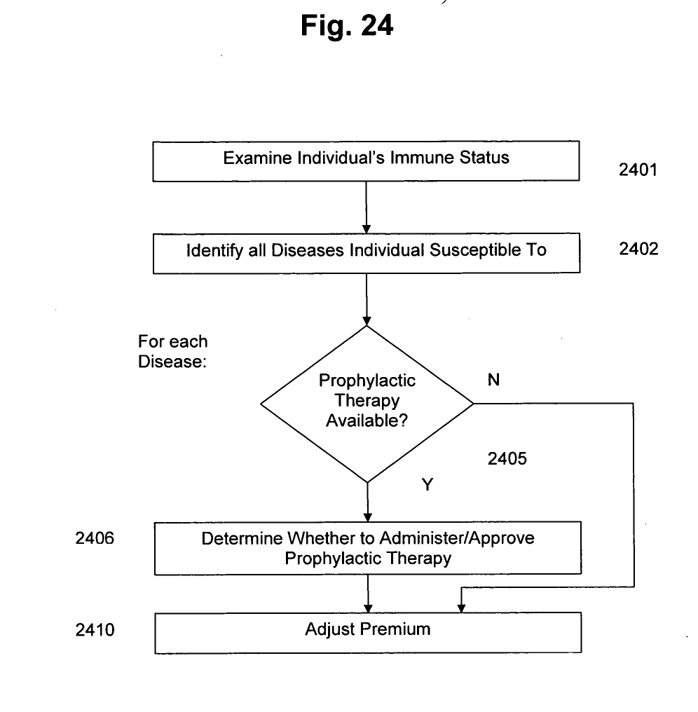

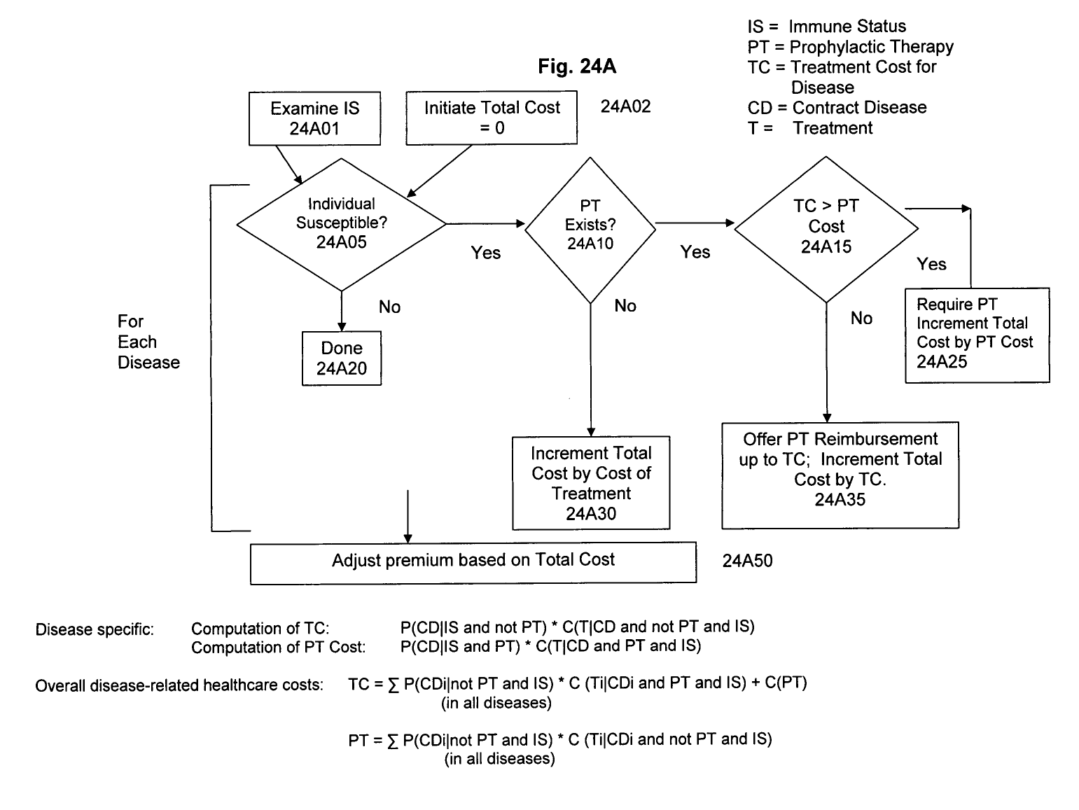

Next described are a number of process flow charts which illustrate exemplary process flow according to various embodiments of the present invention applied to the healthcare management applications. FIG. 24 is an alternative process flow to that depicted in FIG. 22, is concerned with adjusting an insurance premium or an HMO participation fee for an individual based upon identification of diseases using ImmunoScore diagnostics.

The context of FIG. 24 could arise, for example, in the context of an insurance company or HMO requiring an annual ImmunoScore diagnostic panel as a condition of maintaining insurance coverage or participation under a healthcare plan. Such annual requirement would be akin to the annual information questionnaires that automobile insurance companies require of all of their insureds wherein an insured must state if he has had any serious health problems, if he has been involved in any accidents, or if other out of the ordinary events have occurred. With reference to FIG. 24 at 2401, the individual's immune status can be examined and at 2402, based upon the results of such examination, all diseases that the individual is susceptible to can be identified. 2405 is a decision tree which is applied to each disease identified at 2402. Thus, at 2405, for each disease a decision is made as to whether a prophylactic therapy is available. If no, flow terminates at 2410 where the insured's premium is adjusted upwards, to account for the additional risk the insurance company is taking in continuing to cover this individual. If, at 2405 there is a prophylactic therapy available then the flow moves to 2406 where it is determined whether to administer or approve the prophylactic therapy. Based upon this decision the premium can also be adjusted.

FIG. 24A is a more detailed version of the analyses described in connection with FIGS. 22 and 24.

With reference to FIG. 24A at 24A01 the immune status of an individual is examined and at 24A02 the initial total cost is set to zero. 24A02 through 24A35 are then applied in a loop which cycles over all of the diseases tested for in the examination at 24A01. Such identified diseases are those indicated by the assays conducted, as described in detail in Section I above. For each potential disease, at 24A05 it is determined whether the individual is susceptible or not based upon the assay results. If no, the process flow terminates as to that disease at 24A20 and no incrementation of cost occurs. If yes, flow moves to 24A10 where it is determined whether a prophylactic therapy exists. If a prophylactic therapy does not exist, at 24A30 the total cost is incremented by the cost of treatment. If it does exist, at 24A05 it is determined whether the treatment cost from the disease is greater than the cost of the prophylactic therapy. If no, then at 24A35 the prophylactic therapy is offered to be reimbursed up to the treatment cost and the total cost is incremented by the treatment cost. If yes, then at 24A25 the individual is required to take the prophylactic therapy and the total cost is incremented by the prophylactic therapy's cost. After grouping through all of the tested-for diseases, at 24A50 the premium is adjusted based upon the total cost. The computation of total cost and prophylactic therapy cost at both the disease specific level and the over all levels can be given by the following rules:

Disease Specific:

Computation of TC: P(CD|IS and not PT)*C(T|CD and not PT and IS)

Computation of PT Cost: P(CD|IS and PT)*C(T|CD and PT and IS)

Overall Disease-Related Healthcare Costs:

TC=ΣP(CDi|not PT and IS)*C (Ti|CDi and PT and IS)+C(PT) (in all diseases)

PT=ΣP(CDi|not PT and IS)*C (Ti|CDi and not PT and IS)

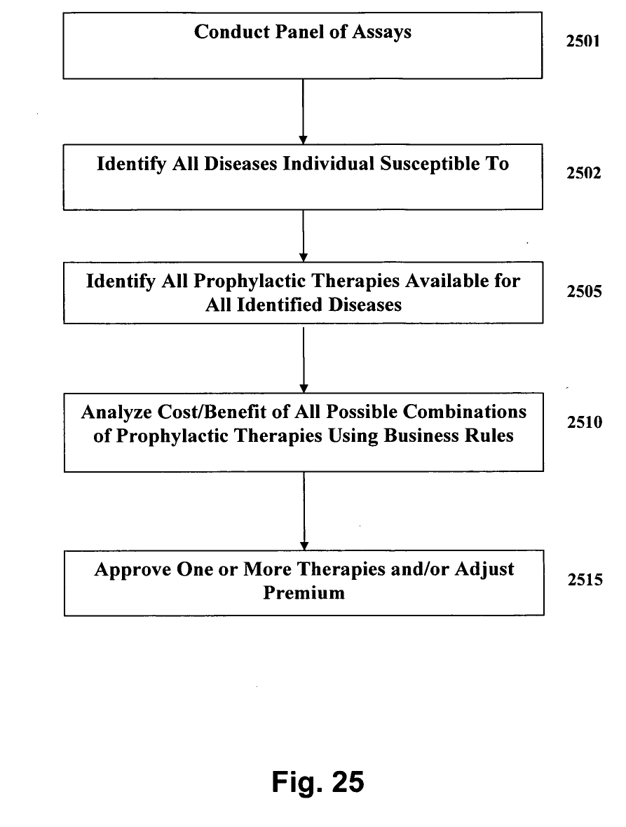

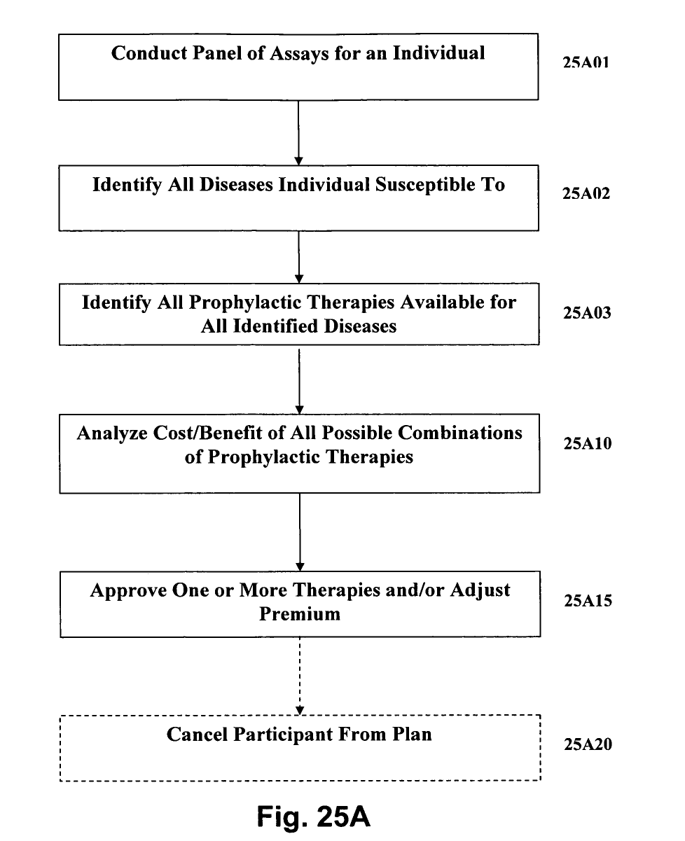

The various exemplary implementations of healthcare management described above have considered each disease individually. FIG. 25 addresses a more complicated situation where all of the potential diseases are identified and all prophylactic therapies available for all of the identified diseases are also identified in all possible combinations of diseases and prophylactic therapies are analyzed using a cost benefit approach. With reference to FIG. 25, at 2501 a panel of assays can be conducted. At 2502, based upon the results of such assays all diseases the individuals are susceptible to are identified. At 2505 all prophylactic therapies which are available for each of the identified diseases are also identified, and at 2510 a cost benefit analysis of all possible combinations of prophylactic therapies and diseases is undertaken using business rules. This functionality represents a much more complex level of analysis in order to implement it, it is necessary to first define all possible combinations of diseases and prophylactic therapies. For example, if the individual is susceptible to five diseases and a prophylactic therapy exists for each of them but these prophylactic therapies vary widely in cost, it is useful to a healthcare manager or a healthcare insurance underwriter can know whether it may be more economical to only administer some of the identified prophylactic therapies and run the risk of the individual contracting the diseases for which prophylactic therapies are not administered. For each of the possible combinations a cost in terms of cost of administering the prophylactic therapy and expected cost of treatment without the therapy is assessed and at 2515 one or more therapies can be approved and/or the insured's premium or the individual's insurance premium adjusted.

It is understood that in the description of the various possible algorithms which can be used in an ImmunoScore analysis for healthcare management that the term individual, insured, and healthcare plan participant are functionally equivalent. While some algorithms are expressed in terms of health insurance context that can easily the same analysis represented by them can be applied to HMO management or management of other healthcare plans. As will be described below, the same techniques can be applied where the entire population is covered under a healthcare plan such as in a socialized medicine jurisdiction. Alternatively, the same techniques can be applied where a large population of some mutual affinity is covered by a single healthcare plan such as United States Veterans whose healthcare is provided by the Veteran's Administration. Thus, it is understood that any particular algorithm or method described in one context also applies to the other.

FIG. 25A is identical to FIG. 25 except that it offers an additional option. At 25A20 if in fact the minimum cost, which is simply the total cost of the least costly permutation at 25A10, is too great for underwriting limits or healthcare management criteria at 25A20, the participant can, for example, be canceled from the plan.

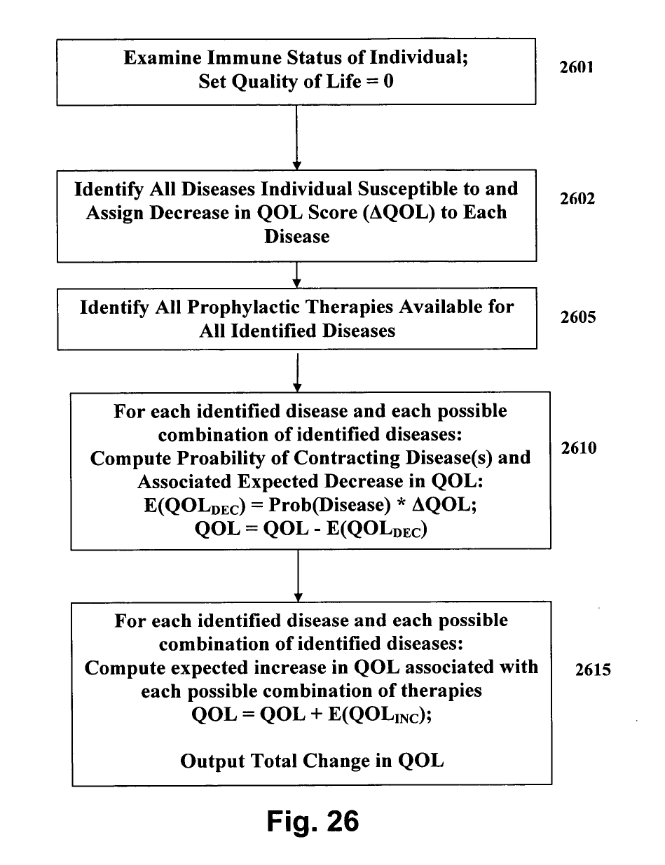

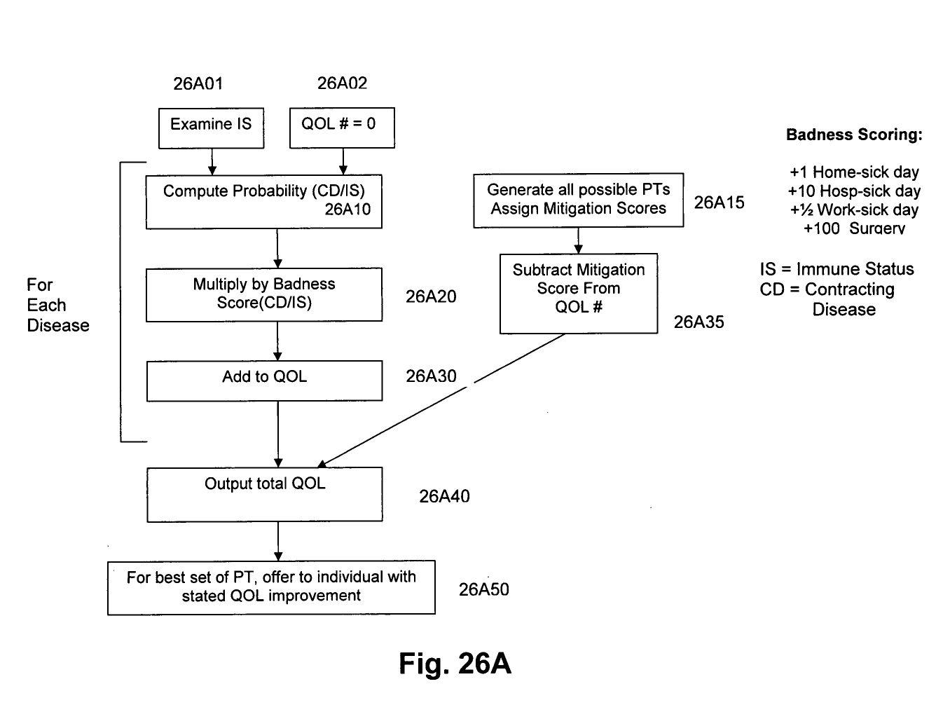

FIG. 26 depicts an exemplary process flow for use in healthcare management applications. FIG. 26 is not concerned with dollar costs but rather quality of life cost. Such an analysis would be useful where dollar costs is less important than quality of life such an exemplary embodiments where a supplemental insurance company insures a minimum quality of life and undertakes to provide for whatever healthcare cost are necessary to maintain that quality of life. Additionally, socialized medicine jurisdiction could have a minimum quality of life which it seeks to provide to each citizen as a basic human right which that jurisdiction sees all its citizens as having. With reference to FIG. 26, at 2601 immune status of an individual is examined and the quality of life is set to zero. For the purposes of FIG. 26, a higher quality of life score translates to a higher quality of life. At 2602 all diseases to which the individual is susceptible are identified and a decrease in QOL score is assigned to each disease. The data which assigning the scoring data would be stored in a business rules database such as is depicted in FIG. 2A. Such a decrease in quality of life score can be, for example, a measure of unexpected pain and suffering, a measure of how many sick days are generally associated with it or whether the sick days are at home, taken at the hospital, or taken while still at work, and finally whether surgery is involved. At 2605, all prophylactic therapies which are available for all of the identified diseases at 2602 are also identified. At 2610 for each identified disease and each possible combination of identified diseases (assuming that the individual could contract more than one disease either simultaneously or in succession) the probability of contract the disease is computed and from that probability an associated expected decrease in quality of life is also computed. As provided in FIG. 26, an exemplary formula which can be used is the following:
E(QOL_{DEC})=Prob(Disease)*ΔQOL;
QOL=QOL−E(QOL_{DEC})

At 2615 an increase in quality of life can be assessed for each identified disease or combination of identified diseases for which either prophylactic therapies or therapeutic therapies exist. Thus the quality of life can be incremented looping through each disease by the expected increase in quality of life associated with either providing a prophylactic therapy or a therapeutic measure to mitigate the loss and quality of life due to contracting the disease. For example, not every disease for which there is a prophylactic therapy can be totally obviated. Some diseases to which individuals are susceptible can be mitigated but not prevented by prophylactic therapies. For example, when people feel they onset of a cold they often take echinacea. Echinacea tends to lower the amount of time one is symptomatic but rarely totally prevents contracting the cold. Alternatively, if a prophylactic therapy completely obviates the individual from contracting the disease then the E(QOL_{inc}) should exactly equal the E(QOL_{dec}). If the prophylactic therapy happens in fact to bestow other benefits besides preventing the disease, then the expected increase in the QOL associated with undergoing the prophylactic therapy would exceed the E(QOL_{dec}). Similar computations would apply to various possibilities. At the end of the process flow in FIG. 26 a net quality of life figure can be computed.

FIG. 26A is a more detailed process follow for the example illustrated in FIG. 26. At 26A01 the immune status is examined and at 26A02 the quality of life is set to zero. At 26A10 the probability of contracting a disease given the immune status obtained in at 26A01 is computed. At 26A20 the probability of contracting the disease given the immune status is multiplied by a badness score. At 26A30 this product is added to the quality of life score. At 26A10 through 26A35 are repeated for each disease for which susceptibility could be examined given the assays administered at 26A01. In this exemplary process flow a better quality of life is associated with a lower number which is the opposite convention of that adopted in the process flow of FIG. 26. It is for this reason that a badness score is assigned to each disease and a expected badness is added to the quality of life at 26A30. Additionally, at 26A15, all possible physical therapies for the identified disease (26A15 and 26A35 are within the for-each-disease loop as well) generated and the mitigation scores are assigned for each physical therapy or combination thereof. At 26A35, the mitigation score is subtracted from the quality of life score and once the flow is looped from 26A10 through 26A35 for each disease, at 26A40 the total quality of life score can be output. Using this total quality of life score, at 26A50 the best set of prophylactic therapies in terms of higher quality of life can be offered to the individual with the stated quality of life improvement.

It is noted that in FIG. 26A schema a badness score is associated with contracted each identified disease. An exemplary badness scoring system is presented in the upper right of FIG. 26A and comprises +1 for a home sick day, +10 for a hospital sick day, +½ for a work sick day, and +100 for a surgery. Accordingly, the quality of life score would dramatically decrease if the individual was found to susceptible to a number of diseases each of which required surgery if contracted.

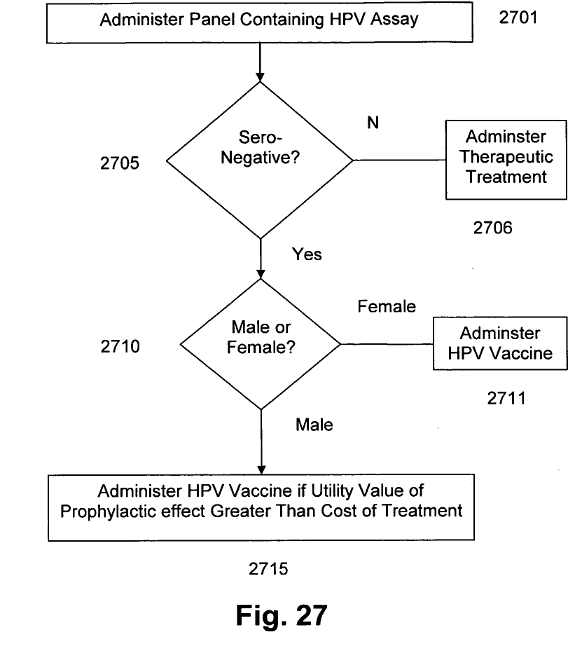

FIG. 27 is a final healthcare management exemplary process flow chart. FIG. 27 is concerned with the newly discovered HPV vaccine which is 100% effective in preventing cervical cancer in women. The question is who should receive the vaccine and when should they be tested. From the point of view of society as a whole, perhaps everybody who has not contracted HPV, should be vaccinated to prevent them from ever contracting it and thus prevent the females amongst them and females in contact with the males amongst them from even contracting cervical cancer.

Of course, this has a greater cost than simply vaccinating on women prior to their exposure to HPV. Therefore, the decision as to who receives the HPV vaccine will often depend upon who is managing healthcare of the population in question. This will be described in connection with the final decision at 2715.

Beginning at 2701, an assay panel containing an HPV assay can be conducted relative to one or more individuals. At 2705 it is determined whether that individual is seronegative or seropositive to the HPV virus. If seronegative, the individual has not yet contracted HPV and flow moves to 2710, where the decision as to the individual's gender is made. If the individual is not seronegative, i.e, seropositive to HPV, then flow terminates at 2706 and any therapeutic treatments that are available are administered. Continuing at 2710 the individual is a female flow terminates at 2711 and the HPV vaccine is always administered. Whether the healthcare manager is an insurance company, an HMO, a socialized medicine jurisdiction or a large scale healthcare management entity such as the Veteran's Administration, any female whose care is being managed should be vaccinated to prevent any healthcare expenditure in treatment expenditure for cervical cancer. However, the question remains what about males? The only utility derived from vaccinating males is that the females are in sexual contact will never contract HPV. If those females are managed by a different healthcare entity there is little utility in protecting men. If those females are protected in the same healthcare management entity, then there is utility in protecting them. Alternatively, even if the females are not provided healthcare or healthcare insurance under a given plan, a government regulating that plan may see a social benefit in wiping out cervical cancer, or at least those cervical cancers attenuated to HPV which are the vast majority of them. Accordingly, given all of these concerns at 2715 to a male, the HPV vaccine is administered if the utility value of the prophylactic affect is greater than the cost of treatment which is simply the cost of the vaccine. The utility value will, as noted above, be a complicated function of number of factors, most prominent of which being who is responsible for the healthcare of the females that this male may come in contact with.

B. Veterans Health Care Management (Variant of Health Care)

A special instance of health care management relates to veteran's care. In the United States the Veterans Health Administration (VHA) provides a broad spectrum of medical, surgical, and rehabilitative care to its customers. Individuals that qualify for Veterans healthcare services include returning Active Duty, National Guard and Reserve service members of Operation Enduring Freedom (OEF) and Operation Iraqi Freedom (OIF). The vision statement of the VHA is that the mission of the Veterans Healthcare System is to serve the needs of America's veterans by providing primary care, specialized care, and related medical and social support services. To accomplish this mission, VHA states that it needs to be a comprehensive, integrated healthcare system that provides excellence in health care value, excellence in service as defined by its customers, and excellence in education and research, and needs to be an organization characterized by exceptional accountability and by being an employer of choice.

Veterans, with their special requirements based on service, can be well served by ImmunoScore diagnostics and data management. As previously described, soldiers have very specific vaccination requirements based on their deployment and area of expertise. ImmunoScore diagnostic panels can be tailored to the needs and context of the individual soldier based upon his or her previous exposure to immunization and also to different infectious agents depending on the relevant theater of deployment. In addition to immune response to infectious agents, veterans are likely candidates for measurement of immune system perturbations induced by Post Traumatic Stress Disorder (PTSD), exposure to unique chemical agents (e.g. Agent Orange), Gulf War Syndrome, and recovery from injuries sustained in service.

The VA Research and Development program (The Office of Research and Development) aspires to lead the Veterans Health Administration in providing unequaled health care value to veterans. The ImmunoScore technology can help contain healthcare costs for veterans by monitoring and analyzing immunologic information.

C. Socialized Medicine Management

A socialized medicine jurisdiction is essentially a health care provider or insurer for an entire population. Thus, the health care management applications of ImmunoScore described above can also be provided to a socialized medicine jurisdiction. Countries with socialized medicine, such as the UK, New Zealand, and particularly Canada would present possible opportunities to stress preventive medicine for the good of the populace (i.e., maximizing QOL for a given health care budget) and the advantages of lower cost healthcare as represented by ImmunoScore managed healthcare. These governments could be provided with healthcare management services via an implementation of the ImmunoScore system. A most likely candidate would be the government of Canada, which seems to have the most socialized medicine program.

D. Supplemental Insurance (AFLAC Model)

AFLAC is the leading provider of supplemental insurance, which provides help with expenses not covered by an individual's major medical plan. The company is the number one provider of guaranteed-renewable insurance in the United States and Japan. Its products provide protection to more than 40 million people and go beyond the traditional insurance by directly paying claimants with cash benefits.

With the cost of health care rising, the challenge for most employers is to satisfy the specialized needs of each employee without having to fund expensive new plans. AFLAC provides products including the following: Accident Disability; Short Term; Disability; Cancer Benefit; Hospital Indemnity.

ImmunoScore diagnostic testing and database storage can provide information for use in just such supplemental insurance programs. ImmunoScore could provide an individual with immune status testing that could be monitored over time and offer the peace of mind that would come from knowing that that patient had a “healthy” immune system. In addition, the insurer would be better able to underwrite premiums for supplemental health insurance with a sounder understanding of the patient's health status.

Additionally, in exemplary embodiments of the present invention, a “immunological insurance plan” could be offered. Such a plan could provide all immunological monitoring and therapeutics to each insured for a fixed annual premium and guarantee a certain quality of life to each insured. Such a plan could utilize one or more of the health care management processes described above.

E. ImmunoScore and the Wellness Industry

In 1994, the U.S. Congress laid the groundwork for the Wellness Industry by passing the Dietary Supplement Health and Education Act (DSHEA). This Act set new standards for the manufacturing, testing and marketing of nutritional products. Products that meet strict government standards earn the title of nutraceuticals. Blurring the line between conventional foods and drugs, nutraceuticals are defined as foods or parts of food that confer health or medicinal value, including the prevention and treatment of disease.

The Food Policy Institute (http://www.foodpolicyinstitute.org) has defined drivers of nutraceutical industry growth. The nutraceutical market was once viewed as largely a counterculture “back to nature” phenomenon, but is now buoyed by a number of solid fundamentals.

Changing consumer demographics. Americans are living longer and emphasizing the importance of quality of life in their later years. As the baby boomers approach ages where personal health becomes more paramount, the demand for mechanisms for conveying health will grow.

Increasing ethnic diversification. The mainstream U.S. nutraceuticals industry is a relatively new phenomenon. However, the use of foods, herbals, and other natural products to convey health and medicinal values has a long history of acceptance by many of the world's cultures.

Paradigm shift in personal health. Americans are taking more responsibility for their personal health, embracing the concept of health maintenance and wellness. Thus, the paradigm is shifting away from disease treatment and towards disease prevention.

Dissatisfaction with Western healthcare. Americans are becoming more reticent about accepting the side effects of synthetic drugs and remedies. Similarly, rising healthcare costs are encouraging Americans to explore alternatives to traditional orthodox medicine.

Increasing acceptance of alternative healthcare practices. There is a growing acceptance among Americans of alternative or complementary therapies and wellness modalities. Recent years have witnessed increased use, for example, of chiropractic care, vitamin therapy, aromatherapy, meditation and relaxation techniques, and acupuncture.

Increased understanding and awareness of diet-disease relationships. Many of the leading causes of premature death in the U.S. are diet-related. Examples include heart disease, diabetes, and many types of cancer. The USDA estimates that diet-related disease and death costs the U.S. in excess of $250 billion each year.

The Food Policy Institute goes on to state that the nutraceutical industry is facing challenges.

Few farmers are producing herbals and other botanical inputs (due to limited market knowledge, technical requirements and other obstacles.

Limited access to finance and capital constrains industry development and expansion.

Ambiguous regulatory framework for ensuring product standardization and efficacy.

Regulatory restrictions on marketing products via health claims impede retail efforts.

Raw material supply issues (consistency of quality and availability) for botanical manufacturers.

Limited endorsement by traditional healthcare practitioners.

Consumers can not differentiate between high and low quality products and are not sufficiently educated to make informed decisions about proper product use.

ImmunoScore diagnoses and database could provide the answers to these challenges. Individuals and populations could be studied with respect to the efficacy of a nutraceutical diet. ImmunoScore would either pave the way for more growth in curtain nutraceuticals, or perhaps point out the sale of “snake oil.” Individual products, or product lines could be endorsed as valid by ImmunoScore measurements.

The Wellness Industry is expected to grow. The Wellness Industry includes the concept of “wellness insurance” to lower health care costs to individuals. This may provide yet another opportunity to leverage ImmunoScore testing and data storage into the insurance industry.

In addition, workplace wellness as a concept has been used extensively in recent years by management in business and industry, health professionals, fitness experts, and others. Well-designed and administered programs deliver positive outcomes for employers as well as employees. Because healthy employees cost less than employees suffering from illness, ImmunoScore can be a part of employee insurance offered by employers wanting the best and most affordable health care for their employees.

Virtual Physicals™—Incorporate ImmunoScore Diagnostic and Database

The Virtual Physical™ is a comprehensive diagnostic screening procedure that uses state-of-the-art technology to take a global look at a patient's body and identify a variety of conditions at early stages where intervention can be most helpful. A Virtual Physical™ may also be viewed as an integral component of a holistic, behavioral medicine program, where the body, and one's diet, exercise, and lifestyle habits are viewed as a whole, determining where problems may exist and where changes might be required.

The Virtual Physical's™ early detection capability can uncover asymptomatic and often life-threatening diseases generally not detectable by physical exam or standard screening tests. This allows the management of disease in early stages, where medical therapy and treatment options are typically less costly, less invasive and more effective.

Virtual Physical's™ comprehensive scan of an individual's body is significantly more detailed than an X-ray. It covers: (a) the heart and arteries, identifying near microscopic amounts of plaque; (b) the lungs at the air cell level showing the earliest stages of smoke damage, emphysema, or lung cancer; (c) the spine, evaluating for osteoporosis, disc disease and other back problems; (d) internal organs for detection of tumors, stones and cysts of all sizes; (e) aneurysms in the abdominal and chest cavities; (f) thyroid and parathyroid disease; (g) joint disease; and (h) uterine, ovarian, and prostate disease.

In the interest of determining a patient's “totality of health,” ImmunoScore screening could accompany a Virtual Physical™ to add an immune health component to the virtual screening. It is possible that insurance will cover a Virtual Physical™ in the future, and ImmunoScore testing and data storage could be incorporated into the patient's records that could be transferred to the patient's primary care physician or specialist.

F. Women of Childbearing Age/Screening of Pregnant Women

A superpanel for women of childbearing age was described above in Section I.

With light thereof, ImmunoScore diagnostic tests and database storage availability in the offices of obstetricians would greatly enable appropriate immunization of pregnant women as well as find correlates of prenatal interest. In addition to screening pregnant women for their immune status regarding vaccine preventable diseases, ImmunoScore diagnoses and data management could also be of value in determining the immune status of pregnant women regarding, for example, group B streptococcal infection, cytomegalovirus (CMV) infection, and other infectious diseases that may adversely effect the newborn, yet are treatable prenatally. Early onset GBS infection has been the leading cause of death attributable to infection in newborn infants for over three decades, with over 6,000 cases a year in the United States (Vallejo, et al. 1994). Antibiotics have been used to good effect to prevent newborn GBS infection. There is also promising preliminary data on an effective intervention to prevent CMV infection in newborns in pregnant women that has been published recently (Nigro, et al. 2005). All these treatments can be more advantageously administered using ImmunoScore technology.

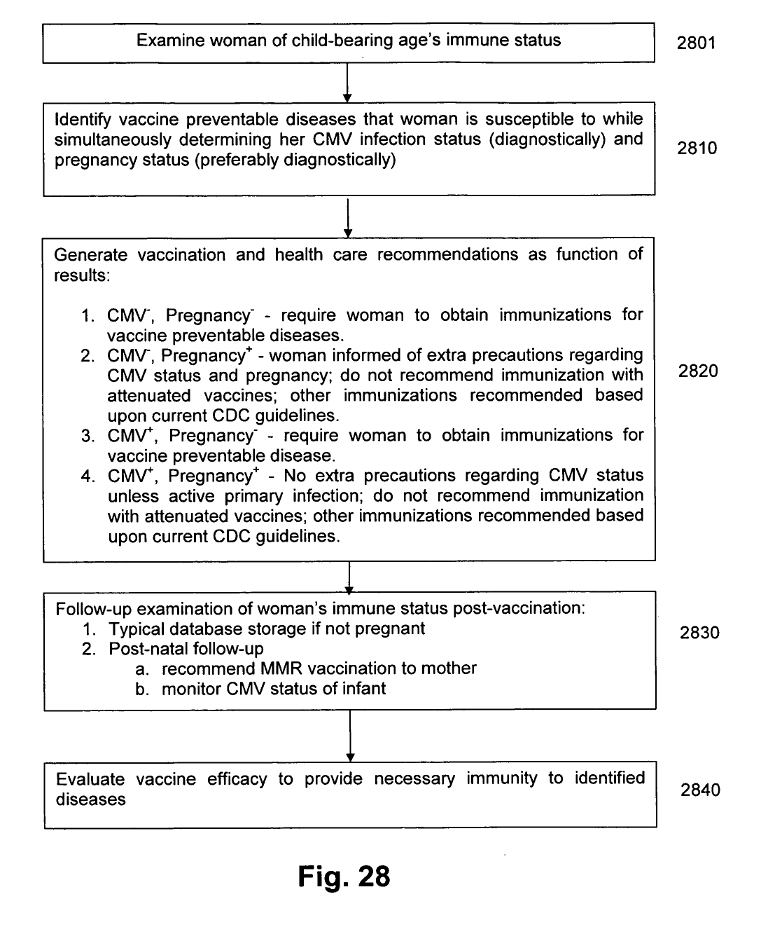

FIG. 28. depicts and exemplary process flow for managing the immune status of women of child-bearing age. Beginning at 2801 the immune status of a women of child-bearing age is examined. At 2810 the vaccine preventable diseases that the woman is susceptible to are identified as well as the woman CMV infection status and pregnancy status. At 2820 these three variables are used to generate healthcare recommendations, as follows. If the woman has not been infected with CMV and is not pregnant she is advised to obtain immunizations for the identified vaccine preventable diseases. If she is an insured under a healthcare insurance plan, or her healthcare is provided by an HMV or socialized medicine entity she can be, for example, required to obtain these immunizations to save future treatment costs as well as to serve the utility of having a healthy population. If she has not been infected with CMV but is pregnant she can be informed of extra precautions regarding CMV status and pregnancy. Moreover, no immunization with attenuated vaccines is recommended or should be performed, however, other immunizations should be recommended based upon current CDC guidelines. If the woman is seropositive to CMV and not pregnant she can be advised or required, as the case may be, to obtain immunizations for the identified vaccine preventable disease. Finally, if she seropositive for CMV and pregnant no extra precautions should be taken regarding the CMV status unless there is an active primary infection. Moreover, no attenuated vaccine should be recommended or administered. However, other immunizations can be recommended or required based upon current CDC guidelines. At 2830 a follow-up examination of the women's immune status post-vaccination can be conducted, and if she is not pregnant the information can simply be stored in a system database. If she is pregnant a post-natal follow-up can be recommended or required, as the case may be, comprising MMR vaccination to the mother and monitoring of CMV status of the child. Finally, at 2840, based upon the post-vaccination follow-up at 2830 the efficacy of the administered vaccines can be evaluated as to whether they provide the necessary immunity to the identified vaccine preventable diseases identified at 2810.

G. Vaccine-O-Mat/Vaccine Distribution Network

In exemplary embodiments of the present invention, ImmunoScore technologies can be used to facilitate the easy dispensing of vaccines to the public as well as giving the public access to their immunologic information. Therefore, in exemplary embodiments of the present invention a business analogous to the “Fotomat” photograph finishing stores, once located in malls and strip malls across America, can be created. For purposes of the present description, this exemplary embodiment of the present invention can be called “Vaccine-o-Mat”. Vaccine-o-Mats can be located in small buildings in corners of malls and strip malls, as concessions in large chain stores such as Target or Wal-Mart, or in appropriate markets they can be located almost anywhere and one day be as ubiquitous as Starbucks Coffee centers. At a Vaccine-o-Mat a member of the public can have his immune status checked and can receive any vaccines that he may be deficient in. If an individual steps on a rusty nail and doesn't remember the last time he had a tetanus booster he can simply drive to the nearest Vaccine-o-Mat, have a panel of assays containing tetanus and any related compliments as conducted and determine then and there whether he needs a vaccine. What makes the Vaccine-o-Mat business possible is instruments which can process large numbers of assays in a relatively short period of time, as noted above. One of such instruments is the M1M analyzer currently marketed by BioVeris Corporation of Gaithersburg, Md., the assignee hereof.

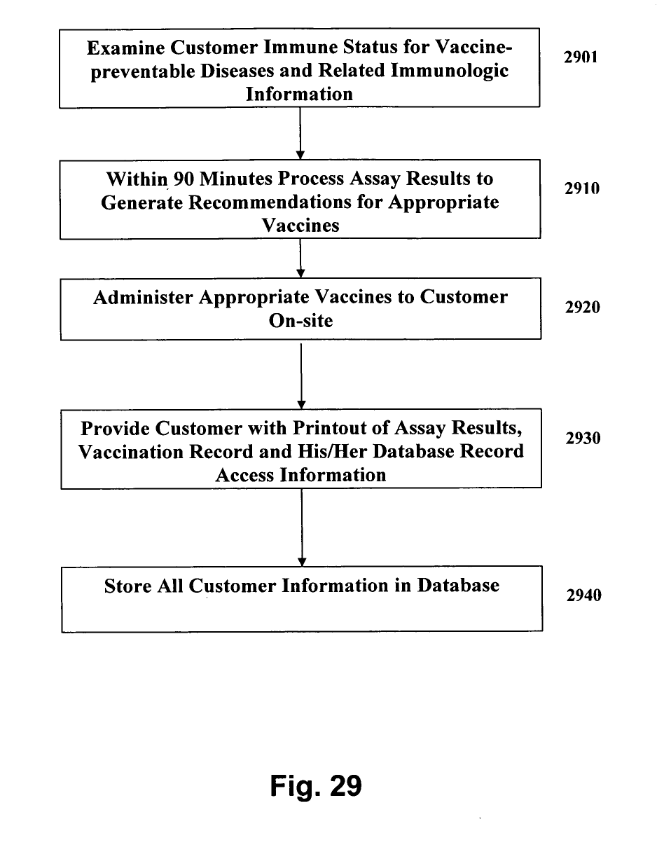

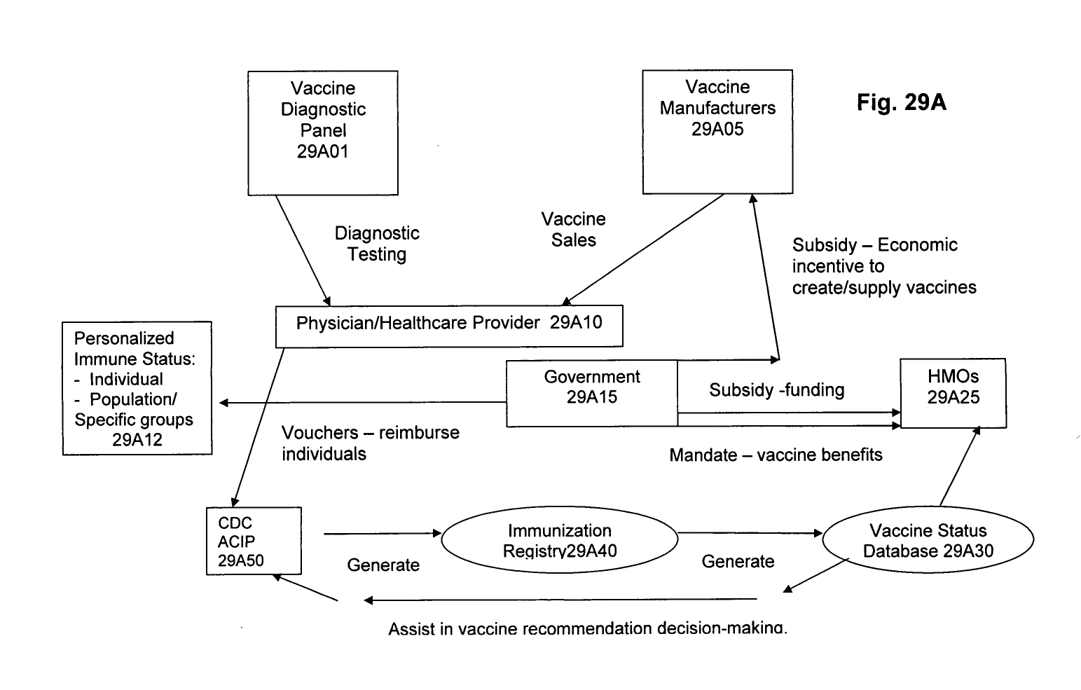

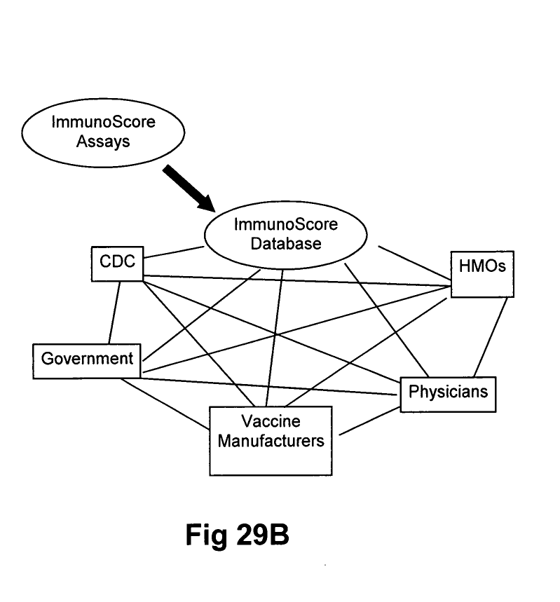

FIG. 29 depicts an exemplary process flow for use at a Vaccine-o-Mat. At 2901, the customer's immune status is examined for vaccine preventable diseases and related immunologic information. It is further contemplated that a particular customer may want to have his bodily fluids assayed for a wide variety of immunologic tests and not have them restricted to vaccine preventable diseases. Therefore 2901 need not to be strictly directed towards vaccine preventable diseases. At 2910, within 90 minutes the assay results can be processed to generate recommendations for appropriate vaccines. This functionality depends upon, as noted above, instruments which can process a large number of assays in a relatively short amount of time. This concept allows for partnering with large chain stores or malls where customers could make their first stop at the Vaccine-o-Mat have their blood tested. They could then continue shopping and then return at the end of their shopping excursion to receive any necessary vaccines and report regarding their immune status. At 2920 appropriate vaccines can be administered to the customer on site, and at 2930 the customer can be provided with a printout of the assay results the updated vaccination record and his or her database record from the ImmunoScore database along with instructions on how to access that information in the future. Finally, at 2940 all of the additionally required customer information resulting from that particular visit is stored in the database for future reference.

One of the benefits of the ImmunoScore technology is the ability to link diagnostic testing of the immune system with rapid delivery of medication at the point of care (ideally, during the course of an office visit). Thus, in exemplary embodiments of the present invention a vaccine distribution network can be set up, for example, to link vaccine manufacturers to physicians offices—or other authorized vaccine dispensing personnel—equipped with diagnostic facilities. Vaccine distribution can also, for example, become part of the ImmunoScore database tracking specific manufacturer's lot numbers to points of sale. This can be important in getting timely information incorporated into the Vaccine Adverse Event Reporting System (VAERS).

FIG. 29A depicts exemplary envisioned interactions between various parties according to an exemplary embodiment of the present invention directed towards vaccine distribution. Information gathered to an exemplary ImmunoScore database can, for example, be shared with the various agencies responsible for dictating vaccination decisions. Unsuspected or unknown relationships regarding immune health or function can be, for example, “fished” or “mined” from a system database using appropriate queries and analysis. In addition, in exemplary embodiments of the present invention, suspected adverse events from vaccination could be addressed and acknowledged or dismissed, based upon information gleaned from the system database.

With reference to FIG. 29A, various entities and institutions which can, for example, be involved in vaccine distribution or vaccine distribution network are depicted. They include any vaccine manufacturers 29A05 who through vaccine sales provide vaccines to physicians or healthcare providers 29A10. The physicians or healthcare providers 29A10 also receive diagnostic testing kits and research services, such as, for example, ImmunoScore vaccine diagnostic panels 29A01. The government 29A15 has a variety of roles in a vaccine distribution network, including subsidizing or providing economic incentives to create or build a supply of vaccines by a transfer of funds to, or via tax incentives to, vaccine manufacturers 29A05. The government can further subsidize or fund HMOs 29A25 and in this context the Veteran's Administration, described above can be considered one of them. Additionally, the government 29A15 can mandate vaccine benefits to certain segments of the population and those can be provided by HMO 29A25 or equivalent. Finally, the government 29A15 can itself access personalized immune status data as to individuals or populations or sub-populations 29A12 for a variety of research or health management purposes. The CDC and ACIP 29A50 can receive input from Physicians/Healthcare Providers 29A10 as well as from a vaccine status database 29A30. Vaccine status database 29A30 can be generated from an Immunization Registry 29A40 set up by the CDC, ACIP or other similar institutions or bodies to maintain immunization records for the population so as to better know who should be vaccinated. FIGS. 29B and 29C, described below illustrate improving connectivity between entities and organizations who could access and utilize ImmunScore information in this context, allowing the benefits of ImmunoScore to be ubiquitously available.

H. Consumer Accessibility to Immunologic Information

Americans are playing a risky game of sexual roulette, according to a new poll that found only 39 percent of respondents always ask a new lover if they are infected with HIV. The poll, taken by Zogby for MSNBC.com also found that 73 percent of respondents were involved in a monogamous relationship, 66 percent of those surveyed had had unprotected sex while under the influence of alcohol. While 39 percent of respondents said they always asked whether a new partner is infected with HIV or other sexually transmitted diseases, 31 percent said they never discuss the touchy issue with a new partner. Moreover, the survey found that 15 percent of Americans had paid for sex, 35 percent of respondents said they had been with between one and five sexual partners, and 19 percent said they had had more than 25 partners.

In exemplary embodiments of the present invention this “risky business” can be ameliorated. Accordingly, at the Vaccine-o-Mat described above, individuals can have their immune status tested by conducting, for example, an STD assay panel, as described in Section I above, which can then be shown to potential sexual partners to fully disclose the immunologic risks that may be involved in any proposed liason. For example, a couple can stop at a Vaccine-o-Mat near a romantic restaurant of their choice. They can have the assays conducted and go off to dine. If things are going well, by the time their coffee has arrived they can obtain each other's immune status and be off—either alone or together—depending upon the ImmunoScore results.

Alternatively, for example, someone worried by past promiscuities can routinely procure his or her immune status at the local Vaccine-o-Mat in 90 minutes, and put any worries to rest, or at least know what they are facing.

I. Immunoscore Connectivity Via Interapplication Translator/Data Integrator

In many exemplary embodiments according to the present invention, the power of an ImmunoScore diagnosis and database lies in the interaction of the database with many different organizations, as shown in FIG. 29B. Use of a web services interconnector to provide this connectivity is illustrated in FIG. 29C, next described. The CDC, the government (or governments, for that matter), health maintenance organizations, vaccine manufacturers, and physicians would all be able to interact with the database and each other to make the best possible decisions regarding the health and welfare of the citizenry.

With reference to FIGS. 29B and 29C, a number of entities and organizations who could access and utilize ImmunScore information are shown. FIG. 29B shows a complicated information exchange structure wherein each entity involved has to set up a separate communications line or pathway to each of the other entities in the network. This can easily be remedied, as shown in FIG. 29C, by utilization of an Interapplication Connectivity Provider 29C50 which can interconnect the various individual and sometimes proprietary computer systems, computer networks, databases, and applications of each of the individual entities participating in the vaccine distribution/creation network so that they can talk to each other. This technology is often referred to as interapplication connectivity or interapplication translation. One example of such a interapplication connectivity provider is the IBM, in particular the IBM Web Services Centers Of Excellence. Additionally, Enterprise Computing service companies, such as, for example, EDS also provide products which link different and disparate computing platforms so that they can exchange data and information in an efficient manner.

J. Immunologic Informatics Based Life Insurance Underwriting

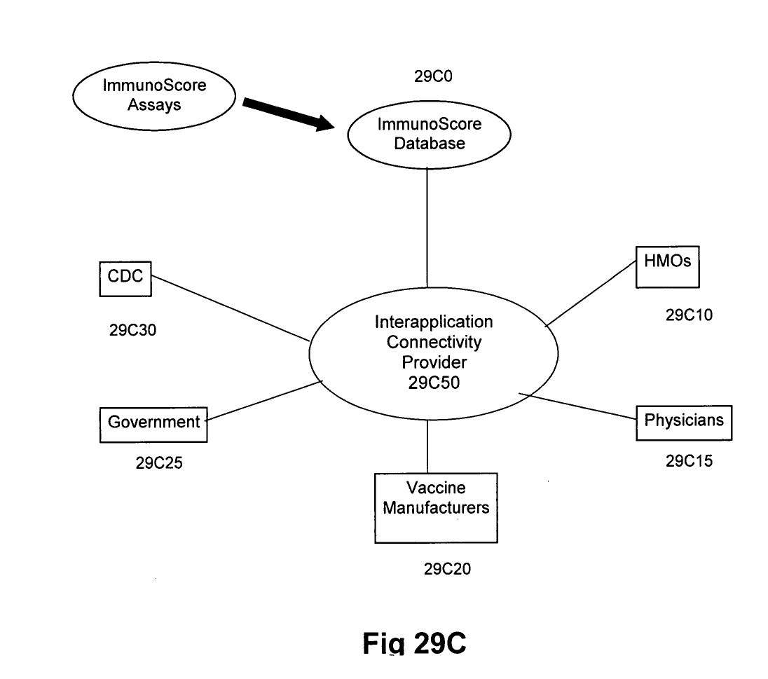

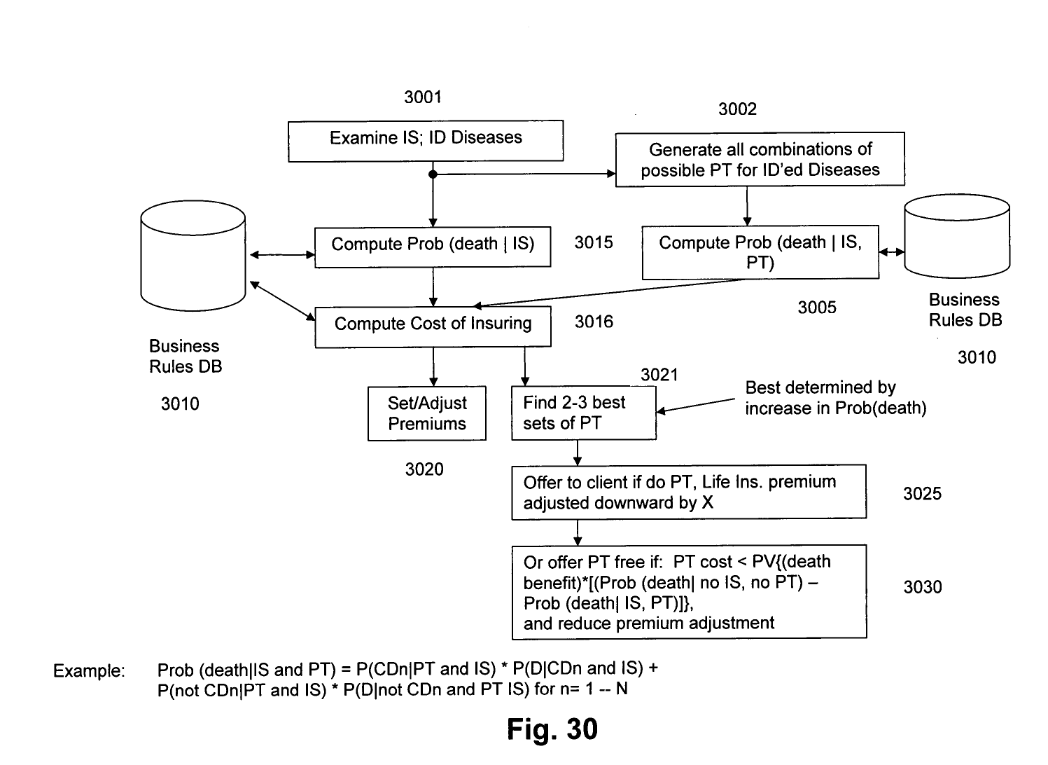

In the exemplary embodiments of the present invention ImmunoScore data can be used to optimize the underwriting of life insurance. Additionally, assuming that regulatory restrictions are not preclusive, ImmunoScore data can be used by companies which provide both life and health insurance to the same clientele. The use of ImmunoScore technology for these purposes is depicted in the exemplary process flow chart of FIG. 30.

With reference to FIG. 30, at 3001 an individual's immune status can be examined and any diseases to which he or she is susceptible identified. At 3015, by accessing Business Rules Database 3010, the probability of death of the individual given the immune status identified at 3001 can be computed. At 3016 the cost of insuring that individual, based on the probability of death of years to death calculated in at 3015 can be computed and premiums can be set at 3020. It is noted that the term “death” appearing in FIG. 30 is a shorthand for “years remaining until death.”

Additionally, at 3002 all combinations of possible prophylactic therapies can be generated given the immune status obtained at 3001. From these combinations, at 3005, the probability of time (generally in years) to death given the immune status and the various combinations of prophylactic therapies can be computed. Such computation, at 3005, exchanges data with Business Rule Database 3010. For convenience, two Business Rules Databases 3010 are depicted n FIG. 30; in exemplary embodiments of the present invention there could be one or many Business Rules Databases each devoted to a specific informational domain. In the depicted exemplary embodiment of FIG. 30 they could most likely be combined inasmuch as they are providing information which allows a system to compute the probable time to death given an immune status. However, the Business Rules Database on the right side of the figure may require more complex information to also factor in the available set of possible preventive therapies for each identified disease.

At 3016, the outputs of 3015 and 3005 are input to allow the exemplary system to compute the cost of insuring the given individual. At 3021 the system can select the two or three best sets of prophylactic therapies from the information generated at 3002, and at 3025 it can offer these prophylactic therapies to the client with a proviso that the life insurance premium set at 3020 in absence of factoring in prophylactic therapies could be lower by (x) if the client chooses to undertake the prophylactic therapies. Alternatively, at 3030 it may be in an insurance company's interest to pay for the prophylactic therapies, i.e., offering them to the insured for free, if the cost of the prophylactic therapies is less than the present value of the expected savings to the life insurance companies by the insured having the prophylactic therapies perform. This can be expressed, for example, as:
PT cost<PV{(death benefit)*[(Prob(death|no IS, no PT)−Prob(death|IS, PT)]}

Thus, if at 3030 such an offer is made, any premium adjustment at 3020 can be diminished or completely reduced. The function of 3030 is to increase the profits to the life insurance company by not only identifying the premium at which it would charge the insured but also, based on the immune status data obtained during the underwriting process (or during an annual audit process), to identify prophylactic treatments that could be offered to increase the time to death for the same individual thus allowing the insurance company to continue to earn the return on the cumulative premiums prior to having to pay the death benefit to the survivors.

It is also noted that at 3021 where the 2-3 best sets of prophylactic therapies are found the term best is really a function of how much the probable time to death is increased. Finally, the availability of probable time to death given a certain immune status and certain prophylactic therapy can be computed using the following equation as noted in FIG. 30:
Prob(death|IS and PT)=P(CD|PT and IS)*P(D|CD and IS)+P(not CD|PT and IS)*P(D|not CD and PT IS)

When offering prophylactic therapies to an insured, unique opportunities arise for insurance companies providing both life and health. A healthier insured lives longer and uses less health care, resulting in twofold savings for an insurer. Because such a life insurance company also approves health care expenditures, there is no red tape or customer effort spent on securing approval for any offered or recommended prophylactic therapies. Thus, in such contexts, the real world optimizations can actually converge on the theoretical optimizations calculated by an ImmunoScore analysis as depicted in FIG. 30. This can, in exemplary embodiments, increase QOL for insureds and profits for the insurers, as well as hopefully.

K. Diagnosing and Managing Immunosenescence in the Elderly

Human aging is associated with progressive decline in immune functions and increased frequency of infections. Morbidity and mortality due to infectious disease is greater in the elderly than in the young, at least partly because of age-associated decreased immune competence, which renders individuals more susceptible to pathogens (Pawelec, et al. 2005). A decline in immune function is a hallmark of aging that affects the ability to resist influenza and respond to vaccination. An accumulation of dysfunctional T cells may be detrimental under conditions of chronic antigenic stress (chronic infection, cancer, autoimmunity). The most important changes occur in T-cell immunity, and are manifested particularly as altered clonal expansion of cells of limited antigen specificity (Fulop, et al. 2005). This is most marked in the CD8⁺ T cell subset, which displays a decrease in both responsiveness and normal function. Normally, CD8⁺ T cells appear to be strongly associated with cytolytic activity, either by direct killing of antigen-bearing target cells by granule-mediated exocytosis or Fas-mediated cytotoxic mechanisms. In addition, it is suggested that antigen-activated CD8⁺ T lymphocytes can eliminate or control viral infection by secretion of antiviral cytokines, such as gamma interferon (IFN-γ) and tumor necrosis factor alpha (TNF-α). IFN-γ production by CD8⁺ T cells can have both local and systemic consequences, whereas cytotoxins such as perforin are cytolytic for the cells that come in direct contact with the cytolytic T lymphocytes (CTL).

The output of the T cell pool is governed by output from the thymus and not by replication (Aspinall and Andrew, 2000). As thymic T cell production diminishes with age, a decline in contribution made by thymic emigrants to the naive T cell pool occurs (Mackall, et al. 1995). Diminution in the size of the naive T cell pool is a common finding with aging, and is a consequence of reduced thymic output (Kurashima, et al. 1995). Thymic atrophy is thought to result from a failure of the thymic microenvironment to support thymopoiesis in old age and recent evidence suggests that a decline in interleukin-7 (IL-7) expression may limit thymocyte development by restricting combinations of survival, proliferation and rearrangement of the beta chain of the T cell receptor (Andrew and Aspinall, 2002). Therapeutic intervention with IL-7 and derivatives has been shown to reverse thymic atrophy in old animals and also lead to improved immune function compared with age and sex matched control animals (Aspinall, 2005).

The CD8⁺ T cell repertoire becomes less diverse in old age due to reduced thymic output and the accumulation of clonally expanded memory CD8⁺ T cells as a consequence of prolonged antigenic stimulation. Clonally expanded T cells are usually CD8+ and show an increased incidence with age, so far it seems that clonal expansion is not due to malignancy but may follow antigen stimulation (Aspinall, 2005). It has been suggested that repeated or persistent infections with viruses such as influenza, cytomegalovirus (CMV), and Epstein-Barr virus (EBV) may drive responses that result in large T cell clones. Longitudinal studies suggest that a set of immune parameters including high percentages of peripheral CD8⁺ CD28⁻ CD57⁺T cells, low CD4⁺ and B cell counts, and poor T cell proliferative responses to mitogens is associated with decreased remaining longevity in the free-living very elderly (>85 years) (Ouyang, et al. 2003). CMV seropositivity is closely associated with increases in the size of the CD57⁺ CD8⁺ T cell pool, which is thought to represent a highly differentiated population of late memory cells. Furthermore, CMV seropositivity is associated with increases in CD8⁺ count in old age and has been documented to have negative influences on immune parameters in the very elderly. A group concluded that the “obsession” of a large fraction of the entire CD8⁺ T cell subset with one single viral epitope may contribute to the increased incidence of infectious disease in the elderly by shrinking the T cell repertoire for responses to other antigens (Ouyang, et al. 2003). Like CMV, EBV manages to persist for the lifetime of the infected host. During chronic asymptomatic infection in healthy individuals, EBV resides in memory T cells (Babcock, et al. 1998). Expansion of peripheral CD8+ CD28− T cells in response to chronic EBV infection has been linked to rheumatoid arthritis (Klatt, et al. 2005). The clinical consequences of these changes are as yet not well defined, except for their extremely important negative impact on defense against infections. Considering the public health consequences of decreased immune competence in old age, strategies for immune response modulation are desirable to decrease the health burden for the elderly and improve their quality of life. (Fulop, et al. 2005).

Features of successful aging have been associated with well-preserved immune function while poor survival is predicted by high CTL counts, low numbers of B cells and poor responses by T cells to polyclonal stimulation. The phenomenon of replicative sensescence has been associated with these changes and relates to a finite number of doublings (25-30 cycles) after which cell cycle arrest occurs. In CTLs, this growth arrest is associated with increased production of several pro-inflammatory cytokines, resistance to apoptosis and loss of the co-stimulatory molecule, CD28, required for optimal stimulation of CTLs. In older adults, greater than 50% of CTLs fail to express CD28 and these cells are resistant to apoptosis.

The loss of CD28 expression due to replicative senescence has been associated with a number of the adverse effects of aging on immune function. Although the frequency of influenza virus-specific CTLs does not appreciably change with age, the decline in CTL activity against influenza may be due to a loss of antigen-specific proliferation and/or diminished lytic activity. Normal loss of CD28 expression during CTL activation and the potential for these cells to undergo activation-induced cell death, may be confused with the loss of CD28 with replicative senescence and resistance of CTLs to apoptosis. Furthermore, the role of cytokines (such as IL-2, IL-7, and IL-15) in preventing activation-induced cell death and age-related changes in the production of these cytokines create a complex array of interactions that may confound the interpretation of in vitro experiments. Understanding the complexity will provide an opportunity to optimize the CTL response to vaccination by manipulating CTLs that retain their replicative capacity in response to appropriate antigenic stimuli.

Currently, influenza vaccination of elderly individuals is recommended worldwide. A recent study looked retrospectively at influenza vaccine efficacy in individuals aged 65 years or older (Jefferson, et al. 2005). They found that in homes for elderly individuals, that vaccines were not significantly effective against influenza, influenza-like illness, or pneumonia. More encouragingly, vaccine performance was improved for admissions to the hospital for influenza or pneumonia, respiratory diseases, and cardiac disease (Jefferson, et al. 2005). This group concluded that the usefulness of influenza and pneumococcal vaccines was modest. On the same day the Jefferson report was published online, the American Medical Directors Association released a special announcement regarding the Jefferson study and influenza vaccine recommendations for the elderly (http://www.amda.com/newsroom/092205_vaccines.htm). While not disagreeing with the tenets of the study, they continued to recommend for vaccination of the elderly because influenza vaccination is effective at preventing severe illness, secondary complications, and deaths. They also reiterated that the CDC recommends influenza vaccination for people age 65 years and over and for all persons in long-term care facilities (http://www.amda.com/newsroom/092205_vaccines.htm). Both groups concluded that better influenza vaccines that offer more protection in older persons are desirable and a high priority of influenza researchers.

The threat of pandemic influenza has increased with the direct transmission of highly pathogenic avian H5N1 viruses to humans. Continued reliance in killed virus or subunit vaccines will leave adults at significantly higher risk of illness, disability and death in the event of an influenza pandemic. Research that increases our understanding of how immunosenescence affects the cell-mediated response to influenza and vaccine responsiveness is critical to the development of effective pandemic influenza vaccines for older people. In the absence of influenza vaccines that target these defects, an influenza pandemic will have a significant impact on older people and quickly overwhelm the health care system.

The CDC has recently (Aug. 8, 2005) stated that the effectiveness of inactivated influenza vaccine depends primarily on the age and the immunocompetence of the vaccine recipient and the degree of similarity between the viruses in the vaccine and those in circulation. When the vaccine and circulating viruses are antigenically similar, influenza vaccine prevents influenza illness among approximately 70-90% of healthy adults aged<65 years. Children aged≧6 months can develop protective levels of anti-influenza antibody against specific influenza virus strains after vaccination, although the antibody response among children at high risk for influenza-related complications might be lower than among healthy children. In addition, no efficacy was demonstrated among children who had received only one dose of influenza vaccine, illustrating the importance of administering two doses of vaccine to previously unvaccinated children aged<9 years. Older persons and persons with certain chronic diseases might develop lower post-vaccination antibody titers than healthy young adults and thus remain susceptible to influenza infection and influenza-related upper respiratory tract illness (http://www.cdc.gov/flu/professionals/vaccination/efficacy.htm). While current vaccines are cost-saving, new influenza vaccines will likely be needed to avoid the crisis anticipated in health care related to the general aging of the population.

Another component to the aging immune system is the relationship between innate immunity and inflammation. During evolution the human was set to live 40 or 50 years; today, however, the immune system must remain active for a much longer time. This very long activity leads to a chronic inflammation that slowly but inexorably damages one or several organs. This is a typical phenomenon linked to aging and it is considered the major risk factor for age-related chronic diseases. Alzheimer's disease, atherosclerosis, diabetes, sarcopenia, and cancer to name several, all have an important inflammatory component, though disease progression seems also dependent on the genetic background of individuals (Licastro, et al. 2005). Inflammatory genotypes are an important and necessary part of the normal host response to pathogens in early life, but the overproduction of inflammatory molecules might also cause immune-related inflammatory diseases and eventual death later (Licastro, et al. 2005).

Most age-related diseases have complex etiology and pathogenic mechanisms. The clinical diagnosis and therapy of these diseases requires a multidisciplinary approach with progressively increased costs. A body of experimental and clinical evidence suggest that the immune system is implicated, with a variable degree of importance, in almost all age-related or associated diseases. Both innate and the clonotypic immune system are usually involved in the pathogenesis of these chronic diseases (Caruso, et al. 2004; Pawelec, et al. 2002). Several functional markers of the immune system may be used either as markers of successful aging or conversely as markers of unsuccessful aging. A combination of high CD8⁺ and low CD4⁺ and poor T cell proliferation has been associated with higher mortality in very old subjects (Caruso, et al. 2004). Old men carrying an anti-inflammatory IL-10 high-producer genotype or a pro-inflammatory IL-2 low-producer genotype show the lowest values of CD8+ cells (Caruso, et al. 2004). This study, however, did not do a functional assessment of T cells.

In a mouse model looking at T cell subset patterns, researchers found that a composite combination of subset values was a significant predictor of longevity among genetically heterogeneous mice, with a strength of association higher in older mice than among the young (Miller and Chrisp, 2002). Developing useful biomarkers of aging has proven to be remarkable difficult, in part because many age-sensitive variables tested as candidate biomarkers are sensitive to genetic and nongenetic influences other than aging. Any individual assay, for example a test of a specific T cell subset in a single blood sample, is likely to have a good deal of uncertainty, but the combination of results from related tests may increase the signal-to-noise ratio and thus provide stronger predictive power than any single assay by itself (Miller and Chrisp, 2002). In humans, ImmunoScore testing would help build the models of T cell subset patterns. Possible courses of therapy would then be ideally tailored to meet the needs of the individual and not a “best guess, one size fits all” course of treatment.

Clearly, the population aged≧65 years would be better served by ImmunoScore diagnostics rather than the current state of affairs. A blanket recommendation for an influenza or pneumococcal vaccination for the entire elderly population may not be in the best interest of an individual being immunized. ImmunoScore diagnostic tests could, for example, first reveal levels of protective antibody to vaccine-preventable diseases. Of particular interest would be antibody levels against influenza, pneumococcal infection, tetanus, diphtheria, pertussis, hepatitis, varicella, CMV, and EBV. Just as important as determination of antibody levels in elderly patient sera, ImmunoScore diagnostic tests could reveal the status of cellular components of the immune system. The proportion of naive/committed T and B cells would be crucial for further recommendations by the attending medical staff. As therapeutic interventions are developed for dealing with immunosenescence, the ImmunoScore diagnostic information regarding individuals and compiled database information will shed valuable light onto the effects of treatments on the immune system. As the population ages, strategies for immune response modulation are desirable to decrease the health burden for the elderly and improve their quality of life.

A preliminary immune risk phenotype (IRP) has been developed from longitudinal studies of the elderly (Wikby, et al. 2005). Immune system measurements consisted of determinations of T-cell subsets, plasma IL-6, IL-2 responsiveness to conconavalin A, and CMV and EBV serology. Regression analyses indicated that the IRP and cognitive impairment together predicted 58% of observed deaths. This type of analysis would be a valuable adjunct to assessing insurance premiums.

The following table captures exemplary desirable analytes to monitor in the population as individuals age. A database storing the results of such assays could ensure that a given individual's analyte levels could be tracked over time rather than merely captured as a snapshot.

TABLE 1
Alterations in the T-cell compartment with age

| Alteration | Analyte |
|---|---|
| ↑ | CD45RO⁺ cells |
| ↑ | CD95⁺ cells |
| ↓ | CD28 expression |
| ↑ | CD152 expression |
| ↑ | killer cell lectin-like receptor G1 |
| ↓ | apoptosis of CD8 cells |
| ↑ | apoptosis of CD4 cells |
| ↓ | IFN-γ production |
| ↓ | IL-2 production |
| ↓ | telomere lengths |
| ↓ | telomerase induction |
| ↑ | DNA damage |
| ↓ | DNA repair |
| ↓ | stress resistance and heat-shock protein expression |

Thus, in exemplary embodiments of the present invention an Immunosenescence supperpanel can be defined, comprising the following panels:

Meningococcal Diagnostic Panel;

Persistent Immunity Induced by Childhood Vaccines; and

Immunosenescence Diagnostic Panel

The first two panels are defined above in Sections IA1 and IA3, and the Immunosenesence panel can be defined as follows.

Human aging is associated with progressive decline in immune functions and increased frequency of infections. A decline in immune function is a hallmark of aging that affects the ability to resist influenza and respond to vaccination. The most important changes occur in T cell immunity. An accumulation of dysfunctional T cells may be detrimental under conditions of chronic antigenic stress (chronic infection, cancer, autoimmunity).

### Exemplary Alterations in T-Cell Compartment to Monitor

| Typical | |
|---|---|
| Alteration | Analyte |
| Increased | CD45RO⁺ cells |
| Increased | CD95⁺ cells |
| Decreased | CD 28 expression |
| Increased | CD152 expression |
| Increased | Killer cell lectin-like receptor G1 |
| Decreased | Apoptosis of CD8⁺ cells |
| Increased | Apoptosis of CD4⁺ cells |
| Decreased | IFN-γ production |
| Decreased | IL-2 production |
| Decreased | Telomere lengths |
| Decreased | Telomerase induction |
| Increased | DNA damage |
| Decreased | DNA repair |
| Decreased | Stress resistance and heat-shock |
| | protein expression |

Other analytes of particular interest in an immunosenescence assay panel can, for example, include:
Antibody to CMV
Antibody to EBV
Antibody to influenza
Antibody to pneumococcal disease
Antibody to pertussis
Antibody to tetanus
Antibody to diphtheria
Plasma levels of IL-6

Th1/Th2 components as described below:

| | |
|---|---|
| Th1 | Th2 |

| Cytokines | Receptors | Cytokines | Receptors |
|---|---|---|---|
| INF-γ | CCR5 | IL-4 | CCR3 |
| TNF-α | CXCR3 | IL-5 | CCR4 |
| IL-2 | CCR1 | IL-6 | CCR8 |
| IL-12 | | IL-10 | CRTh2 |
| | | IL-13 | |

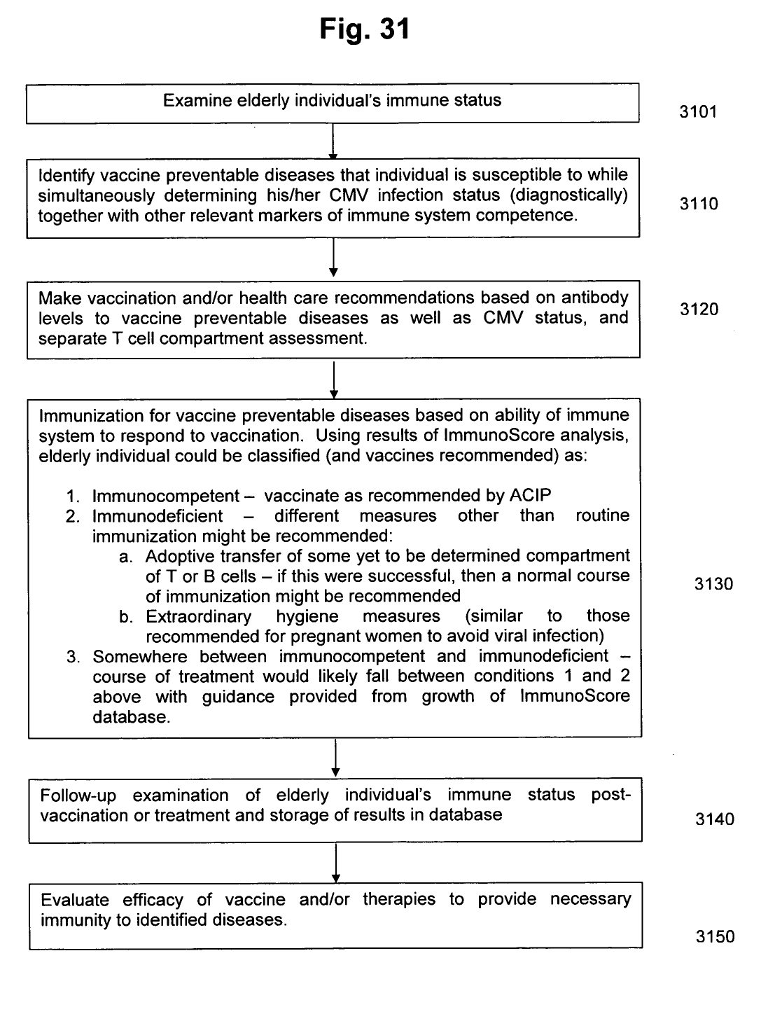

FIG. 31 depicts an exemplary process flow for managing immunosenescent individuals, either in a health care provider or a health care insurer context.

In exemplary embodiments of the present invention Immunosenescense in an individual can be managed using the process exemplary flow depicted in FIG. 31. With reference thereto, at 3101. an elderly individual's immune status can be examined. This can be accomplished by conducting one or more assay panels as described above in Section I. At 3110, the vaccine preventable diseases that the elderly individual is susceptible to can be identified at the same time the individuals CMV infection status together with other relevant markers of an immune system competence can also be determined. At 3120 vaccine and/or other healthcare recommendations can be made based upon the immune status examined at 3101. Additionally, a separate T cell compartment can be assessed. 3130 the individual can be immunized for vaccine preventable disease based upon his or her immune system's ability to response to vaccination. Using the ImmunoScore data, the individual can be classified as either (1) immunocompetent (2) immuno-deficient or (3) somewhere in between immunocompetent or immuno deficient. At 3130 an immuno-competent individual can be vaccinated as recommended by current ACIP recommendations. An immuno-deficient individual would need to be managed using different measures than routine vaccination. Such measures could include, for example, adoptive transfer of a compartment of T ob B cells or extraordinary hygiene measures. The individuals who falls somewhere between immuno-competence and immuno-deficiency need some kind of hybrid health management between standard vaccination and immunoadjuvant therapies such as adoptive transfer of T or B cells and extraordinary hygiene measures. At 3140, the elderly individual's immune status can be followed-up post vaccination or post treatment and these results stored in the system database. At 3150 this information can be used to evaluate the efficacy of the vaccination or other therapies as to their abilities to provide the necessary immunity to the identified diseases.

L. Frozen Storage of Naive Immune Cells (IRP Considerations)

As previously described, the immune risk phenotype (IRP) is an emerging concept—predicting mortality based on CMV seropositivity (Pawelec, et al. 2005). This group maintained that the manner in which CMV and the host immune system interact is critical in determining the IRP and is hence predictive of mortality. The consequences of IRP is early expression of immunosenescence. Immunosenescence leads to: a) decreased T- and B-cell responses to foreign antigen; b) increased responses to self antigens; c) increased morbidity and mortality to infectious disease; and d) decreased response to vaccine antigens.

Greater elucidation of the IRP and its consequences is to be expected in the future. Genetic screening at a very early age could be predictive of immune health at a much more advanced age. The ImmunoScore diagnostic screen could be performed from a heel stick done at birth, and a child's baseline immune status could almost instantaneously be generated. Pre-natal screening tests could also be developed in the future as an immunodiagnostic tool.

Concerned parents may wish to store their child's cord blood as a source of hematopoietic progenitor cells that could be stored (at a cost to the parents or the insurer's) for that child for treatment of developing IRP symptoms much later in life. Umbilical cord blood (UCB) is currently used as a source of these hematopoietic progenitor cells as an alternative to the bone marrow or peripheral blood for treatment of several onco-hematological diseases (Adami, et al. 2005).

On Apr. 18, 2005 the Institute of Medicine (IOM) issued a report recommending that a new cord blood coordinating center—similar to the existing National Marrow Donor Program—be set up to ensure a standardized and interconnected national system to cost-effectively store and distribute these cells.

ImmunoScore diagnostics shows the need for storing cord blood.

Another application for ImmunoScore diagnostics is to link storage and analysis of naive cells of the immune system (innate or adaptive), as next described.

T cells currently used for adoptive immunotherapy trials are selected for their capacity to produce high levels of IFN-γ and for their ability to efficiently and specifically lyse relevant target cells (Dudley and Rosenburg, 2003; Yee, et al. 2002). However, it was found that CD8⁺ T cells that acquire complete effector properties and exhibit increased anti-tumor activity in vitro are less effective at triggering tumor regressions and cures in vivo (Gattinoni, et al. 2005). While the progressive acquisition of terminal effector properties is characterized by pronounced in vitro tumor killing, in vivo T cell activation, proliferation, and survival are progressively impaired. These findings suggest that the current methodology for selecting T cells for transfer is inadequate (Gattinoni, et al. 2005). It is clear that new solutions are needed to generate more effective anti-tumor T cells for the development of experimental human adoptive transfer-based therapies.

The indication is that storage of naive T and B cells is important for individuals who will become immunocompromised later in life, whether those cells come from that individual or from another source. Naive cells would also not necessarily be isolated from cord blood, but could also be isolated from bone marrow or peripheral blood. In addition, screening methods can be used to characterize those immune cells regarding cell surface characteristics and cytokine expression. Here too, ImmunoScore can be used to a distinct advantage.

M. Vaccine Use Outcome/Design

Currently, what the public considers vaccines are designed as a prophylactic means to avoid illness caused by infectious disease. In practice, agents used to promote an immune response as a therapeutic course of action for cancer or immunotherapy have also been termed “vaccines.” It is the intent of the ImmunoScore design to be able to monitor changes in an individual's immune system in relation to a prophylactic or therapeutic vaccine and enable the individual patient and his physician to make the best possible decisions regarding the patient's immune system health regarding prophylactic vaccination, therapeutic vaccination, or other therapeutic treatment in attempt to “shift” the immune system of that patient. In addition, the ImmunoScore database will compile important population data regarding demographics and population genetics.

N. Research Services

In exemplary embodiments of the present invention ImmunoScore technologies can be used to provide research services, such as, for example, for clinical trials in the following areas:

1. vaccines;

2. transplants;

3. adoptive immunotherapy;

4. population modeling; and

5. government applications.

O. Immigration Consulting

Testing the immigrant population for vaccine-preventable diseases is another embodiment of the invention. Governments are very interested in the immunization status of individuals and families immigrating into their countries. The invention can rapidly provide the results of assays to governmental authorities for all required immunizations. There would be no need to rely on paperwork—a diagnostic examination would yield more suitable data regarding immune status. The current vaccination requirements for immigration into the United States are for measles, mumps, rubella, polio, tetanus, diphtheria, pertussis, influenza, hepatitis B and any other vaccinations recommended by the Advisory Committee for Immunization Practices (ACIP). Current recommendations of the ACIP also include varicella, Haemophilus influenzae type B, and pneumococcal vaccines. The current law requires all individuals applying for status as a lawful permanent resident (either by applying for an immigrant visa abroad or for adjustment of status in the United States) to establish that they have been vaccinated. Nonimmigrant (temporary) visa applicants are not required to comply with the vaccination requirements as a condition of visa issuance, but must comply if they apply for adjustment of status at a later date (Immigration and Naturalization Services, 2001).

One or more ImmunoScore diagnostic panels could be provided to INS or other immigration authorities as a means to determine the immune status of immigrants. In practice, the ImmunoScore diagnostic testing would be more cost-effective than a paper record trail and more likely to be reliable as an accurate assessment of immune status of individuals relocating to the United States.

Additionally, the Institute of Medicine (IOM) has concluded that the United States quarantine system is in need of a strategic overhaul. The IOM reports that the United States once had 55 federal quarantine stations, but the perception that microbial threats had been controlled led to dismantling of most of the system in the 1970s. However, nearly 40 new infectious diseases have been identified since 1973, and bioterrorism has become a serious concern. The 25 stations that will make up the expanded quarantine station system now receive more than 75 million international travelers a year, according to IOM reports. The stations screen travelers, refugees, immigrants, animals, and cargo for disease agents shortly before and during their arrival. However, the quarantine system relies on a much broader network that includes local public health agencies, hospitals, customs agents, agricultural inspectors, and others, the IOM said.

The IOM recommended the following:

The quarantine stations, the CDC, and the DGMQ (called the quarantine core) should lead the effort by developing a national strategic plan with uniform principles and outcomes. The quarantine core should shift its main focus from inspecting people and cargo at ports to leading the activities of the overall quarantine system. The strategic plan should help participating government agencies and other groups in the system to prioritize activities and focus resources on the greatest risks.

The quarantine core should work with partners in the quarantine network to define or redefine each group's roles, authority, and communication channels.

The quarantine system needs enhanced skills, more people, more training, more space, and better use of technology to fulfill its evolving role. An example of technology cited in the news release was targeted use of passenger locator cards that could be used on flights to and from countries with disease outbreaks. The cards would log passenger seat numbers and contact information in a scannable format. This could simplify tracking of passengers potentially exposed to disease, such as those who flew to the United States from Sierra Leone in 2004 with a man who later died of Lassa fever.

The quarantine core must review its methodology periodically to ensure stations are in the best places and appropriately staffed.

The quarantine core must have plans, capacity, resources, and “clear and sufficient legal authority” to respond quickly to surges in activity at one or more ports.

The core must define and fund a research agenda to measure the effectiveness of its procedures. The committee found that many routines at quarantine stations are based on experience and tradition and lack a scientific basis.

The core must use scientifically sound methods to measure the effectiveness and quality of its operations, including assessing performance of critical functions throughout the system. It must also address any shortfalls that come to light. (http://www.nap.edu/books/030909951 X/html).

ImmunoScore technology could be useful at such immigration port of entry screening points. There is a need for global health that can not be understated. The cost of failure could be extremely high. There are people moving around the globe and among the states with clear health needs, and they are currently moving without the ability of government authorities to track them.

Additionally, ImmunoScore technologies can be used to discover links between immunological phenomena. For example, from the results of Greenway (discussed above in Section I regarding the immigrant panel) a possible link between TB infection and HepB prevalence in can be investigated by analyzing sera from an immigrant population for both active TB and HepB seropositivity. It is possible that more than one co-infection may be found in this manner. For example, in the following study, A high prevalence of hepatitis B virus infection among tuberculosis patients with and without HIV in Rio de Janeiro, Brazil, Blal C A et al Eur J Clin Microbiol Infect Dis. 2005 January; 24(1):41-3, such a correlation was in fact found.

The Blal study sought to investigate the prevalence and exposure factors associated with hepatitis B infection in tuberculosis patients with and without HIV type 1 co-infection, the presence of hepatitis B virus serological markers was investigated in a retrospective study. The seroprevalence of hepatitis B virus in patients with tuberculosis only was 14.6%, and in tuberculosis patients co-infected with HIV it increased to 35.8%. In patients with HIV and tuberculosis co-infection, homosexuality constituted the principal exposure factor, while in tuberculosis patients without HIV, a gradual increase in hepatitis B virus seroprevalence was noted along with increasing age. These results demonstrate that hepatitis B infection is highly prevalent in tuberculosis patients in Brazil and suggest that a vaccination program for the general population should be considered in order to prevent further hepatitis B infections.

P. Disaster Survivors: Immunizations, Recovery, Prognosis and Treatment

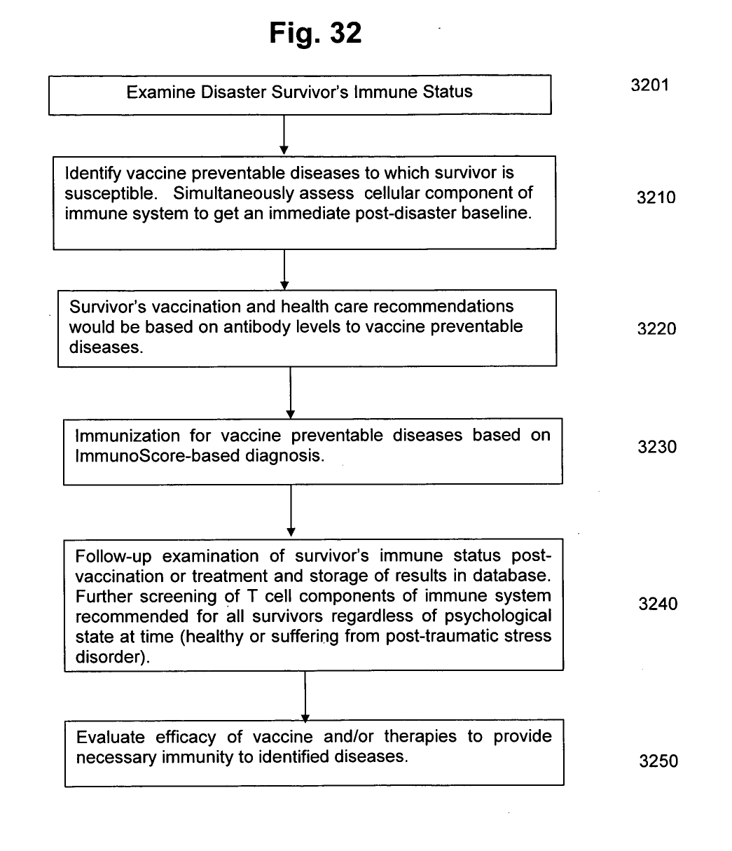

In exemplary embodiments of the present invention, rapid response services to disaster survivors can be provided. FIG. 32 depicts an exemplary process flow for such an application.

At 3201 a disaster survivors immune status can be examined using one or more ImmunoScore assay panels as described above in Section I. At 3210 the vaccine preventable diseases to which the survivor is susceptible can be identified and simultaneously the cellular component of his or her immune system can be assessed to get an immediate post disaster baseline. At 3220 vaccination and healthcare recommendations can be generated based upon antibody levels to the identified to the assay vaccine preventable diseases. At 3230 immunization can be carried out and at 3240 follow-up examination of the survivor's immune status can be administered and the results stored in the system database. Further screening of T cell components of the immune system is recommended for all survivors regardless of their psychological state at the time in order to develop data regarding post-traumatic stress disorder. Finally, at 3250 the efficacy of the vaccine and/or therapies can be evaluated as to their ability to provide necessary immunity to the identified diseases.

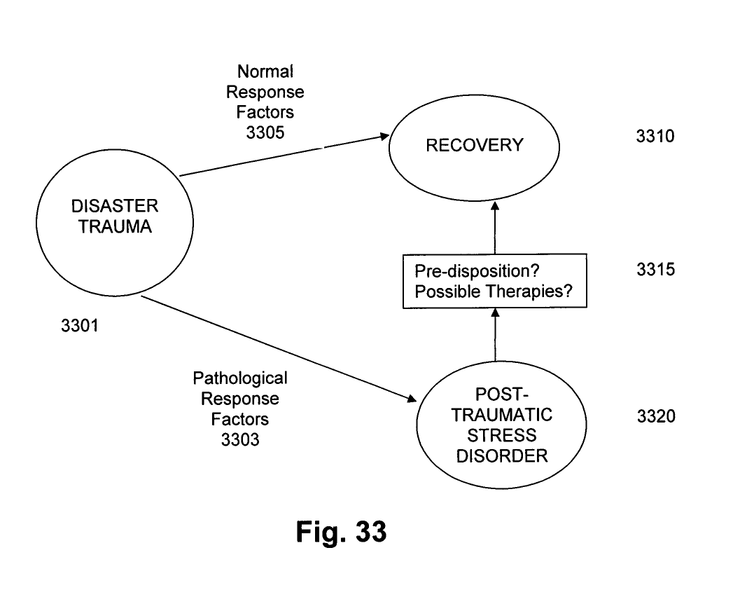

There are many different possible responses of an individual to an event perceived as potentially life-threatening. It is difficult to predict long-term responses to trauma based on the acute response to a traumatic event. If physiological risk factors are important in understanding how psychopathology develops, then ImmunoScore measurements can provide invaluable research information and possibly identify treatments yet to be defined. This could pave the way to personalized medicine. FIG. 33 illustrates possible responses to trauma.

With reference thereto, at 3301 a Disaster Trauma occurs. There are two pathways leading from 3301, namely, Normal Response Factors 3305 and Pathological Response Factors 3303. A Normal Response Factors 3305 pathway from Disaster Trauma 3301 leads to Recovery at 3310. However, Pathological Response Factors 3303 lead an individual from Disaster Trauma 3301 to Post-Traumatic Stress Disorder 3320. It is the job of healthcare personnel to put the individual on a Pathway to Recovery 3310. In exemplary embodiments of the present invention ImmunoScore technologies can be used to determine possible therapies 3315, as well as to track immunological correlates of PTSD to verify diagnosis and evaluate therapeutic efficacies.

In the immediate aftermath of a traumatic event, most people experience a combination of the following symptoms: (a) difficulty sleeping, (b) difficulty concentrating, (c) irritability, (d) nightmares, (e) recurrent thoughts of the trauma, and (f) distress at the reminder of the traumatic event. The question in the determination of a pathological response is when does the continuation of these “normal” responses become pathological, and have serious effect on the health of the individual's immune system?

There are different possible outcomes of trauma exposure. There is an increased risk of: (a) Post-Traumatic Stress Disorder (PTSD), (b) major depression, (c) panic disorder, (d) generalized anxiety disorder, (e) substance abuse, and (f) other somatic symptoms or expressions of physical illness including hypertension, asthma, and chronic pain syndromes. The differential outcomes may rely on different physiological parameters.

Pre-existing cognitive factors may or may not be the cause, result, or correlate of pre-existing biological alterations, either or both setting the stage for an extreme response to the trauma. Clarifying the precise nature and biological correlates of symptoms that appear in the immediate aftermath of a trauma will assist in developing models for potential prophylactic interventions and early treatments. In this regard the ImmunoScore diagnostic panel could initially be used in a research application to track immune system markers and relate them to specific conditions. As a system database evolves, ImmunoScore panels can, for example, be used as a guide to therapeutic treatment.

Individuals currently at the greatest risk for developing PTSD following trauma are those individuals with (a) a family history of psychopathology, (b) a history of childhood abuse, (c) prior trauma exposure, and (d) the cognitive factors of lower IQ, female gender, an poor social support. There is increased concordance for PTSD in monozygotic twins compared with dizygotic twins lending support to the genetic pre-disposition argument of PTSD.

Q. Monitor Adoptive Immunotherapy/Transplants

After adoptive transfer, several events must occur for T cells to cause the regression of established tumors. T cells must be activated in vivo through antigen-specific vaccination. They must then vigorously expand to levels capable of causing the destruction of significant tumor burdens. Finally, anti-tumor T cells must survive long enough to complete the eradication of all tumor cells (Overwijk, et al. 2003). It has been found in an animal model that the progressive differentiation of T cells to a terminal differentiated effector stage results in a series of phenotypic and functional changes that make them less “fit” to perform these functions (Gattinoni, et al. 2005).

In patients under consideration for adoptive immunotherapy and/or transplantation, history and analyses of exposure to CMV, EBV, West Nile Virus, and viral hepatitis in both the donor and recipient are crucial. ImmunoScore diagnoses of both the donor and recipient would examine the immune history of both individuals.

R. Elective Surgery

Many patients opt for elective surgery—plastic surgery, facial plastic surgery, dermatology, cosmetic dentistry, vision, urology, and infertility among others. Whenever undergoing surgery, there is a risk of nosocomial infection. Common organisms that cause nosocomial infections are Apergillus, Candida, Staphylococcus aureus, Staphylococcus epidermidis, Pseudomonas aeruginosa, and Bordetella pertusis. Prior to elective surgery, it would benefit the patient and the attending surgeon to know the level of antibody protection to these infectious agents. An ImmunoScore panel could be tailored to meet these diagnostic needs and immunizations could be provided to those agents with available vaccine. In addition, following surgery patients could be screened for c reactive protein (CRP), tumor necrosis factor-alpha (TNF-α), IL-6, and soluble IL-2 receptor (sIL-2R) as possible early indicators of inflammation leading to sepsis. It is important to screen for a panel of analytes indicating sepsis, as one analyte is often not enough to get a proper diagnosis.

S. Services to Charitable Foundations Promoting Immunological Well Being

Currently, the lack of accurate, affordable, and accessible diagnostic tests significantly impedes global health efforts. The Global Alliance for Vaccines and Immunizations (GAVI) was created in 1999 to protect health and save children's lives throughout the widespread use of modern vaccines. GAVI is a partnership of governments, international organizations, major philanthropists, research institutions, and the private sector that work together to: (a) improve access to sustainable immunization services, (b) expand the safe use of all needed cost-effective vaccines, (c) accelerate research and development efforts for new vaccines needed in developing countries, (d) make immunization coverage a key indicator of development, (e) promote sustainability by adequate financing, and (f) reinforce global and national immunization goals including eradicating polio, eliminating maternal and neonatal tetanus, reducing measles, and increasing access to vitamin A.

Underlying all health care tools—including therapeutic products, vaccines, and other preventative tools—are “platform” technologies that define and facilitate their use. For example, immunochromatography is a technology platform that has enabled the development of affordable, easy-to-use dipstick format diagnostic tools. The ImmunoScore diagnostic panel, a platform technology, can be used to great advantage by GAVI to improve global health efforts

GAVI issues requests for proposal (RFPs) to support research efforts to create diagnostic technology platforms and tolls that enable improved prevention, treatment, and surveillance in developing country settings. The foundation issues RFPs to support the systemic evaluation of sets of genes, proteins, and cellular pathways to determine their potential role in contributing to the development of new vaccines, diagnostics, and drugs for GAVI's priority diseases and conditions. One area of concern is population genetics and how to design drugs and vaccines to discourage the emergence of resistance and to discover how genetics affects the efficacy of drugs and other interventions. The ImmunoScore database would be an ideal tool for GAVI to use to evaluate genetic parameters and immune response to vaccines and drugs under consideration. A second area is applied immunology. Here systematic approaches, such as that provided by the ImmunoScore technology, are needed to measure the human immune response to guide vaccine design and define biological signs that identify early or latent infection.

T. Discovery of Unwanted Immunogenicity of Therapeutics

There is potential of the human immune system to identify biological therapeutic products as foreign and mount an immune response. There are three main areas of concern with the production of antibodies against biological therapeutics in humans:
1. Safety—assurance of safety involves the assessment of whether antibodies induced could have adverse clinical implications.
2. Efficacy—can be affected by the presence of antibodies binding to the product and reducing its potency.
3. Measurement of phamacokinetic/pharmacodynamic parameters—the presence of antibodies can alter these clinical parameters and also interfere in the assays used in their assessment (Koren, et al. 2002).

The immunogenicity of therapeutic proteins can be influenced by many factors, including the genetic background of the patient, the type of disease, the type of protein (human or nonhuman), the presence of conjugates or fragments, the route of administration, dose frequency, and duration of treatment (Schellekens, 2002). Manufacturing, handling, and storage can introduce contaminants, or alter the three dimensional structure of the protein via oxidation or aggregate formation. Various means have been suggested by which therapeutic proteins might be modified to reduce their immunogenicity, including PEGylation, site-specific mutagenesis, exon shuffling, and humanization of monoclonal antibodies (Schellekens, 2002). In the future, it may be possible to predict the immunogenicity of new therapeutic proteins more accurately, using specifically designed animal models, including nonhuman primates and transgenic mice.

ImmunoScore diagnoses and database storage could be instrumental in the development of analytical techniques to monitor both the drugs and the patient population. An individual's tendency to mount an immune challenge to a protein therapeutic could be revealed prior to initiation of the treatment based upon the patient's ImmunoScore profile. In addition, once therapeutic treatment began, ImmunoScore diagnoses and database management could track a patient's immune response to the drug. The drug manufacturers would be able to use the ImmunoScore technology to conduct clinical trials and also to select an appropriate population in which to test the drug. Based upon ImmunoScore population data, the drug could be designated for use based upon the genotype of the individual being treated.

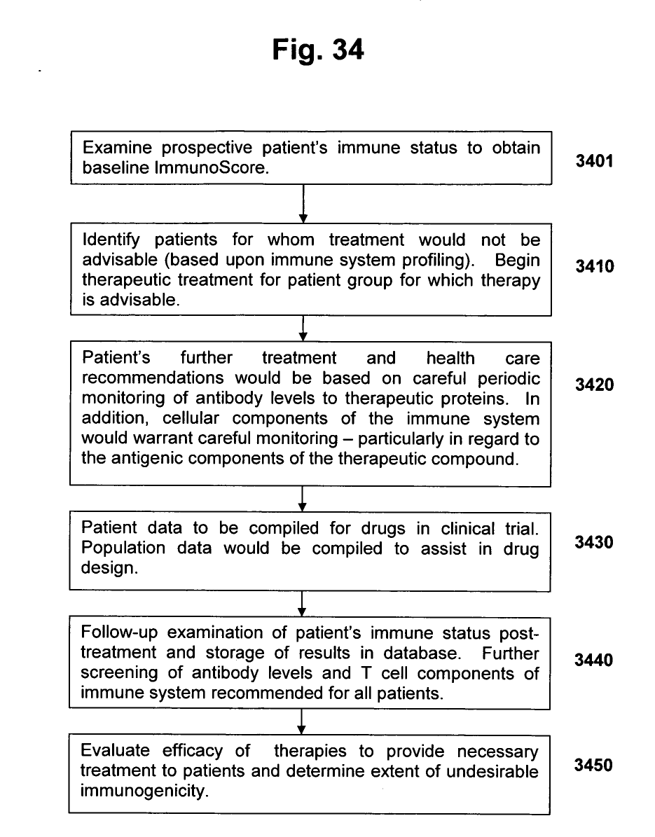

FIG. 34 depicts an exemplary process flow for an ImmunoScore immunogenicity study in exemplary embodiments of the present invention. The exemplary study is directed to immunogenicity of therapeutic proteins.

With reference thereto, at 3401 a prospective patient's immune status can be examined to obtain a baseline ImmunoScore. At 3410 patients for whom treatment would not be advisable (based upon immune system profiling) can be identified, and therapeutic treatment for a patient group for which therapy is advisable can be initiated. At 3420 patients' further treatment and health care recommendations can be made, based on careful periodic monitoring of antibody levels to therapeutic proteins. In addition, cellular components of the immune system would warrant careful monitoring—particularly in regard to the antigenic components of the therapeutic compound. At 3430 patient data can be compiled for drugs in clinical trial. Population data can also be compiled to assist in drug design. At 3440, follow-up examinations of patients' immune status post-treatment can be implemented and the results stored in a system database. Further screening of antibody levels and T-cell components of immune system can be implemented for all patients. Finally, at 3450 the efficacy of therapies to provide necessary treatment to patients can be evaluated, and extent of undesirable immunogenicity can be determined.

The present invention has been described in connection with exemplary embodiments and implementations, as examples only. It will be understood by those having ordinary skill in the pertinent art that modifications to any of the embodiments may be easily made without materially departing from the scope and spirit of the present invention which is defined by the appended claims. Such modifications can include, for example, using other appropriate assays or tests, other rules or analyses of the results thereof, as may be known in the art to assess the immune status of individuals or populations. Additionally, such modifications can include, for example, using various assay devices and techniques as may be known, using various available methods of storing and analyzing data (including various “data mining” techniques) as may be available, and defining various alternative demographic groups and various sets of ImmunoScore test panels to be administered thereto.

### Appendix A

### To

### Systems and Methods for Obtaining, Storing, Processing and Utilizing Immunologic Information of Individuals and Populations

### Appendix A

### REFERENCES

Abramson M J, Puy R M, Weiner J M. Is allergen immunotherapy effective in asthma? A meta-analysis of randomized controlled trials. Am J Respir Crit Care Med. 1995 April; 151 (4):969-74.
Adami V, Malangone W, Falasca E, Marini L, Risso A, Crini S, Toniutti E, Passoni Ferraro E, Del Frate G, Pittino M, Biffoni F, Rinaldi C, Degrassi A. A closed system for the clinical banking of umbilical cord blood. Blood Cells Mol Dis. 2005 Aug. 18; [Epub ahead of print].
Advisory Committee on Immunization Practices. Preventing pneumococcal disease among infants and young children. Recommendations of the Advisory Committee on Immunization Practices (ACIP). MMWR Recomm Rep. 2000 Oct. 6; 49(RR-9): 1-35.
Advisory Committee on Immunization Practices. Prevention of varicella: Recommendations of the Advisory Committee on Immunization Practices (ACIP). Centers for Disease Control and Prevention. MMWR Recomm Rep. 1996 Jul. 12; 45(RR-11):1-36.
Alibek K. Biohazard. 1999. Random House, New York, N.Y.
Amir J, Liang X, Granoff D M. Variability in the functional activity of vaccine-induced antibody to Haemophilus influenzae type b. Pediatr Res. 1990 April; 27(4 Pt 1):358-64.
Amir J, Scott M G, Nahm M H, Granoff D M. Bactericidal and opsonic activity of IgG1 and IgG2 anticapsular antibodies to Haemophilus influenzae type b. J Infect Dis. 1990 July; 162(1):163-71.
Andrew D, Aspinall R. Age-associated thymic atrophy is linked to a decline in IL-7 production. Exp Gerontol. 2002 January-March; 37(2-3):455-63.
Army regulation 40-562. Immunization and Chemoprophylaxis. 1 Nov. 1995. AFJI 48-110. BUMEDINST 6230.15. CG COMDTINST M6230.4E.
Amon S S, Schechter R, Inglesby T V, Henderson D A, Bartlett J G, Ascher M S, Eitzen E, Fine A D, Hauer J, Layton M, Lillibridge S, Osterholm M T, O'Toole T, Parker G, Perl T M, Russell P K, Swerdlow D L, Tonat K; Working Group on Civilian Biodefense. Botulinum toxin as a biological weapon: medical and public health management. JAMA. 2001 Feb. 28; 285(8):1059-70.
Aspinall R. Ageing and the immune system in vivo: commentary on the 16th session of British Society for Immunology Annual Congress, Harrogate, December 2004. Immun Ageing. 2005 Feb. 22; 2(1):5.
Aspinall R, Andrew D. Thymic involution in aging. J Clin Immunol. 2000 July; 20(4):250-6.
Atkinson W L, Markowitz L E, Adams N C, Seastrom G R. Transmission of measles in medical settings—United States, 1985-1989. Am J. Med. 1991 Sep. 16; 91(3B):320S-324S.
Atkinson W L, Pickering L K, Schwartz B, Weniger B G, Iskander J K, Watson J C; Centers for Disease Control and Prevention. General recommendations on immunization. Recommendations of the Advisory Committee on Immunization Practices (ACIP) and the American Academy of Family Physicians (AAFP). MMWR Recomm Rep. 2002 Feb. 8; 51(RR-2):1-35.
Aurelian, L. ed. Herpesvirus, the Immune System and AIDS. 1990. Kluwer Academic Publishers, Boston, Mass.
Ausiello C M, Lande R, Stefanelli P, Fazio C, Fedele G, Palazzo R, Urbani F, Mastrantonio P. T-cell immune response assessment as a complement to serology and intranasal protection assays in determining the protective immunity induced by acellular pertussis vaccines in mice. Clin Diagn Lab Immunol. 2003 July; 10(4):637-42.
Babcock G J, Decker L L, Volk M, Thorley-Lawson D A. EBV persistence in memory B cells in vivo. Immunity. 1998 September; 9(3):395-404.
Baker C J, Paoletti L C, Rench M A, Guttormsen H K, Carey V J, Hickman M E, Kasper D L. Use of capsular polysaccharide-tetanus toxoid conjugate vaccine for type II group B Streptococcus in healthy women. J Infect Dis. 2000 October; 182(4):1129-38. Epub 2000 Sep. 08.
Baker C J, Paoletti L C, Wessels M R, Guttormsen H K, Rench M A, Hickman M E, Kasper D L. Safety and immunogenicity of capsular polysaccharide-tetanus toxoid conjugate vaccines for group B streptococcal types Ia and Ib. J Infect Dis. 1999 January; 179(1):142-50.
Baker C J, Rench M A, McInnes P. Immunization of pregnant women with group B streptococcal type III capsular polysaccharide-tetanus toxoid conjugate vaccine. Vaccine. 2003 Jul. 28; 21(24):3468-72.
Baker-Zander S, Sell S. A histopathologic and immunologic study of the course of syphilis in the experimentally infected rabbit. Demonstration of long-lasting cellular immunity. Am J Pathol. 1980 November; 101(2):387-413.
Balding J, Healy C M, Livingstone W J, White B, Mynett-Johnson L, Cafferkey M, Smith O P. Genomic polymorphic profiles in an Irish population with meningococcaemia: is it possible to predict severity and outcome of disease? Genes Immun. 2003 December; 4(8):533-40.
Baldini M, Lohman I C, Halonen M, Erickson R P, Holt P G, Martinez F D. A Polymorphism* in the 5′ flanking region of the CD14 gene is associated with circulating soluble CD14 levels and with total serum immunoglobulin E. Am J Respir Cell Mol. Biol. 1999 May; 20(5):976-83.
Balin B J, Gerard H C, Arking E J, Appelt D M, Branigan P J, Abrams J T, Whittum-Hudson J A, Hudson A P. Identification and localization of Chlamydia pneumoniae in the Alzheimer's brain. Med Microbiol Immunol (Berl). 1998 June; 187(1):23-42.
Balkovic E S, Goodman R A, Rose F B, Borel C O. Nosocomial influenza A (H1N1) infection. Am J Med Technol. 1980 May; 46(5):318-20.
Ball T M, Serwint J R. Missed opportunities for vaccination and the delivery of preventive care. Arch Pediatr Adolesc Med. 1996 August; 150(8):858-61.
Bandholtz L, Kreuger M R, Svanholm C, Wigzell H, Rottenberg M E. Adjuvant modulation of the immune responses and the outcome of infection with Chlamydia pneumoniae. Clin Exp Immunol. 2002 December; 130(3):393-403.
Bart K J, Orenstein W A, Preblud S R, Hinman A R, Lewis F L Jr, Williams N M. Elimination of rubella and congenital rubella from the United States. Pediatr Infect Dis. 1985 January-February; 4(1):14-21.
Bayne-Jones S, The Evolution of Preventive Medicine in the United States Army, 1607-1939. 1968. Office of the Surgeon General, Department of the Army. Washington, D.C.
Bettelli E, Das M P, Howard E D, Weiner H L, Sobel R A, Kuchroo V K. IL-10 is critical in the regulation of autoimmune encephalomyelitis as demonstrated by studies of IL-10- and IL-4-deficient and transgenic mice. J. Immunol. 1998 Oct. 1; 161(7):3299-306.
Bicknell W J. The case for voluntary smallpox vaccination. N Engl J. Med. 2002 Apr. 25; 346(17):1323-5. Epub 2002 Mar. 28.
Biological and chemical terrorism: strategic plan for preparedness and response. Recommendations of the CDC Strategic Planning Workgroup. MMWR Recomm Rep. 2000 Apr. 21; 49(RR-4):1-14.
Birk R W, Gratchev A, Hakiy N, Politz O, Schledzewski K, Guillot P, Orfanos C E, Goerdt S. [Alternative activation of antigen-presenting cells: concepts and clinical relevance] Hautarzt. 2001 March; 52(3):193-200.
Bjork A, Venge P, Peterson C G. Measurements of ECP in serum and the impact of plasma coagulation. Allergy. 2000 May; 55(5):442-8.
Bjorksten B. Effects of intestinal microflora and the environment on the development of asthma and allergy. Springer Semin Immunopathol. 2004 February; 25(3-4):257-70. Epub 2003 Oct. 24.
Bjorksten B, Naaber P, Sepp E, Mikelsaar M. The intestinal microflora in allergic Estonian and Swedish 2-year-old children. Clin Exp Allergy. 1999 March; 29(3):342-6.
Blasi F, Legnani D, Lombardo V M, Negretto G G, Magliano E, Pozzoli R, Chiodo F, Fasoli A, Allegra L. Chlamydia pneumoniae infection in acute exacerbations of COPD. Eur Respir J. 1993 January; 6(1):19-22.
Bloom S. Coordinating cord blood efforts. J Clin Invest. 2005 June; 115(6):1395-6.
Bohm I, Bauer R. [Th1 cells, Th2 cells and atopic dermatitis] Hautarzt. 1997 April; 48(4):223-7.
Boslego J W, Tramont E C, Chung R C, McChesney D G, Ciak J, Sadoff J C, Piziak M V, Brown J D, Brinton C C Jr, Wood S W. Efficacy trial of a parenteral gonococcal pilus vaccine in men. Vaccine. 1991 March; 9(3): 154-62.
Bousquet J, Becker W M, Hejjaoui A, Chanal I, Lebel B, Dhivert H, Michel F B. Differences in clinical and immunologic reactivity of patients allergic to grass pollens and to multiple-pollen species. II. Efficacy of a double-blind, placebo-controlled, specific immunotherapy with standardized extracts. J Allergy Clin Immunol. 1991 July; 88(1):43-53.
Bradley B L, Azzawi M, Jacobson M, Assoufi B, Collins J V, Irani A M, Schwartz L B, Durham S R, Jeffery P K, Kay A B. Eosinophils, T-lymphocytes, mast cells, neutrophils, and macrophages in bronchial biopsy specimens from atopic subjects with asthma: comparison with biopsy specimens from atopic subjects without asthma and normal control subjects and relationship to bronchial hyperresponsiveness. J Allergy Clin Immunol. 1991 October; 88(4):661-74.
Breuer K, Kapp A, Werfel T. Urine eosinophil protein X (EPX) is an in vitro parameter of inflammation in atopic dermatitis of the adult age. Allergy. 2001 August; 56(8):780-4.
Burd E M. Human papillomavirus and cervical cancer. Clin Microbiol Rev. 2003 January; 16(1):1-17.
Burrage M, Robinson A, Borrow R, Andrews N, Southern J, Findlow J, Martin S, Thornton C, Goldblatt D, Corbel M, Sesardic D, Cartwight K, Richmond P, Miller E. Effect of vaccination with carrier protein on response to meningococcal C conjugate vaccines and value of different immunoassays as predictors of protection. Infect Immun. 2002 September; 70(9):4946-54.
Bush L M, Abrams B H, Beall A, Johnson C C. Index case of fatal inhalational anthrax due to bioterrorism in the United States. N Engl J. Med. 2001 Nov. 29; 345(22):1607-10. Epub 2001 Nov. 08.
Carus W S. Biological warfare threats in perspective. Crit Rev Microbiol. 1998; 24(3): 149-55.
Caruso C, Aquino A, Candore G, Scola L, Colonna-Romano G, Lio D. Looking for immunological risk genotypes. Ann N Y Acad. Sci. 2004 June; 1019:141-6.
Cates W Jr, Wasserheit J N. Genital chlamydial infections: epidemiology and reproductive sequelae. Am J Obstet Gynecol. 1991 June; 164(6 Pt 2):1771-81.
Cates W Jr. Estimates of the incidence and prevalence of sexually transmitted diseases in the United States. American Social Health Association Panel. Sex Transm Dis. 1999 April; 26(4 Suppl):S2-7.
Centers for Disease Control and Prevention. Epidemiology and Prevention of Vaccine-Preventable Diseases. Atkinson W, Hamborsky J, Wolfe S, eds. 8^{th} ed. Washington, D.C.: Public Health Foundation. 2004.
Centers for Disease Control and Prevention. 2002. General recommendations on immunization: recommendations of the Advisory Committee on Immunization Practices (ACIP). MMWR 51 (No. RR-2): 18-19.
Centers for Disease Control and Prevention. 1999. Ten great public health achievements—United States, 1900-1999. Morb. Mortal. Wkly. Rep. 48(12): 241-243.
Chang D M, Su W L, Chu S J. The expression and significance of intracellular T helper cytokines in systemic lupus erythematosus. Immunol Invest. 2002 February; 31(1):1-12.
Chaouat G, Ledee-Bataille N, Dubanchet S, Zourbas S, Sandra O, Martal J. TH1/TH2 paradigm in pregnancy: paradigm lost? Cytokines in pregnancy/early abortion: reexamining the TH1/TH2 paradigm. Int Arch Allergy Immunol. 2004 June; 134(2):93-119. Epub 2004 May 17.
Cheek J E, Baron R, Atlas H, Wilson D L, Crider R D Jr. Mumps outbreak in a highly vaccinated school population. Evidence for large-scale vaccination failure. Arch Pediatr Adolesc Med. 1995 July; 149(7):774-8.
Chen M, Sonnerborg A, Sallberg M. Levels of hepatitis C virus (HCV) RNA in serum and their relationship to levels of immunoglobulin M and G antibodies against HCV core protein. J Clin Microbiol. 1995 March; 33(3):778-80.
Chiba J, Ohba H, Matsuura Y, Watanabe Y, Katayama T, Kikuchi S, Saito I, Miyamura T. Serodiagnosis of hepatitis C virus (HCV) infection with an HCV core protein molecularly expressed by a recombinant baculovirus. Proc Natl Acad Sci USA. 1991 Jun. 1; 88(11):4641-5.
Christie C D, Glover A M, Willke M J, Marx M L, Reising S F, Hutchinson N M. Containment of pertussis in the regional pediatric hospital during the Greater Cincinnati epidemic of 1993. Infect Control Hosp Epidemiol. 1995 October; 16(10):556-63.
Chtanova T, Tangye S G, Newton R, Frank N, Hodge M R, Rolph M S, Mackay C R. T follicular helper cells express a distinctive transcriptional profile, reflecting their role as non-Th1/Th2 effector cells that provide help for B cells. J. Immunol. 2004 Jul. 1; 173(1):68-78.
Cochi S L, Preblud S R, Orenstein W A. Perspectives on the relative resurgence of mumps in the United States. Am J Dis Child. 1988 May; 142(5):499-507.
Cockerill F R 3rd, Smith T F. Response of the clinical microbiology laboratory to emerging (new) and reemerging infectious diseases. J Clin Microbiol. 2004 June; 42(6):2359-65.
Cohen M B, Giannella R A, Bean J, Taylor D N, Parker S, Hoeper A, Wowk S, Hawkins J, Kochi S K, Schiff G, Killeen K P. Randomized, controlled human challenge study of the safety, immunogenicity, and protective efficacy of a single dose of Peru-15, a live attenuated oral cholera vaccine. Infect Immun. 2002 April; 70(4):1965-70.
Combination Vaccines for Childhood Immunization. Recommendations of the Advisory Committee on Immunization Practices (ACIP), the American Academy of Pediatrics (AAP), and the American Academy of Family Physicians (AAFP). MMWR Recomm Rep. 1999 May 14; 48 (RR-5) 1-30.
Conrad M E, Lemon S M. Prevention of endemic icteric viral hepatitis by administration of immune serum gamma globulin. J Infect Dis. 1987 July; 156(1):56-63.
Cookson W. The immunogenetics of asthma and eczema: a new focus on the epithelium. Nat Rev Immunol. 2004 December; 4(12):978-88.
Cosmi L, Annunziato F, Maggi E, Romagnani S, Manetti R. Chemoattractant receptors expressed on type 2 T cells and their role in disease. Int Arch Allergy Immunol. 2001 August; 125(4):273-9.
Cox D L. Culture of Treponema pallidum. Methods Enzymol. 1994; 236:390-405.
Crockett S, Fein A M, Gall S A. Prevention of pneumococcal disease: use of pneumococcal polysaccharide vaccine. Am Med Assoc. 1999.
Crotty S, Felgner P, Davies H, Glidewell J, Villarreal L, Ahmed R. Cutting edge: long-term B cell memory in humans after smallpox vaccination. J. Immunol. 2003 Nov. 15; 171(10):4969-73.
Cunningham A F, Johnston S L, Julious S A, Lampe F C, Ward M E. Chronic Chlamydia pneumoniae infection and asthma exacerbations in children. Eur Respir J. 1998 February; 11(2):345-9.
Cunningham A L, Mikloska Z. The Holy Grail: immune control of human herpes simplex virus infection and disease. Herpes. 2001 March; 8 Suppl 1:6A-10A.
Curiel T J, Coukos G, Zou L, Alvarez X, Cheng P, Mottram P, Evdemon-Hogan M, Conejo-Garcia J R, Zhang L, Burow M, Zhu Y, Wei S, Kryczek I, Daniel B, Gordon A, Myers L, Lackner A, Disis M L, Knutson K L, Chen L, Zou W. Specific recruitment of regulatory T cells in ovarian carcinoma fosters immune privilege and predicts reduced survival. Nat Med. 2004 September; 10(9):942-9.
Da Silva J A, Spector T D. The role of pregnancy in the course and aetiology of rheumatoid arthritis. Clin Rheumatol. 1992 June; 11(2):189-94.
Darling R G, Catlett C L, Huebner K D, Jarrett D G. Threats in bioterrorism. I: CDC category A agents. Emerg Med Clin North Am. 2002 May; 20(2):273-309.
Del Prete G F, De Carli M, Ricci M, Romagnani S. Helper activity for immunoglobulin synthesis of T helper type 1 (Th1) and Th2 human T cell clones: the help of Th1 clones is limited by their cytolytic capacity. J Exp Med. 1991 Oct. 1; 174(4):809-13.
Dennehy P H. Active immunization in the United States: developments over the past decade. Clin Microbiol Rev. 2001 October; 14(4):872-908.
De Silvestri A, Pasi A, Martinetti M, Belloni C, Tinelli C, Rondini G, Salvaneschi L, Cuccia M. Family study of non-responsiveness to hepatitis B vaccine confirms the importance of HLA class III C4A locus. Genes Immun. 2001 November; 2(7):367-72.
Dinarello C A. Biologic basis for interleukin-1 in disease. Blood 1996 March 15; 87: 2095-2147.
Donnadieu E, Jouvin M H, Rana S, Moffatt M F, Mockford E H, Cookson W O, Kinet J P. Competing functions encoded in the allergy-associated F(c)epsilonRIbeta gene. Immunity. 2003 May; 18(5):665-74.
Douglas S D, Durako S, Sullivan K E, Camarca M, Moscicki A B, Wilson C M. TH1 and TH2 cytokine mRNA and protein levels in human immunodeficiency virus (HIV)-seropositive and HIV-seronegative youths. Clin Diagn Lab Immunol. 2003 May; 10(3):399-404.
Doyle A G, Buttigieg K, Groves P, Johnson B J, Kelso A. The activated type 1-polarized CD8(+) T cell population isolated from an effector site contains cells with flexible cytokine profiles. J Exp Med. 1999 Oct. 18; 190(8):1081-92.
Dudley M E, Rosenberg S A. Adoptive-cell-transfer therapy for the treatment of patients with cancer. Nat Rev Cancer. 2003 September; 3(9):666-75.
Eder W, Klimecki W, Yu L, von Mutius E, Riedler J, Braun-Fahrlander C, Nowak D, Martinez F D; ALEX Study Team. Toll-like receptor 2 as a major gene for asthma in children of European farmers. J Allergy Clin Immunol. 2004 March; 113(3):482-8.
Edlich R F, Diallo A O, Buchanan L, Martin M L. Hepatitis B virus: a comprehensive strategy for eliminating transmission in the United States. J Long Term Eff Med Implants. 2003; 13(2):117-25.
Edmonson W P, Purcell R H, Gundelfinger B F, Love J W, Ludwig W, Chanock R M.

Immunization by selective infection with type 4 adenovirus grown in human diploid tissue culture. II. specific protective effect against epidemic disease. JAMA. 1966; 195: 453-459.
Edwards K M, Decker M D, Graham B S, Mezzatesta J, Scott J, Hackell J. Adult immunization with acellular pertussis vaccine. JAMA. 1993 Jan. 6; 269(1):53-6.
Ehrenkranz N J, Meyer K F. Studies on immunization against plague. VIII. Study of three immunizing preparations in protecting primates against pneumonic plague. J Infect Dis. 1955 March-April; 96(2):138-44.
Ehrenstein M R, Evans J G, Singh A, Moore S, Warnes G, Isenberg D A, Mauri C. Compromised function of regulatory T cells in rheumatoid arthritis and reversal by anti-TNFalpha therapy. J Exp Med. 2004 Aug. 2; 200(3):277-85.
El Biaze M, Boniface S, Koscher V, Mamessier E, Dupuy P, Milhe F, Ramadour M, Vervloet D, Magnan A. T cell activation, from atopy to asthma: more a paradox than a paradigm. Allergy. 2003 September; 58(9):844-53.
Elie C M, Holder P K, Romero-Steiner S, Carlone G M. Assignment of additional anticapsular antibody concentrations to the Neisseria meningitidis group A, C, Y, and W-135 meningococcal standard reference serum CDC1992. Clin Diagn Lab Immunol. 2002 May; 9(3):725-6.
Engelkens H J, ten Kate F J, Judanarso J, Vuzevski V D, van Lier J B, Godschalk J C, van der Sluis J J, Stolz E. The localisation of treponemes and characterisation of the inflammatory infiltrate in skin biopsies from patients with primary or secondary syphilis, or early infectious yaws. Genitourin Med. 1993 April; 69(2):102-7.
Esposito S, Agliardi T, Giammanco A, Faldella G, Cascio A, Bosis S, Friscia O, Clerici M, Principi N. Long-term pertussis-specific immunity after primary vaccination with a combined diphtheria, tetanus, tricomponent acellular pertussis, and hepatitis B vaccine in comparison with that after natural infection. Infect Immun. 2001 July; 69(7):4516-20.
European Consensus Group on Hepatitis B Immunity. Are booster immunisations needed for lifelong hepatitis B immunity? European Consensus Group on Hepatitis B Immunity. Lancet. 2000 Feb. 12; 355(9203):561-5.
Evans M E, Friedlander A M. 1997. Tularemia. Medical Aspects of Chemical and Biological Warfare. Textbook of Military Medicine Series. Part I. Warfare, Weaponry, and Casualty. Sidell F R, Takafuji E T, Franz D R, eds. TMM Publications, Washington, D.C.
Fearon D T, Austen K F. Properdin: binding to C3b and stabilization of the C3b-dependent C3 convertase. J Exp Med. 1975 Oct. 1; 142(4):856-63.
Fenner F, Henderson D A, Arita I, Jezek Z, Ladnyi I D. 1988. Smallpox and Its Eradication. World Health Organization. Geneva.
Figueroa J E, Densen P. Infectious diseases associated with complement deficiencies. Clin Microbiol Rev. 1991 July; 4(3):359-95.
Fitzpatrick D R, Shirley K M, Kelso A. Cutting edge: stable epigenetic inheritance of regional IFN-gamma promoter demethylation in CD44highCD8+T lymphocytes. J. Immunol. 1999 May 1; 162(9):5053-7.
Flashner Y, Mamroud E, Tidhar A, Ber R, Aftalion M, Gur D, Lazar S, Zvi A, Bino T, Ariel N, Velan B, Shafferman A, Cohen S. Generation of Yersinia pestis attenuated strains by signature-tagged mutagenesis in search of novel vaccine candidates. Infect Immun. 2004 February; 72(2):908-15.
Fleming D T, McQuillan G M, Johnson R E, Nahmias A J, Aral S O, Lee F K, St Louis M E. Herpes simplex virus type 2 in the United States, 1976 to 1994. N Engl J. Med. 1997 Oct. 16; 337(16):1105-11.
Fletcher M A. Vaccine candidates in STD. Int J STD AIDS. 2002 December; 13 Suppl 2:38-41.
Fogarty K J, Massoudi M S, Gallo W, Averhoff F M, Yusuf H, Fishbein D. Vaccine coverage levels after implementation of a middle school vaccination requirement, Florida, 1997-2000. Public Health Rep. 2004 March-April; 119(2):163-9.
Fox J P. Modes of action of poliovirus vaccines and relation to resulting immunity. Rev Infect Dis. 1984 May-June; 6 Suppl 2:S352-5.
Franco E L. Cancer causes revisited: human papillomavirus and cervical neoplasia. J Natl Cancer Inst. 1995 Jun. 7; 87(11):779-80.
Frank J A Jr, Orenstein W A, Bart K J, Bart S W, el-Tantawy N, Davis R M, Hinman A R. Major impediments to measles elimination. The modern epidemiology of an ancient disease. Am J Dis Child. 1985 September; 139(9):881-8.
Fulop T, Larbi A, Wikby A, Mocchegiani E, Hirokawa K, Pawelec G. Dysregulation of T-cell function in the elderly: scientific basis and clinical implications. Drugs Aging. 2005; 22(7):589-603.
Gattinoni L, Klebanoff C A, Palmer D C, Wrzesinski C, Kerstann K, Yu Z, Finkelstein S E, Theoret M R, Rosenberg S A, Restifo N P. Acquisition of full effector function in vitro paradoxically impairs the in vivo antitumor efficacy of adoptively transferred CD8+ T cells. J Clin Invest. 2005 June; 115(6):1616-26.
Gencay M, Rudiger J J, Tamm M, Soler M, Perruchoud A P, Roth M. Increased frequency of Chlamydia pneumoniae antibodies in patients with asthma. Am J Respir Crit Care Med. 2001 April; 163(5):1097-100.
Gerbase A C, Rowley J T, Mertens T E. Global epidemiology of sexually transmitted diseases. Lancet. 1998; 351 Suppl 3:2-4.
Gergen P J, McQuillan G M, Kiely M, Ezzati-Rice T M, Sutter R W, Virella G. A population-based serologic survey of immunity to tetanus in the United States. N Engl J. Med. 1995 Mar. 23; 332(12):761-6.
Gerli R, Bistoni O, Russano A, Fiorucci S, Borgato L, Cesarotti M E, Lunardi C. In vivo activated T cells in rheumatoid synovitis. Analysis of Th1- and Th2-type cytokine production at clonal level in different stages of disease. Clin Exp Immunol. 2002 September; 129(3):549-55.
Gindler J S, Atkinson W L, Markowitz L E, Hutchins S S. Epidemiology of measles in the United States in 1989 and 1990. Pediatr Infect Dis J. 1992 October; 11(10):841-6.
Goldblatt D, Richmond P, Millard E, Thornton C, Miller E. The induction of immunologic memory after vaccination with Haemophilus influenzae type b conjugate and acellular pertussis-containing diphtheria, tetanus, and pertussis vaccine combination. J Infect Dis. 1999 August; 180(2):538-41.
Goodman R A. Nosocomial hepatitis A. Ann Intern Med. 1985 September; 103(3):452-4.
Granoff D M, Cates K L. Haemophilus influenzae type b polysaccharide vaccines. J Pediatr. 1985 September; 107(3):330-6.
Granoff D M, Gupta R K, Belshe R B, Anderson E L. Induction of immunologic refractoriness in adults by meningococcal C polysaccharide vaccination. J Infect Dis. 1998 September; 178(3):870-4.
Gray G C, Callahan J D, Hawksworth A W, Fisher C A, Gaydos J C. Respiratory diseases among U.S. military personnel: countering emerging threats. Emerg Infect Dis. 1999 May-June; 5(3):379-85.
Gray G C, Goswami P R, Malasig M D, Hawksworth A W, Trump D H, Ryan M A, Schnurr D P. Adult adenovirus infections: loss of orphaned vaccines precipitates military respiratory disease epidemics. For the Adenovirus Surveillance Group. Clin Infect Dis. 2000 September; 31(3):663-70.
Greaves W L, Orenstein W A, Stetler H C, Preblud S R, Hinman A R, Bart K J. Prevention of rubella transmission in medical facilities. JAMA. 1982 Aug. 20; 248(7):861-4.
Hadler S C, Margolis H S. Hepatitis B immunization: vaccine types, efficacy, and indications for immunization. Curr Clin Top Infect Dis. 1992; 12:282-308.
Hahn D L, Dodge R W, Golubjatnikov R. Association of Chlamydia pneumoniae (strain TWAR) infection with wheezing, asthmatic bronchitis, and adult-onset asthma. JAMA. 1991 Jul. 10; 266(2):225-30.
Hamid Q A, Schotman E, Jacobson M R, Walker S M, Durham S R. Increases in IL-12 messenger RNA+ cells accompany inhibition of allergen-induced late skin responses after successful grass pollen immunotherapy. J Allergy Clin Immunol. 1997 February; 99(2):254-60.
Hammarlund E, Lewis M W, Hansen S G, Strelow L I, Nelson J A, Sexton G J, Hanifin J M, Slifka M K. Duration of antiviral immunity after smallpox vaccination. Nat Med. 2003 September; 9(9):1131-7.
Hansen G, Berry G, DeKruyff R H, Umetsu D T. Allergen-specific Th1 cells fail to counterbalance Th2 cell-induced airway hyperreactivity but cause severe airway inflammation. J Clin Invest. 1999 January; 103(2):175-83.
Harber M, Sundstedt A, Wraith D. The role of cytokines in immunological tolerance: potential for therapy. Expert Rev Mol Med. 2000 Nov. 27; 2000:1-20. Print 2000 Nov. 27.
Harro C D, Pang Y Y, Roden R B, Hildesheim A, Wang Z, Reynolds M J, Mast T C, Robinson R, Murphy B R, Karron R A, Dillner J, Schiller J T, Lowy D R. Safety and immunogenicity trial in adult volunteers of a human papillomavirus 16 L1 virus-like particle vaccine. J Natl Cancer Inst. 2001 Feb. 21; 93(4):284-92.
Hemachudha T, Mitrabhakdi E, Wilde H, Vejabhuti A, Siripataravanit S, Kingnate D. Additional reports of failure to respond to treatment after rabies exposure in Thailand. Clin Infect Dis. 1999 January; 28(1):143-4.
Henderson D A, Inglesby T V, Bartlett J G, Ascher M S, Eitzen E, Jahrling P B, Hauer J, Layton M, McDade J, Osterholm M T, O'Toole T, Parker G, Perl T, Russell P K, Tonat K. Smallpox as a biological weapon: medical and public health management. Working Group on Civilian Biodefense. JAMA. 1999 Jun. 9; 281(22):2127-37.
Henderson D A, Inglesby T V, Bartlett J G, Ascher M S, Eitzen E, Jahrling P B, Hauer J, Layton M, McDade J, Osterholm M T, O'Toole T, Parker G, Perl T, Russell P K, Tonat K. Smallpox as a biological weapon: medical and public health management. Working Group on Civilian Biodefense. JAMA. 1999 Jun. 9; 281(22):2127-37.
Henderson D A. Smallpox: clinical and epidemiologic features. Emerg Infect Dis. 1999 July-August; 5(4):537-9.
Hewlett, E. L. (2000). Bordatella species. In Mandell, G. L., Bennett, J. E. and Dolin, R. (Eds.). Mandell, Douglas and Bennett's Principles and Practice of Infectious Disease. [Electronic Version]. (pp. 2413-2422). Churchill Livingstone: Philadelphia.
Hibberd M L, Sumiya M, Summerfield J A, Booy R, Levin M. Association of variants of the gene for mannose-binding lectin with susceptibility to meningococcal disease. Meningococcal Research Group. Lancet. 1999 Mar. 27; 353(9158):1049-53.
Hoare S, El-Shazali O, Clark J E, Fay A, Cant A J. Investigation for complement deficiency following meningococcal disease. Arch Dis Child. 2002 March; 86(3):215-7.
Hogan R J, Mathews S A, Mukhopadhyay S, Summersgill J T, Timms P. Chlamydial persistence: beyond the biphasic paradigm. Infect Immun. 2004 April; 72(4): 1843-55.
Holder P K, Maslanka S E, Pais L B, Dykes J, Plikaytis B D, Carlone G M. Assignment of Neisseria meningitidis serogroup A and C class-specific anticapsular antibody concentrations to the new standard reference serum CDC1992. Clin Diagn Lab Immunol. 1995 March; 2(2):132-7.
Holt P G, Macaubas C, Stumbles P A, Sly P D. The role of allergy in the development of asthma. Nature. 1999 Nov. 25; 402(6760 Suppl):B 12-7.
Holt P G. A potential vaccine strategy for asthma and allied atopic diseases during early childhood. Lancet. 1994 Aug. 13; 344(8920):456-8.
Hooper L V, Gordon J I. Commensal host-bacterial relationships in the gut. Science. 2001 May 11; 292(5519):1115-8.
House R V. Cytokine measurement techniques for assessing hypersensitivity. Toxicology. 2001 Feb. 2; 158(1-2):51-8.
House R V. Theory and practice of cytokine assessment in immunotoxicology. Methods. 1999 September; 19(1):17-27.
Hoyert D L, Kochanek K D, Murphy S L. Deaths: final data for 1997. Natl Vital Stat Rep. 1999 Jun. 30; 47(19):1-104.
Igietseme J U, Eko F O, Black C M. Contemporary approaches to designing and evaluating vaccines against Chlamydia. Expert Rev Vaccines. 2003 February; 2(1): 129-46.
Immunization of health-care workers: recommendations of the Advisory Committee on Immunization Practices (ACIP) and the Hospital Infection Control Practices Advisory Committee (HICPAC). MMWR Recomm Rep. 1997 Dec. 26; 46(RR-18):1-42.
Jefferson T, Rivetti D, Rivetti A, Rudin M, Di Pietrantonj C, Demicheli V. Efficacy and effectiveness of influenza vaccines in elderly people: a systematic review. Lancet. 2005 Oct. 1; 366(9492): 1165-74.
Jodar L, Butler J, Carlone G, Dagan R, Goldblatt D, Kayhty H, Klugman K, Plikaytis B, Siber G, Kohberger R, Chang I, Cherian T. Serological criteria for evaluation and licensure of new pneumococcal conjugate vaccine formulations for use in infants. Vaccine. 2003 Jul. 4; 21(23):3265-72.
Jokhdar H, Borrow R, Sultan A, Adi M, Riley C, Fuller E, Baxter D. Immunologic hyporesponsiveness to serogroup C but not serogroup A following repeated meningococcal A/C polysaccharide vaccination in Saudi Arabia. Clin Diagn Lab Immunol. 2004 January; 11(1):83-8.
Jordan E K, McFarland H I, Lewis B K, Tresser N, Gates M A, Johnson M, Lenardo M, Matis L A, McFarland H F, Frank J A. Serial MR imaging of experimental autoimmune encephalomyelitis induced by human white matter or by chimeric myelin-basic and proteolipid protein in the common marmoset. AJNR Am J Neuroradiol. 1999 June-July; 20(6):965-76.
Kabesch M, Peters W, Carr D, Leupold W, Weiland S K, von Mutius E. Association between polymorphisms in caspase recruitment domain containing protein 15 and allergy in two German populations. J Allergy Clin Immunol. 2003 April; 111(4):813-7.
Kaplan A P. Chemokines, chemokine receptors and allergy. Int Arch Allergy Immunol. 2001 April; 124(4):423-31.
Kaplan K M, Marder D C, Cochi S L, Preblud S R. Mumps in the workplace. Further evidence of the changing epidemiology of a childhood vaccine-preventable disease. JAMA. 1988 Sep. 9; 260(10):1434-8.
Kasper D L, Paoletti L C, Wessels M R, Guttormsen H K, Carey V J, Jennings H J, Baker C J. Immune response to type III group B streptococcal polysaccharide-tetanus toxoid conjugate vaccine. J Clin Invest. 1996 Nov. 15; 98(10):2308-14.
Kaul D, Ogra P L. Mucosal responses to parenteral and mucosal vaccines. Dev Biol Stand. 1998; 95:141-6.
Kelso A, Groves P, Ramm L, Doyle A G. Single-cell analysis by RT-PCR reveals differential expression of multiple type 1 and 2 cytokine genes among cells within polarized CD4+ T cell populations. Int Immunol. 1999 April; 11 (4):617-21.
Kelso A, Groves P. A single peripheral CD8+ T cell can give rise to progeny expressing type 1 and/or type 2 cytokine genes and can retain its multipotentiality through many cell divisions. Proc Natl Acad Sci USA. 1997 Jul. 22; 94(15):8070-5.
Kelso A. Th1 and Th2 subsets: paradigms lost? Immunol Today. 1995 August; 16(8):374-9. Kidd P. Th1/Th2 balance: the hypothesis, its limitations, and implications for health and disease. Altern Med Rev. 2003 August; 8(3):223-46.
Klatt T, Ouyang Q, Flad T, Koetter I, Buhring H J, Kalbacher H, Pawelec G, Muller C A. Expansion of peripheral CD8+ CD28− T cells in response to Epstein-Barr virus in patients with rheumatoid arthritis. J Rheumatol. 2005 February; 32(2):239-51.
Klugman K P, Koornhof H J, Robbins J B, Le Cam N N. Immunogenicity, efficacy and serological correlate of protection of Salmonella typhi Vi capsular polysaccharide vaccine three years after immunization. Vaccine. 1996 April; 14(5):435-8.
Koren E, Zuckerman L A, Mire-Sluis A R. Immune responses to therapeutic proteins in humans—clinical significance, assessment and prediction. Curr Pharm Biotechnol. 2002 December; 3(4):349-60.
Kriegel M A, Lohmann T, Gabler C, Blank N, Kalden J R, Lorenz H M. Defective suppressor function of human CD4+ CD25+ regulatory T cells in autoimmune polyglandular syndrome type II. J Exp Med. 2004 May 3; 199(9):1285-91.
Kunzendorf U, Tran T H, Bulfone-Paus S. The Th1-Th2 paradigm in 1998: law of nature or rule with exceptions. Nephrol Dial Transplant. 1998 October; 13(10):2445-8.
Kuo C C, Jackson L A, Campbell L A, Grayston J T. Chlamydia pneumoniae (TWAR). Clin Microbiol Rev. 1995 October; 8(4):451-61.
Kuo C C, Shor A, Campbell L A, Fukushi H, Patton D L, Grayston J T. Demonstration of Chlamydia pneumoniae in atherosclerotic lesions of coronary arteries. J Infect Dis. 1993 April; 167(4):841-9.
Kuperman D A, Huang X, Koth L L, Chang G H, Dolganov G M, Zhu Z, Elias J A, Sheppard D, Erle D J. Direct effects of interleukin-13 on epithelial cells cause airway hyperreactivity and mucus overproduction in asthma. Nat Med. 2002 August; 8(8):885-9. Epub 2002 Jul. 01.
Kurashima C, Utsuyama M, Kasai M, Ishijima S A, Konno A, Hirokawa K. The role of thymus in the aging of Th cell subpopulations and age-associated alteration of cytokine production by these cells. Int Immunol. 1995 January; 7(1):97-104.
Kurt T L, Yeager A S, Guenette S, Dunlop S. Spread of pertussis by hospital staff. JAMA. 1972 Jul. 17; 221(3):264-7.
Larche M, Robinson D S, Kay A B. The role of T lymphocytes in the pathogenesis of asthma. J Allergy Clin Immunol. 2003 March; 111(3):450-63; quiz 464.
Laurila A L, Von Hertzen L, Saikku P. Chlamydia pneumoniae and chronic lung diseases. Scand J Infect Dis Suppl. 1997; 104:34-6.
Lazarus R, Raby B A, Lange C, Silverman E K, Kwiatkowski D J, Vercelli D, Klimecki W J, Martinez F D, Weiss S T. TOLL-like receptor 10 genetic variation is associated with asthma in two independent samples. Am J Respir Crit Care Med. 2004 Sep. 15; 170(6):594-600. Epub 2004 Jun. 16.
Leach A, Twumasi P A, Kumah S, Banya W S, Jaffar S, Forrest B D, Granoff D M, LiButti D E, Carlone G M, Pais L B, Broome C V, Greenwood B M. Induction of immunologic memory in Gambian children by vaccination in infancy with a group A plus group C meningococcal polysaccharide-protein conjugate vaccine. J Infect Dis. 1997 January; 175(1):200-4.
Lemckert A A, Goudsmit J, Barouch D H. Challenges in the search for an HIV vaccine. Eur J Epidemiol. 2004; 19(6):513-6.
Lemon, S M, Thaul S, Fisseha S, O'Maonaigh H C. eds. Protecting our Forces: Improving Vaccine Acquisition and Availability in the U.S. Military. 2002. The National Academies Press, Washington, D.C.
Leroux-Roels G, Cao T, De Knibber A, Meuleman P, Roobrouck A, Farhoudi A, Vanlandschoot P, Desombere I. Prevention of hepatitis B infections: vaccination and its limitations. Acta Clin Belg. 2001 July-August; 56(4):209-19.
Licastro F, Candore G, Lio D, Porcellini E, Colonna-Romano G, Franceschi C, Caruso C. Innate immunity and inflammation in ageing: a key for understanding age-related diseases. Immun Ageing. 2005 May 18; 2:8.
Lienhardt C, Azzurri A, Amedei A, Fielding K, Sillah J, Sow O Y, Bah B, Benagiano M, Diallo A, Manetti R, Manneh K, Gustafson P, Bennett S, D'Elios M M, McAdam K, Del Prete G. Active tuberculosis in Africa is associated with reduced Th1 and increased Th2 activity in vivo. Eur J. Immunol. 2002 June; 32(6):1605-13.
Lin F Y, Weisman L E, Azimi P H, Philips J B 3rd, Clark P, Regan J, Rhoads G G, Frasch C E, Gray B M, Troendle J, Brenner R A, Moyer P, Clemens J D. Level of maternal IgG anti-group B streptococcus type III antibody correlated with protection of neonates against early-onset disease caused by this pathogen. J Infect Dis. 2004 Sep. 1; 190(5):928-34.
Lin Y C, Chang M H, Ni Y H, Hsu H Y, Chen D S. Long-term immunogenicity and efficacy of universal hepatitis B virus vaccination in Taiwan. J Infect Dis. 2003 Jan. 1; 187(1):134-8. Epub 2002 Dec. 13.
Linton S M, Morgan B P. Properdin deficiency and meningococcal disease—identifying those most at risk. Clin Exp Immunol. 1999 November; 118(2):189-91.
Little S F, Ivins B E, Fellows P F, Pitt M L, Norris S L, Andrews G P. Defining a serological correlate of protection in rabbits for a recombinant anthrax vaccine. Vaccine. 2004 Jan. 2; 22(3-4):422-30.
Lucas A H, Granoff D M. A major crossreactive idiotype associated with human antibodies to the Haemophilus influenzae b polysaccharide. Expression in relation to age and immunoglobulin G subclass. J Clin Invest. 1990 April; 85(4):1158-66.
Lucas A H, Langley R J, Granoff D M, Nahm M H, Kitamura M Y, Scott M G. An idiotypic marker associated with a germ-line encoded kappa light chain variable region that predominates the vaccine-induced human antibody response to the Haemophilus influenzae b polysaccharide. J Clin Invest. 1991 December; 88(6):1811-8.
Lucey D R, Clerici M, Shearer G M. Type 1 and type 2 cytokine dysregulation in human infectious, neoplastic, and inflammatory diseases. Clin Microbiol Rev. 1996 October; 9(4):532-62.
Lukehart S A, Baker-Zander S A, Lloyd R M, Sell S. Characterization of lymphocyte responsiveness in early experimental syphilis. II. Nature of cellular infiltration and Treponema pallidum distribution in testicular lesions. J. Immunol. 1980 January; 124(1):461-7.
Mackall C L, Fleisher T A, Brown M R, Andrich M P, Chen C C, Feuerstein I M, Horowitz M E, Magrath I T, Shad A T, Steinberg S M, et al. Age, thymopoiesis, and CD4+ T-lymphocyte regeneration after intensive chemotherapy. N Engl J. Med. 1995 Jan. 19; 332(3): 143-9.
Mackay C R. Follicular homing T helper (Th) cells and the Th1/Th2 paradigm. J Exp Med. 2000 Dec. 4; 192(11):F31-4.
Magnan A O, Mely L G, Camilla C A, Badier M M, Montero-Julian F A, Guillot C M, Casano B B, Prato S J, Fert V, Bongrand P, Vervloet D. Assessment of the Th1/Th2 paradigm in whole blood in atopy and asthma. Increased IFN-gamma-producing CD8(+) T cells in asthma. Am J Respir Crit Care Med. 2000 June; 161(6):1790-6.
Mannino D M, Homa D M, Pertowski C A, Ashizawa A, Nixon L L, Johnson C A, Ball L B, Jack E, Kang D S. Surveillance for asthma—United States, 1960-1995. MMWR CDC Surveill Summ. 1998 Apr. 24; 47(1):1-27.
Matzinger P. The danger model: a renewed sense of self. Science. 2002 Apr. 12; 296(5566):301-5.
Mays R M, Sturm L A, Zimet G D. Parental perspectives on vaccinating children against sexually transmitted infections. Soc Sci Med. 2004 April; 58(7):1405-13.
Mazzoni A, Segal D M. Controlling the Toll road to dendritic cell polarization. J Leukoc Biol. 2004 May; 75(5):721-30. Epub 2004 Jan 14.
McAdam K P, Recent progress in bacterial vaccines: tuberculosis. Int J Infect Dis 1997; 1:172-8.
McCrumb F R Jr, Snyder M J, Woodward T E. Studies on human infection with Pasteurella tularensis; comparison of streptomycin and chloramphenicol in the prophylaxis of clinical disease. Trans Assoc Am Physicians. 1957; 70:74-9; discussion 79-80.

McGovern T W, Christopher G W, Eitzen E M. Cutaneous manifestations of biological warfare and related threat agents. Arch Dermatol. 1999 March; 135(3):311-22.
McNeill K M, Hendrix R M, Lindner J L, Benton F R, Monteith S C, Tuchscherer M A, Gray G C, Gaydos J C. Large, persistent epidemic of adenovirus type 4-associated acute respiratory disease in U.S. army trainees. Emerg Infect Dis. 1999 November-December; 5(6):798-801.
McIntire J J, Umetsu S E, Macaubas C, Hoyte E G, Cinnioglu C, Cavalli-Sforza L L, Barsh G S, Hallmayer J F, Underhill P A, Risch N J, Freeman G J, DeKruyff R H, Umetsu D T. Immunology: hepatitis A virus link to atopic disease. Nature. 2003 Oct. 9; 425(6958):576.
Meeting Summary: AIDS Vaccine Trials, Considerations for Phase III Trial Design and Endpoints. National Institutes of Health, NIAID, Nov. 16, 2001.
Meltzer M I, Damon I, LeDuc J W, Millar J D. Modeling potential responses to smallpox as a bioterrorist weapon. Emerg Infect Dis. 2001 November-December; 7(6):959-69.
Meltzer M I, Dennis D T, Orloski K A. The cost effectiveness of vaccinating against Lyme disease. Emerg Infect Dis. 1999 May-June; 5(3):321-8.
Middlebrook J L, Franz D R. 1998. Medical Management of Biological Casualties. TMM Publications, Washington D.C.
Miller R A, Chrisp C. T cell subset patterns that predict resistance to spontaneous lymphoma, mammary adenocarcinoma, and fibrosarcoma in mice. J. Immunol. 2002 Aug. 1; 169(3):1619-25.

Milligan G N, Bernstein D I, Bourne N. T lymphocytes are required for protection of the vaginal mucosae and sensory ganglia of immune mice against reinfection with herpes simplex virus type 2. J. Immunol. 1998 Jun. 15; 160(12):6093-100.
Moffatt M F, Cookson W O. Tumour necrosis factor haplotypes and asthma. Hum Mol Genet. 1997 April; 6(4):551-4.
Montaner L J, da Silva R P, Sun J, Sutterwala S, Hollinshead M, Vaux D, Gordon S. Type 1 and type 2 cytokine regulation of macrophage endocytosis: differential activation by IL-4/IL-13 as opposed to IFN-gamma or IL-10. J. Immunol. 1999 Apr. 15; 162(8):4606-13.
Morgan C A, Lukehart S A, Van Voorhis W C. Protection against syphilis correlates with specificity of antibodies to the variable regions of Treponema pallidum repeat protein K. Infect Immun. 2003 October; 71(10):5605-12.
Mortimer E A Jr. Pertussis and its prevention: a family affair. J Infect Dis. 1990 March; 161 (3):473-9.
Mosmann T R, Cherwinski H, Bond M W, Giedlin M A, Coffman R L. Two types of murine helper T cell clone. I. Definition according to profiles of lymphokine activities and secreted proteins. J. Immunol. 1986 Apr. 1; 136(7):2348-57.
Mosmann T R, Coffman R L. TH1 and TH2 cells: different patterns of lymphokine secretion lead to different functional properties. Annu Rev Immunol. 1989; 7:145-73.
Moulder J W. Interaction of chlamydiae and host cells in vitro. Microbiol Rev. 1991 March; 55(1): 143-90.
Murray C J, Lopez A D. Mortality by cause for eight regions of the world: Global Burden of Disease Study. Lancet. 1997 May 3; 349(9061):1269-76.
Neuzil K M, Reed G W, Mitchel E F, Simonsen L, Griffin M R. Impact of influenza on acute cardiopulmonary hospitalizations in pregnant women. Am J Epidemiol. 1998 Dec. 1; 148(11):1094-102.
Newport M J, Huxley C M, Huston S, Hawrylowicz C M, Oostra B A, Williamson R, Levin M. A mutation in the interferon-gamma-receptor gene and susceptibility to mycobacterial infection. N Engl J. Med. 1996 Dec. 26; 335(26):1941-9.
Nigro G, Adler S P, La Torre R, Best A M; Congenital Cytomegalovirus Collaborating Group. Passive immunization during pregnancy for congenital cytomegalovirus infection. N Engl J. Med. 2005 Sep. 29; 353(13):1350-62.
Nishimura H, Nose M, Hiai H, Minato N, Honjo T. Development of lupus-like autoimmune diseases by disruption of the PD-1 gene encoding an ITIM motif-carrying immunoreceptor. Immunity. 1999 August; 11(2):141-51.
Noverr M C, Noggle R M, Toews G B, Huffnagle G B. Role of antibiotics and fungal microbiota in driving pulmonary allergic responses. Infect Immun. 2004 September; 72(9):4996-5003.
Nowalk M P, Bardella I J, Zimmerman R K, Shen S. The physician's office: can it influence adult immunization rates? Am J Manag Care. 2004 January; 10(1):13-9.
Nowalk M P, Zimmerman R K, Feghali J. Missed opportunities for adult immunization in diverse primary care office settings. Vaccine. 2004 Sep. 3; 22(25-26):3457-63.
Nowalk M P, Zimmerman R K, Shen S, Jewell I K, Raymund M. Barriers to pneumococcal and influenza vaccination in older community-dwelling adults (2000-2001). J Am Geriatr Soc. 2004 January; 52(1):25-30.
Ober C, Leavitt S A, Tsalenko A, Howard T D, Hoki D M, Daniel R, Newman D L, Wu X, Parry R, Lester L A, Solway J, Blumenthal M, King R A, Xu J, Meyers D A, Bleecker E R, Cox N J. Variation in the interleukin 4-receptor alpha gene confers susceptibility to asthma and atopy in ethnically diverse populations. Am J Hum Genet. 2000 February; 66(2):517-26.
Oguma T, Palmer L J, Birben E, Sonna L A, Asano K, Lilly C M. Role of prostanoid DP receptor variants in susceptibility to asthma. N Engl J. Med. 2004 Oct. 21; 351(17):1752-63.
Onorato I M, McCray E. Prevalence of human immunodeficiency virus infection among patients attending tuberculosis clinics in the United States. J Infect Dis. 1992 January; 165(1):87-92.
Onorato I M, Modlin J F, McBean A M, Thoms M L, Losonsky G A, Bernier R H. Mucosal immunity induced by enhance-potency inactivated and oral polio vaccines. J Infect Dis. 1991 January; 163(1):1-6.
Orenstein R, Wong E S. Urinary tract infections in adults. Am Fam Physician. 1999 Mar. 1; 59(5):1225-34, 1237.
O'Toole T. Smallpox: An attack scenario. Emerg Infect Dis. 1999 July-August; 5(4):540-6.
Oymar K. High levels of urinary eosinophil protein X in young asthmatic children predict persistent atopic asthma. Pediatr Allergy Immunol. 2001 December; 12(6):312-7.
Ouyang Q, Wagner W M, Wikby A, Walter S, Aubert G, Dodi A I, Travers P, Pawelec G. Large numbers of dysfunctional CD8+ T lymphocytes bearing receptors for a single dominant CMV epitope in the very old. J Clin Immunol. 2003 July; 23(4):247-57.
Overwijk W W, Theoret M R, Finkelstein S E, Surman DR, de Jong L A, Vyth-Dreese F A, Dellemijn T A, Antony P A, Spiess P J, Palmer D C, Heimann D M, Klebanoff C A, Yu Z, Hwang L N, Feigenbaum L, Kruisbeek A M, Rosenberg S A, Restifo N P. Tumor regression and autoimmunity after reversal of a functionally tolerant state of self-reactive CD8+ T cells. J Exp Med. 2003 Aug. 18; 198(4):569-80.
Pakala S V, Kurrer M O, Katz J D. T helper 2 (Th2) T cells induce acute pancreatitis and diabetes in immune-compromised nonobese diabetic (NOD) mice. J Exp Med. 1997 Jul. 21; 186(2):299-306.
Palaniyar N, Nadesalingam J, Reid K B. Pulmonary innate immune proteins and receptors that interact with gram-positive bacterial ligands. Immunobiology. 2002 September; 205(4-5):575-94.
Palazzi D L, Rench M A, Edwards M S, Baker C J. Use of type V group B streptococcal conjugate vaccine in adults 65-85 years old. J Infect Dis. 2004 Aug. 1; 190(3):558-64.
Paoletti L C, in Gram Positive Pathogens. Fischetti V A, ed. 2000. ASM Press. Washington D.C.
Paoletti L C, Kasper D L. Glycoconjugate vaccines to prevent group B streptococcal infections. Expert Opin Biol Ther. 2003 September; 3(6):975-84.
Pawelec G, Akbar A, Caruso C, Solana R, Grubeck-Loebenstein B, Wikby A. Human immunosenescence: is it infectious? Immunol Rev. 2005 June; 205:257-68.
Pawelec G, Barnett Y, Forsey R, Frasca D, Globerson A, McLeod J, Caruso C, Franceschi C, Fulop T, Gupta S, Mariani E, Mocchegiani E, Solana R. T cells and aging, January 2002 update. Front Biosci. 2002 May 1; 7:d1056-183.
Peltola H, Makela H, Kayhty H, Jousimies H, Herva E, Hallstrom K, Sivonen A, Renkonen O V, Pettay O, Karanko V, Ahvonen P, Sarna S. Clinical efficacy of meningococcus group A capsular polysaccharide vaccine in children three months to five years of age. N Engl J. Med. 1977 Sep. 29; 297(13):686-91.

Pier G B, Lyczak J B, Wetzler L M (eds). Immunology, Infection, and Immunity. 2004, ASM Press, American Society for Microbiology, Washington D.C., USA.
Platonov A E, Kuijper E J, Vershinina I V, Shipulin G A, Westerdaal N, Fijen C A, van de Winkel J G. Meningococcal disease and polymorphism of FcgammaRIIa (CD32) in late complement component-deficient individuals. Clin Exp Immunol. 1998 January; 111(1):97-101.
Platonov A E, Vershinina I V, Kuijper E J, Borrow R, Kayhty H. Long term effects of vaccination of patients deficient in a late complement component with a tetravalent meningococcal polysaccharide vaccine. Vaccine. 2003 Oct. 1; 21(27-30):4437-47.
Plotkin S A. Immunologic correlates of protection induced by vaccination. Pediatr Infect Dis J. 2001 January; 20(1):63-75.
Plotkin S A, Loupi E, Lang J. Acellular vaccine efficacy trials [Letter]. Pediatr Infect Dis J 1997; 16:913-4.
Plotkin S A. Rabies. Clin Infect Dis. 2000 January; 30(1):4-12.
Poehling K A, Speroff T, Dittus R S, Griffin M R, Hickson G B, Edwards K M. Predictors of influenza virus vaccination status in hospitalized children. Pediatrics. 2001 December; 108(6):E99.
Poland J D. Plague. In: Infectious Diseases: a modern treatise of infectious processes. 1989. JB Lippincott, Philadelphia, Pa.
Press, W., Teukolsky, S. Vetterling W., Flannery, B. Numerical Recipes in C The Art of Scientific Computing. Second Edition. 1992. Cambridge University Press.
Prokunina L, Castillejo-Lopez C, Oberg F, Gunnarsson I, Berg L, Magnusson V, Brookes A J, Tentler D, Kristjansdottir H, Grondal G, Bolstad A I, Svenungsson E, Lundberg I, Sturfelt G, Jonssen A, Truedsson L, Lima G, Alcocer-Varela J, Jonsson R, Gyllensten U B, Harley J B, Alarcon-Segovia D, Steinsson K, Alarcon-Riquelme M E. A regulatory polymorphism in PDCD1 is associated with susceptibility to systemic lupus erythematosus in humans. Nat Genet. 2002 December; 32(4):666-9. Epub 2002 Oct. 28.
Pulleyn L J, Newton R, Adcock I M, Barnes P J. TGFbeta1 allele association with asthma severity. Hum Genet. 2001 December; 109(6):623-7. Epub 2001 Oct. 27.
Racke M K, Bonomo A, Scott D E, Cannella B, Levine A, Raine C S, Shevach E M, Rocken M. Cytokine-induced immune deviation as a therapy for inflammatory autoimmune disease. J Exp Med. 1994 Nov. 1; 180(5):1961-6.
Read R C, Cannings C, Naylor S C, Timms J M, Maheswaran R, Borrow R, Kaczmarski E B, Duff G W. Variation within genes encoding interleukin-1 and the interleukin-1 receptor antagonist influence the severity of meningococcal disease. Ann Intern Med. 2003 Apr. 1; 138(7):534-41.
Richmond P, Kaczmarski E, Borrow R, Findlow J, Clark S, McCann R, Hill J, Barker M, Miller E. Meningococcal C polysaccharide vaccine induces immunologic hyporesponsiveness in adults that is overcome by meningococcal C conjugate vaccine. J Infect Dis. 2000 February; 181(2):761-4.
Ries L A G, Eisner M P, Kosary C L, Hankey B F, Miller B A, Clegg L, Mariotto A, Feuer E J, Edwards B K (eds). SEER Cancer Statistics Review, 1975-2001, National Cancer Institute. Bethesda, Md., http://seer.cancer.gov/csr/1975_{—}2001/2004.
Rietmeijer C, Lloyd L, Alfonsi, G. Trends in clinic visits and diagnosed C. trachomatis (CT) and N. gonorrhoeae (GC) infections following the introduction of a co-pay in an STD clinic. Presentation at the 2004 STD Prevention Conference. Mar. 8, 2004. Philadelphia, Pa.
Robbins J B, Parke J C Jr, Schneerson R, Whisnant J K. Quantitative measurement of “natural” and immunization-induced Haemophilus influenzae type b capsular polysaccharide antibodies. Pediatr Res. 1973 March; 7(3): 103-10.
Robbins J B. Pertussis in adults: introduction. Clin Infect Dis. 1999 June; 28 Suppl 2:S91-3.
Roden R B, Ling M, Wu T C. Vaccination to prevent and treat cervical cancer. Hum Pathol. 2004 August; 35(8):971-82.
Rogge L. A genomic view of helper T cell subsets. Ann N Y Acad. Sci. 2002 December; 975:57-67.
Romagnani S, Parronchi P, D'Elios M M, Romagnani P, Annunziato F, Piccinni M P, Manetti R, Sampognaro S, Mavilia C, De Carli M, Maggi E, Del Prete G F. An update on human Th1 and Th2 cells. Int Arch Allergy Immunol. 1997 May-July; 113(1-3):153-6.
Romagnani S. Human TH1 and TH2 subsets: doubt no more. Immunol Today. 1991 August; 12(8):256-7.
Romagnani S. The Th1/Th2 paradigm. Immunol Today. 1997 June; 18(6):263-6.
Romagnani S. The increased prevalence of allergy and the hygiene hypothesis: missing immune deviation, reduced immune suppression, or both? Immunology. 2004 July; 112(3):352-63.
Rook G A, Stanford J L. Give us this day our daily germs. Immunol Today. 1998 March; 19(3):113-6.
Rosenblum L S, Villarino M E, Nainan O V, Melish M E, Hadler S C, Pinsky P P, Jarvis W R, Ott C E, Margolis H S. Hepatitis A outbreak in a neonatal intensive care unit: risk factors for transmission and evidence of prolonged viral excretion among preterm infants. J Infect Dis. 1991 September; 164(3):476-82.
Rosenstein N E, Perkins B A, Stephens D S, Popovic T, Hughes J M. Meningococcal disease. N Engl J. Med. 2001 May 3; 344(18):1378-88.
Salk J. One-dose immunization against paralytic poliomyelitis using a noninfectious vaccine. Rev Infect Dis. 1984 May-June; 6 Suppl 2:S444-50.
Sallberg M, Ruden U, Wahren B, Magnius L O. Antigenic regions within the hepatitis C virus envelope 1 and non-structural proteins: identification of an IgG3-restricted recognition site with the envelope 1 protein. Clin Exp Immunol. 1993 March; 91(3):489-94.
Sallusto F, Lenig D, Mackay C R, Lanzavecchia A. Flexible programs of chemokine receptor expression on human polarized T helper 1 and 2 lymphocytes. J Exp Med. 1998 Mar. 16; 187(6):875-83.
Sallusto F, Mackay C R, Lanzavecchia A. The role of chemokine receptors in primary, effector, and memory immune responses. Annu Rev Immunol. 2000; 18:593-620.
Sanchez J L, Binn L N, Innis B L, Reynolds R D, Lee T, Mitchell-Raymundo F, Craig S C, Marquez J P, Shepherd G A, Polyak C S, Conolly J, Kohlhase K F. Epidemic of adenovirus-induced respiratory illness among US military recruits: epidemiologic and immunologic risk factors in healthy, young adults. J Med Virol. 2001 December; 65(4):710-8.
Schaerli P, Loetscher P, Moser B. Cutting edge: induction of follicular homing precedes effector Th cell development. J Immunol. 2001 Dec. 1; 167(11):6082-6.
Schellekens H. Immunogenicity of therapeutic proteins: clinical implications and future prospects. Clin Ther. 2002 November; 24(11):1720-40.
Schmidt B L. Evaluation of a new particle gel immunoassay for determination of antibodies against Treponema pallidum. J Clin Microbiol. 2004 June; 42(6):2833-5.
Schrag S J, Fiore A E, Gonik B, Malik T, Reef S, Singleton J A, Schuchat A, Schulkin J. Vaccination and perinatal infection prevention practices among obstetrician-gynecologists. Obstet Gynecol. 2003 April; 101(4):704-10.
Schulze-Koops H, Kalden J R. The balance of Th1/Th2 cytokines in rheumatoid arthritis. Best Pract Res Clin Rheumatol. 2001 December; 15(5):677-91.
Schwartz D H, Mazumdar A, Winston S, Harkonen S. Utility of various commercially available human immunodeficiency virus (HIV) antibody diagnostic kits for use in conjunction with efficacy trials of HIV-1 vaccines. Clin Diagn Lab Immunol. 1995 May; 2(3):268-71.
Scott P T, Clark J B, Miser W F. Pertussis: an update on primary prevention and outbreak control. Am Fam Physician. 1997 Sep. 15; 56(4):1121-8.
Secrist H, Chelen C J, Wen Y, Marshall J D, Umetsu D T. Allergen immunotherapy decreases interleukin 4 production in CD4+ T cells from allergic individuals. J Exp Med. 1993 Dec. 1; 178(6):2123-30.
Sepp E, Julge K, Vasar M, Naaber P, Bjorksten B, Mikelsaar M. Intestinal microflora of Estonian and Swedish infants. Acta Paediatr. 1997 September; 86(9):956-61.
Shakil A O, Conry-Cantilena C, Alter H J, Hayashi P, Kleiner D E, Tedeschi V, Krawczynski K, Conjeevaram H S, Sallie R, Di Bisceglie A M. Volunteer blood donors with antibody to hepatitis C virus: clinical, biochemical, virologic, and histologic features. The Hepatitis C Study Group. Ann Intern Med. 1995 Sep. 1; 123(5):330-7.
Sheikh A, Strachan D P. The hygiene theory: fact or fiction? Curr Opin Otolaryngol Head Neck Surg. 2004 June; 12(3):232-6.
Shevach E M. Regulatory T cells in autoimmmunity. Annu Rev Immunol. 2000; 18:423-49.
Siber G R, Santosham M, Reid G R, Thompson C, Almeido-Hill J, Morell A, deLange G, Ketcham J K, Callahan E H. Impaired antibody response to Haemophilus influenzae type b polysaccharide and low IgG2 and IgG4 concentrations in Apache children. N Engl J. Med. 1990 Nov. 15; 323(20):1387-92.
Silverman N S, Greif A. Influenza vaccination during pregnancy. Patients' and physicians' attitudes. J Reprod Med. 2001 November; 46(11):989-94.
Singh V K, Mehrotra S, Agarwal S S. The paradigm of Th1 and Th2 cytokines: its relevance to autoimmunity and allergy. Immunol Res. 1999; 20(2):147-61.
Stanberry L R, Cunningham A L, Mindel A, Scott L L, Spruance S L, Aoki F Y, Lacey C J. Prospects for control of herpes simplex virus disease through immunization. Clin Infect Dis. 2000 March; 30(3):549-66.
Stevens C E, Toy P T, Taylor P E, Lee T, Yip H Y. Prospects for control of hepatitis B virus infection: implications of childhood vaccination and long-term protection. Pediatrics. 1992 July; 90(1 Pt 2):170-3.
Strachan D P. Hay fever, hygiene, and household size. BMJ. 1989 Nov. 18; 299(6710):1259-60.
Stumbles P A, Thomas J A, Pimm C L, Lee P T, Venaille T J, Proksch S, Holt P G. Resting respiratory tract dendritic cells preferentially stimulate T helper cell type 2 (Th2) responses and require obligatory cytokine signals for induction of Th1 immunity. J Exp Med. 1998 Dec. 7; 188(11):2019-31.
Sun Q L, Ran W. Review of cytokine profiles in patients with hepatitis. World J Gastroenterol. 2004 Jun. 15; 10(12):1709-15.
Sur D K, Wallis D H, O'Connell T X. Vaccinations in pregnancy. Am Fam Physician. 2003 Jul. 15; 68(2):299-304.
Sutter R W, Pallansch M A, Sawyer L A, Cochi S L, Hadler S C. Defining surrogate serologic tests with respect to predicting protective vaccine efficacy: poliovirus vaccination. Ann N Y Acad. Sci. 1995 May 31; 754:289-99.
Syrbe U, Siveke J, Hamann A. Th1/Th2 subsets: distinct differences in homing and chemokine receptor expression? Springer Semin Immunopathol. 1999; 21(3):263-85.
Tacket C O, Pasetti M F, Sztein M B, Livio S, Levine M M. Immune responses to an oral typhoid vaccine strain that is modified to constitutively express Vi capsular polysaccharide. J Infect Dis. 2004 Aug. 1; 190(3):565-70. Epub 2004 Jun. 30
Thio C L, Thomas D L, Karacki P, Gao X, Marti D, Kaslow R A, Goedert J J, Hilgartner M, Strathdee S A, Duggal P, O'Brien S J, Astemborski J, Carrington M. Comprehensive analysis of class I and class II HLA antigens and chronic hepatitis B virus infection. J. Virol. 2003 November; 77(22):12083-7.
Titball R W, Williamson E D. Vaccination against bubonic and pneumonic plague. Vaccine. 2001 Jul. 20; 19(30):4175-84.
Top F H Jr, Buescher E L, Bancroft W H, Russell P K. Immunization with live types 7 and 4 adenovirus vaccines. II. Antibody response and protective effect against acute respiratory disease due to adenovirus type 7. J Infect Dis. 1971 August; 124(2):155-60.
Tong M, Jacobi C E, van de Rijke F M, Kuijper S, van de Werken S, Lowary T L, Hokke C H, Appelmelk B J, Nagelkerke N J, Tanke H J, van Gijlswijk R P, Veuskens J, Kolk A H, Raap A K. A multiplexed and miniaturized serological tuberculosis assay identifies antigens that discriminate maximally between TB and non-TB sera. J Immunol Methods. 2005 June; 301(1-2):154-63.
Trollfors B, Taranger J, Lagergard T, Lind L, Sundh V, Zackrisson G, Lowe C U, Blackwelder W, Robbins J B. A placebo-controlled trial of a pertussis-toxoid vaccine. N Engl J. Med. 1995 Oct. 19; 333(16):1045-50.
Tugwell B D, Lee L E, Gillette H, Lorber E M, Hedberg K, Cieslak P R. Chickenpox outbreak in a highly vaccinated school population. Pediatrics. 2004 March; 113(3 Pt 1):455-9.
Umetsu D T, DeKruyff R H. TH1 and TH2 CD4+ cells in human allergic diseases. J Allergy Clin Immunol. 1997 July; 100(1):1-6.
Valenti W M, Pincus P H, Messner M K. Nosocomial pertussis: possible spread by a hospital visitor. Am J Dis Child. 1980 May; 134(5):520-1.
Vallejo J G, Baker C J, Edwards M S. Demonstration of circulating group B streptococcal immune complexes in neonates with meningitis. J Clin Microbiol. 1994 September; 32(9):2041-5.
Vandenbark A A, Morgan E, Bartholomew R, Bourdette D, Whitham R, Carlo D, Gold D, Hashim G, Offner H. TCR peptide therapy in human autoimmune diseases. Neurochem Res. 2001 June; 26(6):713-30.
van Deuren M, van der Ven-Jongekrijg J, Vannier E, van Dalen R, Pesman G, Bartelink A K, Dinarello C A, van der Meer J W. The pattern of interleukin-1 beta (IL-1 beta) and its modulating agents IL-1 receptor antagonist and IL-1 soluble receptor type II in acute meningococcal infections. Blood. 1997 Aug. 1; 90(3):1101-8.
Van Huffel, S. and Vandewalle, J. The Total Least Squares Problem Computational Aspects and Analysis. 1991. Society for Industrial and Applied Mathematics.]
Vardinon N, Handsher R, Burke M, Zacut V, Yust I. Poliovirus vaccination responses in HIV-infected patients: correlation with T4 cell counts. J Infect Dis. 1990 July; 162(1):238-41.
Venge P, Bystrom J, Carlson M, Hakansson L, Karawacjzyk M, Peterson C, Seveus L, Trulson A. Eosinophil cationic protein (ECP): molecular and biological properties and the use of ECP as a marker of eosinophil activation in disease. Clin Exp Allergy. 1999 September; 29(9):1172-86.
Venge P. Monitoring the allergic inflammation. Allergy. 2004 January; 59(1):26-32.
Vermont C L, Groot R, Hazelzet J A. Bench-to-bedside review: genetic influences on meningococcal disease. Crit Care. 2002 February; 6(1):60-5. Epub 2001 Nov. 26.
Viglietta V, Baecher-Allan C, Weiner H L, Hafler D A. Loss of functional suppression by CD4+ CD25+regulatory T cells in patients with multiple sclerosis. J Exp Med. 2004 Apr. 5; 199(7):971-9.
Wakeland E K, Liu K, Graham R R, Behrens T W. Delineating the genetic basis of systemic lupus erythematosus. Immunity. 2001 September; 15(3):397-408.
Walboomers J M, Jacobs M V, Manos M M, Bosch F X, Kummer J A, Shah K V, Snijders P J, Peto J, Meijer C J, Munoz N. Human papillomavirus is a necessary cause of invasive cervical cancer worldwide. J Pathol. 1999 September; 189(1):12-9.
Wald A, Zeh J, Selke S, Warren T, Ryncarz A J, Ashley R, Krieger J N, Corey L. Reactivation of genital herpes simplex virus type 2 infection in asymptomatic seropositive persons. N Engl J. Med. 2000 Mar. 23; 342(12):844-50.
Wallis D H, Chin J L, Sur D K. Influenza vaccination in pregnancy: current practices in a suburban community. J Am Board Fam Pract. 2004 July-August; 17(4):287-91.
Ward J, Brenneman G, Letson G W, Heyward W L. Limited efficacy of a Haemophilus influenzae type b conjugate vaccine in Alaska Native infants. The Alaska H. influenzae Vaccine Study Group. N Engl J Med. 1990 Nov. 15; 323(20):1393-401.
Watkins N M, Smith R P Jr, St Germain D L, MacKay D N. Measles (rubeola) infection in a hospital setting. Am J Infect Control. 1987 October; 15(5):201-6.
Watson J C, Hadler S C, Dykewicz C A, Reef S, Phillips L. Measles, mumps, and rubella—vaccine use and strategies for elimination of measles, rubella, and congenital rubella syndrome and control of mumps: recommendations of the Advisory Committee on Immunization Practices (ACIP). MMWR Recomm Rep. 1998 May 22; 47(RR-8):1-57. Whitcher J P, Srinivasan M, Upadhyay M P. Corneal blindness: a global perspective. Bull World Health Organ. 2001; 79(3):214-21.
Whittle H, Jaffar S, Wansbrough M, Mendy M, Dumpis U, Collinson A, Hall A. Observational study of vaccine efficacy 14 years after trial of hepatitis B vaccination in Gambian children. BMJ. 2002 Sep. 14; 325(7364):569.
Wikby A, Ferguson F, Forsey R, Thompson J, Strindhall J, Lofgren S, Nilsson B O, Ernerudh J, Pawelec G, Johansson B. An immune risk phenotype, cognitive impairment, and survival in very late life: impact of allostatic load in Swedish octogenarian and nonagenarian humans. J Gerontol A Biol Sci Med Sci. 2005 May; 60(5):556-65.
Wiktor T J, Gyorgy E, Schlumberger D, Sokol F, Koprowski H. Antigenic properties of rabies virus components. J Immunol. 1973 January; 110(1):269-76.
Williams J N, Jones G R, Christodoulides M, Heckels J E. Serological correlates of protection against meningococci in a cohort of university students, before and during an outbreak of serogroup C infection. J Infect Dis. 2003 May 1; 187(9):1433-41.
Williams P, Wallace D. Unit 731: Japan's Secret Biological Warfare in World War II. 1989. The Free Press, New York, N.Y.
Williamson E D, Eley S M, Stagg A J, Green M, Russell P, Titball R W. A single dose sub-unit vaccine protects against pneumonic plague. Vaccine. 2000 Oct. 15; 19(4-5):566-71.
Wills-Karp M, Santeliz J, Karp C L. The germless theory of allergic disease: revisiting the hygiene hypothesis. Nat Rev Immunol. 2001 October; 1(1):69-75.
Wolf K, Fischer E, Hackstadt T. Ultrastructural analysis of developmental events in Chlamydia pneumoniae-infected cells. Infect Immun. 2000 April; 68(4):2379-85.
Wollenhaupt J, Zeidler H. Undifferentiated arthritis and reactive arthritis. Curr Opin Rheumatol. 1998 July; 10(4):306-13.
Wood D L, Brunell P A. Measles control in the United States: problems of the past and challenges for the future. Clin Microbiol Rev. 1995 April; 8(2):260-7.
Wynn T A. Fibrotic disease and the T(H)1/T(H)2 paradigm. Nat Rev Immunol. 2004 August; 4(8):583-94.
Yang Y, Chung E K, Zhou B, Blanchong C A, Yu C Y, Fust G, Kovacs M, Vatay A, Szalai C, Karadi I, Varga L. Diversity in intrinsic strengths of the human complement system: serum C4 protein concentrations correlate with C4 gene size and polygenic variations, hemolytic activities, and body mass index. J Immunol. 2003 Sep. 1; 171(5):2734-45.
Yang Y, Chung E K, Zhou B, Lhotta K, Hebert L A, Birmingham D J, Rovin B H, Yu C Y. The intricate role of complement component C4 in human systemic lupus erythematosus. Curr Dir Autoimmun. 2004; 7:98-132.
Yang Y, Lhotta K, Chung E K, Eder P, Neumair F, Yu C Y. Complete complement components C4A and C4B deficiencies in human kidney diseases and systemic lupus erythematosus. J Immunol. 2004 Aug. 15; 173(4):2803-14.
Yee C, Thompson J A, Byrd D, Riddell S R, Roche P, Celis E, Greenberg P D. Adoptive T cell therapy using antigen-specific CD8+ T cell clones for the treatment of patients with metastatic melanoma: in vivo persistence, migration, and antitumor effect of transferred T cells. Proc Natl Acad Sci USA. 2002 Dec. 10; 99(25):16168-73.
Yu C Y, Chung E K, Yang Y, Blanchong C A, Jacobsen N, Saxena K, Yang Z, Miller W, Varga L, Fust G. Dancing with complement C4 and the RP-C4-CYP21-TNX (RCCX) modules of the major histocompatibility complex. Prog Nucleic Acid Res Mol Biol. 2003; 75:217-92.
Zackery S, Sutton M, Ciesielski C, Zajackowski M, Santana M, Langley C, Bernard L, Pope V, Fears M, Johnson R, Markowitz L. Rapid diagnostics for syphilis in US clinical settings: a preliminary review of the study data. Presentation at the 2004 STD Prevention Conference. Mar. 8, 2004. Philadelphia, Pa.
Zilinskas R A. Iraq's biological weapons. The past as future? JAMA. 1997 Aug. 6; 278(5):418-24.
Zimmerman R K, Nowalk M P, Bardella I J, Fine M J, Janosky J E, Santibanez T A, Wilson S A, Raymund M. Physician and practice factors related to influenza vaccination among the elderly. Am J Prev Med. 2004 January; 26(1):1-10.

What is claimed:

1. A method for providing selective personalized health care services to one or more individuals in a population of individuals, comprising:
(a) establishing a record of information representative of the immune status of an individual in the population;
(b) analyzing said record according to one or more defined algorithms; and
(c) generating a recommendation to one or more of said individuals based upon said analysis.

2. The method of claim 1, further comprising vaccinating one or more of said individuals in accordance with the recommendation.

3. The method of claim 1, wherein said current information is obtained by administering one or more assay panels to said one or more individuals.

4. The method of claim 1, where said record of information includes at least one of antibody levels to vaccine-preventable diseases, existence of defined genetic factors, existence of defined genetic abnormalities, individual-specific medical information, population-specific medical information, geographic information and demographic information.

5. The method as recited in claim 1, wherein said record is updated from time to time with current information representative of the immune status of said individual.

6. A method of reducing the incidence of vaccine-preventable disease in a population comprising:
(a) establishing records of information representative of the immune status of individuals in the population;
(b) updating said records from time to time with current information representative of the immune status of said individuals;
(c) analyzing the information in an individual's record according to defined standards for vaccinating individuals; and
(d) vaccinating said individual with one or more vaccines in accordance with said analysis.

7. The method of claim 6, wherein said establishing records includes storing the results of one or more assays performed upon the individuals.

8. A method of reducing the costs associated with treating a vaccine-preventable disease in a population, comprising:
(a) establishing records of information representative of the immune status of a plurality of individuals in the population;
(b) updating said records from time to time with current information representative of the immune status of said individuals;
(c) analyzing the information in an individual's record according to defined standards for vaccinating individuals, and thereby generating a recommendation as to whether or not that individual should be vaccinated.

9. A method of generating recommendations for vaccinating an individual in a patient population comprising:
(a) establishing a record of information representative of the immune status of that individual to one or more vaccine-preventable diseases, said record including
(1) current information from one or more assays to determine the immunity of said individual to one or more vaccine-preventable diseases, and
(2) patient-specific information comprising one or more of said patient's medical history, said patient's doctors observations, and/or demographic information relating to said patient; and
(b) processing the information so-collected through one or more algorithms created to determine whether or not to vaccinate said individual; and
thereby generating a recommendation for vaccinating or not vaccinating said individual.

10. A method as recited in claim 9 wherein said record is updated from time to time with current information as recited in (a)(1) and/or (a)(2) and wherein said algorithm is updated from time to time with current standards for determining whether a patient should be vaccinated.

11. A method of optimizing the management of health care for an individual, comprising:
examining insured's immune status;
identifying diseases that the insured is susceptible to and
calculating the risk of contraction for each disease;
indentifying prophylactic therapies that could prevent each identified disease;
calculating, for each disease, expected costs of treatment and costs of associated prophylactic therapies;
requiring prophylactic therapies whose cost is less than expected costs of treatment.

12. The method of claim 11, further comprising requiring prophylactic therapies whose costs are greater than expected treatment costs and assessing additional fees to individual.

13. The method of claim 11, wherein the health care provider is a health insurance company or a health care provider.

14. A method of creating a database useful for generating health care recommendations for an individual in a population comprising:
(a) establishing a record of information representative of the immune status of each individual, said record including
(1) current information from one or more assays to determine the immunity of said individual to one or more antigens and related complements, and
(2) patient-specific information comprising one or more of said patient's medical history, said patients doctors observations, and/or demographic information relating to said patient;
(b) recording updated information as recited in (a)(1) and/or (a)(2) from time to time;
(c) providing one or more algorithms to determine recommended health care therapies based upon defined standards; and
(d) modifying said algorithms from time to time based upon data mining of the database.

15. A method of generating recommendations for vaccinating one or more individuals in a patient population, comprising:
(a) establishing a database comprising a plurality of records of information each representative of the immune status of an individual in the population, to one or more vaccine-preventable diseases, each of said records including
(1) current information from one or more assays to determine the immunity of said individual to one or more vaccine-preventable diseases, and
(2) patient-specific information comprising one or more of said patients medical history, said patients doctors observations and/or demographic information relating to said patient;
(b) processing the information in said database to find trends or patterns relating to the immune status of individuals in said patient population; and
(c) using the said trends or patterns found in (b) in deciding whether or not to vaccinate an individual.

16. A method for generating recommendations for vaccinating one or more individuals in a patient population, comprising:
(a) establishing a database comprising a plurality of records of information each representative of the immune status of an individual in the patient population to one or more vaccine-preventable diseases, each of said records including
(1) current information from one or more assays to determine the immunity of said individual to one or more vaccine-preventable diseases, and
(2) patient-specific information comprising one or more of said patient's medical history, said patients doctors observations and/or demographic information relating to said patient;
(b) providing one or more algorithms to determine whether or not to vaccinate said individuals based upon medical standards;
(c) processing the information in said database to find trends or patterns relating to the immune status of individuals in said patient population and using said trends or patterns to modify said algorithms;
(d) processing the information in a patient's record in said algorithms; and
thereby generating a recommendation as to whether or not that individual should be vaccinated.

17. A method for generating recommendations for vaccinating one or more individuals in a patient population, comprising:
(a) establishing a database comprising a plurality of records of information each representative of the immune status of an individual in the population to one or more vaccine-preventable diseases, each of said records including
(i) current information from one or more assays to determine the immunity of said individual to one or more vaccine-preventable diseases; and
(ii) patient-specific information comprising one or more of said patient's medical history, said patients doctors observations, and/or demographic information relating to said patient; and
(b) processing the information in said database to find trends or patterns relating to the immune status of individuals in said patient population; and
(c) incorporating information comprising said patterns or trends into one or more algorithms created to determine whether or not to vaccinate an individual within said population.

18. A method for generating recommendations for vaccinating one or more individuals in a patient population, comprising:
(a) establishing a database comprising a plurality of records of information each representative of the immune status of an individual in the population, to one or more vaccine-preventable diseases, each of said records including
(1) current information from one or more assays to determine the immunity of said individual to one or more vaccine-preventable diseases, and
(2) patient-specific information comprising one or more of said patients medical history, said patients doctors observations and/or demographic information relating to said patient;
(b) updating said records from time to time with current information as recited in
(a)(1) and/or (a)(2);
(c) providing one or more algorithms to determine whether or not to vaccinate said individual based upon medical standards;
(d) processing the information in said database to find trends or patterns relating to the immune status of individuals in said patient population;
(e) modifying the said algorithms to reflect the patterns and trends found in step (d);
(f) processing the information in a patient's record through said algorithms, and thereby generating a recommendation for vaccinating or not vaccinating said individual.

19. A method for generating recommendations for vaccinating one or more individuals in a patient population, comprising:
(a) establishing a database comprising a plurality of records of information each representative of the immune status of an individual in the population, to one or more vaccine-preventable diseases, each of said records including
(i) current information from one or more assays to determine the immunity of said individual to one or more vaccine-preventable diseases, and
(ii) patient-specific information comprising one or more of said patient's medical history, said patients doctors observations, and/or demographic information relating to said patient;
(b) updating said database from time to time with current information;
(c) providing one or more algorithms to determine whether or not to vaccinate said individuals based upon current medical standards;
(d) processing the information in said database to find trends or patterns relating to the immune status of individuals in said patient population;
(e) incorporating information comprising said patterns or trends into one or more of said algorithms;
(f) processing the information in an individual's record through one or more of said algorithms, and
thereby generating a recommendation for vaccinating said individual.

20. A method for assisting a health care provider in reducing the costs associated with treating vaccine-preventable disease in its patient population by selective vaccination of said patients, comprising the steps of:
(a) establishing records of information representative of the immune status of individuals in the population to one or more vaccine-preventable diseases;
(b) updating said records from time to time with current information representative of the immune status of said individuals;
(c) comparing the information in an individual's record with current standards for vaccinating individuals; and
(d) vaccinating said individual if said individual is in need of vaccination with one or more vaccines.

21. A method of optimizing the management of health care for an individual, comprising:
examining insured's immune status;
identifying diseases that the insured is susceptible to and calculating the risk of contraction for each disease;
indentifying all prophylactic therapies that could prevent each identified disease;
calculating, for all possible combinations of diseases and prophylactic therapies, expected costs of treatment and costs of associated prophylactic therapies;
requiring sets prophylactic therapies optimized for overall cost.
